SEQUENCE LISTING (46 sequences)

SEQ ID NO: 1 - DNA, 469 bp, synthetic construct
  source 1..469: mol_type = other DNA; note = ApoE-AAT promoter
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccc

SEQ ID NO: 2 - DNA, 281 bp, synthetic construct
  source 1..281: mol_type = other DNA; note = hBGi intron
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 3 - DNA, 4425 bp, synthetic construct
  source 1..4425: mol_type = other DNA; note = FVIII-v3 ORF
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 4 - DNA, 382 bp, synthetic construct
  source 1..382: mol_type = other DNA; note = WPRE3
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgg

SEQ ID NO: 5 - DNA, 224 bp, synthetic construct
  source 1..224: mol_type = other DNA; note = bGH polyA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - DNA, 150 bp, synthetic construct
  source 1..150: mol_type = other DNA; note = DTS
gagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 7 - DNA, 449 bp, synthetic construct
  source 1..449: mol_type = other DNA; note = NP Backbone
tggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 8 - protein, 1474 aa, synthetic construct [sequence omitted]

SEQ ID NO: 9 - DNA, 750 bp, synthetic construct
  source 1..750: mol_type = other DNA; note = ApoE-AAT-hbGi
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 10 - DNA, 1389 bp, synthetic construct
  source 1..1389: mol_type = other DNA; note = FIX NA sequence
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatga

SEQ ID NO: 11 - DNA, 126 bp, synthetic construct
  source 1..126: mol_type = other DNA; note = 5 ITR
cctgcaggcagctgcgcgctcgctcgctcactgaggccgcccgggcgtcgggcgacctttggtcgcccggcctcagtgagcgagcgcgcagagagggagtggccaactccatcactaggggttcct

SEQ ID NO: 12 - DNA, 2139 bp, synthetic construct
  source 1..2139: mol_type = other DNA; note = APOE-AAT-hBGi-FIX
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatga

SEQ ID NO: 13 - DNA, 2371 bp, synthetic construct
  source 1..2371: mol_type = other DNA; note = APOE-AAT-hBGi-FIX-bGH-polyA
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgacctcgaggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 14 - DNA, 2918 bp, synthetic construct
  source 1..2918: mol_type = other DNA; note = APOE-AAT-hBGi-FIX WPRE3-bGH-polyA-DTS
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaccctcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 15 - DNA, 3236 bp, synthetic construct
  source 1..3236: mol_type = other DNA; note = ITR-APOE-AAT-hBGi-FIX-WPRE3-bGH-polyA-DTS-ITR
cctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaccctcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcacgtgcggagctgtgcgatccctgctggggactttccgctggggactttccgctggggactttccgccttcagctaaggaagctaccaatatttagaggtacattttgttctagaacaaaatgtaccggtacattttgttctggtacattttgttctatcgatcgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 5955 bp, synthetic construct
  source 1..5955: mol_type = other DNA; note = APOE-AAT-hBGi-FVIII-v3- bGH-polyA
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 17 - DNA, 5955 bp, synthetic construct; identical to SEQ ID NO: 16
  source 1..5955: mol_type = other DNA; note = APOE-AAT-hBGi-FVIII-v3-WPRE3- bGH-polyA-DTS

SEQ ID NO: 18 - DNA, 2918 bp, synthetic construct; identical to SEQ ID NO: 14
  source 1..2918: mol_type = other DNA; note = APOE-AAT-hBGi-FIX-WPRE3- bGH-polyA DTS

SEQ ID NO: 19 - DNA, 5175 bp, synthetic construct
  source 1..5175: mol_type = other DNA; note = APOE-AAT-hbGi-FVIIIv3-
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 20 - DNA, 5955 bp, synthetic construct; identical to SEQ ID NO: 16
  source 1..5955: mol_type = other DNA; note = APOE-AAT-hBGi-FVIIIv3-WPRE3- bGH-polyA DTS

SEQ ID NO: 21 - protein, 461 aa, synthetic construct
  source 1..461: note = FIX AA Sequence
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 22 - DNA, 141 bp, synthetic construct
  source 1..141: mol_type = other DNA; note = 3 ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 449 bp, synthetic construct; identical to SEQ ID NO: 7
  source 1..449: mol_type = other DNA; note = NP Backbone

SEQ ID NO: 24 - protein, 1682 aa, synthetic construct
  source 1..1682: note = FVIII-SQ-N6-F309S-DM
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLTRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNGRAPGNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 25 - protein, 391 aa, synthetic construct
  source 1..391: note = FVIII-SQ-N6-F309S-DM: A1 domain
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIR

SEQ ID NO: 26 - protein, 368 aa, synthetic construct
  source 1..368: note = FVIII-SQ-N6-F309S-DM: A2 domain
SVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPR

SEQ ID NO: 27 - protein, 11 aa, synthetic construct
  source 1..11: note = FVIII-SQ-N6-F309S-DM: SQ domain
SFSQNPPVLTR

SEQ ID NO: 28 - protein, 227 aa, synthetic construct
SFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSST

SEQ ID NO: 29 - protein, 373 aa, synthetic construct
  source 1..373: note = FVIII-SQ-N6-F309S-DM: A3 domain
REITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNGRAPGNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNK

SEQ ID NO: 30 - protein, 153 aa, synthetic construct
CQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNS

SEQ ID NO: 31 - protein, 159 aa, synthetic construct
  source 1..159: note = FVIII-SQ-N6-F309S-DM: C1 domain
CSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 32 - DNA, 5052 bp, synthetic construct
  source 1..5052: mol_type = other DNA; note = FVIII-SQ-N6-F309S-DM: NA Coding Sequence 1
atgcagatcgagctgtctacctgcttcttcctgtgcctgctgcggttctgcttcagcgccaccagaagatattacctgggcgccgtggaactgagctgggactacatgcagtctgacctgggagagctgcccgtggacgctagatttcctccaagagtgcccaagagcttccccttcaacacctccgtggtgtacaagaaaaccctgttcgtggaattcaccgaccacctgttcaatatcgccaagcctcggcctccttggatgggactgctgggacctacaattcaggccgaggtgtacgacaccgtggtcatcaccctgaagaacatggccagccatcctgtgtctctgcacgccgtgggagtgtcttactggaaggcttctgagggcgccgagtacgacgatcagacaagccagagagagaaagaggacgacaaggttttccctggcggcagccacacctatgtctggcaggtcctgaaagaaaacggccctatggcctccgatcctctgtgcctgacatacagctacctgagccacgtggacctggtcaaggacctgaattctggcctgatcggagccctgctcgtgtgtagagaaggcagcctggccaaagagaaaacccagacactgcacaagttcatcctgctgttcgccgtgttcgacgagggcaagagctggcacagcgagacaaagaacagcctgatgcaggacagggatgccgcctctgctagagcttggcctaagatgcacaccgtgaacggctacgtgaacagaagcctgcctggactgatcggctgccacagaaagtccgtgtactggcacgtgatcggcatgggcacaacacctgaggtgcacagcatctttctggaaggacacaccttcctcgtgcggaaccatagacaggccagcctggaaatcagccctatcaccttcctgaccgctcagaccctgctgatggatctgggccagtttctgctgagctgccacatcagctcccaccagcacgatggcatggaagcctacgtgaaggtggacagctgccccgaagaaccccagctgcggatgaagaacaacgaggaagccgaggactacgacgacgacctgaccgactctgagatggacgtcgtcagattcgacgacgataacagccccagcttcatccagatcagaagcgtggccaagaagcaccccaagacctgggtgcactatatcgccgccgaggaagaggactgggattacgctcctctggtgctggcccctgacgacagaagctacaagagccagtacctgaacaacggccctcagcggatcggccggaagtataagaaagtgcggttcatggcctacaccgacgagacattcaagaccagagaggccatccagcacgagagcggaattctgggccctctgctgtatggcgaagtgggcgatacactgctgatcatcttcaagaaccaggccagcagaccctacaacatctaccctcacggcatcaccgatgtgcggcccctgtattctagaaggctgcccaagggcgtgaagcacctgaaggacttccctatcctgcctggcgagatcttcaagtacaagtggaccgtgaccgtggaagatggccccaccaagagcgaccctagatgtctgacacggtactacagcagcttcgtgaacatggaacgcgacctggccagcggcctgattggacctctgctgatctgctacaaagaaagcgtggaccagcggggcaaccagatcatgagcgacaagcggaacgtgatcctgtttagcgtgttcgatgagaaccggtcctggtatctgaccgagaacatccagcggtttctgcccaatcctgctggcgtgcagctggaagatcctgagttccaggcctccaacatcatgcactccatcaatggctatgtgttcgacagcctgcagctgagcgtgtgcctgcacgaagtggcctactggtacatcctgagcattggcgcccagaccgacttcctgtccgtgttctttagcggctacaccttcaagcacaagatggtgtacgaggataccctgacactgttcccattcagcggcgagacagtgttcatgagcatggaaaaccccggcctgtggatcctgggctgtcacaacagcgacttccggaacagaggcatgacagccctgctgaaggtgtccagctgcgacaagaacaccggcgactactacgaggacagctatgaggacatcagcgcctacctgctgagcaagaacaatgccatcgagcccagaagcttctcccaaaatcctccggtcctcacacgttccttcagccagaatagcagacacccctccaccagacagaagcagttcaacgccacaacaatccccgagaacgacatcgagaaaaccgatccttggtttgcccacagaacccctatgcctaagatccagaacgtgtcctccagcgatctgctgatgctcctgagacagagccctacacctcacggactgagcctgtccgatctgcaagaggccaaatacgaaaccttcagcgacgacccttctcctggcgccatcgacagcaacaatagcctgagcgagatgacccacttcagaccacagctgcaccacagcggcgacatggtgtttacacctgagagcggcctccagctgagactgaatgagaagctgggaaccaccgccgccaccgagctgaagaaactggacttcaaggtgtcctctaccagcaacaacctgatcagcacaatcccctccgacaacctggctgccggcaccgacaacacatcttctctgggcccacctagcatgcccgtgcactacgatagccagctggataccacactgttcggcaagaagtctagccctctgacagagtctggcggccctctgtctctgagcgaggaaaacaacgacagcaagctgctggaatccggcctgatgaacagccaagagtcctcctggggcaagaatgtgtccagcaccagagaaatcacccggaccacactgcagagcgaccaagaagagatcgattacgacgataccatcagcgtcgagatgaagaaagaagatttcgacatctacgacgaggacgagaatcagagccctcggagcttccagaagaaaaccaggcactactttattgccgccgtcgagcggctgtgggactacggaatgtctagctctcctcacgtgctgcggaatagagcccagtctggtagcgtgccccagttcaaaaaggtcgtgttccaagagttcaccgacggcagcttcacccagccactgtatagaggcgagctgaacgagcatctgggcctgctgggcccttatatcagagccgaagtggaagataacatcatggtcaccttccggaatcaggcctctcggccctacagcttctacagctccctgatctcctacgaagaggaccagagacagggcgcagagccccggaagaatttcgtgaagcccaacgagactaagacctacttttggaaggtgcagcaccatatggcccctacaaaggacgagttcgactgcaaagcctgggcctacttctccgatgtggaccttgagaaggatgtgcacagcggactcatcggcccactgcttgtgtgccacaccaacacactgaaccccgctcacggcagacaagtgacagtgcaagaattcgccctgtttttcaccatcttcgacgaaacgaagtcctggtacttcaccgaaaacatggaaagaaacggacgcgcgcctggcaacattcagatggaagatcccaccttcaaagagaactaccggttccacgccatcaacggctacatcatggacacactgcccggcctggttatggctcaggatcagagaatccggtggtatctgctgtccatgggctccaacgagaatatccactccatccacttcagcggccacgtgttcaccgtccgcaagaaagaagagtacaagatggccctgtacaatctgtaccccggcgttttcgaaaccgttgagatgctgcctagcaaggccggaatttggagagtggaatgtctgattggagagcacctccacgccgggatgagcaccctgtttctggtgtactccaacaagtgtcagacccctctcggcatggcctctggccacattagagacttccagatcaccgccagcggacagtatggacagtgggcccctaaactggccagactgcactactccggcagcatcaatgcctggtccaccaaagagcctttcagctggatcaaagtggacctgctggctcccatgatcatccacggaatcaagacccagggcgccagacaaaagttcagcagcctgtacatcagccagttcatcatcatgtacagcctggacggaaagaagtggcagacctaccggggcaatagcaccggcacactgatggtgttcttcggcaacgtggactccagcggcattaagcacaacatcttcaaccctccaatcattgcccggtacatccggctgcaccccacacactacagcatcaggtctaccctgagaatggaactgatgggctgcgacctgaacagctgcagcatgcccctcggaatggaaagcaaggccatcagcgacgcccagatcacagcctctagctacttcaccaacatgttcgccacttggagcccctctaaggcccggcttcatctgcaaggcagaagcaacgcttggaggccccaagtgaacaaccccaaagaatggctgcaggtcgactttcagaaaaccatgaaagtgacaggcgtgaccacacagggcgtcaagtccctgctgacctctatgtacgtgaaagagtttctgatcagctccagccaggacggccaccagtggaccctgttcttccagaacggcaaagtgaaagtgttccagggaaatcaggacagcttcacacccgtggtcaactccctggatcctccactgctgacaagatacctgcggattcaccctcagtcttgggtgcaccagattgccctgcggatggaagtgctgggctgtgaagctcaggacctctactgatag

SEQ ID NO: 33 - DNA, 5049 bp, synthetic construct
  source 1..5049: mol_type = other DNA; note = FVIII-SQ-N6-F309S-DM: NA Coding Sequence 2
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatcccccagtgctcacccggtcctttagccagaattctcgccatccctccacccggcaaaagcagttcaacgccactaccatcccagagaacgacatcgaaaaaaccgacccctggttcgcccacagaactcccatgccaaagattcagaatgtttccagttctgacctccttatgttgctgcgccagtctccgacccctcatggactgtccctgagtgacttgcaggaggccaagtacgagacgttctctgatgaccccagcccaggcgcgattgactcaaataactccctgtctgagatgacacattttcgccctcagctccatcacagcggggacatggtgttcactccagagtccggacttcagctgcgcctcaacgagaaactcggtactacagccgcgacagaactcaaaaagctggatttcaaggtttccagcaccagcaataacctgatctctacaattcccagcgataacctggcggctggaaccgacaacacttccagcctgggacctccgtccatgcctgtgcactacgactcccagctcgacaccactctgttcggcaagaaaagtagccccctgaccgaatccggcggtccgctgtccctctccgaagagaataacgatagcaagctcctggaatctgggcttatgaactctcaagagtcatcctggggcaagaacgtttcatcaactagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaacggcagggcccccggcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 34 - protein, 238 aa, synthetic construct
  source 1..238: note = N6-SQ domain
SFSQNPPVLTRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSST

SEQ ID NO: 35 - protein, 1682 aa, synthetic construct
  source 1..1682: note = FVIII (SQ+N6)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLTRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 36 - protein, 391 aa, synthetic construct
  source 1..391: note = FVIII A1
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIR

SEQ ID NO: 37 - protein, 373 aa, synthetic construct
  source 1..373: note = FVIII A3
REITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNK

SEQ ID NO: 38 - DNA, 7342 bp, synthetic construct
  source 1..7342: mol_type = other DNA; note = NP-ITR-ApoE-AAT-hBGi-FVIII-SQ-N6-F309S-DM-WPRE3-DTS -ITR
gaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcacgtgcggagctgtgcgatccctgctggggactttccgctggggactttccgctggggactttccgccttcagctaaggaagctaccaatatttagaggtacattttgttctagaacaaaatgtaccggtacattttgttctggtacattttgttctatcgatcgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattacctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatcccccagtgctcacccggtcctttagccagaattctcgccatccctccacccggcaaaagcagttcaacgccactaccatcccagagaacgacatcgaaaaaaccgacccctggttcgcccacagaactcccatgccaaagattcagaatgtttccagttctgacctccttatgttgctgcgccagtctccgacccctcatggactgtccctgagtgacttgcaggaggccaagtacgagacgttctctgatgaccccagcccaggcgcgattgactcaaataactccctgtctgagatgacacattttcgccctcagctccatcacagcggggacatggtgttcactccagagtccggacttcagctgcgcctcaacgagaaactcggtactacagccgcgacagaactcaaaaagctggatttcaaggtttccagcaccagcaataacctgatctctacaattcccagcgataacctggcggctggaaccgacaacacttccagcctgggacctccgtccatgcctgtgcactacgactcccagctcgacaccactctgttcggcaagaaaagtagccccctgaccgaatccggcggtccgctgtccctctccgaagagaataacgatagcaagctcctggaatctgggcttatgaactctcaagagtcatcctggggcaagaacgtttcatcaactagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaacggcagggcccccggcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgactcga

SEQ ID NO: 39 - DNA, 6181 bp, synthetic construct
  source 1..6181: mol_type = other DNA; note = ApoE-AAT-hBGi-FVIII-N6-F309S-DM-WPRE3-DTS
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgccccgtaagtactagcagctacaatccagctaccattctgcttttattttatggttgggataaggctggattattctgagtccaagctaggcccttttgctaatcatgttcatacctcttatcttcctcccacagatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatcccccagtgctcacccggtcctttagccagaattctcgccatccctccacccggcaaaagcagttcaacgccactaccatcccagagaacgacatcgaaaaaaccgacccctggttcgcccacagaactcccatgccaaagattcagaatgtttccagttctgacctccttatgttgctgcgccagtctccgacccctcatggactgtccctgagtgacttgcaggaggccaagtacgagacgttctctgatgaccccagcccaggcgcgattgactcaaataactccctgtctgagatgacacattttcgccctcagctccatcacagcggggacatggtgttcactccagagtccggacttcagctgcgcctcaacgagaaactcggtactacagccgcgacagaactcaaaaagctggatttcaaggtttccagcaccagcaataacctgatctctacaattcccagcgataacctggcggctggaaccgacaacacttccagcctgggacctccgtccatgcctgtgcactacgactcccagctcgacaccactctgttcggcaagaaaagtagccccctgaccgaatccggcggtccgctgtccctctccgaagagaataacgatagcaagctcctggaatctgggcttatgaactctcaagagtcatcctggggcaagaacgtttcatcaactagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaacggcagggcccccggcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgggagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 40 - DNA, 2363 bp, synthetic construct
  source 1..2363: mol_type = other DNA; note = APOE-AAT-hBGi-FIX -bGH-polyA-DTS
gagctgtgcgatccctgctggggactttccgctggggactttccgctggggactttccgccttcagctaaggaagctaccaatatttagaggtacattttgttctagaacaaaatgtaccggtacattttgttctggtacattttgttcttgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgagtaagtactagcagctacaatccagctaccattctgcttttattttatggttgggataaggctggattattctgagtccaagctaggcccttttgctaatcatgttcatacctcttatcttcctcccacagcgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccc

SEQ ID NO: 41 - DNA, 2595 bp, synthetic construct
  source 1..2595: mol_type = other DNA; note = APOE-AAT-hBGi-FIX WPRE3-bGH-polyA
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgccccgtaagtactagcagctacaatccagctaccattctgcttttattttatggttgggataaggctggattattctgagtccaagctaggcccttttgctaatcatgttcatacctcttatcttcctcccacagatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 42 - DNA, 2472 bp, synthetic construct
  source 1..2472: mol_type = other DNA; note = ITR-APOE-AAT-hBGi-FIX- bGH-polyA-ITR
gctcactcactcactcactgaggcctgcagagcaaagctctgcagtctggggacctttggtccccaggcctcagtgagtgagtgagtgagcagagagggagtggccaactccatcactaggggttcctcgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgccccgtaagtactagcagctacaatccagctaccattctgcttttattttatggttgggataaggctggattattctgagtccaagctaggcccttttgctaatcatgttcatacctcttatcttcctcccacagatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggaacccctagtgatggagttggccactccctctctgctcactcactcactcactgaggcctggggaccaaaggtccccagactgcagagctttgctctgcaggcctcagtgagtgagtgagtgagcaga

SEQ ID NO: 43 - DNA, 5707 bp, synthetic construct
  source 1..5707: mol_type = other DNA; note = ApoE-AAT-hBGi-FVIII-v3-WPRE3-DTS
cgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgggagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 44 - DNA, 5968 bp, synthetic construct
  source 1..5968: mol_type = other DNA; note = ITR-ApoE-AAT-hBGi-FVIII-SQ-N6-F309S-DM-WPRE3-DTS-IT R
cctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgcgtctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtcgactggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatcccccagtgctcacccggtcctttagccagaattctcgccatccctccacccggcaaaagcagttcaacgccactaccatcccagagaacgacatcgaaaaaaccgacccctggttcgcccacagaactcccatgccaaagattcagaatgtttccagttctgacctccttatgttgctgcgccagtctccgacccctcatggactgtccctgagtgacttgcaggaggccaagtacgagacgttctctgatgaccccagcccaggcgcgattgactcaaataactccctgtctgagatgacacattttcgccctcagctccatcacagcggggacatggtgttcactccagagtccggacttcagctgcgcctcaacgagaaactcggtactacagccgcgacagaactcaaaaagctggatttcaaggtttccagcaccagcaataacctgatctctacaattcccagcgataacctggcggctggaaccgacaacacttccagcctgggacctccgtccatgcctgtgcactacgactcccagctcgacaccactctgttcggcaagaaaagtagccccctgaccgaatccggcggtccgctgtccctctccgaagagaataacgatagcaagctcctggaatctgggcttatgaactctcaagagtcatcctggggcaagaacgtttcatcaactagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaacggcagggcccccggcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgactcga

SEQ ID NO: 45 - DNA, 606 bp, synthetic construct
  source 1..606: mol_type = other DNA; note = WPRE3+bGhPolyA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 46 - protein, 1490 aa, synthetic construct
FVIIIVTRANSLATIONMQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNATNVSNNSNTSNDSNVSPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDL

(19) United States
(12) Patent Application Publication
(10) Pub. No.: US 2026/0124227 A1
(43) Pub. Date: May 7, 2026

(54) BIOLOGIC AGENTS AND METHODS OF USE

(71) Applicant: SonoThera, Inc., South San Francisco, CA (US)

(72) Inventors: Ivan KRIVEGA, Burlingame, CA (US); Margarita KRIVEGA, South San Francisco, CA (US); Kenneth GREENBERG, Berkeley, CA (US); Steven B. FEINSTEIN, Highland Park, IL (US)

(21) Appl. No.: 19/353,536

(22) Filed: Oct. 8, 2025

Related U.S. Application Data
(63) Continuation of application No. PCT/US2025/024558, filed on Apr. 14, 2025.
(60) Provisional application No. 63/634,905, filed on Apr. 16, 2024.
(60) Provisional application No. 63/709,966, filed on Oct. 21, 2024.

(51) Int. Cl. A61K 31/7088; A61K 41/00; A61K 47/26; A61K 48/00; C12N 15/11; C12N 15/63

(52) U.S. Cl. CPC A61K 31/7088; A61K 41/0028; A61K 47/26; A61K 48/0058; A61K 48/0083; C12N 15/11; C12N 15/63; C12N 2830/34

(57) ABSTRACT

Disclosed herein are nucleic acid compositions and methods of use. The nucleic acid compositions may have a therapeutic nucleic acid sequence operably linked to a nuclear targeting sequence that increases expression of the therapeutic nucleic acid in a cell by at least 1.25 fold; at least 80% sequence identity to SEQ ID NO: 6; or a first regulatory element comprising a promoter sequence operably linked to a hemoglobin subunit gamma intron (hBGi) sequence, and a second regulatory element comprising a woodchuck hepatitis posttranscriptional regulatory element (WPRE) sequence. The nucleic acid compositions with these features may enhance therapeutic nucleic acid transfection and expression. Also disclosed herein are transgenes optimized for gene therapy applications, including novel FVIII transgene sequences, in which the B domain may be non-naturally occurring the A1 and/or A3 domain may include at least one amino acid substitution.

### CROSS-REFERENCE TO RELATED APPLICATIONS

This application is a continuation of International Application No. PCT/US2025/024558, filed Apr. 14, 2025, which claims the benefit of U.S. Provisional Patent Application No. 63/634,905, filed Apr. 16, 2024, and U.S. Provisional Patent Application No. 63/709,966, filed Oct. 21, 2024, each of which is incorporated herein by reference in its entirety and for all purposes.

### INCORPORATION BY REFERENCE OF SEQUENCE LISTING

The present application is being filed along with a Sequence Listing in electronic format. The Sequence Listing is provided as a file 62668-732301_Sequence_Listing.XML, created Oct. 8, 2025, which is 147,357 bytes in size. The information in the electronic format of the Sequence Listing is incorporated by reference in its entirety.

### BACKGROUND

Gene therapy, in which a functional copy of a gene is transfected or otherwise delivered into a cell, has been proposed as a possible method of treating genetic diseases, however many existing gene therapies suffer from significant shortcomings when used to treat subjects due to failure to achieve or maintain therapeutic levels of transgene expression, which often leaves subjects without an effective treatment due to inability to re-dose many such gene therapies. Vectors for transgene delivery which improve gene transfection, expression, or durability could significantly improve gene therapies and patient outcomes. Similarly, transgenes which provide for improved gene expression or durability, or which otherwise provide for an improved therapeutic effect in a gene therapy treatment, could significantly improve gene therapies and patient outcomes.

### SUMMARY

Vectors for transgene delivery can be improved by the incorporation of regulatory elements which improve gene delivery and transfection. However, the identification of genetic regulatory elements which tend to have a positive therapeutic effect in improving transgene expression remains a challenge. Disclosed herein are nucleic acid compositions comprising genetic regulatory elements operably linked to therapeutic nucleic acid sequences which improve gene transfection and expression. In some cases, the genetic regulatory elements may include novel nuclear targeting sequences which increase nuclear localization of the nucleic acid composition and resulting gene expression. In some cases, the genetic regulatory elements may also include novel combinations of nuclear targeting sequences, promoters, post-transcriptional elements, nuclear targeting sequences, and other genetic regulatory elements, which increase gene transfection and expression.

In addition to improved vectors, transgenes themselves can be modified to improve their transfection and expression in a subject, and can otherwise be modified to provide an improved therapeutic effect to a subject. However, the identification of transgene elements which tend to have a positive therapeutic effect in improving transgene expression or function remains a challenge, especially in cases of very large transgenes where modifications at any point may disrupt gene expression or degrade transgene performance. Aspects disclosed herein provide a Factor VIII polypeptide comprising: an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. Aspects disclosed herein provide a Factor VIII polypeptide comprising an amino acid sequence having up to 3 amino acid substitutions relative to SEQ ID NO: 24. In some embodiments, the FVIII polypeptide disclosed herein may improve over prior FVIII transgenes by providing for increased gene expression, secretion, and coagulation activity of the FVIII polypeptide, among other benefits described herein.

Aspects disclosed herein provide a nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein: the A1 domain comprises substitution of phenylalanine at position 328 to serine when the Factor VIII polypeptide is aligned with SEQ ID NO: 35; and the A3 domain comprises substitution of cysteine at position 1249 to glycine when the Factor VIII polypeptide is aligned with SEQ ID NO: 35 and substitution of cysteine at position 1253 to glycine when the Factor VIII polypeptide is aligned with SEQ ID NO: 35. In some embodiments, the therapeutic nucleic acid sequence encodes a Factor VIII (FVIII) polypeptide comprising an amino acid sequence having up to 2 amino acid substitutions relative to SEQ ID NO: 24. In some embodiments, the B domain comprises no more than 6 consensus sites for N-linked glycosylation. In some embodiments, the B domain comprises an amino acid sequence of SEQ ID NO: 28, and wherein the FVIII polypeptide is not more than 2300 amino acids in length. In some embodiments, the B domain comprises an amino acid sequence of SEQ ID NO: 34. In some embodiments, the A1 domain comprises an amino sequence of SEQ ID NO: 25. In some embodiments, the A3 domain comprises an amino sequence of SEQ ID NO: 29. In some embodiments, the Factor VIII polypeptide comprises an amino acid sequence of SEQ ID NO: 24. In some embodiments, the nucleic acid composition further includes a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence. In some embodiments, the nucleic acid composition further includes a nucleic acid sequence having at least 96% sequence identity to SEQ ID NO: 45. In some embodiments, the nucleic acid composition further includes a nucleic acid sequence having at least 80% sequence identity to SEQ ID NO: 6. In some embodiments, the nucleic acid sequence having at least 80% sequence identity to SEQ ID NO: 6 increases expression of the therapeutic nucleic acid sequence in a cell by at least 1.25-fold as compared to an otherwise identical control composition lacking the nucleic acid sequence having at least 80% sequence identity to SEQ ID NO: 6. In some embodiments, the first regulatory element comprises a sequence of SEQ ID NO: 9. In some embodiments, the second regulatory element comprises a sequence of SEQ ID NO: 45. In some embodiments, the nucleic acid composition comprises a sequence of SEQ ID NO: 39. Aspects disclosed herein provide a nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 34; and wherein: the A1 domain comprises an amino sequence of SEQ ID NO: 25; and the A3 domain comprises an amino sequence of SEQ ID NO: 29, the nucleic acid composition further comprising: a first regulatory element comprising a sequence of SEQ ID NO: 9; a second regulatory element comprising a second regulatory element comprising a sequence of SEQ ID NO: 45; and a nucleic acid sequence of SEQ ID NO: 6. In some embodiments, the nucleic acid composition comprises a sequence of SEQ ID NO: 39.

Aspects disclosed herein provide the nucleic acid composition disclosed herein for use a method of treating a bleeding disorder in a subject in need thereof, the method comprising administering the nucleic acid composition of any one of the preceding claims to the subject. Aspects disclosed herein provide a method of expressing a nucleic acid payload in a cell, the method comprising administering the nucleic acid composition of any one of the preceding claims to a subject. In some embodiments, the method further includes applying an ultrasonic acoustic energy to the cell. In some embodiments, the method further includes administering to the subject a sonoactive agent. In some embodiments, the nucleic acid composition is administered to the subject as a naked nucleic acid. In some embodiments, the nucleic acid composition encodes a full-length polypeptide, wherein the method induces expression of the full-length polypeptide in-vivo. In some embodiments, the method further includes administering to the subject the nucleic acid composition in at least two treatments at least 48 hours apart, thereby achieving a therapeutic level of the clotting factor in the subject. In some embodiments, the method further includes administering to the subject ultrasound acoustic energy in the treatments. In some embodiments, the method further includes administering to the subject a sonoactive agent in the treatments. In some embodiments, one or both of the nucleic acid composition or the sonoactive agent are administered to the subject intravenously through a peripheral vein. In some embodiments, administering the ultrasound acoustic energy comprises applying the ultrasound energy at a mechanical index (MI) of at least 0.8, 1.3, 1.8, 1.9, or 2.2. In some embodiments, administering the ultrasound acoustic energy further comprises applying the ultrasound energy at a mechanical index (MI) of up to 0.4. In some embodiments, the ultrasound energy is applied at an intensity (ISPTA) of at least 200 mW/cm2. In some embodiments, the ultrasound energy is applied at pulse length of at least 20, 200, or 600 microseconds. In some embodiments, the subject is administered at least three treatments. In some embodiments, the at least three treatments are administered at least 48 hours apart. In some embodiments, the subject is administered at least two cycles, each cycle comprising at least two treatments. In some embodiments, the subject is administered at least three cycles, each cycle comprising at least two treatments. In some embodiments, the subject is administered at least three cycles, each cycle comprising at least three treatments. In some embodiments, each cycle is administered to the subject at least 10 days apart. In some embodiments, each cycle is administered to the subject at least 30 days apart. In some embodiments, each cycle is administered to the subject at least 40 days apart. In some embodiments, the method further includes imaging an organ of the subject with ultrasound acoustic energy. In some embodiments, the imaging the organ of the subject comprises verifying presence of the sonoactive agent in the organ or the targeting of the ultrasound acoustic energy towards to organ. In some embodiments, a therapeutic level of the FVIII polypeptide is maintained for at least 10, 20, 30, 40, 50, 60, 70, 80, 90, 100, 110, 120, 130, 140, 145, or 150 days. In some embodiments, vascular flow is not occluded in the subject. In some embodiments, the subject has a bleeding disorder, wherein the therapeutic nucleic acid sequence encodes FVIII, and wherein the method thereby treats the subject having the bleeding disorder. In some embodiments, the bleeding disorder is hemophilia A.

Aspects disclosed herein provide a nucleic acid composition of any one of the preceding claims, for use a method of treating a bleeding disorder in a subject in need thereof, the method comprising administering the nucleic acid composition of any one of the preceding claims to the subject, administering a sonoactive agent to the subject. Aspects disclosed herein provide a cell comprising the nucleic acid composition of any one of the preceding claims. Aspects disclosed herein provide a nucleic acid composition for use a method of treating a bleeding disorder in a subject in need thereof, the nucleic acid composition comprising: a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 34; and wherein: the A1 domain comprises an amino sequence of SEQ ID NO: 25; and the A3 domain comprises an amino sequence of SEQ ID NO: 29, the nucleic acid composition further comprising: a first regulatory element comprising a sequence of SEQ ID NO: 9; a second regulatory element comprising a second regulatory element comprising a sequence of SEQ ID NO: 45; and a nucleic acid sequence of SEQ ID NO: 6 the method comprising: administering to the subject the nucleic acid composition, a sonoactive agent, and ultrasound acoustic energy in a treatment session; administering a cycle comprising at least three treatment sessions to the subject; administering at least three cycles to the subject each cycle at least 10 days apart, thereby achieving a therapeutic level of the FVIII polypeptide in the subject. In some embodiments, the sonoactive agent comprises lipid-stabilized microstructures. In some embodiments, the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble.

Aspects disclosed herein provide a method of manufacturing the nucleic acid composition disclosed herein. Aspects disclosed herein provide a kit comprising: a sonoactive agent; and means for expressing a Factor VIII polypeptide in vivo at a level that is at least 1.5 fold greater than expression of SEQ ID NO. 3. Aspects disclosed herein provide a kit comprising: a sonoactive agent; and the nucleic acid composition disclosed herein. In some embodiments, further included are instructions for administering ultrasound acoustic energy to a subject to facilitate expression of the FVIII polypeptide in vivo. In some embodiments, further included instructions for administering ultrasound acoustic energy to a subject to facilitate delivery of the nucleic acid composition to a subject. In some embodiments, the sonoactive agent comprises lipid-stabilized microstructures. In some embodiments, the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble.

Aspects disclosed herein provide a nucleic acid composition comprising a nucleic acid sequence having at least 80% sequence identity to SEQ ID NO: 6. Aspects disclosed herein provide a nucleic acid composition comprising a nucleic acid sequence having at least 96% sequence identity to SEQ ID NO: 45. Aspects disclosed herein provide a nucleic acid composition a therapeutic nucleic acid sequence, a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence. Aspects disclosed herein provide a cell comprising the nucleic acid compositions disclosed herein. Aspects disclosed herein provide method of manufacturing the nucleic acid compositions disclosed herein. In some embodiments, the nucleic acid composition includes a nucleic acid sequence having at least 80% sequence identity to SEQ ID NO: 6. In some embodiments, the nucleic acid composition a nuclear targeting sequence that increases expression of the therapeutic nucleic acid sequence in a cell by at least 1.25-fold, as compared to an otherwise identical control nucleic acid composition lacking the nuclear targeting sequence. In some embodiments, the nucleic acid composition includes the nuclear targeting sequence comprises a nucleic acid sequence having at least 80% sequence identity to SEQ ID NO: 6. In some embodiments, the nucleic acid composition includes a first regulatory element comprising an APOE-AAT promoter sequence operably linked to a hBGi sequence, and a second regulatory element comprising a WPRE3 sequence. In some embodiments, the nucleic acid composition includes a therapeutic nucleic acid sequence, wherein the nucleic acid sequence having at least 80% sequence identity to SEQ ID NO: 6 increases expression of the therapeutic nucleic acid sequence in a cell by at least 1.25 fold as compared to an otherwise identical control composition lacking the nucleic acid sequence having at least 80% sequence identity to SEQ ID NO: 6. In some embodiments, the nucleic acid composition includes a first regulatory element comprising an APOE-AAT promoter sequence operably linked to a hBGi sequence, and a second regulatory element comprising a WPRE3 sequence. In some embodiments, the first regulatory element is upstream of the therapeutic nucleic acid sequence. In some embodiments, the second regulatory element is downstream of the therapeutic nucleic acid sequence. In some embodiments, the second regulatory element is downstream of the therapeutic nucleic acid sequence. In some embodiments, each of the first regulatory element and the second regulatory element are operably linked to the therapeutic nucleic acid sequence. In some embodiments, the first regulatory element is upstream of the therapeutic nucleic acid sequence, and the second regulatory element is downstream of the therapeutic nucleic acid sequence. In some embodiments, the nucleic acid sequence has at least 80% sequence identity to SEQ ID NO: 6 has at least 81, 82, 83, 84, 85, 86, 87, 88, 89, 90, 91, 92, 93, 94, 95, 96, 97, 98, or 99% sequence identity to SEQ ID NO: 6. In some embodiments, the nucleic acid sequence having at least 80% sequence identity to SEQ ID NO: 6 is operably coupled to a second regulatory element comprising a WPRE3 sequence. In some embodiments, the first regulatory element comprises a sequence of SEQ ID NO: 9. In some embodiments, the second regulatory element comprises a sequence of SEQ ID NO: 4. In some embodiments, the nuclear targeting sequence increases expression of the therapeutic nucleic acid sequence in the cell by at least 1.25, 1.5, 2, 3, 4, 5, 6, 7, or 8 fold, as compared to an otherwise identical control composition that lacks the nuclear targeting sequence. In some embodiments, the cell is a liver cell, a kidney cell, a brain cell, a muscle cell, a cardiac cell, a pancreatic cell, a blood cell, or a tumor cell. In some embodiments, the cell is a hepatocyte. In some embodiments, the therapeutic nucleic acid sequence comprises a sequence encoding a therapeutic transgene. In some embodiments, the therapeutic transgene is a FVIII, FIX, COL4A3, COL4A4, COL4A5, PKD1, or a PKD2. In some embodiments, the therapeutic nucleic acid sequence encodes a CRISPR/Cas system, a Cas protein or homolog or variant thereof, a TALEN, a ZFN, or a template DNA molecule suitable for knock-in via homologous end joining (NHEJ) or homology directed repair (HDR). In some embodiments, the nucleic acid composition comprises a miniplasmid backbone. In some embodiments, the miniplasmid backbone is smaller than 1 kb. In some embodiments, the miniplasmid backbone comprises a sequence of SEQ ID NO: 7. In some embodiments, the therapeutic nucleic acid sequence is a therapeutic agent. In some embodiments, the therapeutic nucleic acid sequence encodes a therapeutic agent. In some embodiments, the therapeutic nucleic acid sequence encodes a protein which provides a therapeutic effect to a subject. In some embodiments, the nucleic acid composition is an isolated nucleic acid molecule. In some embodiments, the nucleic acid composition does not comprise an origin of replication, or a bacterial origin of replication. In some embodiments, the nucleic acid composition further comprises one or more Inverted Terminal Repeat (ITR) sequences. In some embodiments, a first ITR sequence is positioned upstream of the therapeutic nucleic acid sequence, promoter sequence, first regulatory element, and/or nuclear targeting sequence. In some embodiments, a second ITR sequence is positioned downstream of the therapeutic nucleic acid sequence, promoter sequence, first regulatory element, and/or nuclear targeting sequence. In some embodiments, the intron sequence comprises a hemoglobin subunit gamma intron (hBGi) sequence. In some embodiments, a nucleic acid composition further comprises a poly-adenylation signal. In some embodiments, the poly-adenylation signal is positioned downstream of the second regulatory element. In some embodiments, the nucleic acid composition comprises the nucleic acid composition comprises a sequence of SEQ ID NO: 45. In some embodiments, the poly-adenylation signal is positioned downstream of the therapeutic nucleic acid sequence. In some embodiments, the poly-adenylation signal is positioned upstream of the nuclear targeting sequence. In some embodiments, the promoter sequence comprises an Apolipoprotein E-Alpha-1-Antitrypsin (APOE-AAT) promoter sequence. In some embodiments, the first regulatory element is positioned upstream and directly coupled to the therapeutic nucleic acid sequence, the second regulatory element is positioned downstream and directly coupled to the therapeutic nucleic acid sequence, the poly-adenylation signal is positioned downstream and directly coupled to the second regulatory element, and wherein the nuclear targeting sequence is positioned downstream and directly coupled to the poly-adenylation signal. In some embodiments, a nucleic acid composition, such as one disclosed hereincomprises a therapeutic nucleic acid sequence comprising a nucleic acid sequence of SEQ ID NO: 32. In some embodiments, the nucleic acid composition is a non-viral vector. In some embodiments, the nucleic acid composition is at least 5000, 5500, 6000, 6500, or 7000 bp in length, and wherein the non-viral vector encodes a full-length gene. In some embodiments, the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. Aspects disclosed herein provide a nucleic acid composition, wherein the therapeutic nucleic acid sequence encodes a FVIII polypeptide comprising an amino acid sequence having up to 3 amino acid substitutions relative to SEQ ID NO: 24. In some embodiments, the B domain comprises up to 6 consensus sites for N-linked glycosylation. In some embodiments, the B domain comprises an amino acid sequence of SEQ ID NO: 28. In some embodiments, the B domain comprises an amino acid sequence of SEQ ID NO: 34. In some embodiments, the A1 domain comprises substitution of phenylalanine at position 328 to serine when the Factor VIII polypeptide is aligned with SEQ ID NO: 35. In some embodiments, the A1 domain comprises an amino sequence of SEQ ID NO: 25. In some embodiments, the A3 domain comprises substitution of cysteine at position 1249 to glycine when the Factor VIII polypeptide is aligned with SEQ ID NO: 35. In some embodiments, the A3 domain comprises substitution of cysteine at position 1253 to glycine when the Factor VIII polypeptide is aligned with SEQ ID NO: 35. In some embodiments, the A3 domain comprises an amino sequence of SEQ ID NO: 28. In some embodiments, the therapeutic nucleic acid sequence encodes the amino acid sequence of SEQ ID NO: 24.

Aspects disclosed herein provide a method of expressing a nucleic acid payload in a cell, the method comprising administering the nucleic acid composition disclosed herein to a subject. In some embodiments, the method includes applying ultrasonic acoustic energy to the cell. In some embodiments, the method includes administering to the subject a plurality of sonoactive microstructures. In some embodiments, the method includes contacting the cell with a lipid nanoparticle composition. In some embodiments, the nucleic acid composition is administered to the subject in a viral vector. In some embodiments, the method includes to the subject a plurality of magnetic particles and applying a magnetic field to the cell. In some embodiments, the magnetic particles comprise paramagnetic nanoparticles. In some embodiments, the method includes applying an electrical field to the cell. In some embodiments, the nucleic acid composition is administered to the subject using hydrodynamic injection. In some embodiments, the nucleic acid composition is administered to the subject as a naked nucleic acid. In some embodiments, the nucleic acid composition is administered to the subject as an encapsulated nucleic acid. In some embodiments, the nucleic acid composition is encapsulated in a lipid or a polymer. In some embodiments, the nucleic acid composition encodes a full-length polypeptide, wherein the method induces expression of the full-length polypeptide in-vivo. In some embodiments, the subject has a bleeding disorder, wherein the therapeutic nucleic acid sequence of the polypeptide encodes FVIII or FIX, and wherein the method thereby treats the subject having the bleeding disorder. In some embodiments, the bleeding disorder is hemophilia A.

Aspects disclosed herein provide a Factor VIII polypeptide comprising: an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. A Factor VIII polypeptide comprising an amino acid sequence having up to 3 amino acid substitutions relative to SEQ ID NO: 24. In some embodiments, the FVIII polypeptide has an amino acid sequence having up to 3 amino acid substitutions relative to SEQ ID NO: 24. In some embodiments, the FVIII polypeptide has an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 35; and the A3 domain comprises at least two amino acid substitutions as compared to SEQ ID NO: 36. In some embodiments, the B domain comprises up to 6 consensus sites for N-linked glycosylation. In some embodiments, the B domain comprises an amino acid sequence of SEQ ID NO: 28. In some embodiments, the B domain comprises an amino acid sequence of SEQ ID NO: 34. In some embodiments, the A1 domain comprises substitution of phenylalanine at position 328 to seine when the FVIII polypeptide is aligned with SEQ ID NO: 35. In some embodiments, the A1 domain comprises an amino sequence of SEQ ID NO: 25. In some embodiments, the A3 domain comprises substitution of cysteine at position 1249 to glycine when FVIII polypeptide is aligned with SEQ ID NO: 35. In some embodiments, the A3 domain comprises substitution of cysteine at position 1253 to glycine when FVIII polypeptide is aligned with SEQ ID NO: 35. In some embodiments, the A3 domain comprises an amino sequence of SEQ ID NO: 28. In some embodiments, a FVIII polypeptide disclosed herein comprises the amino acid sequence of SEQ ID NO: 24. Aspects disclosed herein provide a kit comprising: a sonoactive agent; and means for expressing a Factor VIII polypeptide in vivo at a level that is at least 1.5 fold greater than expression of SEQ ID NO. 3. Aspects disclosed herein provide a kit comprising: a sonoactive agent; and the nucleic acid composition of any embodiment described herein. Aspects disclosed herein provide a cell comprising the FVIII polypeptide disclosed herein. Aspects disclosed herein provide a nucleic acid encoding the FVIII polypeptide.

Aspects disclosed herein provide a method of treating a bleeding disorder comprising administering to a subject a nucleic acid encoding a human clotting factor in at least two treatments at least 48 hours apart, thereby achieving a therapeutic level of the clotting factor in the subject. In some embodiments, the therapeutic level of the clotting factor is maintained for at least 10, 20, 30, 40, 50, 60, 70, 80, 90, 100, 110, 120, 130, 140, 145, or 150 days. In some embodiments, the method includes administering to the subject ultrasound acoustic energy in the treatments. In some embodiments, the method includes administering to the subject a sonoactive agent in the treatments. In some embodiments, administering the ultrasound acoustic energy comprises applying the ultrasound energy at a mechanical index (MI) of at least 0.8, 1.3, 1.8, 1.9, or 2.2. In some embodiments, administering the ultrasound acoustic energy comprises applying the ultrasound energy at a mechanical index (MI) of up to 0.4. In some embodiments, administering the ultrasound energy comprises applying an acoustic radiation force to the subject. In some embodiments, the ultrasound energy is applied at an intensity (ISPTA) of at least 200 mW/cm2. In some embodiments, the ultrasound energy is applied at pulse length of at least 20 microseconds. In some embodiments, the subject is administered at least three treatments. In some embodiments, the sonoactive agent comprises protein-stabilized microstructures. In some embodiments, the sonoactive agent comprises a shell filled with a perfluorinated gas. In some embodiments, the sonoactive agent comprises lipid-stabilized microstructures. In some embodiments, the at least three treatments are administered at least 48 hours apart. In some embodiments, the subject is administered at least two cycles, each cycle comprising at least two treatments. In some embodiments, the subject is administered at least three cycles, each cycle comprising at least two treatments. In some embodiments, the subject is administered at least three cycles, each cycle comprising at least three treatments. In some embodiments, each cycle is administered to the subject at least 10 days apart. In some embodiments, each cycle is administered to the subject at least 30 days apart. In some embodiments, each cycle is administered to the subject at least 40 days apart.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates vector elements of nucleic acid compositions comprising therapeutic nucleic acid vectors comprising genetic regulatory elements which increase expression of the therapeutic nucleic acid;

FIG. 1B provides data illustrating improved expression of the therapeutic nucleic acid in vectors in-vitro;

FIG. 1C provides data illustrating improved expression of the therapeutic nucleic acid in vectors comprising improved genetic regulatory elements in-vitro;

FIG. 1D provides data illustrating improved expression of the therapeutic nucleic acid in vectors in-vitro;

FIG. 2A illustrates vector elements of nucleic acid compositions comprising a therapeutic nucleic acid in vectors comprising genetic regulatory elements which increase expression of the therapeutic nucleic acid;

FIG. 2B provides data illustrating improved expression of the therapeutic nucleic acid in the vectors comprising improved genetic regulatory elements in-vivo;

FIG. 2C provides data illustrating improved expression of the therapeutic nucleic acid in the vectors comprising improved genetic regulatory elements in-vivo;

FIG. 2D provides data illustrating improved expression of the therapeutic nucleic acid in the vectors comprising improved genetic regulatory elements in-vivo;

FIG. 3 provides data illustrating improved expression of the therapeutic nucleic acid in vectors comprising improved genetic regulatory elements in-vivo;

FIG. 4A provides data illustrating improved expression of a therapeutic nucleic acid comprising a modified transgene in a vector comprising improved genetic regulatory elements in-vitro;

FIG. 4B provides data illustrating improved expression of a therapeutic nucleic acid comprising a modified transgene in a vector comprising improved genetic regulatory elements in-vitro;

FIG. 5 provides data illustrating improved expression of a therapeutic nucleic acid comprising a modified transgene in a vector comprising improved genetic regulatory elements in-vivo;

FIG. 6 provides data illustrating improved expression of a therapeutic nucleic acid comprising a modified transgene in a vector comprising improved genetic regulatory elements in-vivo;

FIG. 7 provides data illustrating improved expression of a therapeutic nucleic acid comprising a modified transgene in a vector comprising improved genetic regulatory elements in-vivo;

FIG. 8 illustrates a modified FVIII transgene disclosed herein;

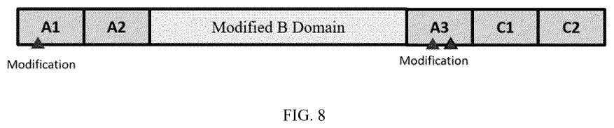

FIG. 9A provides data illustrating improved expression of a modified FVIII polypeptide and its clotting activity measured by chromogenic assay;

FIG. 9B provides data illustrating improved expression of a modified FVIII polypeptide and its clotting activity measured by chromogenic assay;

FIG. 9C provides data illustrating improved expression of a modified FVIII polypeptide and its clotting activity measured by chromogenic assay; and

FIG. 10 provides data illustrating improved expression of a modified FVIII polypeptide delivered in a vector comprising improved genetic regulatory elements in-vivo.

### DETAILED DESCRIPTION

Vectors for transgene delivery can be improved by the incorporation of regulatory elements which improve nucleic acid delivery and transfection. However, the identification of genetic regulatory elements which tend to have a positive therapeutic effect in improving transgene expression remains a challenge within the art. Disclosed herein are nucleic acid compositions comprising genetic regulatory elements operably linked to therapeutic nucleic acid sequences which improve nucleic acid delivery and gene expression. In some cases, the genetic regulatory elements may include novel nuclear targeting sequences which increase nuclear localization of the nucleic acid composition and resulting gene expression, and novel combinations of promoters, post-transcriptional elements, nuclear targeting sequences, and other genetic regulatory elements which increase nucleic acid transfection and expression.

In addition to improved vectors, transgenes themselves can be modified to improve their transfection and expression in a subject, and can otherwise be modified to provide an improved therapeutic effect to a subject. However, the identification of transgene elements which tend to have a positive therapeutic effect in improving transgene expression or function remains a challenge, especially in cases of very large transgenes where modifications at any point may disrupt gene expression or degrade transgene performance. Aspects disclosed herein provide a Factor VIII polypeptide comprising: an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. Aspects disclosed herein provide a Factor VIII polypeptide comprising an amino acid sequence having up to 3 amino acid substitutions relative to SEQ ID NO: 24. Aspects disclosed herein provide a nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 34; and wherein: the A1 domain comprises an amino sequence of SEQ ID NO: 25; and the A3 domain comprises an amino sequence of SEQ ID NO: 29, the nucleic acid composition further comprising: a first regulatory element comprising a sequence of SEQ ID NO: 9; a second regulatory element comprising a second regulatory element comprising a sequence of SEQ ID NO: 45; and a nucleic acid sequence of SEQ ID NO: 6. In some embodiments, the nucleic acid composition comprises a sequence of SEQ ID NO: 39. In some embodiments, the FVIII polypeptide disclosed herein may improve over prior FVIII transgenes by providing for increased gene expression, secretion, and coagulation activity of the FVIII polypeptide, among other benefits described herein.

The construction of nucleic acid delivery vectors for transgene delivery involves the incorporation of various genetic regulatory elements to ensure efficient and controlled gene expression. These elements play important roles in modulating the delivery, transfection, expression, and stability of the transgene. Genetic regulatory elements included in the nucleic acid compositions of the presently disclose may include promoters, enhancers, polyadenylation signals, terminator sequences, transcriptional regulatory elements, nuclear targeting sequences, and viral sequences. By carefully selecting and combining genetic regulatory elements, nucleic acid delivery vectors that increase transgene delivery, transfection, and expression to can be designed, as compared to nucleic acid delivery vectors which do not comprise one or more of the selected genetic regulatory elements.

The delivery vectors disclosed herein may comprise DNA nuclear targeting sequences (DTS) that enhance delivery of the nucleic acid composition and/or the therapeutic nucleic acid sequence to a cell. In some cases, the DTS sequences may comprise recognition sequences for endogenous DNA-binding proteins which lead to increased transfection efficiency of non-viral gene delivery by virtue of enhanced nuclear import of the nucleic acid composition, which can lead to enhanced transgene expression. In some cases, the DTS sequences are recognized by nuclear transport proteins, such as importins, bind the DTS sequences and form an import complex which shields the delivery vector from cytoplasmic nucleases during movement toward the nuclear envelope, and facilitate translocation of the delivery vector through the nuclear pore complex. In some cases, the enhanced nuclear import of the nucleic acid composition avoids degradation of the nucleic acid composition in the cytoplasm, brings nucleic acid composition closer in proximity to the transcriptional machinery, thereby promoting efficient transcription of the delivered transgene. In some cases, the DTS sequence minimizes the loss of transgene during cell division, and increases the likelihood of stable inheritance of the transgene by one or more daughter cells. Various DNA nuclear targeting sequences can be operably linked with nucleic acid sequences comprising the transgene of interest in the delivery vectors disclosed herein. In some cases, the nucleic acid composition may comprise a DNA nuclear targeting sequence having at least about 90%, at least about 95%, at least about 96%, at least about 97%, at least about 98%, at least about 99%, or more, sequence identity to SEQ ID NO: 6. In some cases, the nucleic acid composition comprises a DNA nuclear targeting sequence having 100% sequence identity to SEQ ID NO: 6. In some cases, the DNA nuclear targeting sequence may be downstream or 3′ of the therapeutic nucleic acid sequence. The identification and selection of nuclear targeting sequences (DTS) that enhance delivery of a nucleic acid composition to a cell remains a challenge, as not all nuclear targeting sequences have been shown to be effective in enhancing delivery of a nucleic acid composition to a cell. See V. B. van Gaal et al., DNA Nuclear Targeting Sequences for Non-Viral Gene Delivery, 28 PHARM RES 1707-22 (2011); Prasad and Rao, The Role ofPlasmid Constructs Containing the Sv40 DNA Nuclear-Targeting Sequence in Cationic Lipid-Mediated DNA Delivery, 10 CELLULAR & MOLECULAR BIOLOGY LETTERS 203-15 (2005).

Aspects disclosed herein provide a nucleic acid composition comprising a therapeutic nucleic acid sequence operably linked to a nuclear targeting sequence that increases expression of the therapeutic nucleic acid in a cell by at least 1.25-fold as compared to an otherwise identical control composition that lacks the nuclear targeting sequence. In some embodiments, the nuclear targeting sequence increases expression of the therapeutic nucleic acid sequence in the cell by at least 1.25, 1.5, 2, 3, 4, 5, 6, 7, or 8-fold, as compared to an otherwise identical control composition that lacks the nuclear targeting sequence. Aspects disclosed herein provide a nucleic acid composition comprising a nucleic acid sequence having at least 80% sequence identity to SEQ ID NO: 6. Aspects disclosed herein provide a nucleic acid composition comprising a nucleic acid sequence having at least 96% sequence identity to SEQ ID NO: 6.

Various promoters can be operably linked with a nucleic acid comprising the transgene of interest in the delivery vectors disclosed herein. In some embodiments, the promoter can drive the expression of the protein of interest in a cell. The promoter can be naturally occurring or non-naturally occurring. In some embodiments the promoter is a synthetic promoter. In one embodiment the synthetic promoter comprises sequences that do not exist in nature and which are designed to regulate the activity of an operably linked gene. In another embodiment the synthetic promoter comprises fragments of natural promoters to form new stretches of DNA sequence that do not exist in nature. Synthetic promoters are typically comprised of regulatory elements, promoters, enhancers, introns, splice donors and acceptors that are designed to produce enhanced tissue specific expression. Examples of promoters include, but are not limited to, viral promoters, plant promoters and mammalian promoters. Examples of promoters contemplated herein include, but are not limited to, e.g., CMV promoter, UbC promoter, CAG promoter, EF-1a promoter, ApoE promoter, ApoE-AAT1 promoter, F8 promoter, 3XSERP promoter, or P3-hybrid promoter. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising ApoE-AAT. In some embodiments, the nucleic acid construct comprises a promoter sequence comprising SERP. In some embodiments, the nucleic acid construct comprises a promoter sequence comprising P3. In another embodiment the promoter is a liver specific promoter. Examples of liver specific promoters include LP1, HLP, HCR-AAT, ApoE-AAT, LSP, TBG and TTR.

In some embodiments, the promoter comprises the human alpha anti-trypsin (AAT) promoter complex. In some embodiments, the promoter comprises at least a portion of the AAT promoter. In some embodiments, the promoter comprises a liver-specific apolipoprotein E (ApoE) enhancer. In some embodiments, the promoter comprises at least a portion of the ApoE enhancer. In some embodiments, the promoter comprises a liver specific ApoE enhancer coupled to an AAT promoter. The portion of the ApoE-AAT promoter can comprise a nucleic acid sequence having at least about 90%, at least about 95%, at least about 96%, at least about 97%, at least about 98%, at least about 99%, or more (e.g., 100%), sequence identity to SEQ ID NO: 1. In some cases, ApoE-AAT promoter sequence may be 5′ (or upstream) of the therapeutic nucleic acid sequence. In some embodiments, the nucleic acid composition includes a first regulatory element comprising an APOE-AAT promoter sequence operably linked to a hBGi sequence. In some embodiments, the promoter comprises an F8 promoter sequence. In some embodiments, the intron sequence comprises a hemoglobin subunit gamma intron (hBGi) sequence.

In some embodiments, the nucleic acid delivery vectors disclosed herein comprise one or more polyadenylation signals. Polyadenylation signals can be utilized in nucleic acid delivery vectors to enhance mRNA stability and ensure proper termination of transcription. For example, the polyadenylation signal (PolyA) can mark an end of the mRNA coding sequence and signals the RNA polymerase to stop synthesizing the RNA transcript, allowing for production of a functional mRNA molecule encoding the transgene of interest, thereby enhancing transgene expression. In some cases, a poly(A) tail can contribute to mRNA stability, protecting it from enzymatic degradation, and prolonging mRNA half-life within the cell. In some cases, a poly(A) signal can increase nuclear export of mRNA, for example, by forming a polyadenylation binding protein complex (PAC) with poly(A)-binding proteins (PABPs), linkage to nuclear RNA export factor 1 (NXF1) and cofactor p15, interacting with NXF1 (aka Tap, mRNA export factor) marking the transcribed mRNA for export through the nuclear pore complex, thereby increasing cytoplasmic concentration of mRNA encoding the transgene. In some cases, the poly(A) signal is a bovine growth hormone polyadenylation signal (BGH polyA) and has robust polyadenylation activity leading to consistent addition of a poly(A) tail to the 3′ end of the mRNA transcript, promoting mRNA stability and efficient translation as described herein. In some cases, the BGH polyA regulatory element is active in providing polyadenylation activity across different cell types and species, making it suitable for use in a variety of expression systems, facilitating consistent and reliable transgene expression in different cellular environments. In some cases, the BGH polyA regulatory element provides increased polyadenylation activity across a wider variety of surrounding nucleic acid sequences coupled to the BGH polyA regulatory element, permitting for its incorporation into a wider variety of delivery vectors with greater variations in upstream and downstream sequences. In some cases, BGH polyA regulatory element may be 3′ or downstream of the therapeutic nucleic acid sequence. In some embodiments, the nucleic acid composition further comprises a poly-adenylation signal. In some embodiments, the poly-adenylation signal is positioned downstream of the second regulatory element. In some embodiments, the poly-adenylation signal is positioned downstream of the therapeutic nucleic acid sequence. In some embodiments, the poly-adenylation signal is positioned upstream of the nuclear targeting sequence.

In some embodiments, the nucleic acid delivery vectors disclosed herein comprise one or more intron sequences. In some cases, inclusion of an intron element may enhance expression compared with expression in the absence of the intron element. For example, introns may facilitate processing of the RNA transcript in mammalian host cells, promote the recruitment of RNA polymerase II and other transcription factors, leading to increased transcription of the transgene, and increased expression of the protein of interest. In some cases, the inclusion of introns may contribute to the stability of mRNA transcripts, protect the mRNA from degradation and enhance its half-life within the cell, leading to prolonged and more sustained transgene expression. In some cases, the inclusion of introns can increase export of transcribed mRNA to the cytoplasm and increase transgene expression.

Non-limiting examples of such an intron are promoters of the fetal globin genes introns (hBGi), β-globin intron, A1AT intron and/or hPAH intron. In some embodiments, the intron is a synthetic intron. In some cases, the intron is a fetal globin gene intron (hBGi). In some cases, the fetal globin genes intron (hBGi) can protect the mRNA from degradation and enhance its half-life within the cell, leading to prolonged and more sustained transgene expression, increase export of transcribed mRNA to the cytoplasm, and increase transgene expression, among other benefits. In some cases, the intron can include a nucleotide sequence having at least about 90%, at least about 95%, at least about 96%, at least about 97%, at least about 98%, at least about 99%, or more (e.g., 100%), sequence identity to SEQ ID NO: 2. The location and size of the intron in the vector can vary. In some embodiments, the intron is located between the promoter and the sequence encoding the therapeutic nucleic acid sequence. In some embodiments, the intron is located 5′ of the therapeutic nucleic acid sequence and 3′ of the promoter sequence. In some embodiments, the nucleic acid composition includes a first regulatory element comprising an APOE-AAT promoter sequence operably linked to a hBGi sequence.

In some embodiments, the nucleic acid delivery vectors disclosed herein comprise a Woodchuck Hepatitis Virus Posttranscriptional Regulatory Element 3 (WPRE3). In some cases, the WPRE3 element improves ribosomal recruitment to a transcribed mRNA, enhances translation initiation of a transcribed mRNA, and promotes the expression of the therapeutic nucleic acid sequence. In some cases, the WPRE3 element contains sequences which interacts with cellular translation initiation factors, such as eIF4E (eukaryotic translation initiation factor 4E) and eIF4G (eukaryotic translation initiation factor 4G) and enhances the assembly of the translation initiation complex, facilitating the recruitment of ribosomes to the mRNA. In some cases, the WPRE3 element promotes efficient scanning of ribosomes along the mRNA, increasing initiation of translation at the correct site, and promoting the expression of the therapeutic nucleic acid sequence in the cell. In some cases, the translated WPRE3 element contains sequences that are recognized by ribosomal RNA or elements that stabilize the interaction between ribosomal subunits and the mRNA. In some cases, the translated WPRE3 element can reduce secondary mRNA structures such as hairpins and stem-loops, which can hinder ribosomal recruitment and reduce the efficiency of translation. In some cases, the translated WPRE3 element interacts with ribosomal proteins, promoting their binding to the mRNA, and increasing the stability of association between ribosomes and the mRNA. In some cases, the WPRE3 element can include a nucleotide sequence having at least about 90%, at least about 95%, at least about 96%, at least about 97%, at least about 98%, at least about 99%, or more (e.g., 100%), sequence identity to SEQ ID NO: 4. In some embodiments, the WPRE3 element is located 3′ of the therapeutic nucleic acid sequence. In some embodiments, the nucleic acid composition includes a second regulatory element comprising a WPRE3 sequence.

In some embodiments, the nucleic acid delivery vectors disclosed herein comprise one or more viral sequences. In some cases, the one or more viral sequences may be adeno-associated viruses (AAVs), Inverted Terminal Repeat (ITR) sequences. In some cases, the inclusion of one or more viral sequences may provide for episomal persistence by facilitating circularization of the vector genome within the nucleus of the cell, and can contribute to durable transgene expression without genomic integration. In some cases, the inclusion of one or more viral sequences may facilitate the production of additional vector copies within the host cell and increase expression of the transgene within the cell. In some cases, the inclusion of one or more viral sequences may enhance the delivery, transfection, or expression of a therapeutic nucleic acid sequence by increasing stability and preventing degradation of the nucleic acid delivery vector in vivo, and increasing the delivery, transfection, and/or expression of the therapeutic nucleic acid sequence in the cell. In some cases, the inclusion of one or more viral sequences may enhance delivery of the therapeutic nucleic acid to a host cell in the liver. The inclusion of one or more viral sequences, such as ITR sequences, in the nucleic acid delivery vectors of the present disclosure can enhance the efficacy, safety, and stability of the nucleic acid delivery vectors disclosed herein. In some embodiments, the delivery vector comprises a plurality of ITR sequences located 5′ of the therapeutic nucleic acid sequence and 3′ of the promoter sequence. In some embodiments, the delivery vector comprises a plurality of ITR sequences located at the beginning of the nucleic acid delivery vector, and the end of the nucleic acid delivery vector.

In some embodiments, the nucleic acid composition further comprises one or more Inverted Terminal Repeat (ITR) sequences. In some embodiments, a first ITR sequence is positioned upstream of the therapeutic nucleic acid sequence, promoter sequence, first regulatory element, and/or nuclear targeting sequence. In some embodiments, a second ITR sequence is positioned downstream of the therapeutic nucleic acid sequence, promoter sequence, first regulatory element, and/or nuclear targeting sequence.

The delivery vectors may comprise one or more transgenes. The transgenes may be selected for inducing expression of a therapeutic nucleic acid or therapeutic protein in a cell. Inducing expression of a therapeutic nucleic acid or therapeutic protein may treat one or more genetic diseases in a subject. The transgene is a nucleic acid sequence, heterologous to the vector sequences flanking the transgene, which encodes a polypeptide, protein, or other product of interest. The transgene nucleic acid coding sequence may be operatively linked to regulatory components in a manner which facilitates or enhances transgene delivery, transfection, transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding, green fluorescent protein (GFP), luciferase, β-lactamase, β-galactosidase (LacZ), alkaline phosphatase, thymidine kinase, membrane bound proteins including, for example, CD2, CD4, CD8, fusion proteins comprising a membrane bound protein appropriately fused to an antigen tag domain from, among others, hemagglutinin or Myc. The reporter sequences when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. Where the transgene is green fluorescent protein or luciferase, the vector carrying the signal may be measured visually by color or light production in a luminometer. Where the transgene is green fluorescent protein or luciferase, expression of the transgene may be measured visually by color or light production in a luminometer. Therapeutic delivery vectors may comprise transgenes encoding a therapeutic nucleic acid or therapeutic protein used to correct or ameliorate gene deficiencies, which may include deficiencies in which normal genes are expressed at less than normal levels or deficiencies in which the functional gene product is not expressed. For example, a therapeutic protein or polypeptide which is expressed in a host cell, and, in some cases, secreted from host cell. Suitable transgenes may be readily selected, and selection of the transgene is not considered to be a limitation of the present disclosure. In some embodiments, the transgene is a heterologous protein, and this heterologous protein is a therapeutic protein. Exemplary therapeutic proteins include, but are not limited to, FVIII and FIX. In some embodiments, the therapeutic transgene is a FVIII, FIX, COL4A3, COL4A4, COL4A5, PKD1, or a PKD2. In some embodiments, the therapeutic nucleic acid sequence includes a sequence encoding a therapeutic transgene.

In some embodiments, the therapeutic nucleic acid sequence is a therapeutic cargo other than a transgene. In some embodiments, the therapeutic nucleic acid sequence encodes a CRISPR/Cas system, a Cas protein or homolog or variant thereof, a TALEN, a ZFN, or a template DNA molecule suitable for knock-in via homologous end joining (NHEJ) or homology directed repair (HDR).

The construction of nucleic acid delivery vectors for transgene delivery involves the incorporation of various genetic regulatory elements to ensure efficient and controlled gene expression. These elements play important roles in modulating the delivery, transfection, expression, and stability of the transgene. Genetic regulatory elements included in the nucleic acid compositions of the presently disclosure may include promoters, enhancers, polyadenylation signals, terminator sequences, transcriptional regulatory elements, nuclear targeting sequences, and viral sequences. By carefully selecting and combining genetic regulatory elements, nucleic acid delivery vectors that increase transgene delivery, transfection, and expression to can be designed, as compared to nucleic acid delivery vectors which do not comprise the selected genetic regulatory elements. Further, in addition to the identification of novel sequences of genetic regulatory elements, and the selection of genetic regulatory elements, the combination and arrangement of genetic regulatory elements in a delivery vector can also provide a beneficial technical effect in increasing transgene delivery, transfection, and expression in a host cell.

In some cases, the nucleic acid delivery vectors of the present disclosure may comprise one or more genetic regulatory elements including promoters, enhancers, polyadenylation signals, terminator sequences, transcriptional regulatory elements, nuclear targeting sequences, and viral sequences. In some cases, the nucleic acid delivery vectors or the present disclosure may comprise a promoter sequence operatively linked to a transgene sequence. In some cases, the nucleic acid delivery vectors or the present disclosure may comprise a polyadenylation sequence operatively linked to a transgene sequence. In some cases, the nucleic acid delivery vectors of the present disclosure may comprise an intron sequence operatively linked to a transgene sequence. In some cases, the nucleic acid delivery of vectors of the present disclosure may comprise a post transcriptional regulatory element operatively linked to the transgene sequence. In some cases, the nucleic acid delivery vectors of the present disclosure may comprise a nuclear targeting sequence operatively linked to a transgene sequence. In some cases, the nucleic acid delivery vectors of the present disclosure may comprise one or more viral sequences operatively linked to a transgene sequence.

In some cases, the nucleic acid delivery vectors of the present disclosure may comprise a nuclear targeting sequence positioned downstream of the transgene sequence. In some cases, the nucleic acid delivery vectors of the present disclosure may comprise a nuclear targeting sequence positioned downstream of the transgene and other regulatory elements, for example, a post transcriptional regulatory element, a polyadenylation signal, and/or other regulatory element. In some cases, the nucleic acid delivery vectors may comprise promoter sequences positioned upstream of the transgene sequence. In some embodiments, the nucleic acid delivery vectors may comprise an intron sequence positioned in between a promoter sequence and a transgene sequence. In some cases, the nucleic acid delivery vectors may comprise a post transcriptional regulatory element positioned downstream of a transgene sequence. In some cases, the nucleic acid delivery vectors may comprise polyadenylation sequence positioned downstream of a transgene sequence. In some cases, the nucleic acid delivery vectors may comprise a nuclear targeting sequence positioned downstream of a transgene sequence. In some cases, the nucleic acid delivery vector may comprise one or more viral sequences positioned upstream or downstream of the transgene sequence. In some cases, the nucleic acid delivery vectors may comprise two or more viral sequences, positioned both upstream and downstream of the transgene sequence. In some cases, the nucleic acid delivery of vectors comprise a viral sequence positioned at the beginning and the end of the nucleic acid delivery vector. In some cases, the viral sequences comprise ITR sequences.

In some cases, the nucleic acid delivery vectors comprise a hBGi element positioned in between a promoter sequence and a transgene sequence. In some cases, the hBGi element is operatively linked to the transgene sequence and to the promoter sequence. In some cases, the nucleic acid delivery vectors comprise a WPRE3 element positioned downstream of the transgene. In some cases, the nucleic acid delivery vector comprises a bGH-polyA element position downstream of the WPRE3 element. In some cases, the WPRE3 element is positioned in between the transgene sequence and the bGH-polyA element. In some cases, the WPRE3 element is operably linked to the WPRE3 element and the bGH-polyA element. In some embodiments, a nucleic acid composition further comprises a nucleic acid sequence of SEQ ID NO: 45. Aspects disclosed herein provide a nucleic acid composition comprising a nucleic acid sequence having at least 96% sequence identity to SEQ ID NO: 45. In some cases, SEQ ID NO: 45 provides a beneficial technical effect of improving RNA stability, facilitating attachment to cellular translation initiation factors such as eIF4E (eukaryotic translation initiation factor 4E) and eIF4G (eukaryotic translation initiation factor 4G) and enhances the assembly of the translation initiation complex, facilitating the recruitment of ribosomes to the mRNA, enhancing translation initiation of a transcribed mRNA, and promotes the expression of the therapeutic nucleic acid sequence coupled thereto.

In some cases, the nuclear targeting sequence is positioned downstream of the bGH-polyA element. In some cases, the nuclear targeting sequence is operatively linked to the bGH-polyA element. In some cases, the viral sequences comprise ITR sequences. In some cases, a first ITR sequence is positioned downstream of the nuclear targeting sequence. In some cases, a second ITR sequence is positioned upstream of the promoter sequence. In some cases, the first ITR sequence is operatively linked to the bGH-polyA element, and the second ITR sequence is operatively linked to the nuclear targeting sequence. In some cases, the WPRE3 element and the bGH-polyA element provides a beneficial technical effect of improving RNA stability, facilitating attachment to cellular translation initiation factors such as eIF4E (eukaryotic translation initiation factor 4E) and eIF4G (eukaryotic translation initiation factor 4G) and enhances the assembly of the translation initiation complex, facilitating the recruitment of ribosomes to the mRNA, enhancing translation initiation of a transcribed mRNA, and promotes the expression of the therapeutic nucleic acid sequence coupled thereto.

Aspects disclosed herein provide a nucleic acid composition comprising a therapeutic nucleic acid sequence, a first regulatory element comprising an Apolipoprotein E-Alpha-1-Antitrypsin (APOE-AAT) promoter sequence operably linked to a hemoglobin subunit gamma intron (hBGi) sequence, and a second regulatory element comprising a woodchuck hepatitis posttranscriptional regulatory element (WPRE3) sequence. In some embodiments, the nucleic acid composition includes a nucleic acid sequence having at least 80% sequence identity to SEQ ID NO: 6. In some embodiments, the nucleic acid composition comprises a nuclear targeting sequence that increases expression of the therapeutic nucleic acid sequence in a cell by at least 1.25-fold, as compared to an otherwise identical control nucleic acid composition lacking the nuclear targeting sequence. In some embodiments, the nucleic acid composition includes the nuclear targeting sequence comprises a nucleic acid sequence having at least 80% sequence identity to SEQ ID NO: 6. In some embodiments, the nucleic acid sequence having at least 80% sequence identity to SEQ ID NO: 6 increases expression of the therapeutic nucleic acid sequence in a cell by at least 1.25 fold as compared to an otherwise identical control composition lacking the nucleic acid sequence having at least 80% sequence identity to SEQ ID NO: 6. In some embodiments, the nucleic acid composition includes a first regulatory element comprising an APOE-AAT promoter sequence operably linked to a hBGi sequence, and a second regulatory element comprising a WPRE3 sequence. In some embodiments, the first regulatory element is upstream of the therapeutic nucleic acid sequence. In some embodiments, the second regulatory element is downstream of the therapeutic nucleic acid sequence. In some embodiments, the second regulatory element is downstream of the therapeutic nucleic acid sequence. In some embodiments, each of the first regulatory element and the second regulatory element are operably linked to the therapeutic nucleic acid sequence. In some embodiments, the first regulatory element is upstream of the therapeutic nucleic acid sequence, and the second regulatory element is downstream of the therapeutic nucleic acid sequence. In some embodiments, the nucleic acid sequence has at least 80% sequence identity to SEQ ID NO: 6 has at least 81, 82, 83, 84, 85, 86, 87, 88, 89, 90, 91, 92, 93, 94, 95, 96, 97, 98, or 99% sequence identity (e.g., 100% sequence identity) to SEQ ID NO: 6. In some embodiments, the nucleic acid sequence having at least 80% sequence identity to SEQ ID NO: 6 is operably coupled to a second regulatory element comprising a WPRE3 sequence. In some embodiments, the first regulatory element comprises a sequence of SEQ ID NO: 9. In some embodiments, the second regulatory element comprises a sequence of SEQ ID NO: 4.

Aspects disclosed herein provide a nucleic acid composition a therapeutic nucleic acid sequence, a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence. In some embodiments, the intron sequence comprises a hemoglobin subunit gamma intron (hBGi) sequence. In some embodiments, the nucleic acid composition further comprises a poly-adenylation signal. In some embodiments, the poly-adenylation signal is positioned downstream of the second regulatory element. In some embodiments, the poly-adenylation signal is positioned downstream of the therapeutic nucleic acid sequence. In some embodiments, the poly-adenylation signal is positioned upstream of the nuclear targeting sequence. In some embodiments, the promoter sequence comprises an Apolipoprotein E-Alpha-1-Antitrypsin (APOE-AAT) promoter sequence. In some embodiments, the first regulatory element is positioned upstream and directly coupled to the therapeutic nucleic acid sequence, the second regulatory element is positioned downstream and directly coupled to the therapeutic nucleic acid sequence, the poly-adenylation signal is positioned downstream and directly coupled to the second regulatory element, and wherein the nuclear targeting sequence is positioned downstream and directly coupled to the poly-adenylation signal.

Aspects disclosed herein provide a nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein: the A1 domain comprises substitution of phenylalanine at position 328 to serine when the Factor VIII polypeptide is aligned with SEQ ID NO: 35; and the A3 domain comprises substitution of cysteine at position 1249 to glycine when the Factor VIII polypeptide is aligned with SEQ ID NO: 35 and substitution of cysteine at position 1253 to glycine when the Factor VIII polypeptide is aligned with SEQ ID NO: 35. In some embodiments, the therapeutic nucleic acid sequence encodes a Factor VIII (FVIII) polypeptide comprising an amino acid sequence having up to 2 amino acid substitutions relative to SEQ ID NO: 24. In some cases, the therapeutic nucleic acid sequence encodes a FVIII polypeptide with the combination of a B domain comprises SEQ ID NO: 27; the A1 domain substitution of phenylalanine at position 328 to serine and the A3 domain comprises substitution of cysteine at position 1249 to glycine provides a beneficial technical effect of increasing the coagulation activity of the FVIII protein in a subject. In some cases, the therapeutic nucleic acid sequence encodes a FVIII polypeptide with the combination of a B domain comprising SEQ ID NO: 27; the A1 domain comprising a substitution of phenylalanine at position 328 to serine and the A3 domain comprising a substitution of cysteine at position 1249 to glycine provides a beneficial technical effect of increasing the expression of the FVIII in the subject.

In some embodiments, the B domain comprises no more than 6 consensus sites for N-linked glycosylation. In some embodiments, the B domain comprises an amino acid sequence of SEQ ID NO: 28, and wherein the FVIII polypeptide is not more than 2300 amino acids in length. In some embodiments, the B domain comprises an amino acid sequence of SEQ ID NO: 34. In some embodiments, the A1 domain comprises an amino sequence of SEQ ID NO: 25. In some embodiments, the A3 domain comprises an amino sequence of SEQ ID NO: 29. In some embodiments, the Factor VIII polypeptide comprises an amino acid sequence of SEQ ID NO: 24.

In some embodiments, the nucleic acid composition further includes a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence. In some embodiments, the nucleic acid composition further includes a nucleic acid sequence having at least 96% sequence identity to SEQ ID NO: 45. In some embodiments, the nucleic acid composition further includes a nucleic acid sequence having at least 80% sequence identity to SEQ ID NO: 6. In some embodiments, the nucleic acid sequence having at least 80% sequence identity to SEQ ID NO: 6 increases expression of the therapeutic nucleic acid sequence in a cell by at least 1.25-fold as compared to an otherwise identical control composition lacking the nucleic acid sequence having at least 80% sequence identity to SEQ ID NO: 6. In some embodiments, the first regulatory element comprises a sequence of SEQ ID NO: 9. In some embodiments, the second regulatory element comprises a sequence of SEQ ID NO: 45. In some embodiments, the nucleic acid composition comprises a sequence of SEQ ID NO: 39. In some cases, the combination of the first regulatory element positioned upstream of the therapeutic nucleic acid sequence (e.g., transgene) and the second regulatory element positioned downstream of the therapeutic nucleic acid sequence (e.g., transgene) provides a beneficial technical effect of increasing expression of the therapeutic nucleic acid sequence in a cell. In some cases, the combination of the first regulatory element positioned upstream of the therapeutic nucleic acid sequence (e.g., transgene) and the second regulatory element positioned downstream of the therapeutic nucleic acid sequence (e.g., transgene) and the nuclear targeting sequence having at least 80% sequence identity to SEQ ID NO: 6 provides a beneficial technical effect of increasing delivery of the therapeutic nucleic acid sequence to a nucleus of the cell and resulting expression of the therapeutic nucleic acid sequence in the cell. In some cases, the combination of the first regulatory element positioned upstream of the therapeutic nucleic acid sequence (e.g., transgene) and the second regulatory element positioned downstream of the therapeutic nucleic acid sequence (e.g., transgene) in combination with the post-transcriptional regulatory element of a nucleic acid sequence having at least 96% sequence identity to SEQ ID NO: 45, and the nuclear targeting sequence having at least 80% sequence identity to SEQ ID NO: 6 provides a beneficial technical effect of increasing delivery of the therapeutic nucleic acid sequence to a nucleus of the cell and resulting expression of the therapeutic nucleic acid sequence in the cell.

Aspects disclosed herein provide a nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 34; and wherein: the A1 domain comprises an amino sequence of SEQ ID NO: 25; and the A3 domain comprises an amino sequence of SEQ ID NO: 29, the nucleic acid composition further comprising: a first regulatory element comprising a sequence of SEQ ID NO: 9; a second regulatory element comprising a second regulatory element comprising a sequence of SEQ ID NO: 45; and a nucleic acid sequence of SEQ ID NO: 6. In some embodiments, the nucleic acid composition comprises a sequence of SEQ ID NO: 39. In some cases, the combination of the therapeutic nucleic acid sequence encoding FVIII with a B domain comprising SEQ ID NO: 27; the A1 domain comprising a substitution of phenylalanine at position 328 to serine and the A3 domain comprising a substitution of cysteine at position 1249 to glycine, with the first regulatory element upstream of the therapeutic nucleic acid sequence and the second regulatory element positioned downstream of the therapeutic nucleic acid sequence provides a beneficial technical effect of increasing the activity of the FVIII protein in a subject, and expression of the FVIII protein in the subject. In some cases, the combination of the first regulatory element positioned upstream of the FVIII nucleic acid sequence (e.g., FVIII transgene) and the second regulatory element positioned downstream of the FVIII nucleic acid sequence (e.g., FVIII transgene) in combination with the post-transcriptional regulatory element of a nucleic acid sequence having at least 96% sequence identity to SEQ ID NO: 45, and the nuclear targeting sequence having at least 80% sequence identity to SEQ ID NO: 6 provides a beneficial technical effect of increasing delivery of the FVIII nucleic acid sequence to a nucleus of the cell and resulting expression of FVIII by the cell.

In some embodiments, the therapeutic nucleic acid sequence comprises a nucleic acid sequence of SEQ ID NO: 32. In some embodiments, the nucleic acid composition is a non-viral vector. In some embodiments, the nucleic acid composition is at least 5000, 5500, 6000, 6500, or 7000 bp in length, and wherein the non-viral vector encodes a full-length gene.

In some embodiments, the nucleic acid composition comprises a miniplasmid backbone. In some embodiments, the miniplasmid backbone is smaller than 1 kb. In some embodiments, the miniplasmid backbone comprises a sequence of SEQ ID NO: 7. In some embodiments, the therapeutic nucleic acid sequence is a therapeutic agent. In some embodiments, the therapeutic nucleic acid sequence encodes a therapeutic agent. In some embodiments, the therapeutic nucleic acid sequence encodes a protein which provides a therapeutic effect to a subject.

In some embodiments, the nucleic acid composition is an isolated nucleic acid molecule. In some embodiments, the nucleic acid composition does not comprise an origin of replication, or a bacterial origin of replication. In some embodiments, the nucleic acid composition further comprises one or more Inverted Terminal Repeat (ITR) sequences. In some embodiments, a first ITR sequence is positioned upstream of the therapeutic nucleic acid sequence, promoter sequence, first regulatory element, and/or nuclear targeting sequence. In some embodiments, a second ITR sequence is positioned downstream of the therapeutic nucleic acid sequence, promoter sequence, first regulatory element, and/or nuclear targeting sequence.

In some embodiments, the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. In some embodiments, the nucleic acid composition the therapeutic nucleic acid sequence encodes a FVIII polypeptide comprising an amino acid sequence having up to 3 amino acid substitutions relative to SEQ ID NO: 24. In some embodiments, the B domain comprises up to 6 consensus sites for N-linked glycosylation. In some embodiments, the B domain comprises an amino acid sequence of SEQ ID NO: 28. In some embodiments, the B domain comprises an amino acid sequence of SEQ ID NO: 34. In some embodiments, the A1 domain comprises substitution of phenylalanine at position 328 to serine when the Factor VIII polypeptide is aligned with SEQ ID NO: 35. In some embodiments, the A1 domain comprises an amino sequence of SEQ ID NO: 25. In some embodiments, the A3 domain comprises substitution of cysteine at position 1249 to glycine when the Factor VIII polypeptide is aligned with SEQ ID NO: 35. In some embodiments, the A3 domain comprises substitution of cysteine at position 1253 to glycine when the Factor VIII polypeptide is aligned with SEQ ID NO: 35. In some embodiments, the A3 domain comprises an amino sequence of SEQ ID NO: 28. In some embodiments, the therapeutic nucleic acid sequence encodes the amino acid sequence of SEQ ID NO: 24. In some cases the therapeutic nucleic acid sequence encodes a FVIII polypeptide is a transgene. In some cases, the positioning of a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence, provides a beneficial technical effect of improving transgene delivery and expression. In some cases, the positioning of a nuclear targeting sequence, for example, a nucleic acid sequence having at least 80% sequence identity to SEQ ID NO: 6, provides a beneficial technical effect of improving transgene delivery and expression. In some cases, the poly-adenylation signal positioned downstream of the second regulatory element provides a beneficial technical effect of improving transgene delivery and expression. In some cases, one or more Inverted Terminal Repeat (ITR) sequences provides a beneficial technical effect of improving transgene delivery and expression.

In some cases the where the therapeutic nucleic acid sequence is a FVIII transgene, the first regulatory element is positioned upstream and directly coupled to the therapeutic nucleic acid sequence, the second regulatory element is positioned downstream and directly coupled to the therapeutic nucleic acid sequence, the poly-adenylation signal is positioned downstream and directly coupled to the second regulatory element, and where the nuclear targeting sequence is positioned downstream and directly coupled to the poly-adenylation signal, the combination of elements and relative positioning of elements within the nucleic acid provides a beneficial technical effect of improving transgene delivery and expression.

### Methods of Gene Delivery

Aspects disclosed herein provide the nucleic acid composition disclosed herein for use a method of treating a bleeding disorder in a subject in need thereof, the method comprising administering the nucleic acid composition of any one of the preceding claims to the subject. Aspects disclosed herein provide a method of expressing a nucleic acid payload in a cell, the method comprising administering the nucleic acid composition of any one of the preceding claims to a subject. In some embodiments, the method further includes applying an ultrasonic acoustic energy to the cell. In some embodiments, the method further includes administering to the subject a sonoactive agent. In some embodiments, the nucleic acid composition is administered to the subject as a naked nucleic acid. In some embodiments, the nucleic acid composition encodes a full-length polypeptide, wherein the method induces expression of the full-length polypeptide in-vivo. In some embodiments, the method further includes administering to the subject the nucleic acid composition in at least two treatments at least 48 hours apart, thereby achieving a therapeutic level of the clotting factor in the subject. In some embodiments, the method further includes administering to the subject ultrasound acoustic energy in the treatments. In some embodiments, the method further includes administering to the subject a sonoactive agent in the treatments. In some embodiments, one or both of the nucleic acid composition or the sonoactive agent are administered to the subject intravenously through a peripheral vein. In some embodiments, administering the ultrasound acoustic energy comprises applying the ultrasound energy at a mechanical index (MI) of at least 0.8, 1.3, 1.8, 1.9, or 2.2. In some embodiments, administering the ultrasound acoustic energy further comprises applying the ultrasound energy at a mechanical index (MI) of up to 0.4. In some embodiments, the ultrasound energy is applied at an intensity (ISPTA) of at least 200 mW/cm2. In some embodiments, the ultrasound energy is applied at pulse length of at least 20, 200, or 600 microseconds. In some embodiments, the subject is administered at least three treatments. In some embodiments, the at least three treatments are administered at least 48 hours apart. As used herein a “treatment” refers to administration of the nucleic acid composition to the subject in combination with one or more of ultrasound energy, and/or a sonoactive agent.

In some embodiments, the subject is administered at least two cycles, each cycle comprising at least two treatments. In some embodiments, the subject is administered at least three cycles, each cycle comprising at least two treatments. In some embodiments, the subject is administered at least three cycles, each cycle comprising at least three treatments. In some embodiments, each cycle is administered to the subject at least 10 days apart. In some embodiments, each cycle is administered to the subject at least 30 days apart. In some embodiments, each cycle is administered to the subject at least 40 days apart. As used herein a “cycle” refers to a series of two or more (e.g., three) treatments. In some embodiments, the method further includes imaging an organ of the subject with ultrasound acoustic energy. In some embodiments, the imaging the organ of the subject comprises verifying presence of the sonoactive agent in the organ or the targeting of the ultrasound acoustic energy towards to organ. In some embodiments, a therapeutic level of the FVIII polypeptide is maintained for at least 10, 20, 30, 40, 50, 60, 70, 80, 90, 100, 110, 120, 130, 140, 145, or 150 days. In some embodiments, vascular flow is not occluded in the subject. In some embodiments, the subject has a bleeding disorder, wherein the therapeutic nucleic acid sequence encodes FVIII, and wherein the method thereby treats the subject having the bleeding disorder. In some embodiments, the bleeding disorder is hemophilia A. In some embodiments, the sonoactive agent comprises lipid-stabilized microstructures. In some embodiments, the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble. In some cases, administering repeated treatments to the subject in which the nucleic acid composition encoding the FVIII protein delivered using sonoactive agents disrupted with ultrasound energy provides a beneficial technical effect of increasing the delivery of the nucleic acid composition to cells and resulting expression of the FVIII protein, for example, expressing the FVIII protein at therapeutic levels in a subject for extended periods.

Aspects disclosed herein provide a nucleic acid composition of any one of the preceding claims, for use a method of treating a bleeding disorder in a subject in need thereof, the method comprising administering the nucleic acid composition of any one of the preceding claims to the subject, administering a sonoactive agent to the subject. Aspects disclosed herein provide a cell comprising the nucleic acid composition of any one of the preceding claims. Aspects disclosed herein provide a nucleic acid composition for use a method of treating a bleeding disorder in a subject in need thereof, the nucleic acid composition comprising: a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 34; and wherein: the A1 domain comprises an amino sequence of SEQ ID NO: 25; and the A3 domain comprises an amino sequence of SEQ ID NO: 29, the nucleic acid composition further comprising: a first regulatory element comprising a sequence of SEQ ID NO: 9; a second regulatory element comprising a second regulatory element comprising a sequence of SEQ ID NO: 45; and a nucleic acid sequence of SEQ ID NO: 6 the method comprising: administering to the subject the nucleic acid composition, a sonoactive agent, and ultrasound acoustic energy in a treatment session; administering a cycle comprising at least three treatment sessions to the subject; administering at least three cycles to the subject each cycle at least 10 days apart, thereby achieving a therapeutic level of the FVIII polypeptide in the subject. Aspects disclosed herein provide a method of manufacturing the nucleic acid composition disclosed herein. Aspects disclosed herein provide a kit comprising: a sonoactive agent; and means for expressing a Factor VIII polypeptide in vivo at a level that is at least 1.5 fold greater than expression of SEQ ID NO. 3. Aspects disclosed herein provide a kit comprising: a sonoactive agent; and the nucleic acid composition disclosed herein.

Aspects disclosed herein provide a method of treating a bleeding disorder comprising administering to a subject a nucleic acid encoding a human clotting factor in at least two treatments at least 48 hours apart, thereby achieving a therapeutic level of the clotting factor in the subject. In some embodiments, the therapeutic level of the clotting factor is maintained for at least 10, 20, 30, 40, 50, 60, 70, 80, 90, 100, 110, 120, 130, 140, 145, or 150 days. In some embodiments, the subject is administered at least three treatments. In some embodiments, the sonoactive agent comprises protein-stabilized microstructures. In some embodiments, the sonoactive agent comprises a shell filled with a perfluorinated gas. In some embodiments, the sonoactive agent comprises lipid-stabilized microstructures. In some embodiments, the at least three treatments are administered at least 48 hours apart. In some embodiments, the subject is administered at least two cycles, each cycle comprising at least two treatments. In some embodiments, the subject is administered at least three cycles, each cycle comprising at least two treatments. In some embodiments, the subject is administered at least three cycles, each cycle comprising at least three treatments. In some embodiments, each cycle is administered to the subject at least 10 days apart. In some embodiments, each cycle is administered to the subject at least 30 days apart. In some embodiments, each cycle is administered to the subject at least 40 days apart. In some cases, the nucleic acid compositions encoding a human clotting factor disclosed herein are configured to administration to a subject in at least two treatments at least 48 hours apart, thereby achieving a therapeutic level of the clotting factor in the subject. In some cases, the methods of delivery of the nucleic acids encoding human clotting factors disclosed herein provide a beneficial technical effect of increasing expression of the human clotting factor, for example, achieving or maintaining a therapeutic level of the human clotting factor. In some cases, the at least two treatments and at least two cycles of treatments with nucleic acids encoding human clotting factors disclosed herein provide a beneficial technical effect of achieving or maintaining a therapeutic level of the human clotting factor, thereby treating a bleeding disorder. In some cases, the bleeding disorder is hemophilia A and the clotting factor is human FVIII.

In some embodiments, the method includes administering to the subject ultrasound acoustic energy in the treatments. In some embodiments, the method includes administering to the subject a sonoactive agent in the treatments. In some embodiments, administering the ultrasound acoustic energy comprises applying the ultrasound energy at a mechanical index (MI) of at least 0.8, 1.3, 1.8, 1.9, or 2.2. In some embodiments, administering the ultrasound acoustic energy comprises applying the ultrasound energy at a mechanical index (MI) of up to 0.4. In some embodiments, administering the ultrasound energy comprises applying an acoustic radiation force to the subject. In some embodiments, the ultrasound energy is applied at an intensity (ISPTA) of at least 200 mW/cm2. In some embodiments, the ultrasound energy is applied at pulse length of at least 20 microseconds.

In some cases, nucleic acid compositions disclosed herein are administered as naked plasmid DNA. In some cases, nucleic acid compositions disclosed herein are administered using a viral vector, for example, a parvovirus, retrovirus, lentivirus, a herpes simplex virus, or adeno-virus vector (AAV). In some cases, nucleic acid compositions disclosed herein are administered using lipid-based delivery methods, for example, plasmid DNA may be coated with lipids in an organized structure such as a micelle or a liposome (complexed with DNA as a lipoplex). Anionic and neutral lipids may be used for the construction of lipoplexes for synthetic vectors. In one embodiment, cationic lipids, due to their positive charge, may be used to condense negatively charged DNA molecules to facilitate the encapsulation of DNA into liposomes. Alternatively, methods such as sonoporation may be utilized.

Disclosed herein are methods for treating a subject suffering from a genetic disorder comprising administering to the subject a therapeutically effective amount of the delivery vectors comprising a therapeutic transgene, or a pharmaceutical composition comprising the same. In this instance, a “therapeutically effective amount” is an amount of the delivery vector is an amount that after administration results in the expression of the therapeutic protein in a level sufficient to at least partially and preferably fully ameliorate the symptoms of the genetic disorder.

In some embodiments, the cell is a liver cell, a kidney cell, a brain cell, a muscle cell, a cardiac cell, a pancreatic cell, a blood cell, or a tumor cell. In some embodiments, the cell is a hepatocyte.

In some embodiments, the therapeutic nucleic acid sequence encodes a CRISPR/Cas system, a Cas protein or homolog or variant thereof, a TALEN, a ZFN, or a template DNA molecule suitable for knock-in via homologous end joining (NHEJ) or homology directed repair (HDR).

Aspects disclosed herein provide a method of expressing a nucleic acid payload in a cell, the method comprising administering the nucleic acid composition disclosed herein to a subject. In some embodiments, the method includes applying ultrasonic acoustic energy to the cell. In some embodiments, the method includes administering to the subject a plurality of sonoactive microstructures. In some embodiments, the method includes contacting the cell with a lipid nanoparticle composition. In some embodiments, the nucleic acid composition is administered to the subject in a viral vector. In some embodiments, the method includes to the subject a plurality of magnetic particles and applying a magnetic field to the cell. In some embodiments, the magnetic particles comprise paramagnetic nanoparticles. In some embodiments, the method includes applying an electrical field to the cell. In some embodiments, the nucleic acid composition is administered to the subject using hydrodynamic injection. In some embodiments, the nucleic acid composition is administered to the subject as a naked nucleic acid. In some embodiments, the nucleic acid composition is administered to the subject as an encapsulated nucleic acid. In some embodiments, the nucleic acid composition is encapsulated in a lipid or a polymer. In some embodiments, the nucleic acid composition encodes a full-length polypeptide, wherein the method induces expression of the full-length polypeptide in-vivo. In some embodiments, the subject has a bleeding disorder, wherein the therapeutic nucleic acid sequence of the polypeptide encodes FVIII or FIX, and wherein the method thereby treats the subject having the bleeding disorder. In some embodiments, the bleeding disorder is hemophilia A.

Factor VIII (FVIII) is protein which plays an important role in the blood clotting cascade, and is intrinsic to a pathway of coagulation. FVIII is a large multidomain glycoprotein, synthesized in the liver and endothelial cells. Wild-type FVIII is approximately 2,332 amino acids, making it one of the largest proteins involved in the blood clotting cascade. One known function of FVIII is to act as a cofactor for clotting factor IX in the activation of clotting factor X, which ultimately leads to the conversion of prothrombin to thrombin, thereby providing for a coagulation response. The wild-type human FVIII protein consists of several distinct domains: A1-A2-B-A3-C1-C2. Without being bound by theory, the A1 domain is located at the N-terminal end of FVIII and is involved in the binding of FVIII to von Willebrand factor (VWF). The interaction between FVIII and VWF stabilizes FVIII in circulation by protecting it from proteolytic degradation. The A2 domain contains a binding site for activated factor IX (factor IXa). The interaction between the A2 domain of FVIII and factor Ixa is important for the activation of factor X in the coagulation cascade. The wild-type B domain is a large domain of FVIII of about 740 to 800 amino acids, and is not thought to be essential to coagulation function, and separates the A2 and A3 domains. The A3 domain is located adjacent to the A2 domain and is involved in the assembly of the tenase complex, which consists of factor VIIIa, factor IXa, and calcium ions. The C1 domain is located near the C-terminus of FVIII and is important for the binding of FVIII to phospholipid surfaces, which is helpful for the localization of FVIII to the site of vascular injury. The C2 domain is involved in calcium ion binding, which stabilizes the structure of FVIII and is thought to be necessary for its proper FVIII function.

In gene therapy for hemophilia A, various truncated versions of the Factor VIII (FVIII) protein are utilized to address the deficiencies or mutations in the FVIII gene that lead to the disorder, and truncates are usually utilized in order to fit within the carrying capacity of a viral vector, for example, and adenovirus vector. These truncated forms are intended to retain the essential functional domains of FVIII while minimizing the risk of immunogenicity and improving the expression and secretion of the protein, often by deleting the B-domain. For example, the B-domain of FVIII is not essential for its procoagulant function and can be removed without affecting its activity. BDD FVIII lacks this non-essential B-domain, resulting in a smaller protein with improved secretion and stability. BDD FVIII is often used in gene therapy approaches as it is thought to facilitate higher expression levels, may reduce the risk of immune responses compared to full-length FVIII, and provides for packaging within the size of an AAV vector. While B-Domain Deleted (BDD) Factor VIII (FVIII) has been widely used in gene therapy and provides for packaging within viral vectors with small carrying capacities, there may be disadvantages to such BDD FVIII constructs as well, for example, reduced stability, altered pharmacokinetics, functional changes, and reduced overall therapeutic effect. Further, no gene therapy product currently on the market has been able to successfully deliver a full length or FVIII construct, or a FVIII construct which can exceed the carrying capacity of an AAV, and there remains a need for improved FVIII transgenes which provide for improved expression of the therapeutic protein and long-term beneficial effect to patients. Further, modification to other domains of the FVIII protein which can also improve expression of the therapeutic protein and provide a long-term beneficial effect to patients would also represent an improvement over the current FVIII gene therapy products.

Aspects disclosed herein provide a Factor VIII polypeptide comprising: an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 36; and the A3 domain comprises at least two amino acid substitutions as compared to SEQ ID NO: 37. Aspects disclosed herein provide a Factor VIII polypeptide comprising an amino acid sequence having up to 3 amino acid substitutions relative to SEQ ID NO: 24. In some embodiments, the FVIII polypeptide has an amino acid sequence having up to 3 amino acid substitutions relative to SEQ ID NO: 24. In some cases, the non-naturally occurring B domain and one or more amino acid substitution in the A1 and A3 domain may provide for enhanced expression, secretion, and coagulation activity of FVIII. In some cases, the non-naturally occurring B domain and one or more amino acid substitution in the A1 and A3 domain may significantly increase the expression levels of FVIII in host cells by optimizing transcriptional and translational processes leading to increased yields of functional FVIII protein. In some cases, the non-naturally occurring B domain and one or more amino acid substitution in the A1 and A3 domain may provide for increased stability of the FVIII from host cells into the bloodstream, for example, by improving protein folding and trafficking s compared to FVIII protein lacking these modifications. In some cases, the non-naturally occurring B domain and one or more amino acid substitution in the A1 and A3 domain may stabilize the FVIII protein in circulation and protect it from degradation within the cell or in circulation, thereby providing for a longer functional duration, improving its therapeutic efficacy. In some cases, the non-naturally occurring B domain and one or more amino acid substitution in the A1 and A3 domain may provide for proper protein folding in a conformation that mirrors the native conformation of wild-type FVIII.

In some embodiments, the FVIII polypeptide has an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of: the A1 domain comprises at least one amino acid substitution relative to SEQ ID NO: 35; and the A3 domain comprises at least two amino acid substitutions as compared to SEQ ID NO: 36. In some embodiments, the B domain comprises up to 6 consensus sites for N-linked glycosylation. In some embodiments, the B domain comprises an amino acid sequence of SEQ ID NO: 28. In some embodiments, the B domain comprises an amino acid sequence of SEQ ID NO: 34. In some embodiments, the A1 domain comprises substitution of phenylalanine at position 328 to serine when the FVIII polypeptide is aligned with SEQ ID NO: 35. In some embodiments, the A1 domain comprises an amino sequence of SEQ ID NO: 25. In some embodiments, the A3 domain comprises substitution of cysteine at position 1249 to glycine when FVIII polypeptide is aligned with SEQ ID NO: 35. In some embodiments, the A3 domain comprises substitution of cysteine at position 1253 to glycine when FVIII polypeptide is aligned with SEQ ID NO: 35. In some embodiments, the A3 domain comprises an amino sequence of SEQ ID NO: 28. In some embodiments, the FVIII polypeptide comprises the amino acid sequence of SEQ ID NO: 24. Aspects disclosed herein provide a kit comprising: a sonoactive agent; and means for expressing a Factor VIII polypeptide in vivo at a level that is at least 1.5 fold greater than expression of SEQ ID NO. 3. Aspects disclosed herein provide a kit comprising: a sonoactive agent; and the nucleic acid composition disclosed herein. In some embodiments, further included are instructions for administering ultrasound acoustic energy to a subject to facilitate expression of the FVIII polypeptide in vivo. In some embodiments, further included instructions for administering ultrasound acoustic energy to a subject to facilitate delivery of the nucleic acid composition to a subject. In some embodiments, the sonoactive agent comprises lipid-stabilized microstructures. In some embodiments, the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble. Aspects disclosed herein provide a cell comprising the FVIII polypeptide of any one of the embodiments disclosed herein. Aspects disclosed herein provide a nucleic acid encoding the FVIII polypeptide of any one of the embodiments disclosed herein. In some cases, the combination of the FVIII polypeptide with a B domain comprising SEQ ID NO: 27; the A1 domain comprising a substitution of phenylalanine at position 328 to serine and the A3 domain comprising a substitution of cysteine at position 1249 to glycine provides a beneficial technical effect of increasing the activity of the FVIII protein in a subject, and expression of the FVIII protein in the subject.

Ultrasound refers to the application of electromagnetic energy in the range of greater than 20 kHz up to several gigahertz. Ultrasound is used in many different fields, most commonly in the field of diagnostics and medical imaging for producing images of tissue within the human body. ultrasound acoustic energy can be generated at various frequencies within the 20 kHz up to several gigahertz range, most commonly within the range of about 1 to 10 megahertz when used for diagnostic imaging purposes. Ultrasound is commonly applied using ultrasonic transducers comprising one or more piezoelectric crystals which convert electrical energy into acoustic energy. In addition to imaging applications, ultrasound can also be used for a variety of other diagnostic and therapeutic applications, including determination of tissue elasticity and fibrosis, focused destruction of tissue using ultrasound ablation, and for the delivery of exogenous payloads (e.g., nucleic acids and therapeutic agents) to a cell. Sonoporation refers to the delivery of therapeutic agents, for example nucleic acids, using ultrasound and/or sonoactive microstructures (e.g., sonoactive microstructures) to a cell. Provided in certain embodiments herein are methods of delivering a nucleic acid construct into a target cell or tissue (e.g., of a subject) by applying ultrasonic acoustic energy to a cell, tissue, or organ (e.g., with sonoporation).

B mode ultrasound imaging refers to brightness mode imaging, in which ultrasonic waves are reflected from the tissue of a subject back to the ultrasound probe, and displayed on a 2-dimensional objects that are closer to the ultrasound transducer appear brighter, and objects which are farther away from the ultrasound transducer appear darker. B mode ultrasound imaging generally will focus ultrasonic acoustic energy emitted from a plurality of ultrasound arrays comprising piezoelectric crystals into a focused ultrasound beam which penetrates into the tissue about a vertical axis which is perpendicular to the surface of the ultrasound probe. The focused ultrasound beam reflects off the tissue and back towards the ultrasound transducer, forming a scan line in an ultrasound image. By moving the ultrasound transducer about a surface of tissue, an image of the underlying tissue can be generated using a B mode ultrasound image. B mode ultrasound imaging is the most common form of ultrasound used in the United States for medical imaging, and is what is commonly referred to as diagnostic or imaging ultrasound.

Plane wave imaging refers to an ultrasound imaging technique in which a plurality of ultrasound arrays comprising piezoelectric crystals in an ultrasound transducer are simultaneously fired without directing ultrasonic acoustic energy into a focused ultrasound beam, and which instead direct a large unfocused sheet or wave of ultrasound acoustic energy into a medium or tissue underlying an ultrasound probe. The primary difference between plane wave ultrasound imaging and B mode ultrasound imaging is the number of transducer arrays which are fired. Plain wave imaging typically will fire all arrays within an ultrasound transducer Producing a much larger and less focused wave of ultrasonic energy, while B mode imaging will typically only fire a subset of arrays which focused the ultrasound into a beam producing what is commonly referred to as a scan line. In plane wave imaging, the acoustic radiation pressure is almost uniform over the entire field of view, and lower peak and negative pressures are typically experienced as compared to traditional beam mode focused ultrasound beam imaging.

Acoustic radiation force refers to a static or transient force applied by an acoustic wave on the propagation medium or to an object in the path of the acoustic wave. Acoustic radiation forces can be applied using an ultrasound transducer when applying ultrasonic acoustic energy to a surface of a tissue or a propagation medium with sufficient ultrasound intensity. When applying a sufficient acoustic radiation force to a propagation medium or a tissue, the propagation medium or tissue underlying the ultrasound probe applying the acoustic radiation force may be displaced.

Shear waves, or secondary waves commonly refer to transversely oriented waves which occur in elastic medium that is subjected to a periodic shear. Shear refers to a change in shape without a change of volume of a layer of a propagation medium or tissue produced by a pair of equal forces acting in opposite directions about two faces of the layer or the propagation medium. Shear waves are a type of elastic wave which move through the body of an object or a propagation medium. In an elastic medium, the layer or the tissue will resume its original shape following application of the sheer force, adjacent layers will undergo subsequent shear, and the movement of particles within the medium or tissue will be propagated as a shear wave throughout the propagation medium or tissue. In an elastic medium, shear waves can be produced as a secondary wave following a compressional wave which is transmitted in the propagation medium or tissue. Ultrasound applying an acoustic radiation force can apply a compressional wave to a tissue, which can result in application of shear waves to a tissue when applied with sufficient intensity, at regular intervals, for sufficient periods of time to induce a regular shear in layers of a tissue. A compressional wave displaces tissue in a direction parallel to the propagation of the compressional waves. An ultrasound transducer can induce a compressional wave in a tissue which propagates from the ultrasound transducer about a vector normal to a surface of the ultrasound transducer. In some embodiments, applying the focused acoustic radiation force to the tissue comprises generating a compressional wave in the tissue. As an elastic tissue recovers from displacement due to a compressional wave, shear waves or secondary waves can be generated. In a shear wave, the direction of particle motion is parallel to the direction of propagation of the compressional wave, and the direction of propagation of the shear wave is normal to the direction of propagation of the compressional wave. The direction of particle motion in a shear wave is also normal to the direction of shear wave propagation in an elastic medium. Further, a compressional wave may be followed by a rarefaction wave which is a negative acoustic force in the tissue.

Shear wave elastography refers to a diagnostic technique using ultrasound to determine the elastic modulus of tissue, which is indicative of its fibrotic quality. Diseased tissue with certain fibrotic conditions will result in a significantly reduced elastic modulus of the tissue, as compared to a healthy tissue which is reasonably elastic as compared to diseased tissue in a fibrotic state. Shear wave elastography uses a combination of acoustic radiation force, plane wave imaging, and B mode imaging to provide a clinician with information as to the fibrotic quality of a tissue. Shear wave elastography applies an acoustic radiation force to displace the tissue underlying an ultrasound probe with a compressional wave, thereby generating shear waves in the tissue, applies a plane wave ultrasound to the tissue to monitor the propagation of the shear waves throughout the tissue thereby calculating the elastic modulus, and overlays this data atop a standard B mode ultrasound image to provide a visual representation of tissue stiffness.

Provided in certain embodiments herein are methods of delivering a nucleic acid construct into a target cell or tissue (e.g., of a subject) by applying ultrasonic acoustic energy to a cell, tissue, or organ. Provided in certain embodiments herein are methods of delivering a nucleic acid construct into a target cell or tissue (e.g., of a subject) by delivering an exogenous payload to the subject; and applying a focused acoustic radiation force (ARF) to the subject, thereby generating shear waves in the tissue of the subject, wherein the focused acoustic radiation force enhances delivery of the exogenous payload to the cell in the tissue of an organ of the subject. Disclosed herein are methods of sonoporation in which an exogenous payload is delivered to a cell in a tissue of a subject using a focused acoustic radiation force applied using ultrasound. Aspects of the sonoporation methods disclosed herein may also include inducing displacing the tissue of the subject with the acoustic radiation force to induce propagation of shear waves throughout the tissue of the subject thereby enhancing delivery of a nucleic acid payload to a cell. Disclosed herein are methods of sonoporation in which an exogenous payload is delivered to a cell in a tissue of a subject using a focused acoustic radiation force applied using ultrasound. Aspects of the sonoporation methods disclosed herein may also include inducing displacing the tissue of the subject with the acoustic radiation force to induce propagation of shear waves throughout the tissue of the subject thereby enhancing delivery of a nucleic acid payload to a cell. The method may further include applying the acoustic radiation force to induce propagation of shear waves throughout the tissue in combination with other secondary ultrasound energies such as plane wave ultrasound or focused beam ultrasound in which the secondary ultrasound energy moves sonoactive microstructures endothelial border of a tissue comprising the cell, while applying the focused acoustic radiation force during shear wave propagation induces inertial cavitation of sonoactive microstructures at the endothelial border of the tissue comprising the cell, thereby enhancing delivery of the therapeutic payload to a cell, and, in cases of a nucleic acid payload, resulting gene expression.

In some embodiments, the focused acoustic radiation force is applied using an ultrasound probe applying ultrasound acoustic energy to the tissue. In some embodiments, the ARF displaces the tissue of the subject. In some embodiments, the shear waves displace the tissue of the subject. In some embodiments, a tissue displacement is at least 0.001 mm. In some embodiments, a tissue displacement ranges from at least 0.001 mm to about 5 mm. In some embodiments, a tissue displacement ranges from 0.01 mm to about 1 mm.

In some embodiments, the focused acoustic radiation force is applied using an ultrasound probe applying ultrasound acoustic energy to the tissue. In some embodiments, the ultrasonic acoustic energy is applied at a mechanical index of greater than 0.4. In some embodiments, the ultrasonic acoustic energy is applied at a mechanical index of about 1.4. In some embodiments, the ultrasonic acoustic energy is applied at a mechanical index of at least 1.3. In some embodiments, the ultrasonic acoustic energy is applied at a mechanical index of greater than 0.4 up to about 3.0. In some embodiments, the ultrasonic acoustic energy is applied at a frequency of about 0.1 MHz to about 10 MHz. In some embodiments, the ultrasonic acoustic energy is applied at a frequency of about 2.5 MHz. In some embodiments, the applying ultrasonic acoustic energy to the tissue comprises applying the ultrasound acoustic energy at an ultrasound intensity of at least 100 mW/cm². In some embodiments, the applying ultrasonic acoustic energy to the tissue comprises applying the ultrasound acoustic energy at an ultrasound intensity of about 100 mW/cm² to about 10,000 mW/cm². In some embodiments, the applying ultrasonic acoustic energy to the tissue comprises applying the ultrasound acoustic energy at an ultrasound intensity of about 100 mW/cm² to about 5,000 mW/cm². In some embodiments, the applying ultrasonic acoustic energy to the tissue comprises applying the ultrasound acoustic energy at an ultrasound intensity of about 100 mW/cm² to about 500 mW/cm². In some embodiments, the applying ultrasonic acoustic energy to the tissue comprises applying the ultrasound acoustic energy at an ultrasound intensity of about 110 mW/cm² to about 200 mW/cm². In some embodiments, the applying ultrasonic acoustic energy to the tissue comprises applying the ultrasound acoustic energy at an ultrasound intensity of about 188 mW/cm². In some embodiments, the ultrasound intensity is a spatial-peak temporal average intensity. In some embodiments, the spatial-peak temporal average intensity is calculated in a focal region of the tissue. In some embodiments, the focused acoustic radiation force is applied in two or more pulses, with an interval between each of the two or more pulses. In some embodiments, a plane wave ultrasound is applied to the tissue during the interval. In some embodiments, the one or more pulses are up to 500 microseconds. In some embodiments, the one or more pulses are at least 100 microseconds. In some embodiments, the one or more pulses are about 100 microseconds to about 500 microseconds. In some embodiments, the interval is up to 500 milliseconds. In some embodiments, the interval is up to 100, 500, 1000, 1500, 2000, 2500, 3000, 4000, or 5000 milliseconds. In some embodiments, the interval is from about 100 milliseconds to about 5000 milliseconds. In some embodiments, the applying the focused acoustic radiation force is performed in one or more sequences, wherein a sequence comprises two or more pulses and interval(s) therebetween. In some embodiments, a time between application of the one or more sequences is at least 5, 10, 20, 30, 60, 120, 180, 240, 300, 360, 420, 480, 540, or 600 seconds. In some embodiments, a time between application of the one or more sequences ranges from about 5 to about 300 seconds. In some embodiments, a time between application of the one or more sequences ranges from about 10 to about 60 seconds. In some embodiments, the focused acoustic radiation force is applied for at least 10, 20, 30, 60, 120, 180, 240, 300, 360, 420, 480, 540, or 600 seconds. In some embodiments, applying ultrasonic acoustic energy to the tissue comprises applying the ultrasound acoustic energy at a focal depth of up to 10, 8, 6, or 4 cm from the ultrasound transducer. In some embodiments, applying ultrasonic acoustic energy to the tissue comprises applying the ultrasound acoustic energy at a focal depth of about 1 to about 10 cm from the ultrasound transducer. In some embodiments, applying ultrasonic acoustic energy to the tissue comprises applying the ultrasound acoustic energy at a focal depth of about 4 to about 10 cm from the ultrasound transducer. In some embodiments, applying ultrasonic acoustic energy to the tissue comprises applying the ultrasound acoustic energy at a focal depth of about 4 cm from the ultrasound transducer. In some embodiments, applying ultrasonic acoustic energy to the tissue comprises applying the ultrasound acoustic energy at a focal depth of about 6 cm from the ultrasound transducer.

In some embodiments, applying the focused wave ultrasound results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell. In some embodiments, the focused acoustic radiation force increases internalization of the exogenous payload in the cell. In some embodiments, the shear waves increase internalization of the exogenous payload in the cell. In some embodiments, inducing inertial cavitation the sonoactive microstructures increases internalization of the exogenous payload in the cell.

In some embodiments, a process provided herein provides sonoporation at two or more different ultrasonic acoustic energies (e.g., a first and second ultrasonic acoustic energy having a first and second MI, respectively). In certain embodiments, a process provided herein provides a process wherein an ultrasonic acoustic energy is continuously applied (e.g., ultrasonic acoustic energy transitions from the first ultrasonic acoustic energy to the second ultrasonic acoustic energy, without a period of no ultrasonic acoustic energy being applied). In certain embodiments, a transitory (e.g., third, fourth, etc.) ultrasonic acoustic energy is applied between application of the first and second ultrasonic acoustic energies. Provided in certain embodiments herein are methods of delivering a nucleic acid construct into a target cell or tissue (e.g., of a subject) by applying a first ultrasonic acoustic energy to a cell, tissue, or organ, and applying a second ultrasonic acoustic energy to the cell, tissue, or organ. In specific embodiments herein are methods for transfecting a nucleic acid construct into a target cell or tissue by applying a first ultrasonic acoustic energy having a first mechanical index (MI) and applying a second ultrasonic acoustic energy having a second mechanical index (MI). The present disclosure provides methods for enhancing transfection of a nucleic acid construct into the target cell or tissue by applying alternating ultrasonic acoustic energy, the alternating acoustic energy alternating between a first mechanical index (MI) and a second MI. Application of ultrasonic acoustic energy can be repeated several times during sonoporation, such as to increase the efficiency of nucleic acid construct transfection and/or delivery.

In some embodiments, the sonoporation treatment comprises applying an ultrasonic acoustic energy to the target cell (e.g., of a tissue or organ of the subject) (e.g., the ultrasonic acoustic energy having a mechanical index (MI)). In some embodiments, applying an ultrasonic acoustic energy to the target cell comprises applying a first ultrasonic acoustic energy to the target cell and applying a second ultrasonic acoustic energy to the target cell. In some embodiments, the (e.g., first or second) ultrasonic acoustic energy has a first mechanical index (MI). In certain embodiments, (e.g., the other of the first or second) ultrasonic energy has a second mechanical index (MI). In some embodiments, the (e.g., first or second) MI is less than 0.4. In certain embodiments (e.g., the other of the first or second) MI is greater than 0.4 (e.g., and less than 2.0). In specific embodiments, a first ultrasonic acoustic energy and a second ultrasonic acoustic energy are applied sequentially in a repeated manner. In some embodiments, a first MI is a Low MI (e.g., less than 0.4). In certain embodiments, a second MI is a High MI (e.g., 0.4 or greater). In some embodiments, a first MI is a Low MI (e.g., less than 0.4) and a second MI is a High MI (e.g., 0.4 or greater). In some embodiments, a second MI is a Low MI (e.g., less than 0.4). In certain embodiments, a first MI is a High MI (e.g., 0.4 or greater). In specific embodiments, a second MI is a Low MI (e.g., less than 0.4) and a first MI is a High MI (e.g., 0.4 or greater). In some embodiments, a Low MI is less than 0.3. In specific embodiments, a Low MI is less than 0.2. In more specific embodiments, a Low MI is less than 0.1. In still more specific embodiments, a Low MI is about 0.09. In still more specific embodiments, a Low MI is about 0.04. In still more specific embodiments, a Low MI is about 0.03. In some embodiments, a High MI is greater than 0.5. In specific embodiments, a High MI is 0.5 to 2.0 or is between 0.5 and 2.0. In more specific embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is greater than 0.4. In some embodiments, a High MI is greater than 0.5. In more specific embodiments, a High MI is 0.5 to 1 or is between 0.5 and 2.0. In some embodiments, a High MI is 1.5. In some embodiments, a High MI is 1.8. In some embodiments, a High MI is 2.0. In certain embodiments, any process provided herein (e.g., a sonoporation treatment) comprises administering of a continuous ultrasonic acoustic energy (which may have varying energy levels) that alternates (e.g., in identical, similar, or variable periods) between Low MI and High MI. In some embodiments, a low MI (e.g., less than 0.1) (e.g., first) ultrasonic acoustic energy (also referred to herein as a Low MI) is administered to the subject, and a set number pulses (e.g., of less than 30 seconds) of High MI (e.g., second) ultrasonic acoustic energy (also referred to herein as a High MI) is administered to the subject. In some embodiments, a process provided herein comprises administration of a plurality of pulses of high MI (e.g., second) ultrasonic acoustic energy, e.g., during an otherwise continuous administration of a low MI (e.g., first) ultrasonic acoustic energy. In specific embodiments, the number of High MI pulses is about 4 or more, such as up to about 12, or an unlimited number of pulses. In specific embodiments the number of High MI pulses is 6-30. In still more specific embodiments, the number of High MI pulses is between 8, 9, 12, 15, or 18, or any number therebetween. In some embodiments, at least 8, 9, 12, 15, or 18 high MI pulses are administered to the subject in between applications of low MI ultrasound acoustic energy.

In certain embodiments, the first (either High MI or Low MI) ultrasonic acoustic energy is applied before or after administration of any other agent, such as the nucleic acid and/or sonoactive structure. In some embodiments, the first ultrasonic acoustic energy is applied after administration of the sonoactive structure to the subject. In certain embodiments, the first ultrasonic acoustic energy is applied after administration of the nucleic acid to the subject. In some embodiments, the first ultrasonic acoustic energy is applied after administration of both the nucleic acid and the sonoactive structure(s).

In some embodiments, high MI ultrasound acoustic energy is administered in a pulse. In specific embodiments, a pulse length is any suitable length, such as less than 30 seconds. In more specific embodiments, a pulse length is less than 15 seconds. In still more specific embodiments, a pulse length is less than 10 seconds. In yet more specific embodiments, a pulse length is less than 5 seconds. In more specific embodiments, a pulse length is less than 2 seconds. In still more specific embodiments, a pulse length is less than 1 second and/or may be greater than or equal to 1 microsecond. In some embodiments, a pulse length ranges from 100 to 300 microseconds. In some embodiments, a pulse length is up to about 200 microseconds. In some embodiments, a pulse length is up to about 500 microseconds. In some embodiments, a pulse length ranges from 1 to 500 microseconds.

In various embodiments, a High MI ultrasonic acoustic energy is provided first temporally (e.g., first in order). In other embodiments, a Low MI ultrasonic acoustic energy is provided second temporally (e.g., second in order).

In some embodiments, any process provided herein further comprises administering (e.g., systemically administering, such as via infusion) a nucleic acid (e.g., any nucleic acid provided herein) to a subject (e.g., to whom the ultrasonic acoustic energies are applied).

In some embodiments, any process provided herein further comprises administering (e.g., systemically administering, such as via infusion) a sonoactive structure (e.g., any sonoactive structure or microbubble described herein) to a subject (e.g., to whom the ultrasonic acoustic energies are applied).

In certain embodiments, provided herein is a method of delivering a nucleic acid payload in a target cell (e.g., of a tissue or organ) of a subject, the method comprising: (a) administering to the subject a nucleic acid construct comprising the nucleic acid payload; (b) administering to the subject a plurality of sonoactive microstructures; and (c) administering an ultrasound acoustic energy, thereby delivering a sonoporation treatment.

In some embodiments, a sonoporation treatment (e.g., application of a first ultrasonic acoustic energy, a second ultrasonic acoustic energy, a single cycle of a first ultrasonic acoustic energy and a second ultrasonic acoustic energy, or series of cycles comprising a plurality of applications of a first ultrasonic acoustic energy and a plurality of applications of a second acoustic energy) can last for a few seconds (e.g., 1-100 seconds) or more, such as up to a few minutes (e.g., 1-3 minutes). In specific embodiments, a sonoporation treatment lasts for 1-30 seconds. In some specific embodiments, a sonoporation treatment lasts for 5-100 seconds. In certain embodiments, a sonoporation treatment lasts for at least 1 minute (e.g., 1-30 minutes).

In some embodiments, the first ultrasonic acoustic energy is administered within 60 minutes of administration of the nucleic acid and/or sonoactive structure(s). In specific embodiments, the first ultrasonic acoustic energy is administered within 30 minutes of administration of the nucleic acid and/or sonoactive structure(s). In more specific embodiments, the first ultrasonic acoustic energy is administered within 5 minutes of administration of the nucleic acid and/or sonoactive structure(s). In still more specific embodiments, the first ultrasonic acoustic energy is administered within 2 minutes of administration of the nucleic acid and/or sonoactive structure(s). In still more specific embodiments, the first ultrasonic acoustic energy may be applied simultaneously with administration of the nucleic acid and/or sonoactive structure(s).

In specific embodiments, the first (e.g., High MI) ultrasonic acoustic energy is applied immediately upon administration (e.g., infusion) or a period of time after administration (e.g., infusion) of the sonoactive structure(s) and/or nucleic acid.

In some embodiments, either the first or second ultrasonic acoustic energy is an ultrasonic acoustic energy (e.g., Low MI) that when applied to a cell, tissue, or organ of a subject results in stable cavitation (or stable vibrational cavitation) of the sonoactive structure and/or a change in the average diameter of the sonoactive structure(s), for example, due to inherent resonance properties of the microbubbles.

In certain embodiments, the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In certain embodiments, either the first or second ultrasonic acoustic energy is an ultrasonic acoustic energy (e.g., Low MI) that when applied to a cell, tissue, or organ of a subject results in stable cavitation (or stable vibrational cavitation) and/or a change in the average diameter of the sonoactive structure(s), and the other of the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In some instances, disruption of cell membrane allows target cells to become permeable to circulating agents such as nucleic acid constructs. In certain instances, such circulating agents can then enter the target cells, tissues or organs, such as in a more rapid manner (e.g., relative to either Low MI or High MI ultrasonic acoustic energy application alone, or in the absence of ultrasonic acoustic energy application).

In some embodiments, the methods herein comprise alternating the ultrasonic acoustic energy applied between a first ultrasonic acoustic energy having a first MI and a second ultrasonic acoustic energy having a second MI. In some embodiments, applying alternating ultrasonic acoustic energy administered to a subject between a first MI and a second MI is performed repeatedly over a number of times, such as to enhance gene transfection into the target cells, tissue or organ (e.g., relative to a similar process wherein a first and second ultrasonic acoustic energy are not used and/or are not alternately applied and/or are not alternately applied repeatedly).

In certain embodiments, changing parameters of the ultrasound acoustic energy or MI can be performed to induce and/or enhance an expression of a transgene in a cell or an organ of a subject. In one aspect, provided herein are methods of transfection by alternating the ultrasonic acoustic energy using a first MI and a second MI. In some embodiments, the first MI that results in stable vibrational cavitation is applied prior to the second MI, which results in inertial cavitation. In some embodiments, the ultrasonic acoustic energy using the first MI and the second MI are reapplied a number of times to increase transfection efficiency at the target cell. In some embodiments, during the application of sonoporation, the ultrasonic acoustic energy is applied at the first MI continuously except for when the ultrasonic acoustic energy is applied at the second MI. For example, applying an ultrasonic acoustic energy to the target cell at the first MI then applying an ultrasonic acoustic energy to the target cell at the second MI are repeated between 4 to 18 times. In some embodiments, applying an ultrasonic acoustic energy to the target cell at the first MI then applying an ultrasonic acoustic energy to the target cell at the second MI are repeated an unlimited number of times. In one aspect, during this time, the ultrasonic acoustic energy of the first MI is applied continuously except for when the ultrasonic acoustic energy of the second MI is applied.

In some embodiments, the first MI ranges from about 0.05 to about 0.4. In some embodiments, the first MI ranges from about 0.05 to about 0.3. In some embodiments, the first MI ranges from about 0.05 to about 0.4. In some embodiments, the first MI ranges from about 0.09 to about 0.3.

In some embodiments, the second MI ranges from about 0.5 to about 2.0. In some embodiments, the second MI ranges from greater than 1.4 to about 1.8. In some embodiments, the second MI ranges from greater than 1.4 to about 2.0. In some embodiments, the second MI ranges from about 1.5 to about 2.0.

In some embodiments, applying the ultrasonic acoustic energy at the first MI, and the applying the ultrasonic acoustic energy at the second MI are repeated at least 4, 6, 8, 12, 18, 20, 25, 30, 40, or 50 times. In some embodiments, applying the ultrasonic acoustic energy at the first MI, and the applying the ultrasonic acoustic energy at the second MI are repeated between 4 and 18 times. In some embodiments, applying the ultrasonic acoustic energy at the first MI, and the applying the ultrasonic acoustic energy at the second MI are repeated between 6 and 12 times. In some embodiments, applying the ultrasonic acoustic energy at the first MI, and the applying the ultrasonic acoustic energy at the second MI are repeated between 8 and 10 times. In some embodiments, applying the ultrasonic acoustic energy at the first MI, and the applying the ultrasonic acoustic energy at the second MI are repeated between 8 and 18 times.

In some embodiments, the applying the ultrasound acoustic energy comprises applying the ultrasonic acoustic energy at the first MI, and the applying the ultrasonic acoustic energy at the second MI, without ceasing applying the ultrasonic acoustic energy at the first MI, and the applying the ultrasonic acoustic energy at the second MI. In some embodiments, the applying the ultrasound acoustic energy comprises applying the ultrasonic acoustic energy at the first MI except for when the ultrasonic acoustic energy is applied at the second MI. In some embodiments, an ultrasound probe applying the ultrasonic acoustic energy is in constant contact with the surface of the subject's skin at the location of application (e.g., abdomen, chest wall, skull, etc.). In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In certain embodiments, a transitory (e.g., third, fourth, etc.) ultrasonic acoustic energy is applied between application of the first and second ultrasonic acoustic energies. In certain embodiments, applying the ultrasound acoustic energy comprises applying the ultrasonic acoustic energy without regard to an EKG gating signal regulating the application of the ultrasound acoustic energy. In certain embodiments, applying the ultrasound acoustic energy comprises applying the ultrasonic acoustic energy without turning off power to the ultrasound transducer off. In some embodiments, applying the ultrasound acoustic energy comprises an ultrasound transducer sending ultrasound acoustic energy or receiving reflected ultrasound acoustic energy at least 95% of a period of time in which an ultrasound transducer continuously is contacting the subject.

In some instances, the ultrasonic acoustic energy of the second MI (e.g., high MI) is applied using a pulse. In some instances, a pulse comprises applying the ultrasonic acoustic energy in a short pulse (e.g., microsecond length pulse). In some cases, the high MI is applied with the pulse, results in induces inertial cavitation and destruction of the sonoactive microstructure, resulting in the disruption of cell membrane and vascular endothelial integrity, transducing the nucleic acid payload to the cell. In some instances, the pulse is applied with a duration of about 1 μs to about 200 μs. In some instances, the pulse is applied with a duration of about 1 μs to about 200 μs or greater.

In some embodiments, applying the ultrasonic acoustic energy at the second MI comprises applying the ultrasonic acoustic energy at the second MI using a pulse. In some instances, the duration of the second MI applied ranges from 0.1 μs to about 200 μs. In some instances, the duration of the second MI applied ranges from 1 μs to about 200 μs or greater. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 2.3 μs. In some embodiments, applying the ultrasonic acoustic energy at the second MI comprises applying the ultrasonic acoustic energy at the second MI using a pulse with a duration of at least 2.3 μs. In some embodiments, applying the ultrasonic acoustic energy at the second MI comprises applying the ultrasonic acoustic energy at the second MI using a pulse with a duration ranging from 1-500 μs. In some embodiments, applying the ultrasonic acoustic energy at the second MI comprises applying the ultrasonic acoustic energy at the second MI using a pulse with a duration ranging from 0.1-500 μs.

In some cases, alternating the ultrasonic acoustic energy between the first MI and the second MI for a number of times also allows reperfusion of the sonoactive microstructures and the nucleic acid constructs to the target cell, tissue, or organ, following disruption of the sonoactive microstructures within or proximal to the target cell, tissue, or organ.

In some embodiments, the repeating application of ultrasonic acoustic energy between the first MI and the second MI comprises applying the ultrasonic acoustic energy at the first MI for an amount of time sufficient to permit reperfusion of the sonoactive microstructures in a tissue comprising the target cell before reapplying the ultrasonic acoustic energy at the second MI.

In some embodiments, the method comprises applying the ultrasonic acoustic energy at the first MI for 1-30 seconds before repeating the applying the ultrasound acoustic energy of (d). In some embodiments, the method comprises applying the ultrasonic acoustic energy at the first MI for 5-15 seconds before repeating the applying the ultrasound acoustic energy of reapplying the ultrasonic acoustic energy at the second MI. In some embodiments, the method comprises applying the ultrasonic acoustic energy at the first MI for 10 seconds before repeating the applying the ultrasound acoustic energy of reapplying the ultrasonic acoustic energy at the second MI.

In some instances, the duration of the first MI applied ranges from about 2 s to about 30 s. In some embodiments, applying the ultrasonic acoustic energy at the first MI comprises initially applying the ultrasonic acoustic energy at the first MI from about 2 s to about 30 s.

In some embodiments, applying the ultrasonic acoustic energy at the first MI, and the applying the ultrasonic acoustic energy at the second MI are repeated for a total amount of time ranging from about 1 s to about 60 m. In some embodiments, applying the ultrasonic acoustic energy at the first MI, and the applying the ultrasonic acoustic energy at the second MI are repeated for a total amount of time ranging from about 60 s to about 120 s.

In some embodiments, applying the ultrasonic acoustic energy at the first MI induces stable vibration cavitation of the sonoactive microstructures. In some embodiments, applying the ultrasonic acoustic energy at the first MI does not induce substantial disruption of the sonoactive microstructures. In some embodiments, applying the ultrasonic acoustic energy at the first MI does not induce substantial disruption of the sonoactive microstructures in a vasculature space and an extravascular space, or induces stable vibration cavitation of the sonoactive microstructures in a vasculature space and an extravascular space.

In some embodiments, applying the ultrasonic acoustic energy at the first MI induces formation of an intercellular gap or an interendothelial gap or endocytosis. In some embodiments, the intercellular gap or the interendothelial gap ranges from about 10 nm to about 10 μm. In some embodiments, the stable vibration cavitation of the sonoactive microstructures moves the nucleic acid construct from an intravenous space into an interstitial space or into the cytoplasm.

In some embodiments, applying the ultrasonic acoustic energy at the second MI induces inertial cavitation of the sonoactive microstructures to disrupt the sonoactive microstructures. In some embodiments, applying the ultrasonic acoustic energy at the second MI induces inertial cavitation of the sonoactive microstructures to disrupt the sonoactive microstructures in a vasculature space and an extravascular space. In some embodiments, the extravascular spaces comprise an interstitial space, a subcutaneous space, intramuscular or a lymphatic space. In some embodiments, the extravascular spaces comprise an extravascular tissue. In some embodiments, the extravascular tissue comprises an interstitial space, a cytoplasmic space, a subcutaneous, a lymph tissues, muscular or combinations thereof.

In some embodiments, applying the ultrasonic acoustic energy at the second MI induces formation of a pore in a membrane of the cell. In some embodiments, the formation of a pore in a membrane of the cell ranges from about 10 nm to about 10 um.

In some embodiments, the ultrasound acoustic energy is applied using an ultrasound probe applying ultrasound acoustic energy to the tissue. In some embodiments, the acoustic radiation force is applied using an ultrasound probe applying ultrasound acoustic energy to the tissue. In some embodiments, the ultrasound probe comprises a plurality of piezoelectric elements configured to emit ultrasound acoustic energy. In some embodiments, portions of the plurality of piezoelectric elements are arranged in one or more arrays. In some embodiments, the ultrasound probe is a phased array transducer comprising a plurality of piezoelectric elements configured to emit ultrasound acoustic energy. In some embodiments, the ultrasound probe is a phased array ultrasound probe, a linear ultrasound probe, a curvilinear ultrasound probe, a convex array ultrasound probe, an endocavitary ultrasound probe, a 3D ultrasound probe, a 4D ultrasound probe, a Doppler ultrasound probe, or a color doppler ultrasound probe.

In some embodiments, the nucleic acid construct comprises a miniplasmid backbone. As used herein, the term “miniplasmid (mpDNA)” refers to nucleic acid constructs that are smaller in size (i.e., contain fewer base pairs (bp)) than conventional plasmids or pDNA. In some embodiments, mpDNA constructs comprise a backbone smaller than 1 kb. In some embodiments, mpDNA constructs are smaller than 1000 bp excluding an expression cassette. In some embodiments, mpDNA constructs comprise a backbone smaller than 0.5 kb. In some embodiments, mpDNA constructs are smaller than 500 bp excluding an expression cassette. In some embodiments, the miniplasmid does not comprise a bacterial origin of replication. As used herein, the term “Nanoplasmid™” (e.g., Nanoplasmid sourced from Aldevron, Fargo, South Dakota.) refers to a small mpDNA construct that has a plasmid backbone that is less than 500 bp and does not contain an antibiotic resistance gene.

Miniplasmid DNA nucleic acid constructs can be utilized to deliver an expression cassette, a transgene, or a nonendogenous gene to cells in target cell-types, tissues or organs. In some embodiments, the miniplasmid comprises less than 1000 base pairs excluding an expression cassette. In some embodiments, the miniplasmid comprises less than 500 base pairs excluding an expression cassette. In some embodiments, the miniplasmid does not comprise antibiotic resistant genes. In some embodiments, the miniplasmid does not comprise a bacterial genome. In some embodiments, the miniplasmid comprises a therapeutic transgene and/or a regulatory element. In some embodiments, the miniplasmid is a nanoplasmid. In some embodiments, the miniplasmid construct enhances the expression of the nonendogenous gene or a therapeutic transgene when used in conjunction with the disclosed methods and ultrasound acoustic profiles. In some embodiments, the nanoplasmid construct enhances the expression of the nonendogenous gene or a therapeutic transgene. In some embodiments, durability of expression of a protein encoded by the nucleic acid payload may be increased relative to expression of the same protein in a larger plasmid (e.g., a plasmid of greater than 2 kb in length, excluding the transgene). In some embodiments, durability of expression of a protein encoded by the nucleic acid payload may be increased relative to expression of the same protein in another nucleic acid construct.

In some embodiments, the nucleic acid construct is a miniplasmid e.g., a construct comprising a backbone of less than 1000 bp or less than 500 bp) coupled to a nucleic acid payload.

In some embodiments, the miniplasmid further comprises an expression cassette. As used herein, an expression cassette comprises nucleic acid sequences encoding nucleic acid payload, e.g., an expression cassette comprising a transgene. The expression cassette further comprises a regulatory element such as a promoter, enhancer, ribosome binding site, or transcription termination signal.

In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell. In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell.

In some embodiments, the method further includes increasing expression of the nucleic acid payload by increasing the dosage of the nucleic acid payload administered to the subject. In some embodiments, the method further includes increasing expression of the nucleic acid payload by increasing the dosage of the nucleic acid payload administered to the subject in a linear manner. In some embodiments, the method further includes increasing expression of the nucleic acid payload by administering at least 5, 50, 250, or 500 μg of the nucleic acid payload to the subject.

In some embodiments, the nucleic acid payload comprises an expression cassette. In some embodiments, the expression cassette comprises a transgene. In some embodiments, the nucleic acid payload comprises a transgene (endogenous or non-endogenous). In some embodiments, the transgene comprises a therapeutic transgene. In some embodiments, inducing expression of the nucleic acid payload comprises inducing expression of the therapeutic transgene. In some embodiments, the transgene comprises a detectible marker. In some embodiments, the transgene comprises luciferase. In some embodiments, inducing expression of the nucleic acid payload comprises inducing expression of luciferase.

In some embodiments, a nucleic acid payload comprises a regulatory element such as a promoter, (e.g., APOE-ATT). In some embodiments, a total amount (e.g., dose) of DNA administered to a subject for purposes of sonoporation can range from 100 microgram to 200 mg.

In some embodiments, the therapeutic payload is a nonendogenous gene. In some embodiments, the nucleic acid payload is configured to perform gene augmentation, gene replacement, gene editing, gene knockdown, or gene knockout.

In some embodiments, the nucleic acid construct comprises one or more regulatory elements, such as a promoter, enhancer, ribosome binding site, or transcription termination signal. Examples of promoters contemplated herein include, but are not limited to, e.g., CMV promoter, UbC promoter, CAG promoter, EF-1α promoter, ApoE promoter, ApoE-AAT1 promoter, 3XSERP promoter, or P3-hybrid promoter. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising ApoE-AAT. In some embodiments, the nucleic acid construct comprises a promoter sequence comprising SERP. In some embodiments, the nucleic acid construct comprises a promoter sequence comprising P3.

In some embodiments, inducing expression of the nucleic acid payload comprises inducing production of RNA encoded by the payload. In some embodiments, inducing expression of the nucleic acid payload comprises inducing production of protein encoded by the payload.

In some embodiments, the therapeutic nucleic acid sequence is referred to a payload. In some embodiments, the therapeutic nucleic acid sequence comprises a payload which comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro-RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides). In some embodiments, the nucleic acid construct is configured to perform gene augmentation, gene replacement, base editing, base knockdown, gene editing gene knockdown, or gene knockout. In some embodiments, delivering the nucleic acid payload to the target cell of the subject increases or decreases expression of a gene in the target cell.

In some embodiments, the therapeutic nucleic acid sequence is referred to a payload. In some embodiments, the therapeutic nucleic acid sequence comprises a payload which comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease a regulatory protein or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR).

Sonoactive microstructures (also referred to as acoustic microspheres or “microbubbles”) contemplated herein include, but are not limited to, those used as ultrasonic imaging contrast agents. In some embodiments, the sonoactive microstructures comprise a phospholipid stabilized microstructure. In some embodiments, the phospholipid stabilized microstructure comprises a high molecular wight gas core, or a perflutran core. Examples of sonoactive microstructures include, but are not limited to, OPTISON (GE Healthcare), Sonazoid (GE Healthcare), or DEFINITY and Definity RT (Lantheus Medical Imaging, Inc). In some embodiments, the sonoactive microstructures are LUMASON (Bracco) (sulfur hexafluoride lipid-type A microspheres). In some embodiments, the sonoactive microstructures are SonoVue (sulfur hexafluoride microbubbles). In some embodiments, the sonoactive microstructures comprise a protein stabilized microstructure. In some embodiments, the sonoactive microstructures are Optison microbubbles.

The sonoactive microstructures can be administered prior to, after, or simultaneous (e.g., co-administered) with the administration of the nucleic acid construct (or nucleic acid payload). In some embodiments, the nucleic acid construct and the sonoactive microstructures are coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occur sequentially. In some embodiments, the administering of the nucleic acid construct and the sonoactive microstructures occurs continuously.

In some embodiments, the nucleic acid construct is administered at a dosage of about 0.5 mg/kg to about 500 mg/kg. In some embodiments, at least 2×10¹³ copies of the nucleic acid construct are administered to the subject. In some embodiments, about 2×10¹³ to about 3×10¹³ copies of the nucleic acid construct are administered to the subject. In some embodiments, each nucleic acid construct comprises a copy of a transgene.

In some embodiments, the sonoactive microstructures are administered at a dosage of about 1-50 mL, for example 1 mL of Optison. The sonoactive microstructures may be administered at a concentration of about 5M to about 8M microstructures per mL. In some embodiments, the sonoactive microstructures are administered at a concentration of about 5×10\hat{ }8 to about 1.2×10\hat{ }9 microstructures/mL, for example 1×10\hat{ }9 of Definity RT. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of about 5×10\hat{ }8 to about 8×10\hat{ }8 microstructures/mL.

As used herein, concentrations of microstructures/mL refers to the concentration of the sonoactive microstructures in a pharmaceutical composition immediately prior to administration to the subject. In some embodiments, the sonoactive microstructures are administered at a concentration of about 5×10⁸ to about 1.2×10¹⁰ microstructures/mL. In some embodiments, the sonoactive microstructures are administered at a dosage of about 1-50 mL, for example 1 mL of a protein-stabilized sonoactive microstructure (e.g., Optison). In some embodiments, the protein-stabilized sonoactive microstructure (e.g., Optison) has a diameter of 3-4.5 micrometers. The sonoactive microstructures may be administered at a concentration of about 5M (million) to about 8M microstructures per mL. In some embodiments, 1×10⁹ of phospholipid stabilized sonoactive microstructures (e.g., Sonazoid) are administered. In some embodiments, the phospholipid stabilized sonoactive microstructures (e.g., Sonazoid) comprise a diameter of 1-5 micrometers. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of at least 5×10\hat{ }8 microstructures per mL. In some embodiments, the sonoactive microstructures are administered at a concentration of up to 1.2×10\hat{ }10 microstructures/mL. In some embodiments, the sonoactive microstructures are administered at a concentration of 5×10\hat{ }8 to 8×10\hat{ }8 microstructures/mL.

In some embodiments, the nucleic acid construct and the sonoactive microstructures are mixed prior to being coadministered. In some instances, the sonoactive microstructures are mixed with the nucleic acid constructs before administering to the subject. In some instances, the sonoactive microstructures are mixed with the nucleic acid constructs along with additional buffers or agents such as saline or other biocompatible solutions with varying electrostatic charges and surface chemistries and ligands before administering to the subject. For example, Optison sonoactive microstructures can be mixed with a Nanoplasmid comprising a promoter operatively linked to a transgene (e.g., APOE-Fluc) and saline and are administered together. In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures.

In some embodiments, the administering of the nucleic acid construct and the sonoactive microstructures is by intravenous administration or subcutaneous or intramuscular or intra-arterial or inter-osseus or direct organ puncture. In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures. In some cases, mixtures may comprise a ratio of one part solution comprising the nucleic acid constructs to four or more parts solution comprising the sonoactive microstructures.

In some embodiments, after administering of the nucleic acid construct and sonoactive microstructures, the ultrasound acoustic energy is applied at the target cell, tissue, or organ.

In some embodiments, inducing expression of the nucleic acid payload comprises inducing expression within about 3 to about 12 hours of administering the payload. In some embodiments, inducing expression of the nucleic acid payload comprises inducing expression within about 3 hours of administration. In some embodiments, inducing expression of the nucleic acid payload comprises inducing expression within about 6 hours of administration. In some embodiments, inducing expression of the nucleic acid payload comprises inducing expression within about 12 hours of administration.

Undesirable effects on living cells or tissues can occur due to ultrasound applications. In some embodiments, the present disclosure provides methods for improvement of gene transfection and not result in substantial DNA or cell damage in the target cells, tissues, or organs, using sonoporation by alternating ultrasonic acoustic energy between the first MI and the second MI. In some embodiments, the method does not result in substantial cellular damage to the target cell. In some embodiments, the method results in less than 1%, 5%, or 10% of target cells undergoing apoptosis.

Cellular damage can be detected using apoptotic biomarkers. For example, in liver, detection of released hepatocellular transaminases, e.g., serum alanine aminotransferase (ALT) or aspartate aminotransferase (AST), can be an indicator of apoptotic hepatocytes. Additional apoptotic biomarkers comprise interleukin 6 (IL6) or B-cell lymphoma 2 (BCL2 or BCL2 apoptosis regulator). In some embodiments, the following biomarkers for cellular damage are not detected at apoptotic levels following delivering the nucleic acid payload to the target cell of the subject: ALT, AST, IL6, BCL2, or combinations thereof. In some embodiments, the following biomarkers for cellular damage are not clinically elevated following delivering the nucleic acid payload to the target cell of the subject: ALT, AST, IL6, BCL2, or combinations thereof. In some embodiments, the following biomarkers for cellular damage are not detected at apoptotic levels following delivering the nucleic acid payload to the target cell of the subject: ALT, AST, IL6, BCL2, or combinations thereof, and, optionally wherein the target cell is in a liver. In some embodiments, the following biomarkers for cellular damage are not clinically elevated following delivering the nucleic acid payload to the target cell of the subject: ALT, AST, 1L6, BCL2, or combinations thereof, and, optionally wherein the target cell is in a liver. In some embodiments, the following biomarkers for cellular damage are not clinically elevated following delivering the nucleic acid payload to the target cell of the subject: creatinine levels in urine, albumin to creatine ratio in urine, creatinine levels in blood, a glomerular filtration rate, blood in urine, protein levels in urine, or an osmolality of urine, and, optionally wherein the target cell is in a kidney. In some embodiments, the following biomarkers for cellular damage are not clinically elevated following delivering the nucleic acid payload to the target cell of the subject: troponin levels in blood, or creatinine phosphokinase, and, optionally wherein the target cell is in a heart or skeletal muscle. In some embodiments, ALT is not detected at levels exceeding 60, 65, 70, 75, 80, 85, 90, 95, 100, 125, 150, 175, or 200 U/L following delivering the nucleic acid payload to the target cell of the subject. In some embodiments, AST is not detected at levels exceeding 225, 250, 275, or 300 U/L following delivering the nucleic acid payload to the target cell of the subject. In some embodiments, IL6 is not detected at levels exceeding 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, or 6 μg/mL following delivering the nucleic acid payload to the target cell of the subject.

A sonoporation treatment using the methods described herein can be used to induce expression of a nucleic acid payload in a cell in a liver or a cell in a kidney.

In another aspect, the present disclosure provides a kit to perform the methods described herein. In some embodiments, the kit comprises: (a) a first container comprising microbubbles for sonoporation; and (b) a second container comprising nucleic acids (e.g., miniplasmids) comprising a transgene and a mixture chamber (reservoir, syringe, Y-port, etc.). In some embodiments, the kit further comprises instructions for administration of ultrasound acoustic energy in connection with administration of the microbubbles and the nucleic acids.

In some embodiments, the first container and second container are configured to induce the expression of the transgene in the target cell of the subject within 20 hours after the transfection.

In some embodiments, the kit further comprises instructions for software and hardware directions for the safe and effective operation of an ultrasound machine sufficient to disrupt the sonoactive microstructures to generate the sonoporation processes which include but are not limited to the following: disrupting the microstructures, inducing inertial and stable cavitation, promoting endocytosis and inter-endothelial gap formation, microstreaming at cell surfaces, thereby increasing transfection of a nucleic acid payload to a cell. In some embodiments, the instructions described methods for improvement of gene transfection using sonoporation by applying alternating ultrasonic acoustic energy applying an acoustic radiation force to a subject and inducing a shear wave in a tissue comprising the target cell. In some embodiments, the instructions described methods for improvement of gene transfection using sonoporation by applying alternating ultrasonic acoustic energy between a first MI then a second MI. In some embodiments, the kit further comprises instructions for administration of the first container and the second container. Aspects disclosed herein provide a kit comprising: a sonoactive agent; and means for expressing a Factor VIII polypeptide in vivo at a level that is at least 1.5 fold greater than expression of SEQ ID NO. 3 Aspects disclosed herein provide a kit comprising: a sonoactive agent; and the nucleic acid composition of any embodiment disclosed herein. Aspects disclosed herein provide a cell comprising the FVIII polypeptide of any embodiment disclosed herein. Aspects disclosed herein provide a nucleic acid encoding the FVIII polypeptide. In some cases, the means for increasing expression of a Factor VIII polypeptide in vivo, for example, at a level that is at least 1.5 fold greater than expression of SEQ ID NO. 3, includes: a nucleic acid sequence having at least 80% sequence identity to SEQ ID NO: 6; a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence; a first regulatory element positioned upstream and directly coupled to the therapeutic nucleic acid sequence, a second regulatory element positioned downstream and directly coupled to the therapeutic nucleic acid sequence, the poly-adenylation signal positioned downstream and directly coupled to the second regulatory element, and the nuclear targeting sequence is positioned downstream and directly coupled to the poly-adenylation signal, one or more Inverted Terminal Repeat (ITR) sequences; a nucleic acid sequence comprising a sequence of any one of SEQ ID NO: 38, 39, or 44; and combinations thereof.

### Exemplary Embodiments

Among the exemplary embodiments are:

1. A nucleic acid composition comprising a nucleic acid sequence having at least 80% sequence identity to SEQ ID NO: 6.
2. A nucleic acid composition comprising a therapeutic nucleic acid sequence, a first regulatory element comprising a promoter sequence operably linked to an intron sequence comprising a hemoglobin subunit gamma intron (hBGi) sequence positioned upstream of the therapeutic nucleic acid sequence, and a second regulatory element comprising a woodchuck hepatitis posttranscriptional regulatory element 3 (WPRE3) sequence positioned downstream of the therapeutic nucleic acid sequence.
3. A nucleic acid composition comprising a nucleic acid sequence having at least 96% sequence identity to SEQ ID NO: 45.
4. The nucleic acid composition of embodiment 2, further comprising a nucleic acid sequence having at least 80% sequence identity to SEQ ID NO: 6.
5. The nucleic acid composition of embodiment 1, further comprising a therapeutic nucleic acid sequence, wherein the nucleic acid sequence having at least 80% sequence identity to SEQ ID NO: 6 increases expression of the therapeutic nucleic acid sequence in a cell by at least 1.25-fold as compared to an otherwise identical control composition lacking the nucleic acid sequence having at least 80% sequence identity to SEQ ID NO: 6.
6. The nucleic acid composition of embodiment 1 or embodiment 4, further comprising a first regulatory element comprising a promoter sequence operably linked to an intron sequence, and a second regulatory element comprising a WPRE3 sequence.
7. The nucleic acid composition of any one of embodiments 2 or 6, wherein the first regulatory element is upstream of the therapeutic nucleic acid sequence.
8. The nucleic acid composition of any one of embodiments 2, 6, or 7, wherein the second regulatory element is downstream of the therapeutic nucleic acid sequence.
9. The nucleic acid composition of embodiment 8, wherein the each of the first regulatory element and the second regulatory element are operably linked to the therapeutic nucleic acid sequence.
10. The nucleic acid composition of any one of embodiments 1 or 4, wherein the nucleic acid sequence having at least 80% sequence identity to SEQ ID NO: 6 has at least 81, 82, 83, 84, 85, 86, 87, 88, 89, 90, 91, 92, 93, 94, 95, 96, 97, 98, or 99% sequence identity or has 100% sequence identity to SEQ ID NO: 6.
11. The nucleic acid composition of any one of embodiments 1, 4 or 10, wherein the nucleic acid sequence having at least 80% sequence identity to SEQ ID NO: 6 is operably coupled to a second regulatory element comprising a WPRE3 sequence.
12. The nucleic acid composition of any one of embodiments 2, or 6-9, wherein the first regulatory element comprises a sequence of SEQ ID NO: 9.
13. The nucleic acid composition of any one of embodiments 2, or 6-10, wherein the second regulatory element comprises a sequence of SEQ ID NO: 4.
14. The nucleic acid composition of any one of embodiments 1, 4, or 10-11, wherein the nucleic acid sequence having at least 80% sequence identity to SEQ ID NO: 6 comprises a nuclear targeting sequence, wherein the nuclear targeting sequence increases expression of the therapeutic nucleic acid sequence in a cell by at least 1.25, 1.5, 2, 3, 4, 5, 6, 7, or 8-fold, as compared to an otherwise identical control composition that lacks the nuclear targeting sequence.
15. The nucleic acid composition of any one of embodiments 5 or 14, wherein the cell is a liver cell, a kidney cell, a brain cell, a muscle cell, a cardiac cell, a pancreatic cell, a blood cell, or a tumor cell.
16. The nucleic acid composition of embodiment 15, wherein the cell is a hepatocyte.
17. The nucleic acid composition of any one of embodiments 2, 5, or 7-9, or 14 wherein the therapeutic nucleic acid sequence comprises a sequence encoding a therapeutic transgene.
18. The nucleic acid composition of embodiment 17, wherein the therapeutic transgene encodes a FVIII, FIX, COL4A3, COL4A4, COL4A5, PKD1, or a PKD2 protein.
19. The nucleic acid composition of any one of embodiments 2, 5, or 7-9, or 14, wherein the therapeutic nucleic acid sequence comprises a CRISPR/Cas system, a DNA molecule suitable for knock-in via homologous end joining (NHEJ), a DNA molecule suitable for homology directed repair (HDR), a gRNA, an RNA interference (RNAi) agent, a micro-RNA (miRNA), a short interfering RNA (siRNA), short hairpin RNA (shRNA), or a triplex-forming oligonucleotide, a catalytically active RNA molecule, a transfer RNA (tRNA), a base editor, a prime editor, a recombinase, an integrase, a transposase, a reverse transcriptase, a spliceosomal RNA, an adenosine deaminase, or combinations thereof.
20. The nucleic acid composition of any one of embodiments 2, 5, or 7-9, or 14, wherein the therapeutic nucleic acid sequence encodes a therapeutic agent comprising a Cas protein or homolog or variant thereof, a TALEN, a ZFN, or combinations thereof.
21. The nucleic acid composition of any one of the preceding embodiments, wherein the nucleic acid composition comprises a miniplasmid backbone.
22. The nucleic acid composition of embodiment 21, wherein the miniplasmid backbone is smaller than 1 kb.
23. The nucleic acid composition of embodiment 21, wherein the miniplasmid backbone comprises a sequence of SEQ ID NO: 7.
24. The nucleic acid composition of any one of embodiments 2, 5, or 7-9, or 14, wherein the therapeutic nucleic acid sequence encodes a protein which provides a therapeutic effect to a subject.
25. The nucleic acid composition of any one of the preceding embodiments, wherein the nucleic acid composition is an isolated nucleic acid molecule.
26. The nucleic acid composition of any one of the preceding embodiments, wherein the nucleic acid composition does not comprise an origin of replication or a bacterial origin of replication.
27. The nucleic acid composition of any one of embodiments 2 or 6, wherein the intron sequence comprises a hemoglobin subunit gamma intron (hBGi) sequence.
28. The nucleic acid composition of any one of the preceding embodiments, further comprising a poly-adenylation signal.
29. The nucleic acid composition of embodiment 28, wherein the poly-adenylation signal is positioned downstream of the second regulatory element.
30. The nucleic acid composition of embodiment 28, wherein the poly-adenylation signal is positioned downstream of the therapeutic nucleic acid sequence.
31. The nucleic acid composition of embodiment 28, wherein the poly-adenylation signal is positioned upstream of a nuclear targeting sequence.
32. The nucleic acid composition of any one of embodiments 2 or 6, wherein the promoter sequence comprises an Apolipoprotein E-Alpha-1-Antitrypsin (APOE-AAT) promoter sequence.
33. The nucleic acid composition of embodiment 31, wherein the first regulatory element is positioned upstream and directly coupled to the therapeutic nucleic acid sequence, the second regulatory element is positioned downstream and directly coupled to the therapeutic nucleic acid sequence, the poly-adenylation signal is positioned downstream and directly coupled to the second regulatory element, and wherein the nuclear targeting sequence is positioned downstream and directly coupled to the poly-adenylation signal.
34. The nucleic acid composition of any one of the preceding embodiments, wherein the nucleic acid composition further comprises one or more Inverted Terminal Repeat (ITR) sequences.
35. The nucleic acid composition of embodiment 34, wherein a first ITR sequence is positioned upstream of the therapeutic nucleic acid sequence, promoter sequence, first regulatory element, and/or nuclear targeting sequence.
36. The nucleic acid composition of embodiment 35, wherein a second ITR sequence is positioned downstream of the therapeutic nucleic acid sequence, promoter sequence, first regulatory element, and/or nuclear targeting sequence.
37. The nucleic acid composition of any one of the preceding embodiments, wherein the therapeutic nucleic acid sequence comprises a nucleic acid sequence of SEQ ID NO: 32, or of SEQ ID NO: 33.
38. The nucleic acid composition of any one of the preceding embodiments, wherein the nucleic acid composition is a non-viral vector.
39. The nucleic acid composition of embodiment 37, wherein the nucleic acid composition is at least 5000, 5500, 6000, 6500, or 7000 bp in length.
40. The nucleic acid composition of any one of the preceding embodiments, wherein the nucleic acid composition encodes a full-length gene.
41. The nucleic acid composition of any one of the preceding embodiments, wherein the nucleic acid composition encodes a nucleic acid sequence of SEQ ID NO: 38 or SEQ ID NO:
44.
42. The nucleic acid composition of any one of the preceding embodiments, wherein the nucleic acid composition encodes a nucleic acid sequence of SEQ ID NO: 43.
43. The nucleic acid composition of any one of the preceding embodiments, wherein the nucleic acid composition comprises a sequence of SEQ ID NO: 45.
44. The nucleic acid composition of any one of the preceding embodiments, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of:
(a) the A1 domain comprises at least one amino acid substitution relative to SEQ ID NO: 36; and
(b) the A3 domain comprises at least two amino acid substitutions as compared to SEQ ID NO: 37.
   45. A nucleic acid composition comprising a therapeutic nucleic acid sequence, wherein the therapeutic nucleic acid sequence encodes a FVIII polypeptide having an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and wherein one or both of:
(a) the A1 domain comprises at least one amino acid substitution relative to SEQ ID NO: 36; and
(b) the A3 domain comprises at least two amino acid substitutions as compared to SEQ ID NO: 37.
   46. The nucleic acid composition of embodiment 44 or 45, wherein the therapeutic nucleic acid sequence encodes a Factor VIII (FVIII) polypeptide comprising an amino acid sequence having up to 2 amino acid substitutions relative to SEQ ID NO: 24.
   47. The nucleic acid composition of any one of embodiments 44-46, wherein the B domain comprises no more than 6 consensus sites for N-linked glycosylation.
   48. The nucleic acid composition of any one of embodiments 44-47, wherein the B domain comprises an amino acid sequence of SEQ ID NO: 28, and wherein the FVIII polypeptide is not more than 2300 amino acids in length.
   49. The nucleic acid composition of any one of embodiments 44-48, wherein the B domain comprises an amino acid sequence of SEQ ID NO: 34.
   50. The nucleic acid composition of any one of embodiments 44-49, wherein the A1 domain comprises substitution of phenylalanine at position 328 to serine when the Factor VIII polypeptide is aligned with SEQ ID NO: 35.
   51. The nucleic acid composition of any one of embodiments 44-50, wherein the A1 domain comprises an amino sequence of SEQ ID NO: 25.
   52. The nucleic acid composition of any one of embodiments 44-51, wherein the A3 domain comprises substitution of cysteine at position 1249 to glycine when the Factor VIII polypeptide is aligned with SEQ ID NO: 35.
   53. The nucleic acid composition of any one of embodiments 44-52, wherein the A3 domain comprises substitution of cysteine at position 1253 to glycine when the Factor VIII polypeptide is aligned with SEQ ID NO: 35.
   54. The nucleic acid composition of any one of embodiments 44-53, wherein the A3 domain comprises an amino sequence of SEQ ID NO: 29.
   55. The nucleic acid composition of any one of embodiments 44-54, comprising an amino acid sequence of SEQ ID NO: 24.
   56. The nucleic acid composition of any one of embodiments 44-55, for use a method of treating a genetic disorder requiring a gene therapy or a protein replacement therapy in a subject in need thereof, the method comprising administering the nucleic acid composition of any one of embodiments 44-55 to the subject.
   57. The nucleic acid composition of any one of embodiments 44-55, for use a method of treating a bleeding disorder in a subject in need thereof, the method comprising administering the nucleic acid composition of any one of embodiments 44-55 to the subject.
   58. The nucleic acid composition of embodiment 57, wherein the bleeding disorder is hemophilia A.
   59. A cell comprising the nucleic acid composition of any one of the preceding embodiments.
   60. A method of expressing a nucleic acid payload in a cell, the method comprising administering the nucleic acid composition of any one of the preceding embodiments to a subject.
   61. The method of embodiment 60, further comprising applying an ultrasonic acoustic energy to the cell.
   62. The method of any one of embodiments 61, further comprising administering to the subject a sonoactive agent.
   63. The method of any one of embodiments 60, wherein the nucleic acid composition is administered to the subject as a naked nucleic acid.
   64. The method of embodiment 60, further comprising contacting the cell with a lipid nanoparticle composition.
   65. The method of embodiment 60, wherein the nucleic acid composition is administered to the subject as an encapsulated nucleic acid.
   66. The method of embodiment 65, wherein the nucleic acid composition is encapsulated in a lipid or a polymer.
   67. The method of embodiment 60, wherein the nucleic acid composition is administered to the subject as cationic polymer.
   68. The method of embodiment 60, wherein the nucleic acid composition is administered to the subject in a viral vector.
   69. The method of embodiment 61, further comprising administering to the subject a plurality of magnetic particles and applying a magnetic field to the cell.
   70. The method of embodiment 69, wherein the magnetic particles comprise paramagnetic nanoparticles.
   71. The method of any one of embodiments 60, or 68-69, further comprising applying an electrical field to the cell.
   72. The method of any one of embodiments 60-71, wherein the nucleic acid composition is administered to the subject using a hydrodynamic injection.
   73. The method of any one of embodiments 60-72, wherein the nucleic acid composition encodes a full-length polypeptide, wherein the method induces expression of the full-length polypeptide in-vivo.
   74. The method of any one of embodiments 60-73, wherein the subject has a bleeding disorder, wherein the therapeutic nucleic acid sequence encodes FVIII or FIX, and wherein the method thereby treats the subject having the bleeding disorder.
   75. The method of embodiment 74, wherein the bleeding disorder is hemophilia A.
   76. A method of manufacturing the nucleic acid composition of any one of embodiments 1-55.
   77. A Factor VIII polypeptide comprising:
an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and
wherein one or both of:
(a) the A1 domain comprises at least one amino acid substitution relative to SEQ ID NO: 36; and
(b) the A3 domain comprises at least two amino acid substitutions as compared to SEQ ID NO: 37.
   78. A Factor VIII polypeptide comprising an amino acid sequence having no more than 2 amino acid substitutions relative to SEQ ID NO: 24.
   79. The FVIII polypeptide of embodiment 77, comprising an amino acid sequence having no more than 2 amino acid substitutions relative to SEQ ID NO: 24.
   80. The FVIII polypeptide of embodiment 78, comprising:
an A1 domain, and A2 domain, a non-naturally occurring B domain (B), an A3 domain, a C1 domain, and a C2 domain, positioned from N- to C-terminus as A1-A2-B-A3-C1-C2, wherein the B domain comprises SEQ ID NO: 27; and
wherein one or both of:
(a) the A1 domain comprises at least one amino acid substitution relative to SEQ ID NO: 35; and
(b) the A3 domain comprises at least two amino acid substitutions as compared to SEQ ID NO: 36.
   81. The FVIII polypeptide of any one of embodiments 77-80, wherein the B domain comprises no more than 6 consensus sites for N-linked glycosylation.
   82. The FVIII polypeptide of any one of embodiments 77-81, wherein the B domain comprises an amino acid sequence of SEQ ID NO: 28 and wherein the FVIII polypeptide is not more than 2300 amino acids in length.
   83. The FVIII polypeptide of any one of embodiments 77-82, wherein the B domain comprises an amino acid sequence of SEQ ID NO: 34.
   84. The FVIII polypeptide of any one of embodiments 77-83, wherein the A1 domain comprises substitution of phenylalanine at position 328 to serine when the FVIII polypeptide is aligned with SEQ ID NO: 35.
   85. The FVIII polypeptide of any one of embodiments 77-84, wherein the A1 domain comprises an amino sequence of SEQ ID NO: 25.
   86. The FVIII polypeptide of any one of embodiments 77-85, wherein the A3 domain comprises substitution of cysteine at position 1249 to glycine when the FVIII polypeptide is aligned with SEQ ID NO: 35.
   87. The FVIII polypeptide of any one of embodiments 77-86, wherein the A3 domain comprises substitution of cysteine at position 1253 to glycine when the FVIII polypeptide is aligned with SEQ ID NO: 35.
   88. The FVIII polypeptide of any one of embodiments 77-87, wherein the A3 domain comprises an amino sequence of SEQ ID NO: 29.
   89. The FVIII polypeptide of any one of embodiments 77-88, comprising the amino acid sequence of SEQ ID NO: 24.
   89A. The FVIII polypeptide of any one of embodiments 77-89, wherein the FVIII polypeptide comprises an improved coagulation activity as compared to a FVIII polypeptide lacking one or more of: a B domain other than SEQ ID NO: 27, an A1 domain lacking substitution of phenylalanine at position 328 to serine, or an A3 domain lacking substitution of cysteine at position 1249 to glycine.
   90. The FVIII polypeptide of any one of embodiments 77-89A, for use a method of treating a bleeding disorder in a subject in need thereof, the method comprising administering the FVIII polypeptide to the subject.
   91. A kit comprising:
a. a sonoactive agent; and
b. means for expressing a Factor VIII polypeptide in vivo at a level that is at least 1.5 fold greater than expression of SEQ ID NO. 3.
   92. A kit comprising:
a. a sonoactive agent; and
b. the nucleic acid composition of any one of embodiments 1-55.
   93. A cell comprising the FVIII polypeptide of any one of the preceding embodiments.
   94. A nucleic acid encoding the FVIII polypeptide of any one of the preceding embodiments.
   95. The nucleic acid composition of any one of the preceding embodiments, wherein the therapeutic nucleic acid sequence encodes the FVIII polypeptide of any one of the preceding embodiments.
   96. A cell comprising the nucleic acid of any one of embodiments 94-95.
   97. A method of treating a bleeding disorder comprising administering to a subject a nucleic acid encoding a human clotting factor in at least two treatments at least 48 hours apart, thereby achieving a therapeutic level of the clotting factor in the subject.
   98. The method of embodiment 97, wherein the therapeutic level of the clotting factor is maintained for at least 10, 20, 30, 40, 50, 60, 70, 80, 90, 100, 110, 120, 130, 140, 145, or 150 days.
   99. The method of any one of embodiments 97-98, further comprising administering to the subject ultrasound acoustic energy in the treatments.
   100. The method of any one of embodiments 97-99, further comprising administering to the subject a sonoactive agent in the treatments.
   101. The method of embodiment 99, wherein administering the ultrasound acoustic energy comprises applying the ultrasound energy at a mechanical index (MI) of at least 0.8, 1.3, 1.8, 1.9, or 2.2.
   102. The method of embodiment 99, wherein administering the ultrasound acoustic energy comprises applying the ultrasound energy at a mechanical index (MI) of up to 0.4.
   103. The method of embodiment 99, wherein administering the ultrasound energy comprises applying an acoustic radiation force to the subject.
   104. The method of embodiment 103, wherein the ultrasound energy is applied at an intensity (ISPTA) of at least 200 mW/cm2.
   105. The method of embodiment 103, wherein the ultrasound energy is applied at pulse length of at least 20 microseconds.
   106. The method of embodiment 100, wherein the subject is administered at least three treatments.
   107. The method of embodiment 100, wherein the sonoactive agent comprises protein-stabilized microstructures.
   108. The method of embodiment 100, wherein the sonoactive agent comprises a shell filled with a perfluorinated gas.
   109. The method of embodiment 100, wherein the sonoactive agent comprises lipid-stabilized microstructures.
   110. The method of embodiment 106, wherein the at least three treatments are administered at least 48 hours apart.
   111. The method of embodiment 100, wherein the subject is administered at least two cycles, each cycle comprising at least two treatments.
   112. The method of embodiment 100, wherein the subject is administered at least three cycles, each cycle comprising at least two treatments.
   113. The method of embodiment 100, wherein the subject is administered at least three cycles, each cycle comprising at least three treatments.
   114. The method of any one of embodiments 111-113, wherein each cycle is administered to the subject at least 10 days apart.
   115. The method of any one of embodiments 111-113, wherein each cycle is administered to the subject at least 30 days apart.
   116. The method of any one of embodiments 111-113, wherein each cycle is administered to the subject at least 40 days apart.

### EXAMPLES

### Example 1. In Vitro Expression of FVIII and FIX Coding Plasmids in HepG2 Cell Lines

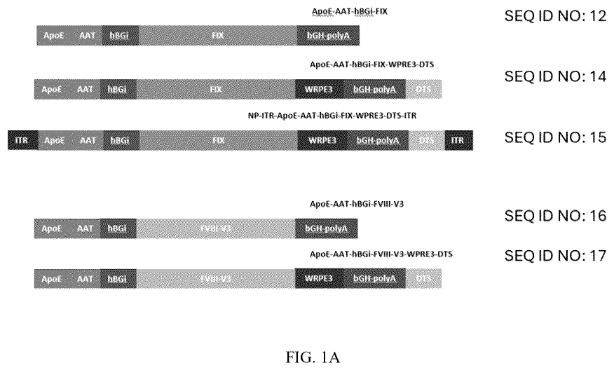

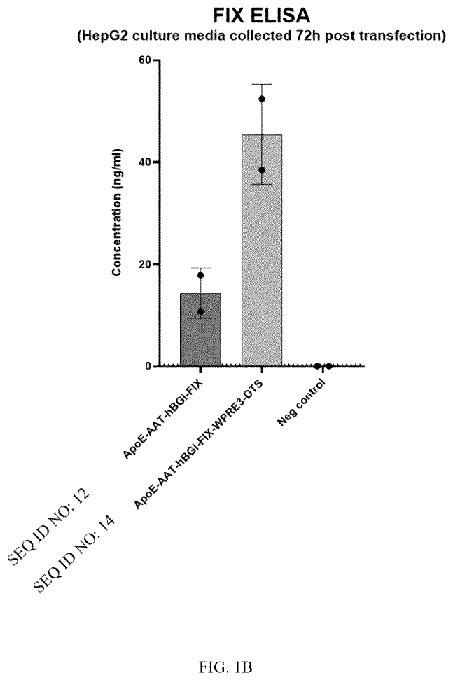

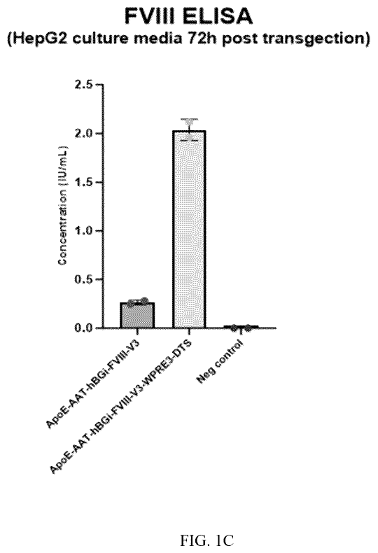

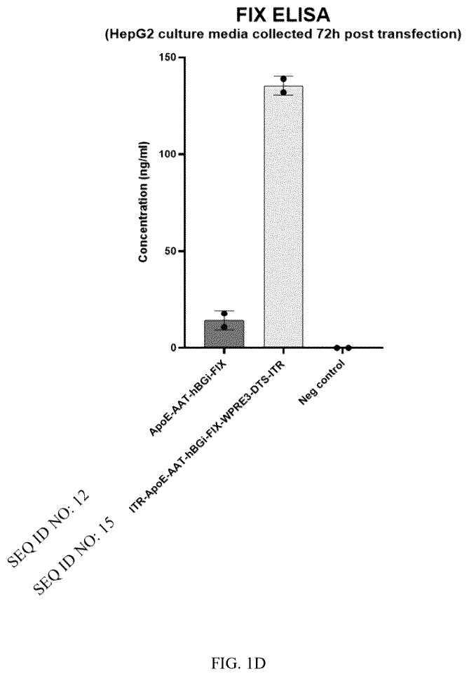

In this example, in-vitro expression of therapeutic nucleic acids encoding FIX and FVIII coding nucleic acid sequences in vectors comprising genetic regulatory elements which increase expression transfected to Human Liver Hepatocellular Cell Line HepG2 were evaluated. Plasmid DNA (pDNA) encoding FIX and FVIII coupled to promoters and genetic regulatory elements were produced. Circular DNA sequences encoding the sequence of SEQ ID NO: 12 (APOE-AAT-hbGi-FIX), SEQ ID NO: 14 (APOE-AAT-hbGi-FIX-WPRE3-bGH-polyA-DTS), SEQ ID NO: 15 (APOE-AAT-hbGi-FIX WPRE3-bGH-polyA-DTS-ITR), SEQ ID NO: 16 (APOE-AAT-hbGi-FVIII-v3-bGH-polyA), and SEQ ID NO: 17 (APOE-AAT-hbGi-FVIII-v3-WPRE3-bGH-polyA-DTS) were manufactured. Vector maps of nucleic acid sequences encoding the therapeutic nucleic acid in vectors comprising genetic regulatory elements which increase expression of the therapeutic nucleic acid were illustrated in FIG. 1A. Parts of the DNA vectors were synthesized (GeneScript) and combined in final pDNA vectors by NEBuilder® HiFi DNA Assembly (NEB) following manufacture's protocol. In brief, DNA fragments were amplified by PCR reaction utilized Phusion® High-Fidelity DNA Polymerase and primers designed with NEBuilder Assembly Tool. PCR fragments were purified from agarose gel utilizing NED Monarch Gel Extraction kit (NEB) and assembled in HiFi DNA assembly reaction. pDNA sequence integrity was confirmed by next generation sequencing methods.

Following confirmation of the correct pDNA sequences, the pDNA constructs were transfected to HepG2 cell lines using a lipid-nanoparticle based Lipofectamine® P3000 transfection reagent. 100 ng of pDNA, with 5 μL of Opti-MEM™ Reduced-Serum Medium 0.1 uL, and 0.1 of P3000 transfection reagent were combined in a 96 well plate and incubating at room temperature for 5 min. Negative controls were prepared by combining 5 μL of Opti-MEM™ Reduced-Serum Medium 0.1 uL, and 0.15 of P3000 transfection reagent, and incubating at room temperature for 5 min. The HepG2 cell were then added, were cultured, and media collected at 72 hours post transfection and frozen at −80 C.

FIX samples were later defrosted, diluted 5-fold, and analyzed using ELISA Green Mountain Antibodies for detection of human FIX. Concentration of secreted transgenic human FIX in culture media 72 hours post transfection was measured by ELISA utilizing Green Mountain Antibodies. Approximately 100 μL of capture antibody (GMA-102) was incubated at 4 C overnight. The stock solution of the capture antibody was diluted to 2 μg/mL antibody solution. A 96 well plate was washed three times with 300 μL of wash buffer, and 300 μL of block buffer was added, and incubated at room temperature for 30 min. A standard serial dilutions were prepared using a recombinant FIX (Abcam 1.8 mg/mL). 100 uL samples were 5-fold. A 96 well plate was washed three times with 300 μL of wash buffer, and 100 μL of block buffer and FIX standard were added, and incubated at room temperature for 60 min. The 96 well plate was again washed three times with 300 μL of wash buffer, and 100 μL of detection antibody (GMA-184) per well was added and incubated at room temperature for 60 minutes. The 96 well plate was washed three times with 300 μL of wash buffer, and 100 μL of streptavidin peroxidase per well was added, an incubated at room temperature for 30 minutes. The 96 well plate was washed three times with 300 μL of wash buffer, and 100 μL of o-Phenylenediamine (OPD) substrate (dissolved 1 tablet (5 mg) in 12 mL citrate buffer, then added 5 ul 30% H₂O₂) was added and incubated at room temperature for 15 min. 50 μL of 2N H2SO4 was added, and absorbance measurements were taken on the SpectraMax at 490 nm and 570 nm. An asymmetric sigmoidal, 5PL, x=concentration analysis was used to interpolate sample values based on the standard curves, and a limit of detection of 0.8 ng/mL of FIX was established for this ELISA plate.

FVIII samples were later defrosted, diluted 5-fold, and analyzed using ELISA Green Mountain Antibodies for detection of human FVIII. Concentration of secreted transgenic human FVIII in culture media 72 hours post transfection was measured by ELISA utilizing Green Mountain Antibodies. Approximately 100 μL of capture antibody (GMA-8024) was incubated at 4 C overnight. The stock solution of the capture antibody was diluted to 2 μg/mL antibody solution. A 96 well plate was washed three times with 300 μL of wash buffer, and 300 μL of block buffer was added, and incubated at room temperature for 30 min. A standard serial dilutions were prepared using a recombinant FVIII (Syd Labs 200 IU/mL). 100 uL samples were diluted 5-fold. A 96 well plate was washed three times with 300 μL of wash buffer, and 100 μL of block buffer and FVIII standard were added, and incubated at room temperature for 60 min. The 96 well plate was again washed three times with 300 μL of wash buffer, and 100 μL of detection antibody (GMA-8023) per well was added and incubated at room temperature for 60 minutes. The 96 well plate was washed three times with 300 μL of wash buffer, and 100 μL of streptavidin peroxidase per well was added, an incubated at room temperature for 30 minutes. The 96 well plate was washed three times with 300 μL of wash buffer, and 100 μL of o-Phenylenediamine (OPD) substrate (dissolved 1 tablet (5 mg) in 12 mL citrate buffer, then added 5 ul 30% H2O2) was added and incubated at room temperature for 15 min. 50 μL of 2N H2SO4 was added, and absorbance measurements were taken on the SpectraMax at 490 nm and 570 nm. An asymmetric sigmoidal, 5PL, x=concentration analysis was used to interpolate sample values based on the standard curves, and a limit of detection of 0.000994 IU/mL of FVIII with a limit of quantification of 0.14 IU/ML was established for this ELISA plate.

As is shown in FIGS. 1B-1D, the combination of the genetic regulatory element SEQ ID NO: 4 (WPRE3) and the nuclear targeting sequence SEQ ID NO: 6 (DTS) enhances the expression of FVIII and FIX in-vitro, in HepG2 cell lines.

Cells transfected with the pDNA comprising the sequence of SEQ ID NO: 12 (APOE-AAT-hbGi-FIX) encoding FIX coupled to an APOE-AAT promoter sequence produced FIX at a concentration of about 15 ng/mL, while cells transfected with pDNA comprising the sequence of SEQ ID NO. 14 (APOE-AAT-hbGi-FIX-WPRE3-bGH-polyA-DTS) encoding FIX 5′ coupled to the same APOE-AAT promoter sequence but further being coupled to the WPRE3 and DTS elements expressed FIX at nearly 3× the levels at a concentration of about 45 ng/mL.

Cells transfected with the pDNA comprising the sequence of SEQ ID NO: 16 (APOE-AAT-hbGi-FVIII-v3-bGH-polyA) encoding FVIII coupled to an APOE-AAT promoter sequence produced FVIII at a concentration of about 0.25 ng/mL, while cells transfected with pDNA comprising the sequence of SEQ ID NO. 17 (APOE-AAT-hbGi-FVIII-v3-WPRE3-bGH-polyA-DTS) encoding FVIII coupled to the same APOE-AAT promoter sequence but further being coupled to the DTS nuclear targeting sequence of SEQ ID NO: 6 expressed FVIII at nearly 8× the levels at a concentration of about 2.0 ng/mL.

In FIG. 1D, cells that were transfected with the pDNA comprising the sequence of SEQ ID NO: 15 (APOE-AAT-hbGi-FIX WPRE3-bGH-polyA-DTS-ITR) encoding FIX 5′ coupled to the same APOE-AAT promoter sequence but further being coupled to the WPRE3 and DTS and ITR elements expressed FIX at approximately 12× the levels at a concentration of about 130 ng/mL, when compared to cells transfected with the pDNA comprising the sequence of SEQ ID NO: 12 (APOE-AAT-hbGi-FIX).

### Example 2. In-Vivo Expression of FIX in Murine Models Via Sonoporation

In this example, in-vivo expression of FIX transfected in vectors comprising genetic regulatory elements which increase expression of the therapeutic nucleic acid to the murine liver in C57 mice were evaluated. Prior to the experiment, each mouse was implanted with a jugular vein catheter (JVC), through which the sonoactive microstructures and nucleic acid constructs were administered. Circular DNA encoding FIX coupled to promoters and genetic regulatory elements were produced. Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction utilized Phusion® High-Fidelity DNA Polymerase and primers designed with NEBuilder Assembly Tool. PCR fragments were purified from agarose gel utilizing NED Monarch Gel Extraction kit (NEB) and assembled in HiFi DNA assembly reaction. Circular DNA comprising the sequence of SEQ ID NO: 12 (APOE-AAT-hbGi-FIX), SEQ ID NO: 18 (APOE-AAT-hbGi-FIX-WPRE3-DTS) were manufactured, and their sequences confirmed using Sanger sequencing, and next generation sequencing: methods. Following confirmation of the correct pDNA sequences, transfection of the nucleic acid constructs to the C57 mice was performed and evaluated in three experimental groups of 5 C57 mice: a first negative control in which no nucleic acids were administered; a second experimental group transfected with a pDNA sequence comprising the sequence of SEQ ID NO: 12 (APOE-AAT-hbGi-FIX); and a third experimental group transfected with a DNA sequence comprising the sequence of SEQ ID NO: 18 (APOE-AAT-hbGi-FIX-WPRE3-DTS).

A dose of sonoactive microstructure and DNA solution was readied by first preparing the sonoactive microstructures (Optison) as instructed on the label: remove from 4 C storage and roll between the palms for 20 seconds; removing protective plastic and aluminum covering from Optison vial; placing 25G needle through the rubber gasket to provide a pressure vent; and using 1.5 inch 18G needle to draw up 200 μL of Optison into a syringe (dead space of the needle (about 50 microliters (uL)) included in the calculations). With the same needle and syringe, 50 μL of solution comprising 250 μg of pDNA payload was drawn into the syringe to combine the DNA and Optison. The Optison microbubbles and DNA payload were mixed in the syringe by rolling the syringe between the fingers until the solution was homogenous at a 1:4 volumetric ratio of DNA to microbubble solution. The DNA+Optison solution was drawn out of the needle dead space. Then the 18G needle was exchanged for a 25G blunt needle for injection into a subject JVC.

Approximately one-third of the microbubble-DNA solution were administered to the subject in a bolus injection over about 10 seconds through the jugular vein catheter, and ultrasound energy was applied to the subject over the liver using an alternating mechanical index protocol. Ultrasound energy was transcutaneously applied to the liver region of the subject with a L6-24 ultrasound probe, alternating between a first MI of 0.07, and a second MI of 1.5, at a focal depth of 2 cm, and a gain of 28. Nine flashes of high MI ultrasound energy were delivered at an MI of 1.5 with an interval of 4 seconds between each flash with the administration of the 9 pulses being repeated three times over a single liver location. The high MI pulse duration was about 0.82 microseconds. Following administration of ultrasound energy, the second one-third dose of the microbubble-DNA solution was administered to the subject in a bolus injection, and ultrasound energy was transcutaneously applied to a second region liver using the same ultrasound parameters. Following completion of the second administration of ultrasound energy, the final one-third dose of the microbubble-DNA solution was administered to the subject in a bolus injection, and ultrasound energy was transcutaneously applied to a third region liver using the same ultrasound parameters. Plasma samples were collected from the subject 72 hours following administration of the sonoporation treatment.

FIX samples were later defrosted, diluted 5-fold, and analyzed using ELISA Green Mountain Antibodies for detection of human FIX. Concentration of secreted transgenic human FIX in culture media 72 hours post transfection was measured by ELISA utilizing Green Mountain Antibodies. Approximately 100 μL of capture antibody (GMA-102) was incubated at 4 C overnight. The stock solution of the capture antibody was diluted to 2 μg/mL antibody solution. A 96 well plate was washed three times with 300 μL of wash buffer, and 300 μL of block buffer was added, and incubated at room temperature for 30 min. A standard serial dilutions were prepared using a recombinant FIX (Abcam 1.8 mg/mL). 100 uL samples were 5-fold. A 96 well plate was washed three times with 300 μL of wash buffer, and 100 μL of block buffer and FIX standard were added, and incubated at room temperature for 60 min. The 96 well plate was again washed three times with 300 μL of wash buffer, and 100 μL of detection antibody (GMA-184) per well was added and incubated at room temperature for 60 minutes. The 96 well plate was washed three times with 300 μL of wash buffer, and 100 μL of streptavidin peroxidase per well was added, an incubated at room temperature for 30 minutes. The 96 well plate was washed three times with 300 μL of wash buffer, and 100 μL of o-Phenylenediamine (OPD) substrate (dissolved 1 tablet (5 mg) in 12 mL citrate buffer, then added 5 ul 30% H2O2) was added and incubated at room temperature for 15 min. 50 μL of 2N H2SO4 was added, and absorbance measurements were taken on the SpectraMax at 490 nm and 570 nm. An asymmetric sigmoidal, 5PL, x=concentration analysis was used to interpolate sample values based on the standard curves, and a limit of detection of 0.8 ng/mL of FIX was established for this ELISA plate.

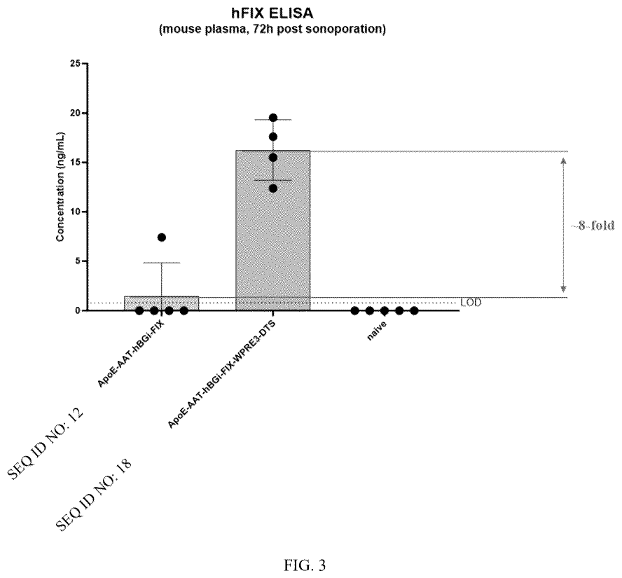

Results are shown in FIG. 3, in which it is shown that the control group of naïve mice administered no nucleic acids exhibited no FIX expression, the second experimental group administered a pDNA sequence comprising the sequence of SEQ ID NO: 12 (APOE-AAT-hbGi-FIX) exhibited FIX expression at about 2.0 ng/mL, while the third experimental group administered a pDNA sequence comprising the sequence of SEQ ID NO: 18 (APOE-AAT-hbGi-FIX-WPRE3-DTS) exhibited FIX expression at about 16.0 ng/mL—an eight fold expression increase over the second experimental group administered the FIX plasmid not comprising the DTS nuclear targeting sequence of SEQ ID NO: 6.

### Example 3. In-Vivo Expression of FIX in Murine Models Via Sonoporation

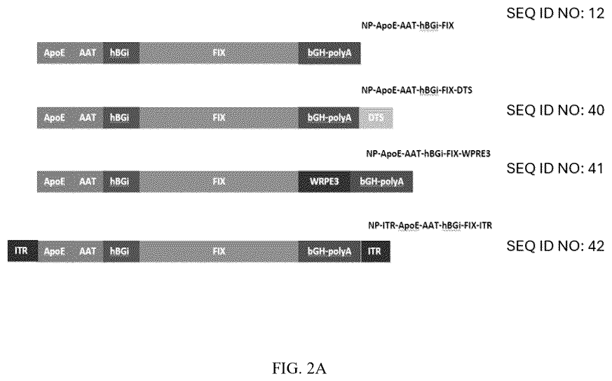

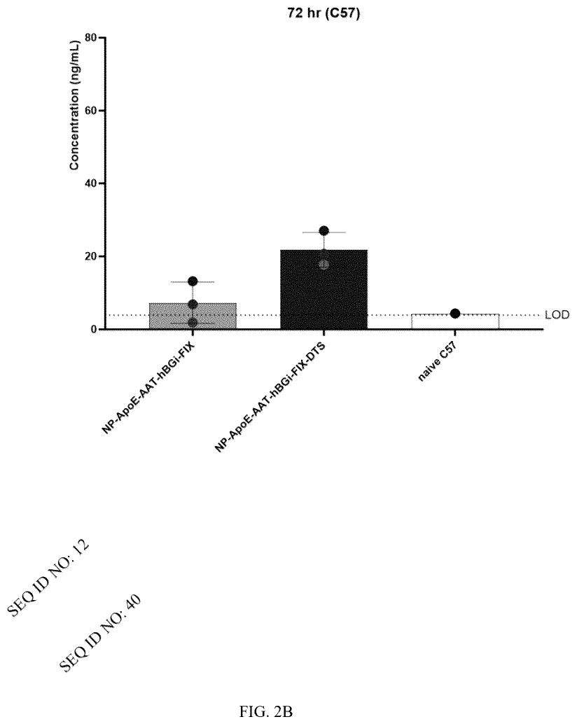

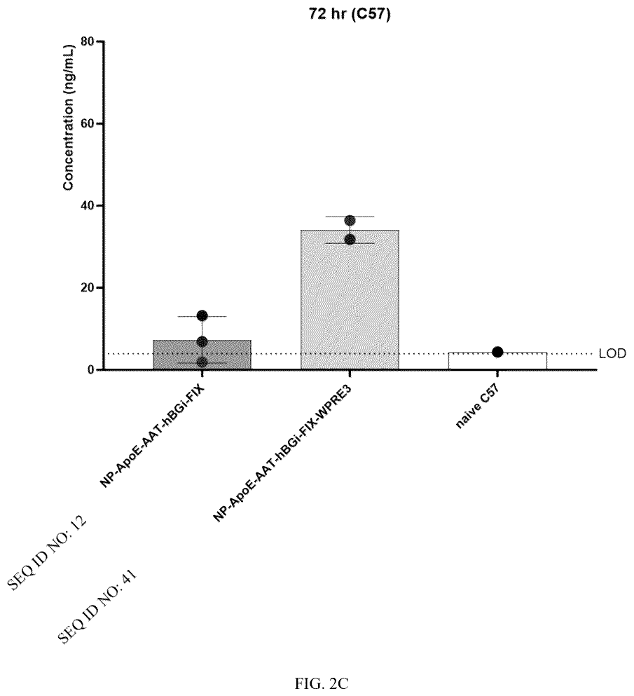

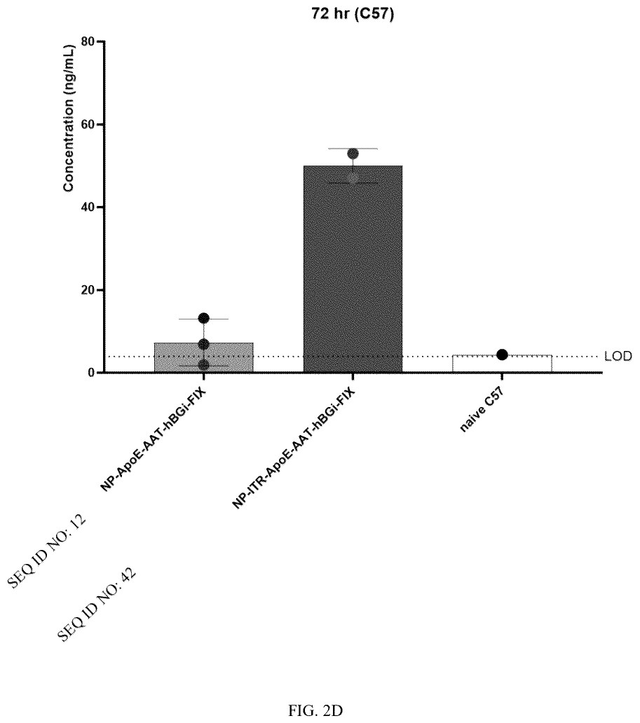

In this example, in-vivo expression FIX transfected in vectors comprising genetic regulatory elements which increase expression of the therapeutic nucleic acid to the murine liver in C57 mice was evaluated. Prior to the experiment, each mouse was implanted with a jugular vein catheter (JVC), through which the sonoactive microstructures and nucleic acid constructs were administered. Circular DNA encoding FIX coupled to promoters and genetic regulatory elements were produced. Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction utilized Phusion® High-Fidelity DNA Polymerase and primers designed with NEBuilder Assembly Tool. PCR fragments were purified from agarose gel utilizing NED Monarch Gel Extraction kit (NEB) and assembled in HiFi DNA assembly reaction. Circular DNA comprising the sequence of SEQ ID NO: 13 (APOE-AAT-hBGi-FIX-bGH-polyA), SEQ ID NO: 40 (APOE-AAT-hBGi-FIX-bGH-polyA-DTS), SEQ ID NO: 41 (APOE-AAT-hBGi-FIX WPRE3-bGH-polyA), and SEQ ID NO: 42 (ITR-APOE-AAT-hBGi-FIX-bGH-polyA-ITR), were manufactured, and their sequences confirmed using Sanger sequencing, and next generation sequencing. Vector maps of nucleic acid sequences comprising the therapeutic nucleic acid in vectors comprising genetic regulatory elements which increase expression of the therapeutic nucleic acid are illustrated in FIG. 2A.

Following confirmation of the correct circular DNA sequences, transfection of the nucleic acid constructs to the C57 mice was performed and evaluated in five experimental groups of three C57 mice: a first negative control in which no nucleic acids were administered; a second experimental group serving as a positive control transfected with a circular DNA sequence comprising the sequence of SEQ ID NO: 13 (APOE-AAT-hBGi-FIX-bGH-polyA), a third experimental group transfected with a circular DNA sequence comprising the sequence of SEQ ID NO: 40 (APOE-AAT-hBGi-FIX-bGH-polyA-DTS), a fourth experimental group transfected with a circular DNA sequence comprising the sequence of SEQ ID NO: 41 (APOE-AAT-hBGi-FIX WPRE3-bGH-polyA), and a fifth experimental group transfected with a circular DNA sequence comprising the sequence of SEQ ID NO: 42 (ITR-APOE-AAT-hBGi-FIX-bGH-polyA-ITR).

A dose of sonoactive microstructure and DNA solution was readied by first preparing the sonoactive microstructures (Optison) as instructed on the label: remove from 4 C storage and roll between the palms for 20 seconds; removing protective plastic and aluminum covering from Optison vial; placing 25G needle through the rubber gasket to provide a pressure vent; and using 1.5 inch 18G needle to draw up 200 μL of Optison into a syringe (dead space of the needle (about 50 microliters (uL)) included in the calculations). With the same needle and syringe, 50 μL of solution comprising 250 μg of pDNA payload was drawn into the syringe to combine the DNA and Optison. The Optison microbubbles and DNA payload were mixed in the syringe by rolling the syringe between the fingers until the solution was homogenous at a 1:4 volumetric ratio of DNA to microbubble solution. The DNA+Optison solution was drawn out of the needle dead space. Then the 18G needle was exchanged for a 25G blunt needle for injection into a subject JVC.

Approximately one-third of the microbubble-DNA solution were administered to the subject in a bolus injection over about 10 seconds through the jugular vein catheter, and ultrasound energy was applied to the subject over the liver using an alternating mechanical index protocol. Ultrasound energy was transcutaneously applied to the liver region of the subject with a L6-24 ultrasound probe, alternating between a first MI of 0.07, and a second MI of 1.5, at a focal depth of 2 cm, and a gain of 28. Nine flashes of high MI ultrasound energy were delivered at an MI of 1.5 with an interval of 4 seconds between each flash with the administration of the 9 pulses being repeated three times over a single liver location. The high MI pulse duration was about 0.82 microseconds. Following administration of ultrasound energy, the second one-third dose of the microbubble-DNA solution was administered to the subject in a bolus injection, and ultrasound energy was transcutaneously applied to a second region liver using the same ultrasound parameters. Following completion of the second administration of ultrasound energy, the final one-third dose of the microbubble-DNA solution was administered to the subject in a bolus injection, and ultrasound energy was transcutaneously applied to a third region liver using the same ultrasound parameters. Blood samples were collected from the subjects 72 hours following administration of the sonoporation treatment.

FIX samples were later defrosted, diluted 5-fold, and analyzed using ELISA Green Mountain Antibodies for detection of human FIX. Concentration of secreted transgenic human FIX in culture media 72 hours post transfection was measured by ELISA utilizing Green Mountain Antibodies. Approximately 100 μL of capture antibody (GMA-102) was incubated at 4 C overnight. The stock solution of the capture antibody was diluted to 2 μg/mL antibody solution. A 96 well plate was washed three times with 300 μL of wash buffer, and 300 μL of block buffer was added, and incubated at room temperature for 30 min. A standard serial dilutions were prepared using a recombinant FIX (Abcam 1.8 mg/mL). 100 uL samples were 5-fold. A 96 well plate was washed three times with 300 μL of wash buffer, and 100 μL of block buffer and FIX standard were added, and incubated at room temperature for 60 min. The 96 well plate was again washed three times with 300 μL of wash buffer, and 100 μL of detection antibody (GMA-184) per well was added and incubated at room temperature for 60 minutes. The 96 well plate was washed three times with 300 μL of wash buffer, and 100 μL of streptavidin peroxidase per well was added, an incubated at room temperature for 30 minutes. The 96 well plate was washed three times with 300 μL of wash buffer, and 100 μL of o-Phenylenediamine (OPD) substrate (dissolved 1 tablet (5 mg) in 12 mL citrate buffer, then added 5 ul 30% H2O2) was added and incubated at room temperature for 15 min. 50 μL of 2N H2SO4 was added, and absorbance measurements were taken on the SpectraMax at 490 nm and 570 nm. An asymmetric sigmoidal, 5PL, x=concentration analysis was used to interpolate sample values based on the standard curves, and a limit of detection of 0.8 ng/mL of FIX was established for this ELISA plate.

Results are shown in FIGS. 2B-2D, in which it is shown that the control group of naïve mice administered no nucleic acids exhibited no detectable FIX expression, that the second experimental group serving as a positive control transfected with nucleic acid composition comprising the sequence of SEQ ID NO: 13 (APOE-AAT-hBGi-FIX-bGH-polyA) exhibited low levels of FIX expression of about 5 ng/mL, while experimental groups 3-5 exhibited increased FIX expression ranging from about 25 ng/mL up to about 50 ng/mL.

In FIG. 2B, it is illustrated that the third experimental group transfected with nucleic acid composition comprising the sequence of SEQ ID NO: 40 (APOE-AAT-hBGi-FIX-bGH-polyA-DTS) including the nuclear target sequence described herein exhibited increased FIX levels of about 25 ng/mL, a 5× increase over the second experimental group serving as a positive control transfected with nucleic acid composition comprising the sequence of SEQ ID NO: 13 (APOE-AAT-hBGi-FIX-bGH-polyA), indicating that the DTS nuclear targeting sequence (SEQ ID NO: 6) provided a beneficial technical effect in increasing expression of the therapeutic transgene.

In FIG. 2C, it is illustrated that the fourth experimental group transfected with a circular DNA sequence comprising the sequence of SEQ ID NO: 41 (APOE-AAT-hBGi-FIX WPRE3-bGH-polyA) comprising the WPRE3 post-transcriptional regulatory element exhibited increased FIX levels of about 35 ng/mL, a 7× increase over the second experimental group serving as a positive control transfected with nucleic acid composition comprising the sequence of SEQ ID NO: 13 (APOE-AAT-hBGi-FIX-bGH-polyA), indicating that the combination of the hemoglobin subunit gamma intron (hBGi) sequence upstream of the therapeutic nucleic acid sequence and WPRE3 post-transcriptional regulatory element (SEQ ID NO: 4) downstream of the therapeutic nucleic acid sequence provided a beneficial technical effect in increasing expression of the therapeutic transgene.

In FIG. 2C, it is illustrated that the fourth experimental group transfected with a circular DNA sequence comprising the sequence of SEQ ID NO: 41 (APOE-AAT-hBGi-FIX WPRE3-bGH-polyA) comprising the WPRE3 post-transcriptional regulatory element exhibited increased FIX levels of about 35 ng/mL, a 7× increase over the second experimental group serving as a positive control transfected with nucleic acid composition comprising the sequence of SEQ ID NO: 13 (APOE-AAT-hBGi-FIX-bGH-polyA), indicating that the post-transcriptional regulatory element of SEQ ID NO: 45 provided a beneficial technical effect in increasing expression of the therapeutic transgene.

In FIG. 2D, it is illustrated that the fifth experimental group transfected with a circular DNA sequence comprising the sequence of SEQ ID NO: 42 (ITR-APOE-AAT-hBGi-FIX-bGH-polyA-ITR) comprising the ITR sequences exhibited increased FIX levels of about 50 ng/mL, a 10× increase over the second experimental group serving as a positive control transfected with nucleic acid composition comprising the sequence of SEQ ID NO: 13 (APOE-AAT-hBGi-FIX-bGH-polyA), indicating that the ITR sequences (SEQ ID NO: 4) provided a beneficial technical effect in increasing expression of the therapeutic transgene.

### Example 4. In-Vitro Expression of Novel FVIII Transgenes in HepG2 and Hepa1-6 Cell Lines

In this example, in-vitro expression of therapeutic nucleic acids encoding FVIII coding nucleic acid sequences in vectors comprising genetic regulatory elements which increase expression transfected to Human Liver Hepatocellular Cell Line HepG2 and Murine (C57L) Hepatocellular Cell Line Hepa1-6 were evaluated. Each cell line was tested using two experimental groups. A first experimental group was administered a nucleic acid composition comprising the sequence of SEQ ID NO: 43 (ApoE-AAT-hBGi-FVIII-v3-WPRE3-DTS), a FVIII sequence known within the art. A second experimental group was administered a nucleic acid composition comprising the sequence of SEQ ID NO:39 (ApoE-AAT-hBGi-FVIII-N6-F309S-DM-WPRE3-DTS), a novel and modified version of the FVIII transgene disclosed herein.

Circular DNA encoding the differing FVIII variants were coupled to a same combination of promoters and genetic regulatory elements were produced using methods known within the art. Circular DNA sequences comprising the sequence of SEQ ID NO: 39 (ApoE-AAT-hBGi-FVIII-N6-F309S-DM-WPRE3-DTS), and SEQ ID NO: 43 (ApoE-AAT-hBGi-FVIII-v3-WPRE3-DTS), were manufactured. Parts of the DNA vectors were synthesized (GeneScript) and combined in final pDNA vectors by NEBuilder® HiFi DNA Assembly (NEB) following manufacture's protocol. In brief, DNA fragments were amplified by PCR reaction utilized Phusion® High-Fidelity DNA Polymerase and primers designed with NEBuilder Assembly Tool. PCR fragments were purified from agarose gel utilizing NED Monarch Gel Extraction kit (NEB) and assembled in HiFi DNA assembly reaction. Circular DNA sequence integrity was confirmed by next generation sequencing methods.

Following confirmation of the correct circular DNA sequences, the nucleic acid compositions were transfected to Human Liver Hepatocellular Cell Line HepG2, and Murine (C57L) Hepatocellular Cell Line Hepa1-6 using a lipid-nanoparticle based Lipofectamine® P3000 transfection reagent. 100 ng of DNA, with 5 μL of Opti-MEM™ Reduced-Serum Medium 0.1 uL, and 0.1 of P3000 transfection reagent were combined in a 96 well plate and incubating at room temperature for 5 min. Negative controls were prepared by combining 5 μL of Opti-MEM™ Reduced-Serum Medium 0.1 uL, and 0.15 of P3000 transfection reagent, and incubating at room temperature for 5 min. The HepG2 cell were then added, were cultured, and media collected at 72 hours post transfection and frozen at −80 C.

FVIII samples were later defrosted, diluted 5-fold, and analyzed using ELISA Green Mountain Antibodies for detection of human FVIII. Concentration of secreted transgenic human FVIII in culture media 72 hours post transfection was measured by ELISA utilizing Green Mountain Antibodies. Approximately 100 μL of capture antibody (GMA-8024) was incubated at 4 C overnight. The stock solution of the capture antibody was diluted to 2 μg/mL antibody solution. A 96 well plate was washed three times with 300 μL of wash buffer, and 300 μL of block buffer was added, and incubated at room temperature for 30 min. A standard serial dilutions were prepared using a recombinant FVIII (Syd Labs 200 IU/mL). 100 uL samples were diluted 5-fold. A 96 well plate was washed three times with 300 μL of wash buffer, and 100 μL of block buffer and FVIII standard were added, and incubated at room temperature for 60 min. The 96 well plate was again washed three times with 300 μL of wash buffer, and 100 μL of detection antibody (GMA-8023) per well was added and incubated at room temperature for 60 minutes. The 96 well plate was washed three times with 300 μL of wash buffer, and 100 μL of streptavidin peroxidase per well was added, an incubated at room temperature for 30 minutes. The 96 well plate was washed three times with 300 μL of wash buffer, and 100 μL of o-Phenylenediamine (OPD) substrate (dissolved 1 tablet (5 mg) in 12 mL citrate buffer, then added 5 ul 30% H₂02) was added and incubated at room temperature for 15 min. 50 μL of 2N H2SO4 was added, and absorbance measurements were taken on the SpectraMax at 490 nm and 570 nm. An asymmetric sigmoidal, 5PL, x=concentration analysis was used to interpolate sample values based on the standard curves, and a limit of detection of 0.000994 IU/mL of FVIII with a limit of quantification of 0.14 IU/ML was established for this ELISA plate.

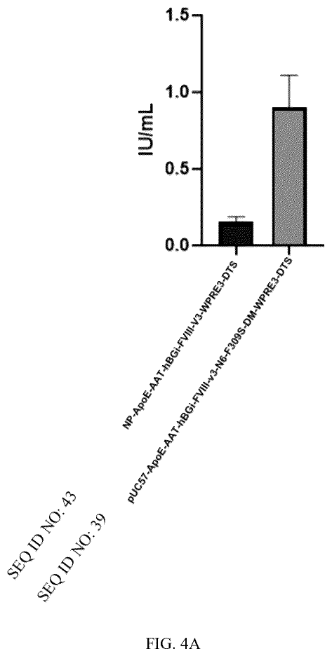

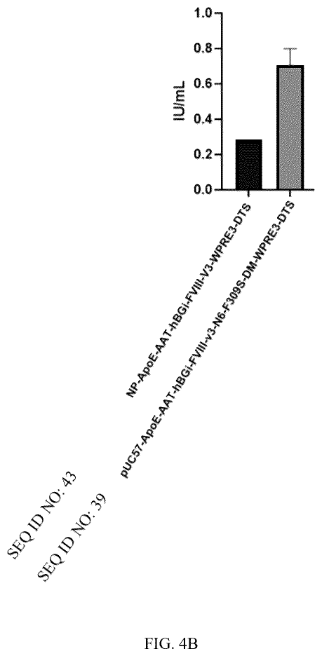

As is shown in FIGS. 4A-4B, the modifications to the FVIII polypeptide sequence increase expression of FVIII in both the Human Liver Hepatocellular Cell Line HepG2 and Murine (C57L) Hepatocellular Hepa1-6 Cell Line.

In FIG. 4A, it illustrated that expression in the Murine (C57L) Hepatocellular Hepa1-6 Cell Line is increased. The first experimental group administered the nucleic acid composition comprising the sequence of SEQ ID NO: 43 (ApoE-AAT-hBGi-FVIII-v3-WPRE3-DTS), a FVIII sequence known within the art, exhibits FVIII levels of about 0.15 IU/mL. The second experimental group administered the nucleic acid composition comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), exhibits FVIII levels of about 0.9 IU/mL, a 6-fold (6×) increase over the first experimental group, indicating that the modifications to the FVIII transgene disclosed herein in SEQ ID NO:39 provide a beneficial technical effect in increasing expression of FVIII.

In FIG. 4B, it illustrated that expression in the Human Liver Hepatocellular Cell Line HepG2 is increased. The first experimental group administered the nucleic acid composition comprising the sequence of SEQ ID NO: 43 (ApoE-AAT-hBGi-FVIII-v3-WPRE3-DTS), a FVIII sequence known within the art, exhibits FVIII levels of about 0.3 IU/mL. The second experimental group administered the nucleic acid composition comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), exhibits FVIII levels of about 0.7 IU/mL, a 2.3-fold (2.3×) increase over the first experimental group, indicating that the modifications to the FVIII transgene disclosed herein in SEQ ID NO:39 provide a beneficial technical effect in increasing expression of FVIII.

### Example 5. In-Vivo Expression of FVIII in Murine Models Via Sonoporation

In this example, in-vivo expression FVIII transfected to Rag2 mice was evaluated. Prior to the experiment, each mouse was implanted with a jugular vein catheter (JVC), through which the sonoactive microstructures and nucleic acid constructs were administered. Circular DNA encoding FVIII coupled to promoters and genetic regulatory elements were produced: circular DNA comprising the sequence of SEQ ID NO: 43 (ApoE-AAT-hBGi-FVIII-v3-WPRE3-DTS), a FVIII sequence known within the art; and circular DNA comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24).

Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction utilized Phusion® High-Fidelity DNA Polymerase and primers designed with NEBuilder Assembly Tool. PCR fragments were purified from agarose gel utilizing NED Monarch Gel Extraction kit (NEB) and assembled in HiFi DNA assembly reaction.

Sequences were confirmed using Sanger sequencing, and next-generation sequencing methods. Following confirmation of the correct pDNA sequences, transfection of the nucleic acid constructs to the Rag2 mice was performed and evaluated in four experimental groups of three or four Rag2 mice each: a first experimental group was administered 250 μg of a nucleic acid composition comprising the sequence of SEQ ID NO: 43 (ApoE-AAT-hBGi-FVIII-v3-WPRE3-DTS); and a second experimental group was administered 250 μg of a nucleic acid composition comprising the sequence of SEQ ID NO:39 (ApoE-AAT-hBGi-FVIII-N6-F309S-DM-WPRE3-DTS), a nucleic acid expression vector encoding a modified version of the FVIII transgene; a third experimental group was administered 100 μg of a nucleic acid composition comprising the sequence of SEQ ID NO: 43 (ApoE-AAT-hBGi-FVIII-v3-WPRE3-DTS); and a fourth experimental group was administered 100 μg of a nucleic acid composition comprising the sequence of SEQ ID NO:39 (ApoE-AAT-hBGi-FVIII-N6-F309S-DM-WPRE3-DTS), a modified FVIII transgene.

A dose of sonoactive microstructure and DNA solution was readied by first preparing the sonoactive microstructures (Optison) as instructed on the label: remove from 4 C storage and roll between the palms for 20 seconds; removing protective plastic and aluminum covering from Optison vial; placing 25G needle through the rubber gasket to provide a pressure vent; and using 1.5 inch 18G needle to draw up 200 μL of Optison into a syringe (dead space of the needle (about 50 microliters (uL)) included in the calculations). With the same needle and syringe, 50 μL of solution comprising either 250 μg or 100 ug (depending on experimental group) of pDNA payload was drawn into the syringe to combine the DNA and Optison. The Optison microbubbles and DNA payload were mixed in the syringe by rolling the syringe between the fingers until the solution was homogenous at a 1:4 volumetric ratio of DNA to microbubble solution. The DNA+Optison solution was drawn out of the needle dead space. Then the 18G needle was exchanged for a 25G blunt needle for injection into a subject JVC.

The microbubble-DNA solution was administered to the subject in a single bolus injection over about 30 seconds through the jugular vein catheter, and ultrasound energy was applied to the subject over the liver. Ultrasound energy was transcutaneously applied to the liver region of the subject with a C1-6 ultrasound probe, and scans liver of each mouse were performed with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Gain 44, Depth 4 cm, AO %100, Gain 55, T 8, SVD 6.0, AO %100%, +50-400 Hz, GEN. 100% push output and 100% Track output. Mice were administered 150 μL of protein stabilized Optison sonoactive microstructures and 250 pg of nucleic acid in 50 μL of solution, for a total injection volume of about 200 uL, and an acoustic radiation force protocol was applied for 10 seconds, followed by 20 seconds with no application of ultrasound during which the ultrasound probe was removed from the subject, followed again by 10 seconds of an acoustic radiation force protocol, up to the amount of total time of radiation force protocol application indicated in the below table. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was applied using an alternating imaging technique applying focused ultrasound and generating a B-mode ultrasound image at a mechanical index of 1.4, and a frequency of 2.5 MHz with the ultrasound probe removed from contacting the subject as indicated.

After the administration of the sonoporation treatment, FVIII expression and secretion was measured in subject plasma samples collected at 72 hours following the sonoporation treatment and frozen at −80 C.

Transgenic FVIII level in mouse plasma was measured by MSD assay. Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921U/ml to 0.01 IU/ml. 2-fold diluted samples and standards were added to the wells in 96-well plate. Incubated 2 hours at room temperature and washed 3 times. The detection was performed by incubating samples with GMA-8023 antibody during 2 hours following triple wash. Signal was developed by Sulfo-TAG and detected by MSD machine.

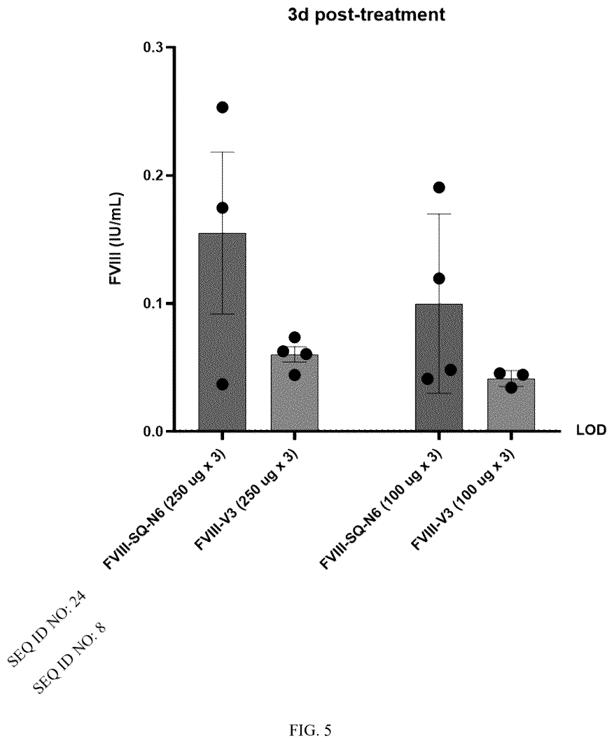

Results are shown in FIG. 5, in which it is shown that the first experimental group administered 250 μg of a nucleic acid composition comprising the sequence of SEQ ID NO: 43 (ApoE-AAT-hBGi-FVIII-v3-WPRE3-DTS) exhibits FVIII levels of about 0.06 IU/mL; the second experimental group was administered 250 μg of a nucleic acid composition comprising the sequence of SEQ ID NO:39 (ApoE-AAT-hBGi-FVIII-N6-F309S-DM-WPRE3-DTS), a modified FVIII transgene, exhibits FVIII levels of about 1.5 IU/mL; the third experimental group administered 100 μg of a nucleic acid composition comprising the sequence of SEQ ID NO: 43 (ApoE-AAT-hBGi-FVIII-v3-WPRE3-DTS) exhibits FVIII levels of about 0.04 IU/mL; and the fourth experimental group administered 100 μg of a nucleic acid composition comprising the sequence of SEQ ID NO:39 (ApoE-AAT-hBGi-FVIII-N6-F309S-DM-WPRE3-DTS), a modified FVIII transgene exhibits FVIII levels of about 0.1 IU/mL. Comparing the 250 ug dose groups, it was observed that the second experimental group administered 250 μg of the novel and modified version FVIII transgene of SEQ ID NO: 39 exhibited approximately 3-fold (3×) higher expression levels of FVIII as compared to the second experimental group administered 250 μg of the FVIII sequence of SEQ ID NO: 43, indicating that the modifications to the FVIII transgene disclosed herein in SEQ ID NO:39 provide a beneficial technical effect in increasing expression of FVIII in-vivo. Comparing the 100 ug dose groups, it was observed that the fourth experimental group administered 100 μg of the novel and modified version of the FVIII transgene disclosed herein (SEQ ID NO: 39) exhibited approximately 2.5-fold (2.5×) higher expression levels of FVIII as compared to the third experimental group administered 100 μg of the FVIII sequence of SEQ ID NO: 43, indicating that the modifications to the FVIII transgene disclosed herein in SEQ ID NO:39 provide a beneficial technical effect in increasing expression of FVIII in-vivo.

### Example 6. In-Vivo Expression of FVIII in Murine Models Via Sonoporation

In this example, in-vivo expression of a modified FVIII transgene transfected to Rag2 mice was evaluated. Prior to the experiment, each mouse was implanted with a jugular vein catheter (JVC), through which the sonoactive microstructures and nucleic acid constructs were administered. Circular DNA encoding FVIII coupled to promoters and genetic regulatory elements were produced: circular DNA comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), was evaluated in for long term expression in a triple repeated treatment administration protocol.

Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction using Phusion® High-Fidelity DNA Polymerase and primers were designed with NEBuilder Assembly Tool. PCR fragments were purified from agarose gel utilizing NED Monarch Gel Extraction kit (NEB) and assembled in HiFi DNA assembly reaction.

Sequences were confirmed using Sanger sequencing, and next-generation sequencing methods. Following confirmation of the correct pDNA sequences, transfection of the nucleic acid constructs to the Rag2 mice was performed and evaluated in four experimental groups of three or four Rag2 mice each: an experimental group was administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified version of the FVIII transgene.

The treatment protocol is described in the following paragraphs. Each subject was administered three treatments constituting a cycle, each treatment administered 48 hours apart. Each subject was administered a second cycle 40 days following the completion of the third treatment in the first cycle. Then, each subject was administered a third cycle, 50 days following the completion of the third treatment in the second cycle.

A dose of sonoactive microstructure and DNA solution was readied by first preparing the sonoactive microstructures (Optison) as instructed on the label: remove from 4 C storage and roll between the palms for 20 seconds; removing protective plastic and aluminum covering from Optison vial; placing 25G needle through the rubber gasket to provide a pressure vent; and using 1.5 inch 18G needle to draw up 200 μL of Optison into a syringe. With the same needle and syringe, 50 μL of solution comprising 50 μg of DNA payload was drawn into the syringe to combine the DNA and Optison. The Optison microbubbles and DNA payload were mixed in the syringe by rolling the syringe between the fingers until the solution was homogenous at a 1:4 volumetric ratio of DNA to microbubble solution. The DNA+Optison solution was drawn out of the needle dead space. Then the 18G needle was exchanged for a 25G blunt needle for injection into a subject JVC.

The microbubble-DNA solution was administered to the subject in a single bolus injection over about 30 seconds through the jugular vein catheter, and ultrasound energy was applied to the subject over the liver. Ultrasound energy was transcutaneously applied to the liver region of the subject with a C1-6 ultrasound probe, and scans liver of each mouse were performed with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Gain 44, Depth 4 cm, AO %100, Gain 55, T 8, SVD 6.0, AO %100%, +50-400 Hz, GEN. 100% push output 100% and Track output 0%. Mice were administered 150 μL of protein stabilized Optison sonoactive microstructures and 50 pg of nucleic acid in 50 μL of solution, for a total injection volume of about 200 uL, and an acoustic radiation force protocol was applied for 10 seconds, followed by 20 seconds with no application of ultrasound during which the ultrasound probe was removed from the subject, followed again by 10 seconds of an acoustic radiation force protocol, for 40 seconds of total ultrasound application. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was applied using an alternating imaging technique applying focused ultrasound and generating a B-mode ultrasound image at a mechanical index of 0.4, and a frequency of 2.5 MHz with the ultrasound probe removed from contacting the subject as indicated.

After the administration of the sonoporation treatment, FVIII expression and secretion was measured in subject plasma samples collected approximately every 7 days following the sonoporation treatment and frozen at −80 C.

Transgenic FVIII level in mouse plasma was measured by immunoassay (MESO SCALE DIAGNOSTICS, LLC). Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921U/ml to 0.01 IU/ml. 2-fold diluted samples and standards were added to the wells in 96-well plate. The plate was incubated 2 hours at room temperature and washed 3 times. The detection was performed by incubating samples with GMA-8023 antibody during 2 hours following triple wash. Signal was developed by Sulfo-TAG and detected by MSD machine.

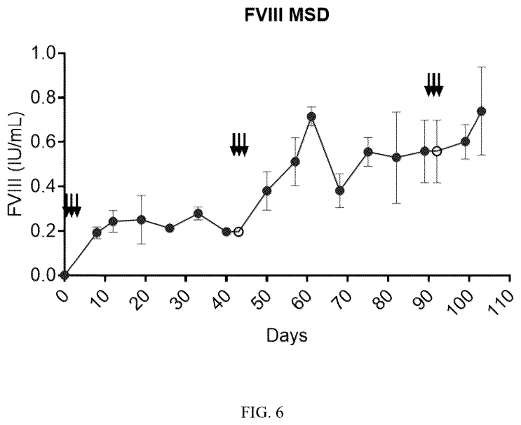

Results are shown in FIG. 6, in which it is shown that the experimental group administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified FVIII transgene, exhibits: FVIII levels of about 0.2 IU/mL following the administration of the first cycle with expression levels maintained at this level up to administration of the second cycle; FVIII levels of about 0.5 IU/mL (ranging from 0.2 to 0.7 IU/mL) following the administration of the second cycle with expression levels maintained at about 0.5 IU/mL up to administration of the third cycle; and FVIII levels of about 0.7 IU/mL following the administration of the third cycle with expression levels maintained at about 0.7 IU/mL past 100 days of the study. The results shown herein indicate that the modifications to the nucleic acid expression vector disclosed herein encoding the modified FVIII transgene disclosed herein in SEQ ID NO:39 provide a beneficial technical effect in increasing expression of FVIII in-vivo and maintaining a stable level of FVIII expression in-vivo following a multiple treatment protocol.

### Example 7. In-Vivo Expression of FVIII in Murine Models Via Sonoporation

In this example, in-vivo expression of FVIII transfected to Rag2 mice was evaluated. Prior to the experiment, each mouse was implanted with a jugular vein catheter (JVC), through which the sonoactive microstructures and nucleic acid constructs were administered. Circular DNA encoding FVIII coupled to promoters and genetic regulatory elements were produced: circular DNA comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), was evaluated in for short term expression in a triple repeated treatment administration protocol with a lipid stabilized sonoactive agent.

Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction using Phusion® High-Fidelity DNA Polymerase and primers were designed with NEBuilder Assembly Tool. PCR fragments were purified from agarose gel utilizing NED Monarch Gel Extraction kit (NEB) and assembled in HiFi DNA assembly reaction.

Sequences were confirmed using Sanger sequencing, and next-generation sequencing methods. Following confirmation of the correct pDNA sequences, transfection of the nucleic acid constructs to the Rag2 mice was performed and evaluated in four experimental groups of three or four Rag2 mice each: an experimental group was administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified version of the FVIII transgene.

The treatment protocol is described in the following paragraphs. Each subject was administered three treatments constituting a cycle.

A dose of sonoactive microstructure and DNA solution was readied by first preparing the sonoactive microstructures (Sonazoid, phospholipid stabilized perfluorobutane microbubbles) as instructed on the label. In brief sterile water was drawn up into a syringe, and the vial was penetrated under aseptic conditions. The sterile water was gently injected into the vial, avoiding forceful handling that could damage the microbubbles. Next, the vial was swirled in a gentle, circular motion for several seconds, ensuring the Sonazoid powder dissolves and forms a uniform suspension without creating excess foam or bubbles. Once reconstituted, the suspension is visually inspected for clarity and absence of particulate matter or discoloration. With the same needle and syringe, 50 μL of solution comprising 50 μg of DNA payload was drawn into the syringe to combine the DNA and Sonazoid. The Sonazoid microbubbles and DNA payload were mixed in the syringe by rolling the syringe between the fingers until the solution was homogenous. The DNA+Sonazoid solution was drawn out of the needle dead space in preparation for injection into a subject JVC.

The microbubble-DNA solution was administered to the subject in a single bolus injection over about 30 seconds through the jugular vein catheter, and ultrasound energy was applied to the subject over the liver. Ultrasound energy was transcutaneously applied to the liver region of the subject with a C1-6 ultrasound probe, and scans liver of each mouse were performed with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Gain 44, Depth 4 cm, AO %100, Gain 55, T 8, SVD 6.0, AO %100%, +50-400 Hz, GEN. 100% push output 100% and Track output 0%. Mice were administered the sonoactive agent and DNA suspension, and an acoustic radiation force protocol was applied for 10 seconds, followed by 20 seconds with no application of ultrasound during which the ultrasound probe was removed from the subject, followed again by 10 seconds of an acoustic radiation force protocol, for 40 seconds of total ultrasound application. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was applied using an alternating imaging technique applying focused ultrasound and generating a B-mode ultrasound image at a mechanical index of 0.4, and a frequency of 2.5 MHz with the ultrasound probe removed from contacting the subject as indicated.

After the administration of the sonoporation treatment, FVIII expression and secretion was measured in subject plasma samples collected approximately at days 9 and 13 following the sonoporation treatment and frozen at −80 C.

Transgenic FVIII level in mouse plasma was measured by immunoassay (MESO SCALE DIAGNOSTICS, LLC). Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921U/ml to 0.01 IU/ml. 2-fold diluted samples and standards were added to the wells in 96-well plate. Incubated 2 hours at room temperature and washed 3 times. The detection was performed by incubating samples with GMA-8023 antibody for 2 hours following triple wash. Signal was developed by Sulfo-TAG and detected by MSD machine.

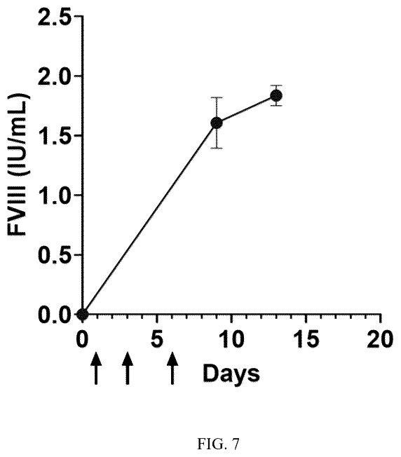

Results are shown in FIG. 7, in which it is shown that the experimental group administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified FVIII transgene, exhibits: FVIII levels of about 1.37 IU/mL at day 9 and 1.72 IU/mL at day 13 following the administration of the first cycle. The results shown herein indicate that the modifications to the nucleic acid expression vector disclosed herein encoding the modified FVIII transgene disclosed herein in SEQ ID NO:39 provide a beneficial technical effect in increasing expression of FVIII in-vivo and maintaining a stable level of FVIII expression in-vivo following a multiple treatment protocol.

### Example 8. Chromogenic Assay Evaluating Clotting Activity of FVIII in a Murine Model of Hemophilia A

In this example, in-vivo expression of FVIII transgenes of the present disclosure transfected to 129S-F8 mice (a FVIII knock out mouse which is used as a murine model of Hemophilia A) were evaluated, and the clotting activity of secreted FVIII protein was evaluated using a chromogenic assay. Circular DNA encoding FVIII coupled to promoters and genetic regulatory elements were produced: circular DNA comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), was evaluated in for short term expression and clotting activity of the FVIII protein as compared to a clinically validated FVIII protein control.

Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction using Phusion® High-Fidelity DNA Polymerase and primers were designed with NEBuilder Assembly Tool. PCR fragments were purified from agarose gel utilizing NED Monarch Gel Extraction kit (NEB) and assembled in HiFi DNA assembly reaction.

Sequences were confirmed using Sanger sequencing, and next-generation sequencing methods. Following confirmation of the correct pDNA sequences, transfection of the nucleic acid constructs to the 129S-F8 mice was performed and evaluated in three experimental groups of one (for the naive control group) or four 129S-F8 mice each: Group 1 was a naive control not administered any ultrasound or nucleic acids; Group 2 was administered 50 μg of a nucleic acid composition comprising the sequence of SEQ ID NO:39 (ApoE-AAT-hBGi-FVIII-N6-F309S-DM-WPRE3-DTS) which encodes a FVIII polypeptide having a sequence of SEQ ID NO: 24; and Group 3 was administered 50 μg of a nucleic acid composition encoding a clinically validated FVIII transgene (FVIII v3) having a sequence of SEQ ID NO: 46. The 50 ug dose of the nucleic acid composition was administered to the mice through tail vein injection using a hydrodynamic delivery technique in which a solution of 50 μL of the nucleic acid composition and 1950 μL of saline were rapidly injected into the tail vein over about 1 second to drive delivery into the liver.

After the administration of the sonoporation treatment, FVIII expression and secretion was measured in subject plasma samples collected approximately 24 hours following the hydrodynamic delivery.

Transgenic FVIII level in mouse plasma was measured by immunoassay (MESO SCALE DIAGNOSTICS, LLC). Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921U/ml to 0.01 IU/ml. 2-fold diluted samples and standards were added to the wells in 96-well plate. Incubated 2 hours at room temperature and washed 3 times. The detection was performed by incubating samples with GMA-8023 antibody for 2 hours following triple wash. Signal was developed by Sulfo-TAG and detected by MSD machine.

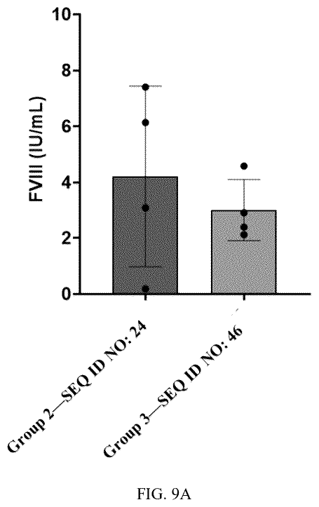

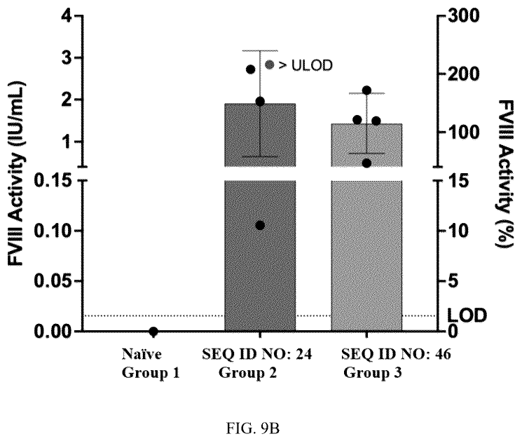

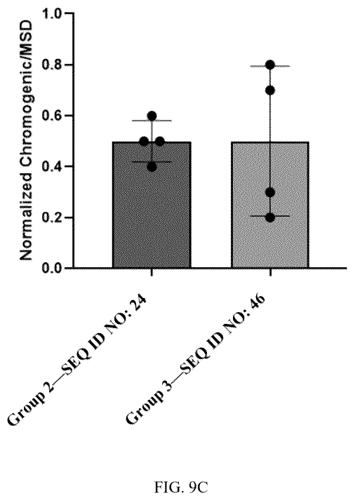

Results are shown in FIG. 9A, in which it is shown that the experimental Group2 administered 50 μg of a nucleic acid composition comprising the sequence of SEQ ID NO:39 (ApoE-AAT-hBGi-FVIII-N6-F309S-DM-WPRE3-DTS) and encoding a FVIII polypeptide of SEQ ID NO: 24, a nucleic acid expression vector encoding a modified FVIII transgene, exhibits average FVIII levels of about 4 IU/mL; experimental Group 3 administered 50 μg of a nucleic acid composition encoding a clinically validated FVIII transgene (FVIII v3) having a sequence of SEQ ID NO: 46 exhibits FVIII levels of about 3.25 IU/mL.

The FVIII chromogenic assay is a laboratory method used to measure the functional activity of Factor VIII through a two-stage process: first, patient plasma is incubated with activated Factor IX, Factor X, phospholipids, and calcium, allowing active Factor VIII to facilitate the conversion of Factor X to Factor Xa; second, the amount of Factor Xa produced is quantified by adding a chromogenic substrate that releases a colored product upon cleavage by Factor Xa, with the color intensity being directly proportional to the Factor VIII activity in the sample.

Next, the FVIII chromogenic assay was performed on remaining plasma samples to assess the clotting activity of the novel transgenic FVIII protein. Initially, all required reagents and consumables are prepared, ensuring that if the assay kit is brand new, all components are reconstituted correctly to yield sufficient material for two full plates. Prior to commencing the assay, the CaCl₂) and S-2765+I-2581 solutions are pre-warmed to 37° C., and the shaking incubator is set to the same temperature. Standard curves are established using several control materials: stock FACT plasma (from George King Bio-Medical, lot 7662) standardized at 100% with a CoA chromogenic FVIII activity of 92%, naive C57 plasma collected with K₂-EDTA, and stock Xyntha (lot GX0072) at 62 IU/mL. For sample preparation, 5 μL of each sample or standard is diluted into 400 μL of 1× Buffer Solution in a deep-well plate, and the mixtures are briefly agitated using the shaking incubator set to a short mixing cycle. Next, 25 μL of the diluted sample or standard is transferred into the wells of a 96-well plate, followed by the addition of 50 μL of the IXa/X/Phospholipid reagent. The plate is then sealed and incubated at 37° C. for 5 minutes. Subsequently, 25 μL of CaCl₂ is added, the plate is resealed, and the incubation at 37° C. continues for another 5 minutes. Thereafter, 50 μL of the combined S-2765+I-2581 solution is introduced, and following sealing, the incubation at 37° C. is maintained for an additional 5 minutes. To terminate the enzymatic reaction, 25 μL of 20% acetic acid (serving as the stop buffer) is added, the plate is sealed once more, and a brief shake using the “Short” button ensures thorough mixing. Finally, the plate is immediately read on a SpectraMax Plate Reader at wavelengths of 405 nm and 490 nm to determine the chromogenic response, which correlates with the Factor VIII activity in the tested samples.

Results are shown in FIG. 9B, where it is shown that experimental Group2 administered 50 μg of a nucleic acid composition comprising the sequence of SEQ ID NO:39 (ApoE-AAT-hBGi-FVIII-N6-F309S-DM-WPRE3-DTS) and encoding a FVIII polypeptide of SEQ ID NO: 24, a nucleic acid expression vector encoding a modified FVIII transgene, exhibits average FVIII activity levels of about 2 IU/mL and 175% FVIII activity; and experimental Group 3 administered 50 μg of a nucleic acid composition encoding a clinically validated FVIII transgene (FVIII v3) having a sequence of SEQ ID NO: 46 exhibits average FVIII activity levels of about 1.25 IU/mL and 110% FVIII activity. Additional results are shown in FIG. 9C where the FVIII activity levels are normalized to the FVIII plasma levels to show the relative clotting activity of each FVIII polypeptide to allow for assessment of the specific activity of the protein, distinguishing whether differences in the coagulation function was due to a reduced quantity of Factor VIII or to qualitative defects in the protein's functionality. As can be shown in FIG. 9C, the FVIII polypeptide of SEQ ID NO: 24 exhibits a normalized FVIII activity level (normalized to MSD FVIII plasma protein level) of about 0.5, which is approximate equivalent to clinically validated FVIII transgene (FVIII v3) having a sequence of SEQ ID NO: 46. The results shown herein indicate that the modifications to the modified FVIII transgene disclosed herein in SEQ ID NO:24 maintains coagulation activity of FVIII.

### Example 9. In-Vivo Expression of FVIII in Murine Models Via Sonoporation

In this example, in-vivo expression of a modified FVIII transgene transfected to Rag2 mice was evaluated. Prior to the experiment, each mouse was implanted with a jugular vein catheter (JVC), through which the sonoactive microstructures and nucleic acid constructs were administered. Circular DNA encoding FVIII coupled to promoters and genetic regulatory elements were produced: circular DNA comprising the sequence of SEQ ID NO:39 (ApoE-AAT-hBGi-FVIII-N6-F309S-DM-WPRE3-DTS), encoding a modified version of the FVIII transgene disclosed herein (SEQ ID NO: 24), and was evaluated in for long term expression in a triple treatment administration protocol.

Parts of the DNA vectors were synthesized (GeneScript) and combined in final circular DNA vectors by NEBuilder® HiFi DNA Assembly (NEB) following manufacture's protocol. In brief, DNA fragments were amplified by PCR reaction using Phusion® High-Fidelity DNA Polymerase and primers were designed with NEBuilder Assembly Tool. PCR fragments were purified from agarose gel utilizing NED Monarch Gel Extraction kit (NEB) and assembled in HiFi DNA assembly reaction.

Sequences were confirmed using Sanger sequencing, and next-generation sequencing methods. Following confirmation of the correct pDNA sequences, transfection of the nucleic acid constructs to the Rag2 mice was performed and evaluated in four experimental groups of five Rag2 mice each: an experimental group was administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified version of the FVIII transgene (SEQ ID NO: 24).

A dose of sonoactive microstructure and DNA solution was readied by first preparing the sonoactive microstructures (Sonazoid, phospholipid stabilized perfluorobutane microbubbles) as instructed on the label. In brief sterile water was drawn up into a syringe, and the vial was penetrated under aseptic conditions. The sterile water was gently injected into the vial, avoiding forceful handling that could damage the microbubbles. Next, the vial was swirled in a gentle, circular motion for several seconds, ensuring the Sonazoid powder dissolves and forms a uniform suspension without creating excess foam or bubbles. Once reconstituted, the suspension is visually inspected for clarity and absence of particulate matter or discoloration. With the same needle and syringe, 20 μL of solution comprising 50 μg of DNA payload was drawn into the syringe to combine the DNA and Sonazoid microbubbles. The Sonazoid microbubbles and DNA payload were mixed in the syringe by rolling the syringe between the fingers until the solution was homogenous at a 1:4 volumetric ratio of DNA to microbubble solution. The DNA+Optison solution was drawn out of the needle dead space. Then the 18G needle was exchanged for a 25G blunt needle for injection into a subject JVC.

The microbubble-DNA solution was administered to the subject in a single bolus injection over about 30 seconds through the jugular vein catheter, and ultrasound energy was applied to the subject over the liver. Ultrasound energy was transcutaneously applied to the liver region of the subject with a C1-6 ultrasound probe, and scans liver of each mouse were performed with a GE LOGIQ e10 ultrasound system equipped with a C1-6 probe, and use of the “ELASTO” software, Shear Wave, using shear wave elastography parameters: GE LOGIQe10 probe C1-6, Abdomen, CHI, Frame rate 55, MI 1.4, Frequency 2.5 MHz, Gain 44, Depth 4 cm, AO %100, Gain 55, T 8, SVD 6.0, AO %100%, +50-400 Hz, GEN. 100% push output 100% and Track output 0%. Mice were administered 150 μL of protein stabilized Optison sonoactive microstructures and 50 pg of nucleic acid in 50 μL of solution, for a total injection volume of about 200 uL, and an acoustic radiation force protocol was applied for 10 seconds, followed by 20 seconds with no application of ultrasound during which the ultrasound probe was removed from the subject, followed again by 10 seconds of an acoustic radiation force protocol, for 40 seconds of total ultrasound application. The shear wave ultrasound acoustic radiation force protocol was applied at an ultrasound intensity of 117.5 to 187.9 mW/cm² (I_{SPTA}) (spatial-peak temporal average intensity), at a mechanical index of 1.4, and a frequency of 2.5 MHz. Ultrasound energy was applied using an alternating imaging technique applying focused ultrasound and generating a B-mode ultrasound image at a mechanical index of 0.4, and a frequency of 2.5 MHz with the ultrasound probe removed from contacting the subject as indicated.

After the administration of the sonoporation treatment, FVIII expression and secretion was measured in subject plasma samples collected and frozen at −80 C approximately every 10 days following the sonoporation treatment for the first forty days, and then every forty days for the next 3 sample collections.

Transgenic FVIII level in mouse plasma was measured by immunoassay (MESO SCALE DIAGNOSTICS, LLC). Briefly capture antibody (GMA-8024) was loaded to the 96-well plate overnight at 4 C. Next the plate was washed three times with wash buffer and incubated with blocking buffer for 30 min at room temperature. 8 point serial dilution standard were prepared using Xinta® ranging from 0.921U/ml to 0.01 IU/ml. 2-fold diluted samples and standards were added to the wells in 96-well plate. The plate was incubated 2 hours at room temperature and washed 3 times. The detection was performed by incubating samples with GMA-8023 antibody during 2 hours following triple wash. Signal was developed by Sulfo-TAG and detected by MSD machine.

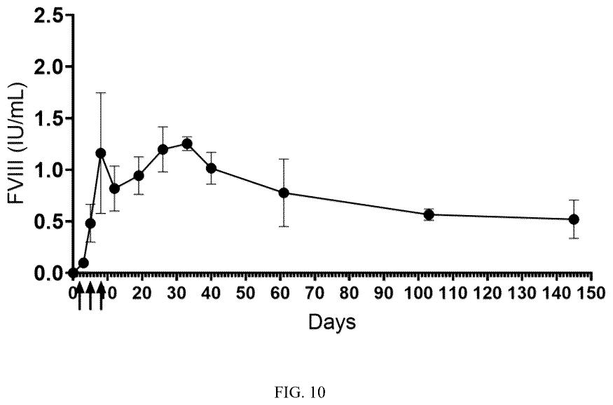

Results are shown in FIG. 10, in which it is shown that the experimental group administered 50 μg of a nucleic acid composition comprising the sequence of SEQ ID NO:39 (ApoE-AAT-hBGi-FVIII-N6-F309S-DM-WPRE3-DTS), a nucleic acid expression vector encoding a modified FVIII transgene (SEQ ID NO: 24), exhibits: FVIII levels of about 1.16 IU/mL following the administration of the cycle of three doses with expression levels maintained from a range of 0.8 to 1.25 IU/mL through day 40, with FVIII levels of 0.77, 0.56, 0.52 IU/mL being observed at days 61, 103, and 145 respectively.

The results shown herein indicate that the modifications to the nucleic acid expression vector (SEQ ID NO:39) disclosed herein encoding the modified FVIII transgene (SEQ ID NO: 24) disclosed herein in provide a beneficial technical effect in increasing expression of FVIII in-vivo and maintaining a stable level of FVIII expression in-vivo following a multiple treatment protocol for at least 145 days.

### Definitions

As used herein, the term “effective amount” refers to an amount sufficient to affect beneficial or desirable biological and/or clinical results.

As used herein, the term “full-length” as applied to a gene, transgene, or polypeptide refers to an amino acid sequence (or a nucleic acid that encodes an amino acid sequence) that comprises all domains of the polypeptide that are needed for functional biological activity. Thus, a full-length sequence may refer to a truncated or modified version of a protein (or a nucleic acid that directly encodes the protein) so long as the resulting protein retains functional biological activity. However, neither a non-functional protein nor a nucleic acid that encodes that encodes only non-functional portions of a polypeptide are full-length proteins.

As used herein, the term “sonoactive agent” or “sonoactive microstructure” refers to ultrasound agents which undergo cavitation when exposed to ultrasound acoustic energy and which may be used to deliver a sonoporation treatment to a subject.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “isolated” when used in relation to a nucleic acid molecule of the present disclosure typically refers to a nucleic acid sequence that is identified and separated from at least one contaminant nucleic acid with which it is ordinarily associated in its natural source. Isolated nucleic acid may be present in a form or setting that is different from that in which it is found in nature. Isolated nucleic acid molecules therefore are distinguished from the nucleic acid molecule as they exist in natural cells.

The term “identity,” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions, they share identity within that region. An “aligned” sequence refers to multiple polynucleotide or protein (amino acid) sequences, often containing corrections for missing or additional bases or amino acids (gaps) as compared to a reference sequence.

As used herein the term “sequence identity” refers to the percentage identity calculated as the matching residues divided by the total number of residues in the total alignment when performing a consensus alignment of two sequences, with gaps in the alignment scored as a mismatching residue.

As used herein “therapeutic protein” refers to a polypeptide that has a biological activity that replaces or compensates for the loss or reduction of activity of an endogenous protein. For example, functional human factor VIII is a therapeutic protein for hemophilia A.

As used herein “FVIII polypeptide” refers to a full length FVIII polypeptide or any biologically active fragment or variant of a FVIII polypeptide.

What has been described and illustrated herein is an example along with some of its variations. The terms, descriptions and figures used herein are set forth by way of illustration only and are not meant as limitations.

SEQUENCE LISTING

| Element | Sequence | # |
|---|---|---|
| ApoE-AAT | CGCGTCTGCAGGCTCAGAGGCACACAGGAGTTTCTGGGCTCACCCTGCCCCCTTCCAACCCCTCA | 1 |
| promoter | GTTCCCATCCTCCAGCAGCTGTTTGTGTGCTGCCTCTGAAGTCCACACTGAACAAACTTCAGCCT | |
| | ACTCATGTCCCTAAAATGGGCAAACATTGCAAGCAGCAAACAGCAAACACACAGCCCTCCCTGC | |
| | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCC | |
| hBGi intron | TCTGGATCCACTGCTTAAATACGGACGAGGACAGGGCCCTGTCTCCTCAGCTTCAGGCACCACC | 2 |
| | ACTGACCTGGGACAGTGAATCGTAAGTACTAGCAGCTACAATCCAGCTACCATTCTGCTTTTATT | |
| | TTATGGTTGGGATAAGGCTGGATTATTCTGAGTCCAAGCTAGGCCCTTTTGCTAATCATGTTCAT | |
| | ACCTCTTATCTTCCTCCCACAGCTCCTGGGCAACGTGCTGGTCTGTGTGCTGGCCCATCACTTTGG | |
| | CAAAGAATTGCGATCGCCACC | |
| FVIII-v3 | ATGCAGATTGAGCTGAGCACCTGCTTCTTCCTGTGCCTGCTGAGGTTCTGCTTCTCTGCCACCAG | 3 |
| ORF | GAGATACTACCTGGGGGCTGTGGAGCTGAGCTGGGACTACATGCAGTCTGACCTGGGGGAGCTG | |
| | CCTGTGGATGCCAGGTTCCCCCCCAGAGTGCCCAAGAGCTTCCCCTTCAACACCTCTGTGGTGTA | |
| | CAAGAAGACCCTGTTTGTGGAGTTCACTGACCACCTGTTCAACATTGCCAAGCCCAGGCCCCCCT | |
| | GGATGGGCCTGCTGGGCCCCACCATCCAGGCTGAGGTGTATGACACTGTGGTGATCACCCTGAA | |
| | GAACATGGCCAGCCACCCTGTGAGCCTGCATGCTGTGGGGGTGAGCTACTGGAAGGCCTCTGAG | |
| | GGGGCTGAGTATGATGACCAGACCAGCCAGAGGGAGAAGGAGGATGACAAGGTGTTCCCTGGG | |
| | GGCAGCCACACCTATGTGTGGCAGGTGCTGAAGGAGAATGGCCCCATGGCCTCTGACCCCCTGT | |
| | GCCTGACCTACAGCTACCTGAGCCATGTGGACCTGGTGAAGGACCTGAACTCTGGCCTGATTGG | |
| | GGCCCTGCTGGTGTGCAGGGAGGGCAGCCTGGCCAAGGAGAAGACCCAGACCCTGCACAAGTT | |
| | CATCCTGCTGTTTGCTGTGTTTGATGAGGGCAAGAGCTGGCACTCTGAAACCAAGAACAGCCTG | |
| | ATGCAGGACAGGGATGCTGCCTCTGCCAGGGCCTGGCCCAAGATGCACACTGTGAATGGCTATG | |
| | TGAACAGGAGCCTGCCTGGCCTGATTGGCTGCCACAGGAAGTCTGTGTACTGGCATGTGATTGG | |
| | CATGGGCACCACCCCTGAGGTGCACAGCATCTTCCTGGAGGGCCACACCTTCCTGGTCAGGAAC | |
| | CACAGGCAGGCCAGCCTGGAGATCAGCCCCATCACCTTCCTGACTGCCCAGACCCTGCTGATGG | |
| | ACCTGGGCCAGTTCCTGCTGTTCTGCCACATCAGCAGCCACCAGCATGATGGCATGGAGGCCTA | |
| | TGTGAAGGTGGACAGCTGCCCTGAGGAGCCCCAGCTGAGGATGAAGAACAATGAGGAGGCTGA | |
| | GGACTATGATGATGACCTGACTGACTCTGAGATGGATGTGGTGAGGTTTGATGATGACAACAGC | |
| | CCCAGCTTCATCCAGATCAGGTCTGTGGCCAAGAAGCACCCCAAGACCTGGGTGCACTACATTG | |
| | CTGCTGAGGAGGAGGACTGGGACTATGCCCCCCTGGTGCTGGCCCCTGATGACAGGAGCTACAA | |
| | GAGCCAGTACCTGAACAATGGCCCCCAGAGGATTGGCAGGAAGTACAAGAAGGTCAGGTTCAT | |
| | GGCCTACACTGATGAAACCTTCAAGACCAGGGAGGCCATCCAGCATGAGTCTGGCATCCTGGGC | |
| | CCCCTGCTGTATGGGGAGGTGGGGGACACCCTGCTGATCATCTTCAAGAACCAGGCCAGCAGGC | |
| | CCTACAACATCTACCCCCATGGCATCACTGATGTGAGGCCCCTGTACAGCAGGAGGCTGCCCAA | |
| | GGGGGTGAAGCACCTGAAGGACTTCCCCATCCTGCCTGGGGAGATCTTCAAGTACAAGTGGACT | |
| | GTGACTGTGGAGGATGGCCCCACCAAGTCTGACCCCAGGTGCCTGACCAGATACTACAGCAGCT | |
| | TTGTGAACATGGAGAGGGACCTGGCCTCTGGCCTGATTGGCCCCCTGCTGATCTGCTACAAGGA | |
| | GTCTGTGGACCAGAGGGGCAACCAGATCATGTCTGACAAGAGGAATGTGATCCTGTTCTCTGTG | |
| | TTTGATGAGAACAGGAGCTGGTACCTGACTGAGAACATCCAGAGGTTCCTGCCCAACCCTGCTG | |
| | GGGTGCAGCTGGAGGACCCTGAGTTCCAGGCCAGCAACATCATGCACAGCATCAATGGCTATGT | |
| | GTTTGACAGCCTGCAGCTGTCTGTGTGCCTGCATGAGGTGGCCTACTGGTACATCCTGAGCATTG | |
| | GGGCCCAGACTGACTTCCTGTCTGTGTTCTTCTCTGGCTACACCTTCAAGCACAAGATGGTGTAT | |
| | GAGGACACCCTGACCCTGTTCCCCTTCTCTGGGGAGACTGTGTTCATGAGCATGGAGAACCCTG | |
| | GCCTGTGGATTCTGGGCTGCCACAACTCTGACTTCAGGAACAGGGGCATGACTGCCCTGCTGAA | |
| | AGTCTCCAGCTGTGACAAGAACACTGGGGACTACTATGAGGACAGCTATGAGGACATCTCTGCC | |
| | TACCTGCTGAGCAAGAACAATGCCATTGAGCCCAGGAGCTTCAGCCAGAATGCCACTAATGTGT | |
| | CTAACAACAGCAACACCAGCAATGACAGCAATGTGTCTCCCCCAGTGCTGAAGAGGCACCAGA | |
| | GGGAGATCACCAGGACCACCCTGCAGTCTGACCAGGAGGAGATTGACTATGATGACACCATCTC | |
| | TGTGGAGATGAAGAAGGAGGACTTTGACATCTACGACGAGGACGAGAACCAGAGCCCCAGGAG | |
| | CTTCCAGAAGAAGACCAGGCACTACTTCATTGCTGCTGTGGAGAGGCTGTGGGACTATGGCATG | |
| | AGCAGCAGCCCCCATGTGCTGAGGAACAGGGCCCAGTCTGGCTCTGTGCCCCAGTTCAAGAAGG | |
| | TGGTGTTCCAGGAGTTCACTGATGGCAGCTTCACCCAGCCCCTGTACAGAGGGGAGCTGAATGA | |
| | GCACCTGGGCCTGCTGGGCCCCTACATCAGGGCTGAGGTGGAGGACAACATCATGGTGACCTTC | |
| | AGGAACCAGGCCAGCAGGCCCTACAGCTTCTACAGCAGCCTGATCAGCTATGAGGAGGACCAG | |
| | AGGCAGGGGGCTGAGCCCAGGAAGAACTTTGTGAAGCCCAATGAAACCAAGACCTACTTCTGG | |
| | AAGGTGCAGCACCACATGGCCCCCACCAAGGATGAGTTTGACTGCAAGGCCTGGGCCTACTTCT | |
| | CTGATGTGGACCTGGAGAAGGATGTGCACTCTGGCCTGATTGGCCCCCTGCTGGTGTGCCACAC | |
| | CAACACCCTGAACCCTGCCCATGGCAGGCAGGTGACTGTGCAGGAGTTTGCCCTGTTCTTCACCA | |
| | TCTTTGATGAAACCAAGAGCTGGTACTTCACTGAGAACATGGAGAGGAACTGCAGGGCCCCCTG | |
| | CAACATCCAGATGGAGGACCCCACCTTCAAGGAGAACTACAGGTTCCATGCCATCAATGGCTAC | |
| | ATCATGGACACCCTGCCTGGCCTGGTGATGGCCCAGGACCAGAGGATCAGGTGGTACCTGCTGA | |
| | GCATGGGCAGCAATGAGAACATCCACAGCATCCACTTCTCTGGCCATGTGTTCACTGTGAGGAA | |
| | GAAGGAGGAGTACAAGATGGCCCTGTACAACCTGTACCCTGGGGTGTTTGAGACTGTGGAGATG | |
| | CTGCCCAGCAAGGCTGGCATCTGGAGGGTGGAGTGCCTGATTGGGGAGCACCTGCATGCTGGCA | |
| | TGAGCACCCTGTTCCTGGTGTACAGCAACAAGTGCCAGACCCCCCTGGGCATGGCCTCTGGCCA | |
| | CATCAGGGACTTCCAGATCACTGCCTCTGGCCAGTATGGCCAGTGGGCCCCCAAGCTGGCCAGG | |
| | CTGCACTACTCTGGCAGCATCAATGCCTGGAGCACCAAGGAGCCCTTCAGCTGGATCAAGGTGG | |
| | ACCTGCTGGCCCCCATGATCATCCATGGCATCAAGACCCAGGGGGCCAGGCAGAAGTTCAGCAG | |
| | CCTGTACATCAGCCAGTTCATCATCATGTACAGCCTGGATGGCAAGAAGTGGCAGACCTACAGG | |
| | GGCAACAGCACTGGCACCCTGATGGTGTTCTTTGGCAATGTGGACAGCTCTGGCATCAAGCACA | |
| | ACATCTTCAACCCCCCCATCATTGCCAGATACATCAGGCTGCACCCCACCCACTACAGCATCAGG | |
| | AGCACCCTGAGGATGGAGCTGATGGGCTGTGACCTGAACAGCTGCAGCATGCCCCTGGGCATGG | |
| | AGAGCAAGGCCATCTCTGATGCCCAGATCACTGCCAGCAGCTACTTCACCAACATGTTTGCCAC | |
| | CTGGAGCCCCAGCAAGGCCAGGCTGCACCTGCAGGGCAGGAGCAATGCCTGGAGGCCCCAGGT | |
| | CAACAACCCCAAGGAGTGGCTGCAGGTGGACTTCCAGAAGACCATGAAGGTGACTGGGGTGAC | |
| | CACCCAGGGGGTGAAGAGCCTGCTGACCAGCATGTATGTGAAGGAGTTCCTGATCAGCAGCAGC | |
| | CAGGATGGCCACCAGTGGACCCTGTTCTTCCAGAATGGCAAGGTGAAGGTGTTCCAGGGCAACC | |
| | AGGACAGCTTCACCCCTGTGGTGAACAGCCTGGACCCCCCCCTGCTGACCAGATACCTGAGGAT | |
| | TCACCCCCAGAGCTGGGTGCACCAGATTGCCCTGAGGATGGAGGTGCTGGGCTGTGAGGCCCAG | |
| | GACCTGTACTGA | |
| WPRE3 | AATCAACCTCTGGATTACAAAATTTGTGAAAGATTGACTGGTATTCTTAACTATGTTGCTCCTTTT | 4 |
| | ACGCTATGTGGATACGCTGCTTTAATGCCTTTGTATCATGCTATTGCTTCCCGTATGGCTTTCATT | |
| | TTCTCCTCCTTGTATAAATCCTGGTTGCTGTCTCTTTATGAGGAGTTGTGGCCCGTTGTCAGGCAA | |
| | CGTGGCGTGGTGTGCACTGTGTTTGCTGACGCAACCCCCACTGGTTGGGGCATTGCCACCACCTG | |
| | TCAGCTCCTTTCCGGGACTTTCGCTTTCCCCCTCCCTATTGCCACGGCGGAACTCATCGCCGCCTG | |
| | CCTTGCCCGCTGCTGGACAGGGGCTCGGCTGTTGGGCACTGACAATTCCGTGG | |
| bGH poly A | TGTGCCTTCTAGTTGCCAGCCATCTGTTGTTTGCCCCTCCCCCGTGCCTTCCTTGACCCTGGAAGG | 5 |
| | TGCCACTCCCACTGTCCTTTCCTAATAAAATGAGGAAATTGCATCGCATTGTCTGAGTAGGTGTC | |
| | ATTCTATTCTGGGGGGTGGGGTGGGGCAGGACAGCAAGGGGGAGGATTGGGAAGACAATAGCA | |
| | GGCATGCTGGGGATGCGGTGGGCTCTATGG | |
| DTS | GAGCTGTGCGATCCCTGCTGGGGACTTTCCGCTGGGGACTTTCCGCTGGGGACTTTCCGCCTTCA | 6 |
| | GCTAAGGAAGCTACCAATATTTAGAGGTACATTTTGTTCTAGAACAAAATGTACCGGTACATTTT | |
| | GTTCTGGTACATTTTGTTCT | |
| NP | TGGCTTGTTGTCCACAACCATTAAACCTTAAAAGCTTTAAAAGCCTTATATATTCTTTTTTTTCTT | 7 |
| Backbone | ATAAAACTTAAAACCTTAGAGGCTATTTAAGTTGCTGATTTATATTAATTTTATTGTTCAAACAT | |
| | GAGAGCTTAGTACGTGAAACATGAGAGCTTAGTACATTAGCCATGAGAGCTTAGTACATTAGCC | |
| | ATGAGGGTTTAGTTCATTAAACATGAGAGCTTAGTACATTAAACATGAGAGCTTAGTACATTAA | |
| | ACATGAGAGCTTAGTACATACTATCAACAGGTTGAACTGCTGATCTGTACAGTAGAATTGGTAA | |
| | AGAGAGTTGTGTAAAATATTGAGTTCGCACATCTTGTTGTCTGATTATTGATTTTTGGCGAAACC | |
| | ATTTGATCATATGACAAGATGTGTATCTACCTTAACTTAATGATTTTGATAAAAATCATTA | |
| FVIII-v3 | MQIELSTCFFLCLLRFCFSATRRYYLGAVELSWDYMQSDLGELPVDARFPPRVPKSFPFNTSVVYKKT | 8 |
| AA | LFVEFTDHLFNIAKPRPPWMGLLGPTIQAEVYDTVVITLKNMASHPVSLHAVGVSYWKASEGAEYD | |
| sequence | DQTSQREKEDDKVFPGGSHTYVWQVLKENGPMASDPLCLTYSYLSHVDLVKDLNSGLIGALLVCRE | |
| | GSLAKEKTQTLHKFILLFAVFDEGKSWHSETKNSLMQDRDAASARAWPKMHTVNGYVNRSLPGLIG | |
| | CHRKSVYWHVIGMGTTPEVHSIFLEGHTFLVRNHRQASLEISPITFLTAQTLLMDLGQFLLFCHISSHQ | |
| | HDGMEAYVKVDSCPEEPQLRMKNNEEAEDYDDDLTDSEMDVVRFDDDNSPSFIQIRSVAKKHPKT | |
| | WVHYIAAEEEDWDYAPLVLAPDDRSYKSQYLNNGPQRIGRKYKKVRFMAYTDETFKTREAIQHESG | |
| | ILGPLLYGEVGDTLLIIFKNQASRPYNIYPHGITDVRPLYSRRLPKGVKHLKDFPILPGEIFKYKWTVTV | |
| | EDGPTKSDPRCLTRYYSSFVNMERDLASGLIGPLLICYKESVDQRGNQIMSDKRNVILFSVFDENRSW | |
| | YLTENIQRFLPNPAGVQLEDPEFQASNIMHSINGYVFDSLQLSVCLHEVAYWYILSIGAQTDFLSVFFS | |
| | GYTFKHKMVYEDTLTLFPFSGETVFMSMENPGLWILGCHNSDFRNRGMTALLKVSSCDKNTGDYYE | |
| | DSYEDISAYLLSKNNAIEPRSFSQNATNVSNNSNTSNDSNVSPPVLKRHQREITRTTLQSDQEEIDYDD | |
| | TISVEMKKEDFDIYDEDENQSPRSFQKKTRHYFIAAVERLWDYGMSSSPHVLRNRAQSGSVPQFKKV | |
| | VFQEFTDGSFTQPLYRGELNEHLGLLGPYIRAEVEDNIMVTFRNQASRPYSFYSSLISYEEDQRQGAEP | |
| | RKNFVKPNETKTYFWKVQHHMAPTKDEFDCKAWAYFSDVDLEKDVHSGLIGPLLVCHTNTLNPAH | |
| | GRQVTVQEFALFFTIFDETKSWYFTENMERNCRAPCNIQMEDPTFKENYRFHAINGYIMDTLPGLVM | |
| | AQDQRIRWYLLSMGSNENIHSIHFSGHVFTVRKKEEYKMALYNLYPGVFETVEMLPSKAGIWRVEC | |
| | LIGEHLHAGMSTLFLVYSNKCQTPLGMASGHIRDFQITASGQYGQWAPKLARLHYSGSINAWSTKEP | |
| | FSWIKVDLLAPMIIHGIKTQGARQKFSSLYISQFIIMYSLDGKKWQTYRGNSTGTLMVFFGNVDSSGI | |
| | KHNIFNPPIIARYIRLHPTHYSIRSTLRMELMGCDLNSCSMPLGMESKAISDAQITASSYFTNMFATWS | |
| | PSKARLHLQGRSNAWRPQVNNPKEWLQVDFQKTMKVTGVTTQGVKSLLTSMYVKEFLISSSQDGH | |
| | QWTLFFQNGKVKVFQGNQDSFTPVVNSLDPPLLTRYLRIHPQSWVHQIALRMEVLGCEAQDLY* | |
| ApoE-AAT- | CGCGTCTGCAGGCTCAGAGGCACACAGGAGTTTCTGGGCTCACCCTGCCCCCTTCCAACCCCTCA | 9 |
| hbGi | GTTCCCATCCTCCAGCAGCTGTTTGTGTGCTGCCTCTGAAGTCCACACTGAACAAACTTCAGCCT | |
| | ACTCATGTCCCTAAAATGGGCAAACATTGCAAGCAGCAAACAGCAAACACACAGCCCTCCCTGC | |
| | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACC | |
| FIX NA | ATGCAGCGCGTGAACATGATCATGGCAGAATCACCAGGCCTCATCACCATCTGCCTTTTAGGAT | 10 |
| sequence | ATCTACTCAGTGCTGAATGTACAGTTTTTCTTGATCATGAAAACGCCAACAAAATTCTGAATCGG | |
| | CCAAAGAGGTATAATTCAGGTAAATTGGAAGAGTTTGTTCAAGGGAACCTTGAGAGAGAATGTA | |
| | TGGAAGAAAAGTGTAGTTTTGAAGAAGCACGAGAAGTTTTTGAAAACACTGAAAGAACAACTG | |
| | AATTTTGGAAGCAGTATGTTGATGGAGATCAGTGTGAGTCCAATCCATGTTTAAATGGCGGCAG | |
| | TTGCAAGGATGACATTAATTCCTATGAATGTTGGTGTCCCTTTGGATTTGAAGGAAAGAACTGTG | |
| | AATTAGATGTAACATGTAACATTAAGAATGGCAGATGCGAGCAGTTTTGTAAAAATAGTGCTGA | |
| | TAACAAGGTGGTTTGCTCCTGTACTGAGGGATATCGACTTGCAGAAAACCAGAAGTCCTGTGAA | |
| | CCAGCAGTGCCATTTCCATGTGGAAGAGTTTCTGTTTCACAAACTTCTAAGCTCACCCGTGCTGA | |
| | GACTGTTTTTCCTGATGTGGACTATGTAAATTCTACTGAAGCTGAAACCATTTTGGATAACATCA | |
| | CTCAAAGCACCCAATCATTTAATGACTTCACTCGGGTTGTTGGTGGAGAAGATGCCAAACCAGG | |
| | TCAATTCCCTTGGCAGGTTGTTTTGAATGGTAAAGTTGATGCATTCTGTGGAGGCTCTATCGTTA | |
| | ATGAAAAATGGATTGTAACTGCTGCCCACTGTGTTGAAACTGGTGTTAAAATTACAGTTGTCGCA | |
| | GGTGAACATAATATTGAGGAGACAGAACATACAGAGCAAAAGCGAAATGTGATTCGAATTATT | |
| | CCTCACCACAACTACAATGCAGCTATTAATAAGTACAACCATGACATTGCCCTTCTGGAACTGG | |
| | ACGAACCCTTAGTGCTAAACAGCTACGTTACACCTATTTGCATTGCTGACAAGGAATACACGAA | |
| | CATCTTCCTCAAATTTGGATCTGGCTATGTAAGTGGCTGGGGAAGAGTCTTCCACAAAGGGAGA | |
| | TCAGCTTTAGTTCTTCAGTACCTTAGAGTTCCACTTGTTGACCGAGCCACATGTCTTCGATCTACA | |
| | AAGTTCACCATCTATAACAACATGTTCTGTGCTGGCTTCCATGAAGGAGGTAGAGATTCATGTCA | |
| | AGGAGATAGTGGGGGACCCCATGTTACTGAAGTGGAAGGGACCAGTTTCTTAACTGGAATTATT | |
| | AGCTGGGGTGAAGAGTGTGCAATGAAAGGCAAATATGGAATATATACCAAGGTATCCCGGTAT | |
| | GTCAACTGGATTAAGGAAAAAACAAAGCTCACTTAATGA | |
| 5′ITR | CCTGCAGGCAGCTGCGCGCTCGCTCGCTCACTGAGGCCGCCCGGGCGTCGGGCGACCTTTGGTC | 11 |
| | GCCCGGCCTCAGTGAGCGAGCGCGCAGAGAGGGAGTGGCCAACTCCATCACTAGGGGTTCCT | |
| APOE- | CGCGTCTGCAGGCTCAGAGGCACACAGGAGTTTCTGGGCTCACCCTGCCCCCTTCCAACCCCTCA | 12 |
| AAT-hBGi- | GTTCCCATCCTCCAGCAGCTGTTTGTGTGCTGCCTCTGAAGTCCACACTGAACAAACTTCAGCCT | |
| FIX | ACTCATGTCCCTAAAATGGGCAAACATTGCAAGCAGCAAACAGCAAACACACAGCCCTCCCTGC | |
| | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGCGCGTGAACATGATCATGGC | |
| | AGAATCACCAGGCCTCATCACCATCTGCCTTTTAGGATATCTACTCAGTGCTGAATGTACAGTTT | |
| | TTCTTGATCATGAAAACGCCAACAAAATTCTGAATCGGCCAAAGAGGTATAATTCAGGTAAATT | |
| | GGAAGAGTTTGTTCAAGGGAACCTTGAGAGAGAATGTATGGAAGAAAAGTGTAGTTTTGAAGA | |
| | AGCACGAGAAGTTTTTGAAAACACTGAAAGAACAACTGAATTTTGGAAGCAGTATGTTGATGGA | |
| | GATCAGTGTGAGTCCAATCCATGTTTAAATGGCGGCAGTTGCAAGGATGACATTAATTCCTATG | |
| | AATGTTGGTGTCCCTTTGGATTTGAAGGAAAGAACTGTGAATTAGATGTAACATGTAACATTAA | |
| | GAATGGCAGATGCGAGCAGTTTTGTAAAAATAGTGCTGATAACAAGGTGGTTTGCTCCTGTACT | |
| | GAGGGATATCGACTTGCAGAAAACCAGAAGTCCTGTGAACCAGCAGTGCCATTTCCATGTGGAA | |
| | GAGTTTCTGTTTCACAAACTTCTAAGCTCACCCGTGCTGAGACTGTTTTTCCTGATGTGGACTATG | |
| | TAAATTCTACTGAAGCTGAAACCATTTTGGATAACATCACTCAAAGCACCCAATCATTTAATGAC | |
| | TTCACTCGGGTTGTTGGTGGAGAAGATGCCAAACCAGGTCAATTCCCTTGGCAGGTTGTTTTGAA | |
| | TGGTAAAGTTGATGCATTCTGTGGAGGCTCTATCGTTAATGAAAAATGGATTGTAACTGCTGCCC | |
| | ACTGTGTTGAAACTGGTGTTAAAATTACAGTTGTCGCAGGTGAACATAATATTGAGGAGACAGA | |
| | ACATACAGAGCAAAAGCGAAATGTGATTCGAATTATTCCTCACCACAACTACAATGCAGCTATT | |
| | AATAAGTACAACCATGACATTGCCCTTCTGGAACTGGACGAACCCTTAGTGCTAAACAGCTACG | |
| | TTACACCTATTTGCATTGCTGACAAGGAATACACGAACATCTTCCTCAAATTTGGATCTGGCTAT | |
| | GTAAGTGGCTGGGGAAGAGTCTTCCACAAAGGGAGATCAGCTTTAGTTCTTCAGTACCTTAGAG | |
| | TTCCACTTGTTGACCGAGCCACATGTCTTCGATCTACAAAGTTCACCATCTATAACAACATGTTC | |
| | TGTGCTGGCTTCCATGAAGGAGGTAGAGATTCATGTCAAGGAGATAGTGGGGGACCCCATGTTA | |
| | CTGAAGTGGAAGGGACCAGTTTCTTAACTGGAATTATTAGCTGGGGTGAAGAGTGTGCAATGAA | |
| | AGGCAAATATGGAATATATACCAAGGTATCCCGGTATGTCAACTGGATTAAGGAAAAAACAAA | |
| | GCTCACTTAATGA | |
| APOE- | CGCGTCTGCAGGCTCAGAGGCACACAGGAGTTTCTGGGCTCACCCTGCCCCCTTCCAACCCCTCA | 13 |
| AAT-hBGi- | GTTCCCATCCTCCAGCAGCTGTTTGTGTGCTGCCTCTGAAGTCCACACTGAACAAACTTCAGCCT | |
| FIX-bGH- | ACTCATGTCCCTAAAATGGGCAAACATTGCAAGCAGCAAACAGCAAACACACAGCCCTCCCTGC | |
| poly A | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGCGCGTGAACATGATCATGGC | |
| | AGAATCACCAGGCCTCATCACCATCTGCCTTTTAGGATATCTACTCAGTGCTGAATGTACAGTTT | |
| | TTCTTGATCATGAAAACGCCAACAAAATTCTGAATCGGCCAAAGAGGTATAATTCAGGTAAATT | |
| | GGAAGAGTTTGTTCAAGGGAACCTTGAGAGAGAATGTATGGAAGAAAAGTGTAGTTTTGAAGA | |
| | AGCACGAGAAGTTTTTGAAAACACTGAAAGAACAACTGAATTTTGGAAGCAGTATGTTGATGGA | |
| | GATCAGTGTGAGTCCAATCCATGTTTAAATGGCGGCAGTTGCAAGGATGACATTAATTCCTATG | |
| | AATGTTGGTGTCCCTTTGGATTTGAAGGAAAGAACTGTGAATTAGATGTAACATGTAACATTAA | |
| | GAATGGCAGATGCGAGCAGTTTTGTAAAAATAGTGCTGATAACAAGGTGGTTTGCTCCTGTACT | |
| | GAGGGATATCGACTTGCAGAAAACCAGAAGTCCTGTGAACCAGCAGTGCCATTTCCATGTGGAA | |
| | GAGTTTCTGTTTCACAAACTTCTAAGCTCACCCGTGCTGAGACTGTTTTTCCTGATGTGGACTATG | |
| | TAAATTCTACTGAAGCTGAAACCATTTTGGATAACATCACTCAAAGCACCCAATCATTTAATGAC | |
| | TTCACTCGGGTTGTTGGTGGAGAAGATGCCAAACCAGGTCAATTCCCTTGGCAGGTTGTTTTGAA | |
| | TGGTAAAGTTGATGCATTCTGTGGAGGCTCTATCGTTAATGAAAAATGGATTGTAACTGCTGCCC | |
| | ACTGTGTTGAAACTGGTGTTAAAATTACAGTTGTCGCAGGTGAACATAATATTGAGGAGACAGA | |
| | ACATACAGAGCAAAAGCGAAATGTGATTCGAATTATTCCTCACCACAACTACAATGCAGCTATT | |
| | AATAAGTACAACCATGACATTGCCCTTCTGGAACTGGACGAACCCTTAGTGCTAAACAGCTACG | |
| | TTACACCTATTTGCATTGCTGACAAGGAATACACGAACATCTTCCTCAAATTTGGATCTGGCTAT | |
| | GTAAGTGGCTGGGGAAGAGTCTTCCACAAAGGGAGATCAGCTTTAGTTCTTCAGTACCTTAGAG | |
| | TTCCACTTGTTGACCGAGCCACATGTCTTCGATCTACAAAGTTCACCATCTATAACAACATGTTC | |
| | TGTGCTGGCTTCCATGAAGGAGGTAGAGATTCATGTCAAGGAGATAGTGGGGGACCCCATGTTA | |
| | CTGAAGTGGAAGGGACCAGTTTCTTAACTGGAATTATTAGCTGGGGTGAAGAGTGTGCAATGAA | |
| | AGGCAAATATGGAATATATACCAAGGTATCCCGGTATGTCAACTGGATTAAGGAAAAAACAAA | |
| | GCTCACTTAATGACCTCGAGGTGTGCCTTCTAGTTGCCAGCCATCTGTTGTTTGCCCCTCCCCCGT | |
| | GCCTTCCTTGACCCTGGAAGGTGCCACTCCCACTGTCCTTTCCTAATAAAATGAGGAAATTGCAT | |
| | CGCATTGTCTGAGTAGGTGTCATTCTATTCTGGGGGGTGGGGTGGGGCAGGACAGCAAGGGGGA | |
| | GGATTGGGAAGACAATAGCAGGCATGCTGGGGATGCGGTGGGCTCTATGG | |
| APOE- | CGCGTCTGCAGGCTCAGAGGCACACAGGAGTTTCTGGGCTCACCCTGCCCCCTTCCAACCCCTCA | 14 |
| AAT-hBGi- | GTTCCCATCCTCCAGCAGCTGTTTGTGTGCTGCCTCTGAAGTCCACACTGAACAAACTTCAGCCT | |
| FIX | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| bGH- | CACTCGACCCCTTGGAATTTCGGTGGAGAGGAGCAGAGGTTGTCCTGGCGTGGTTTAGGTAGTG | |
| poly A-DTS | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGCGCGTGAACATGATCATGGC | |
| | AGAATCACCAGGCCTCATCACCATCTGCCTTTTAGGATATCTACTCAGTGCTGAATGTACAGTTT | |
| | TTCTTGATCATGAAAACGCCAACAAAATTCTGAATCGGCCAAAGAGGTATAATTCAGGTAAATT | |
| | GGAAGAGTTTGTTCAAGGGAACCTTGAGAGAGAATGTATGGAAGAAAAGTGTAGTTTTGAAGA | |
| | AGCACGAGAAGTTTTTGAAAACACTGAAAGAACAACTGAATTTTGGAAGCAGTATGTTGATGGA | |
| | GATCAGTGTGAGTCCAATCCATGTTTAAATGGCGGCAGTTGCAAGGATGACATTAATTCCTATG | |
| | AATGTTGGTGTCCCTTTGGATTTGAAGGAAAGAACTGTGAATTAGATGTAACATGTAACATTAA | |
| | GAATGGCAGATGCGAGCAGTTTTGTAAAAATAGTGCTGATAACAAGGTGGTTTGCTCCTGTACT | |
| | GAGGGATATCGACTTGCAGAAAACCAGAAGTCCTGTGAACCAGCAGTGCCATTTCCATGTGGAA | |
| | GAGTTTCTGTTTCACAAACTTCTAAGCTCACCCGTGCTGAGACTGTTTTTCCTGATGTGGACTATG | |
| | TAAATTCTACTGAAGCTGAAACCATTTTGGATAACATCACTCAAAGCACCCAATCATTTAATGAC | |
| | TTCACTCGGGTTGTTGGTGGAGAAGATGCCAAACCAGGTCAATTCCCTTGGCAGGTTGTTTTGAA | |
| | TGGTAAAGTTGATGCATTCTGTGGAGGCTCTATCGTTAATGAAAAATGGATTGTAACTGCTGCCC | |
| | ACTGTGTTGAAACTGGTGTTAAAATTACAGTTGTCGCAGGTGAACATAATATTGAGGAGACAGA | |
| | ACATACAGAGCAAAAGCGAAATGTGATTCGAATTATTCCTCACCACAACTACAATGCAGCTATT | |
| | AATAAGTACAACCATGACATTGCCCTTCTGGAACTGGACGAACCCTTAGTGCTAAACAGCTACG | |
| | TTACACCTATTTGCATTGCTGACAAGGAATACACGAACATCTTCCTCAAATTTGGATCTGGCTAT | |
| | GTAAGTGGCTGGGGAAGAGTCTTCCACAAAGGGAGATCAGCTTTAGTTCTTCAGTACCTTAGAG | |
| | TTCCACTTGTTGACCGAGCCACATGTCTTCGATCTACAAAGTTCACCATCTATAACAACATGTTC | |
| | TGTGCTGGCTTCCATGAAGGAGGTAGAGATTCATGTCAAGGAGATAGTGGGGGACCCCATGTTA | |
| | CTGAAGTGGAAGGGACCAGTTTCTTAACTGGAATTATTAGCTGGGGTGAAGAGTGTGCAATGAA | |
| | AGGCAAATATGGAATATATACCAAGGTATCCCGGTATGTCAACTGGATTAAGGAAAAAACAAA | |
| | GCTCACTTAATGACCCTCGAGAATCAACCTCTGGATTACAAAATTTGTGAAAGATTGACTGGTAT | |
| | TCTTAACTATGTTGCTCCTTTTACGCTATGTGGATACGCTGCTTTAATGCCTTTGTATCATGCTAT | |
| | TGCTTCCCGTATGGCTTTCATTTTCTCCTCCTTGTATAAATCCTGGTTGCTGTCTCTTTATGAGGA | |
| | GTTGTGGCCCGTTGTCAGGCAACGTGGCGTGGTGTGCACTGTGTTTGCTGACGCAACCCCCACTG | |
| | GTTGGGGCATTGCCACCACCTGTCAGCTCCTTTCCGGGACTTTCGCTTTCCCCCTCCCTATTGCCA | |
| | CGGCGGAACTCATCGCCGCCTGCCTTGCCCGCTGCTGGACAGGGGCTCGGCTGTTGGGCACTGA | |
| | CAATTCCGTGGGGTGTGCCTTCTAGTTGCCAGCCATCTGTTGTTTGCCCCTCCCCCGTGCCTTCCT | |
| | TGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGG | |
| | GAAGACAATAGCAGGCATGCTGGGGATGCGGTGGGCTCTATGGGAAGATGTCTACTGAGCTGTG | |
| | CGATCCCTGCTGGGGACTTTCCGCTGGGGACTTTCCGCTGGGGACTTTCCGCCTTCAGCTAAGGA | |
| | AGCTACCAATATTTAGAGGTACATTTTGTTCTAGAACAAAATGTACCGGTACATTTTGTTCTGGT | |
| | ACATTTTGTTCT | |
| ITR-APOE- | CCTGCAGGCAGCTGCGCGCTCGCTCGCTCACTGAGGCCGCCCGGGCGTCGGGCGACCTTTGGTC | 15 |
| AAT-hBGi- | GCCCGGCCTCAGTGAGCGAGCGCGCAGAGAGGGAGTGGCCAACTCCATCACTAGGGGTTCCTGC | |
| FIX- | GGCCGCACGCGTCTAGTTATTAATAGTAATCGAATTCGCGTCTGCAGGCTCAGAGGCACACAGG | |
| WPRE3- | AGTTTCTGGGCTCACCCTGCCCCCTTCCAACCCCTCAGTTCCCATCCTCCAGCAGCTGTTTGTGTG | |
| bGH- | CTGCCTCTGAAGTCCACACTGAACAAACTTCAGCCTACTCATGTCCCTAAAATGGGCAAACATTG | |
| poly A-DTS- | CAAGCAGCAAACAGCAAACACACAGCCCTCCCTGCCTGCTGACCTTGGAGCTGGGGCAGAGGTC | |
| ITR | AGAGACCTCTCTGGGCCCATGCCACCTCCAACATCCACTCGACCCCTTGGAATTTCGGTGGAGA | |
| | GGAGCAGAGGTTGTCCTGGCGTGGTTTAGGTAGTGTGAGAGGGGTCGACTGGACACAGGACGCT | |
| | GTGGTTTCTGAGCCAGGGGGCGACTCAGATCCCAGCCAGTGGACTTAGCCCCTGTTTGCTCCTCC | |
| | GATAACTGGGGTGACCTTGGTTAATATTCACCAGCAGCCTCCCCCGTTGCCCCTCTGGATCCACT | |
| | GCTTAAATACGGACGAGGACAGGGCCCTGTCTCCTCAGCTTCAGGCACCACCACTGACCTGGGA | |
| | CAGTGAATCGTAAGTACTAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATACCTCTTATCTTC | |
| | CTCCCACAGCTCCTGGGCAACGTGCTGGTCTGTGTGCTGGCCCATCACTTTGGCAAAGAATTGCG | |
| | ATCGCCACCATGCAGCGCGTGAACATGATCATGGCAGAATCACCAGGCCTCATCACCATCTGCC | |
| | TTTTAGGATATCTACTCAGTGCTGAATGTACAGTTTTTCTTGATCATGAAAACGCCAACAAAATT | |
| | CTGAATCGGCCAAAGAGGTATAATTCAGGTAAATTGGAAGAGTTTGTTCAAGGGAACCTTGAGA | |
| | GAGAATGTATGGAAGAAAAGTGTAGTTTTGAAGAAGCACGAGAAGTTTTTGAAAACACTGAAA | |
| | GAACAACTGAATTTTGGAAGCAGTATGTTGATGGAGATCAGTGTGAGTCCAATCCATGTTTAAA | |
| | TGGCGGCAGTTGCAAGGATGACATTAATTCCTATGAATGTTGGTGTCCCTTTGGATTTGAAGGAA | |
| | AGAACTGTGAATTAGATGTAACATGTAACATTAAGAATGGCAGATGCGAGCAGTTTTGTAAAAA | |
| | TAGTGCTGATAACAAGGTGGTTTGCTCCTGTACTGAGGGATATCGACTTGCAGAAAACCAGAAG | |
| | TCCTGTGAACCAGCAGTGCCATTTCCATGTGGAAGAGTTTCTGTTTCACAAACTTCTAAGCTCAC | |
| | CCGTGCTGAGACTGTTTTTCCTGATGTGGACTATGTAAATTCTACTGAAGCTGAAACCATTTTGG | |
| | ATAACATCACTCAAAGCACCCAATCATTTAATGACTTCACTCGGGTTGTTGGTGGAGAAGATGC | |
| | CAAACCAGGTCAATTCCCTTGGCAGGTTGTTTTGAATGGTAAAGTTGATGCATTCTGTGGAGGCT | |
| | CTATCGTTAATGAAAAATGGATTGTAACTGCTGCCCACTGTGTTGAAACTGGTGTTAAAATTACA | |
| | GTTGTCGCAGGTGAACATAATATTGAGGAGACAGAACATACAGAGCAAAAGCGAAATGTGATT | |
| | CGAATTATTCCTCACCACAACTACAATGCAGCTATTAATAAGTACAACCATGACATTGCCCTTCT | |
| | GGAACTGGACGAACCCTTAGTGCTAAACAGCTACGTTACACCTATTTGCATTGCTGACAAGGAA | |
| | TACACGAACATCTTCCTCAAATTTGGATCTGGCTATGTAAGTGGCTGGGGAAGAGTCTTCCACAA | |
| | AGGGAGATCAGCTTTAGTTCTTCAGTACCTTAGAGTTCCACTTGTTGACCGAGCCACATGTCTTC | |
| | GATCTACAAAGTTCACCATCTATAACAACATGTTCTGTGCTGGCTTCCATGAAGGAGGTAGAGA | |
| | TTCATGTCAAGGAGATAGTGGGGGACCCCATGTTACTGAAGTGGAAGGGACCAGTTTCTTAACT | |
| | GGAATTATTAGCTGGGGTGAAGAGTGTGCAATGAAAGGCAAATATGGAATATATACCAAGGTAT | |
| | CCCGGTATGTCAACTGGATTAAGGAAAAAACAAAGCTCACTTAATGACCCTCGAGAATCAACCT | |
| | CTGGATTACAAAATTTGTGAAAGATTGACTGGTATTCTTAACTATGTTGCTCCTTTTACGCTATGT | |
| | GGATACGCTGCTTTAATGCCTTTGTATCATGCTATTGCTTCCCGTATGGCTTTCATTTTCTCCTCCT | |
| | TGTATAAATCCTGGTTGCTGTCTCTTTATGAGGAGTTGTGGCCCGTTGTCAGGCAACGTGGCGTG | |
| | GTGTGCACTGTGTTTGCTGACGCAACCCCCACTGGTTGGGGCATTGCCACCACCTGTCAGCTCCT | |
| | TTCCGGGACTTTCGCTTTCCCCCTCCCTATTGCCACGGCGGAACTCATCGCCGCCTGCCTTGCCCG | |
| | CTGCTGGACAGGGGCTCGGCTGTTGGGCACTGACAATTCCGTGGGGTGTGCCTTCTAGTTGCCAG | |
| | CCATCTGTTGTTTGCCCCTCCCCCGTGCCTTCCTTGACCCTGGAAGGTGCCACTCCCACTGTCCTT | |
| | TCCTAATAAAATGAGGAAATTGCATCGCATTGTCTGAGTAGGTGTCATTCTATTCTGGGGGGTGG | |
| | GGTGGGGCAGGACAGCAAGGGGGAGGATTGGGAAGACAATAGCAGGCATGCTGGGGATGCGGT | |
| | GGGCTCTATGGGCACGTGCGGAGCTGTGCGATCCCTGCTGGGGACTTTCCGCTGGGGACTTTCCG | |
| | CTGGGGACTTTCCGCCTTCAGCTAAGGAAGCTACCAATATTTAGAGGTACATTTTGTTCTAGAAC | |
| | AAAATGTACCGGTACATTTTGTTCTGGTACATTTTGTTCTATCGATCGAGCGGCCGCAGGAACCC | |
| | CTAGTGATGGAGTTGGCCACTCCCTCTCTGCGCGCTCGCTCGCTCACTGAGGCCGGGCGACCAA | |
| | AGGTCGCCCGACGCCCGGGCTTTGCCCGGGCGGCCTCAGTGAGCGAGCGAGCGCGCAGCTGCCT | |
| | GCAGG | |
| APOE- | CGCGTCTGCAGGCTCAGAGGCACACAGGAGTTTCTGGGCTCACCCTGCCCCCTTCCAACCCCTCA | 16 |
| AAT-hBGi- | GTTCCCATCCTCCAGCAGCTGTTTGTGTGCTGCCTCTGAAGTCCACACTGAACAAACTTCAGCCT | |
| FVIII-v3- | ACTCATGTCCCTAAAATGGGCAAACATTGCAAGCAGCAAACAGCAAACACACAGCCCTCCCTGC | |
| bGH-poly A | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGATTGAGCTGAGCACCTGCTTC | |
| | TTCCTGTGCCTGCTGAGGTTCTGCTTCTCTGCCACCAGGAGATACTACCTGGGGGCTGTGGAGCT | |
| | GAGCTGGGACTACATGCAGTCTGACCTGGGGGAGCTGCCTGTGGATGCCAGGTTCCCCCCCAGA | |
| | GTGCCCAAGAGCTTCCCCTTCAACACCTCTGTGGTGTACAAGAAGACCCTGTTTGTGGAGTTCAC | |
| | TGACCACCTGTTCAACATTGCCAAGCCCAGGCCCCCCTGGATGGGCCTGCTGGGCCCCACCATCC | |
| | AGGCTGAGGTGTATGACACTGTGGTGATCACCCTGAAGAACATGGCCAGCCACCCTGTGAGCCT | |
| | GCATGCTGTGGGGGTGAGCTACTGGAAGGCCTCTGAGGGGGCTGAGTATGATGACCAGACCAGC | |
| | CAGAGGGAGAAGGAGGATGACAAGGTGTTCCCTGGGGGCAGCCACACCTATGTGTGGCAGGTG | |
| | CTGAAGGAGAATGGCCCCATGGCCTCTGACCCCCTGTGCCTGACCTACAGCTACCTGAGCCATG | |
| | TGGACCTGGTGAAGGACCTGAACTCTGGCCTGATTGGGGCCCTGCTGGTGTGCAGGGAGGGCAG | |
| | CCTGGCCAAGGAGAAGACCCAGACCCTGCACAAGTTCATCCTGCTGTTTGCTGTGTTTGATGAG | |
| | GGCAAGAGCTGGCACTCTGAAACCAAGAACAGCCTGATGCAGGACAGGGATGCTGCCTCTGCC | |
| | AGGGCCTGGCCCAAGATGCACACTGTGAATGGCTATGTGAACAGGAGCCTGCCTGGCCTGATTG | |
| | GCTGCCACAGGAAGTCTGTGTACTGGCATGTGATTGGCATGGGCACCACCCCTGAGGTGCACAG | |
| | CATCTTCCTGGAGGGCCACACCTTCCTGGTCAGGAACCACAGGCAGGCCAGCCTGGAGATCAGC | |
| | CCCATCACCTTCCTGACTGCCCAGACCCTGCTGATGGACCTGGGCCAGTTCCTGCTGTTCTGCCA | |
| | CATCAGCAGCCACCAGCATGATGGCATGGAGGCCTATGTGAAGGTGGACAGCTGCCCTGAGGA | |
| | GCCCCAGCTGAGGATGAAGAACAATGAGGAGGCTGAGGACTATGATGATGACCTGACTGACTCT | |
| | GAGATGGATGTGGTGAGGTTTGATGATGACAACAGCCCCAGCTTCATCCAGATCAGGTCTGTGG | |
| | CCAAGAAGCACCCCAAGACCTGGGTGCACTACATTGCTGCTGAGGAGGAGGACTGGGACTATGC | |
| | CCCCCTGGTGCTGGCCCCTGATGACAGGAGCTACAAGAGCCAGTACCTGAACAATGGCCCCCAG | |
| | AGGATTGGCAGGAAGTACAAGAAGGTCAGGTTCATGGCCTACACTGATGAAACCTTCAAGACCA | |
| | GGGAGGCCATCCAGCATGAGTCTGGCATCCTGGGCCCCCTGCTGTATGGGGAGGTGGGGGACAC | |
| | CCTGCTGATCATCTTCAAGAACCAGGCCAGCAGGCCCTACAACATCTACCCCCATGGCATCACT | |
| | GATGTGAGGCCCCTGTACAGCAGGAGGCTGCCCAAGGGGGTGAAGCACCTGAAGGACTTCCCC | |
| | ATCCTGCCTGGGGAGATCTTCAAGTACAAGTGGACTGTGACTGTGGAGGATGGCCCCACCAAGT | |
| | CTGACCCCAGGTGCCTGACCAGATACTACAGCAGCTTTGTGAACATGGAGAGGGACCTGGCCTC | |
| | TGGCCTGATTGGCCCCCTGCTGATCTGCTACAAGGAGTCTGTGGACCAGAGGGGCAACCAGATC | |
| | ATGTCTGACAAGAGGAATGTGATCCTGTTCTCTGTGTTTGATGAGAACAGGAGCTGGTACCTGA | |
| | CTGAGAACATCCAGAGGTTCCTGCCCAACCCTGCTGGGGTGCAGCTGGAGGACCCTGAGTTCCA | |
| | GGCCAGCAACATCATGCACAGCATCAATGGCTATGTGTTTGACAGCCTGCAGCTGTCTGTGTGCC | |
| | TGCATGAGGTGGCCTACTGGTACATCCTGAGCATTGGGGCCCAGACTGACTTCCTGTCTGTGTTC | |
| | TTCTCTGGCTACACCTTCAAGCACAAGATGGTGTATGAGGACACCCTGACCCTGTTCCCCTTCTC | |
| | TGGGGAGACTGTGTTCATGAGCATGGAGAACCCTGGCCTGTGGATTCTGGGCTGCCACAACTCT | |
| | GACTTCAGGAACAGGGGCATGACTGCCCTGCTGAAAGTCTCCAGCTGTGACAAGAACACTGGGG | |
| | ACTACTATGAGGACAGCTATGAGGACATCTCTGCCTACCTGCTGAGCAAGAACAATGCCATTGA | |
| | GCCCAGGAGCTTCAGCCAGAATGCCACTAATGTGTCTAACAACAGCAACACCAGCAATGACAGC | |
| | AATGTGTCTCCCCCAGTGCTGAAGAGGCACCAGAGGGAGATCACCAGGACCACCCTGCAGTCTG | |
| | ACCAGGAGGAGATTGACTATGATGACACCATCTCTGTGGAGATGAAGAAGGAGGACTTTGACAT | |
| | CTACGACGAGGACGAGAACCAGAGCCCCAGGAGCTTCCAGAAGAAGACCAGGCACTACTTCAT | |
| | TGCTGCTGTGGAGAGGCTGTGGGACTATGGCATGAGCAGCAGCCCCCATGTGCTGAGGAACAGG | |
| | GCCCAGTCTGGCTCTGTGCCCCAGTTCAAGAAGGTGGTGTTCCAGGAGTTCACTGATGGCAGCTT | |
| | CACCCAGCCCCTGTACAGAGGGGAGCTGAATGAGCACCTGGGCCTGCTGGGCCCCTACATCAGG | |
| | GCTGAGGTGGAGGACAACATCATGGTGACCTTCAGGAACCAGGCCAGCAGGCCCTACAGCTTCT | |
| | ACAGCAGCCTGATCAGCTATGAGGAGGACCAGAGGCAGGGGGCTGAGCCCAGGAAGAACTTTG | |
| | TGAAGCCCAATGAAACCAAGACCTACTTCTGGAAGGTGCAGCACCACATGGCCCCCACCAAGGA | |
| | TGAGTTTGACTGCAAGGCCTGGGCCTACTTCTCTGATGTGGACCTGGAGAAGGATGTGCACTCTG | |
| | GCCTGATTGGCCCCCTGCTGGTGTGCCACACCAACACCCTGAACCCTGCCCATGGCAGGCAGGT | |
| | GACTGTGCAGGAGTTTGCCCTGTTCTTCACCATCTTTGATGAAACCAAGAGCTGGTACTTCACTG | |
| | AGAACATGGAGAGGAACTGCAGGGCCCCCTGCAACATCCAGATGGAGGACCCCACCTTCAAGG | |
| | AGAACTACAGGTTCCATGCCATCAATGGCTACATCATGGACACCCTGCCTGGCCTGGTGATGGC | |
| | CCAGGACCAGAGGATCAGGTGGTACCTGCTGAGCATGGGCAGCAATGAGAACATCCACAGCAT | |
| | CCACTTCTCTGGCCATGTGTTCACTGTGAGGAAGAAGGAGGAGTACAAGATGGCCCTGTACAAC | |
| | CTGTACCCTGGGGTGTTTGAGACTGTGGAGATGCTGCCCAGCAAGGCTGGCATCTGGAGGGTGG | |
| | AGTGCCTGATTGGGGAGCACCTGCATGCTGGCATGAGCACCCTGTTCCTGGTGTACAGCAACAA | |
| | GTGCCAGACCCCCCTGGGCATGGCCTCTGGCCACATCAGGGACTTCCAGATCACTGCCTCTGGCC | |
| | AGTATGGCCAGTGGGCCCCCAAGCTGGCCAGGCTGCACTACTCTGGCAGCATCAATGCCTGGAG | |
| | CACCAAGGAGCCCTTCAGCTGGATCAAGGTGGACCTGCTGGCCCCCATGATCATCCATGGCATC | |
| | AAGACCCAGGGGGCCAGGCAGAAGTTCAGCAGCCTGTACATCAGCCAGTTCATCATCATGTACA | |
| | GCCTGGATGGCAAGAAGTGGCAGACCTACAGGGGCAACAGCACTGGCACCCTGATGGTGTTCTT | |
| | TGGCAATGTGGACAGCTCTGGCATCAAGCACAACATCTTCAACCCCCCCATCATTGCCAGATAC | |
| | ATCAGGCTGCACCCCACCCACTACAGCATCAGGAGCACCCTGAGGATGGAGCTGATGGGCTGTG | |
| | ACCTGAACAGCTGCAGCATGCCCCTGGGCATGGAGAGCAAGGCCATCTCTGATGCCCAGATCAC | |
| | TGCCAGCAGCTACTTCACCAACATGTTTGCCACCTGGAGCCCCAGCAAGGCCAGGCTGCACCTG | |
| | CAGGGCAGGAGCAATGCCTGGAGGCCCCAGGTCAACAACCCCAAGGAGTGGCTGCAGGTGGAC | |
| | TTCCAGAAGACCATGAAGGTGACTGGGGTGACCACCCAGGGGGTGAAGAGCCTGCTGACCAGC | |
| | ATGTATGTGAAGGAGTTCCTGATCAGCAGCAGCCAGGATGGCCACCAGTGGACCCTGTTCTTCC | |
| | AGAATGGCAAGGTGAAGGTGTTCCAGGGCAACCAGGACAGCTTCACCCCTGTGGTGAACAGCCT | |
| | GGACCCCCCCCTGCTGACCAGATACCTGAGGATTCACCCCCAGAGCTGGGTGCACCAGATTGCC | |
| | CTGAGGATGGAGGTGCTGGGCTGTGAGGCCCAGGACCTGTACTGATAACTCGAGAATCAACCTC | |
| | TGGATTACAAAATTTGTGAAAGATTGACTGGTATTCTTAACTATGTTGCTCCTTTTACGCTATGTG | |
| | GATACGCTGCTTTAATGCCTTTGTATCATGCTATTGCTTCCCGTATGGCTTTCATTTTCTCCTCCTT | |
| | GTATAAATCCTGGTTGCTGTCTCTTTATGAGGAGTTGTGGCCCGTTGTCAGGCAACGTGGCGTGG | |
| | TGTGCACTGTGTTTGCTGACGCAACCCCCACTGGTTGGGGCATTGCCACCACCTGTCAGCTCCTT | |
| | TCCGGGACTTTCGCTTTCCCCCTCCCTATTGCCACGGCGGAACTCATCGCCGCCTGCCTTGCCCG | |
| | CTGCTGGACAGGGGCTCGGCTGTTGGGCACTGACAATTCCGTGGGGTGTGCCTTCTAGTTGCCAG | |
| | CCATCTGTTGTTTGCCCCTCCCCCGTGCCTTCCTTGACCCTGGAAGGTGCCACTCCCACTGTCCTT | |
| | TCCTAATAAAATGAGGAAATTGCATCGCATTGTCTGAGTAGGTGTCATTCTATTCTGGGGGGTGG | |
| | GGTGGGGCAGGACAGCAAGGGGGAGGATTGGGAAGACAATAGCAGGCATGCTGGGGATGCGGT | |
| | GGGCTCTATGGGAAGATGTCTACTGAGCTGTGCGATCCCTGCTGGGGACTTTCCGCTGGGGACTT | |
| | TCCGCTGGGGACTTTCCGCCTTCAGCTAAGGAAGCTACCAATATTTAGAGGTACATTTTGTTCTA | |
| | GAACAAAATGTACCGGTACATTTTGTTCTGGTACATTTTGTTCT | |
| APOE- | CGCGTCTGCAGGCTCAGAGGCACACAGGAGTTTCTGGGCTCACCCTGCCCCCTTCCAACCCCTCA | 17 |
| AAT-hBGi- | GTTCCCATCCTCCAGCAGCTGTTTGTGTGCTGCCTCTGAAGTCCACACTGAACAAACTTCAGCCT | |
| FVIII-v3- | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| bGH- | CACTCGACCCCTTGGAATTTCGGTGGAGAGGAGCAGAGGTTGTCCTGGCGTGGTTTAGGTAGTG | |
| poly A-DTS | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGATTGAGCTGAGCACCTGCTTC | |
| | TTCCTGTGCCTGCTGAGGTTCTGCTTCTCTGCCACCAGGAGATACTACCTGGGGGCTGTGGAGCT | |
| | GAGCTGGGACTACATGCAGTCTGACCTGGGGGAGCTGCCTGTGGATGCCAGGTTCCCCCCCAGA | |
| | GTGCCCAAGAGCTTCCCCTTCAACACCTCTGTGGTGTACAAGAAGACCCTGTTTGTGGAGTTCAC | |
| | TGACCACCTGTTCAACATTGCCAAGCCCAGGCCCCCCTGGATGGGCCTGCTGGGCCCCACCATCC | |
| | AGGCTGAGGTGTATGACACTGTGGTGATCACCCTGAAGAACATGGCCAGCCACCCTGTGAGCCT | |
| | GCATGCTGTGGGGGTGAGCTACTGGAAGGCCTCTGAGGGGGCTGAGTATGATGACCAGACCAGC | |
| | CAGAGGGAGAAGGAGGATGACAAGGTGTTCCCTGGGGGCAGCCACACCTATGTGTGGCAGGTG | |
| | CTGAAGGAGAATGGCCCCATGGCCTCTGACCCCCTGTGCCTGACCTACAGCTACCTGAGCCATG | |
| | TGGACCTGGTGAAGGACCTGAACTCTGGCCTGATTGGGGCCCTGCTGGTGTGCAGGGAGGGCAG | |
| | CCTGGCCAAGGAGAAGACCCAGACCCTGCACAAGTTCATCCTGCTGTTTGCTGTGTTTGATGAG | |
| | GGCAAGAGCTGGCACTCTGAAACCAAGAACAGCCTGATGCAGGACAGGGATGCTGCCTCTGCC | |
| | AGGGCCTGGCCCAAGATGCACACTGTGAATGGCTATGTGAACAGGAGCCTGCCTGGCCTGATTG | |
| | GCTGCCACAGGAAGTCTGTGTACTGGCATGTGATTGGCATGGGCACCACCCCTGAGGTGCACAG | |
| | CATCTTCCTGGAGGGCCACACCTTCCTGGTCAGGAACCACAGGCAGGCCAGCCTGGAGATCAGC | |
| | CCCATCACCTTCCTGACTGCCCAGACCCTGCTGATGGACCTGGGCCAGTTCCTGCTGTTCTGCCA | |
| | CATCAGCAGCCACCAGCATGATGGCATGGAGGCCTATGTGAAGGTGGACAGCTGCCCTGAGGA | |
| | GCCCCAGCTGAGGATGAAGAACAATGAGGAGGCTGAGGACTATGATGATGACCTGACTGACTCT | |
| | GAGATGGATGTGGTGAGGTTTGATGATGACAACAGCCCCAGCTTCATCCAGATCAGGTCTGTGG | |
| | CCAAGAAGCACCCCAAGACCTGGGTGCACTACATTGCTGCTGAGGAGGAGGACTGGGACTATGC | |
| | CCCCCTGGTGCTGGCCCCTGATGACAGGAGCTACAAGAGCCAGTACCTGAACAATGGCCCCCAG | |
| | AGGATTGGCAGGAAGTACAAGAAGGTCAGGTTCATGGCCTACACTGATGAAACCTTCAAGACCA | |
| | GGGAGGCCATCCAGCATGAGTCTGGCATCCTGGGCCCCCTGCTGTATGGGGAGGTGGGGGACAC | |
| | CCTGCTGATCATCTTCAAGAACCAGGCCAGCAGGCCCTACAACATCTACCCCCATGGCATCACT | |
| | GATGTGAGGCCCCTGTACAGCAGGAGGCTGCCCAAGGGGGTGAAGCACCTGAAGGACTTCCCC | |
| | ATCCTGCCTGGGGAGATCTTCAAGTACAAGTGGACTGTGACTGTGGAGGATGGCCCCACCAAGT | |
| | CTGACCCCAGGTGCCTGACCAGATACTACAGCAGCTTTGTGAACATGGAGAGGGACCTGGCCTC | |
| | TGGCCTGATTGGCCCCCTGCTGATCTGCTACAAGGAGTCTGTGGACCAGAGGGGCAACCAGATC | |
| | ATGTCTGACAAGAGGAATGTGATCCTGTTCTCTGTGTTTGATGAGAACAGGAGCTGGTACCTGA | |
| | CTGAGAACATCCAGAGGTTCCTGCCCAACCCTGCTGGGGTGCAGCTGGAGGACCCTGAGTTCCA | |
| | GGCCAGCAACATCATGCACAGCATCAATGGCTATGTGTTTGACAGCCTGCAGCTGTCTGTGTGCC | |
| | TGCATGAGGTGGCCTACTGGTACATCCTGAGCATTGGGGCCCAGACTGACTTCCTGTCTGTGTTC | |
| | TTCTCTGGCTACACCTTCAAGCACAAGATGGTGTATGAGGACACCCTGACCCTGTTCCCCTTCTC | |
| | TGGGGAGACTGTGTTCATGAGCATGGAGAACCCTGGCCTGTGGATTCTGGGCTGCCACAACTCT | |
| | GACTTCAGGAACAGGGGCATGACTGCCCTGCTGAAAGTCTCCAGCTGTGACAAGAACACTGGGG | |
| | ACTACTATGAGGACAGCTATGAGGACATCTCTGCCTACCTGCTGAGCAAGAACAATGCCATTGA | |
| | GCCCAGGAGCTTCAGCCAGAATGCCACTAATGTGTCTAACAACAGCAACACCAGCAATGACAGC | |
| | AATGTGTCTCCCCCAGTGCTGAAGAGGCACCAGAGGGAGATCACCAGGACCACCCTGCAGTCTG | |
| | ACCAGGAGGAGATTGACTATGATGACACCATCTCTGTGGAGATGAAGAAGGAGGACTTTGACAT | |
| | CTACGACGAGGACGAGAACCAGAGCCCCAGGAGCTTCCAGAAGAAGACCAGGCACTACTTCAT | |
| | TGCTGCTGTGGAGAGGCTGTGGGACTATGGCATGAGCAGCAGCCCCCATGTGCTGAGGAACAGG | |
| | GCCCAGTCTGGCTCTGTGCCCCAGTTCAAGAAGGTGGTGTTCCAGGAGTTCACTGATGGCAGCTT | |
| | CACCCAGCCCCTGTACAGAGGGGAGCTGAATGAGCACCTGGGCCTGCTGGGCCCCTACATCAGG | |
| | GCTGAGGTGGAGGACAACATCATGGTGACCTTCAGGAACCAGGCCAGCAGGCCCTACAGCTTCT | |
| | ACAGCAGCCTGATCAGCTATGAGGAGGACCAGAGGCAGGGGGCTGAGCCCAGGAAGAACTTTG | |
| | TGAAGCCCAATGAAACCAAGACCTACTTCTGGAAGGTGCAGCACCACATGGCCCCCACCAAGGA | |
| | TGAGTTTGACTGCAAGGCCTGGGCCTACTTCTCTGATGTGGACCTGGAGAAGGATGTGCACTCTG | |
| | GCCTGATTGGCCCCCTGCTGGTGTGCCACACCAACACCCTGAACCCTGCCCATGGCAGGCAGGT | |
| | GACTGTGCAGGAGTTTGCCCTGTTCTTCACCATCTTTGATGAAACCAAGAGCTGGTACTTCACTG | |
| | AGAACATGGAGAGGAACTGCAGGGCCCCCTGCAACATCCAGATGGAGGACCCCACCTTCAAGG | |
| | AGAACTACAGGTTCCATGCCATCAATGGCTACATCATGGACACCCTGCCTGGCCTGGTGATGGC | |
| | CCAGGACCAGAGGATCAGGTGGTACCTGCTGAGCATGGGCAGCAATGAGAACATCCACAGCAT | |
| | CCACTTCTCTGGCCATGTGTTCACTGTGAGGAAGAAGGAGGAGTACAAGATGGCCCTGTACAAC | |
| | CTGTACCCTGGGGTGTTTGAGACTGTGGAGATGCTGCCCAGCAAGGCTGGCATCTGGAGGGTGG | |
| | AGTGCCTGATTGGGGAGCACCTGCATGCTGGCATGAGCACCCTGTTCCTGGTGTACAGCAACAA | |
| | GTGCCAGACCCCCCTGGGCATGGCCTCTGGCCACATCAGGGACTTCCAGATCACTGCCTCTGGCC | |
| | AGTATGGCCAGTGGGCCCCCAAGCTGGCCAGGCTGCACTACTCTGGCAGCATCAATGCCTGGAG | |
| | CACCAAGGAGCCCTTCAGCTGGATCAAGGTGGACCTGCTGGCCCCCATGATCATCCATGGCATC | |
| | AAGACCCAGGGGGCCAGGCAGAAGTTCAGCAGCCTGTACATCAGCCAGTTCATCATCATGTACA | |
| | GCCTGGATGGCAAGAAGTGGCAGACCTACAGGGGCAACAGCACTGGCACCCTGATGGTGTTCTT | |
| | TGGCAATGTGGACAGCTCTGGCATCAAGCACAACATCTTCAACCCCCCCATCATTGCCAGATAC | |
| | ATCAGGCTGCACCCCACCCACTACAGCATCAGGAGCACCCTGAGGATGGAGCTGATGGGCTGTG | |
| | ACCTGAACAGCTGCAGCATGCCCCTGGGCATGGAGAGCAAGGCCATCTCTGATGCCCAGATCAC | |
| | TGCCAGCAGCTACTTCACCAACATGTTTGCCACCTGGAGCCCCAGCAAGGCCAGGCTGCACCTG | |
| | CAGGGCAGGAGCAATGCCTGGAGGCCCCAGGTCAACAACCCCAAGGAGTGGCTGCAGGTGGAC | |
| | TTCCAGAAGACCATGAAGGTGACTGGGGTGACCACCCAGGGGGTGAAGAGCCTGCTGACCAGC | |
| | ATGTATGTGAAGGAGTTCCTGATCAGCAGCAGCCAGGATGGCCACCAGTGGACCCTGTTCTTCC | |
| | AGAATGGCAAGGTGAAGGTGTTCCAGGGCAACCAGGACAGCTTCACCCCTGTGGTGAACAGCCT | |
| | GGACCCCCCCCTGCTGACCAGATACCTGAGGATTCACCCCCAGAGCTGGGTGCACCAGATTGCC | |
| | CTGAGGATGGAGGTGCTGGGCTGTGAGGCCCAGGACCTGTACTGATAACTCGAGAATCAACCTC | |
| | TGGATTACAAAATTTGTGAAAGATTGACTGGTATTCTTAACTATGTTGCTCCTTTTACGCTATGTG | |
| | GATACGCTGCTTTAATGCCTTTGTATCATGCTATTGCTTCCCGTATGGCTTTCATTTTCTCCTCCTT | |
| | GTATAAATCCTGGTTGCTGTCTCTTTATGAGGAGTTGTGGCCCGTTGTCAGGCAACGTGGCGTGG | |
| | TGTGCACTGTGTTTGCTGACGCAACCCCCACTGGTTGGGGCATTGCCACCACCTGTCAGCTCCTT | |
| | TCCGGGACTTTCGCTTTCCCCCTCCCTATTGCCACGGCGGAACTCATCGCCGCCTGCCTTGCCCG | |
| | CTGCTGGACAGGGGCTCGGCTGTTGGGCACTGACAATTCCGTGGGGTGTGCCTTCTAGTTGCCAG | |
| | CCATCTGTTGTTTGCCCCTCCCCCGTGCCTTCCTTGACCCTGGAAGGTGCCACTCCCACTGTCCTT | |
| | TCCTAATAAAATGAGGAAATTGCATCGCATTGTCTGAGTAGGTGTCATTCTATTCTGGGGGGTGG | |
| | GGTGGGGCAGGACAGCAAGGGGGAGGATTGGGAAGACAATAGCAGGCATGCTGGGGATGCGGT | |
| | GGGCTCTATGGGAAGATGTCTACTGAGCTGTGCGATCCCTGCTGGGGACTTTCCGCTGGGGACTT | |
| | TCCGCTGGGGACTTTCCGCCTTCAGCTAAGGAAGCTACCAATATTTAGAGGTACATTTTGTTCTA | |
| | GAACAAAATGTACCGGTACATTTTGTTCTGGTACATTTTGTTCT | |
| APOE- | CGCGTCTGCAGGCTCAGAGGCACACAGGAGTTTCTGGGCTCACCCTGCCCCCTTCCAACCCCTCA | 18 |
| AAT-hBGi- | GTTCCCATCCTCCAGCAGCTGTTTGTGTGCTGCCTCTGAAGTCCACACTGAACAAACTTCAGCCT | |
| FIX- | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| bGH-poly A | CACTCGACCCCTTGGAATTTCGGTGGAGAGGAGCAGAGGTTGTCCTGGCGTGGTTTAGGTAGTG | |
| DTS | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGCGCGTGAACATGATCATGGC | |
| | AGAATCACCAGGCCTCATCACCATCTGCCTTTTAGGATATCTACTCAGTGCTGAATGTACAGTTT | |
| | TTCTTGATCATGAAAACGCCAACAAAATTCTGAATCGGCCAAAGAGGTATAATTCAGGTAAATT | |
| | GGAAGAGTTTGTTCAAGGGAACCTTGAGAGAGAATGTATGGAAGAAAAGTGTAGTTTTGAAGA | |
| | AGCACGAGAAGTTTTTGAAAACACTGAAAGAACAACTGAATTTTGGAAGCAGTATGTTGATGGA | |
| | GATCAGTGTGAGTCCAATCCATGTTTAAATGGCGGCAGTTGCAAGGATGACATTAATTCCTATG | |
| | AATGTTGGTGTCCCTTTGGATTTGAAGGAAAGAACTGTGAATTAGATGTAACATGTAACATTAA | |
| | GAATGGCAGATGCGAGCAGTTTTGTAAAAATAGTGCTGATAACAAGGTGGTTTGCTCCTGTACT | |
| | GAGGGATATCGACTTGCAGAAAACCAGAAGTCCTGTGAACCAGCAGTGCCATTTCCATGTGGAA | |
| | GAGTTTCTGTTTCACAAACTTCTAAGCTCACCCGTGCTGAGACTGTTTTTCCTGATGTGGACTATG | |
| | TAAATTCTACTGAAGCTGAAACCATTTTGGATAACATCACTCAAAGCACCCAATCATTTAATGAC | |
| | TTCACTCGGGTTGTTGGTGGAGAAGATGCCAAACCAGGTCAATTCCCTTGGCAGGTTGTTTTGAA | |
| | TGGTAAAGTTGATGCATTCTGTGGAGGCTCTATCGTTAATGAAAAATGGATTGTAACTGCTGCCC | |
| | ACTGTGTTGAAACTGGTGTTAAAATTACAGTTGTCGCAGGTGAACATAATATTGAGGAGACAGA | |
| | ACATACAGAGCAAAAGCGAAATGTGATTCGAATTATTCCTCACCACAACTACAATGCAGCTATT | |
| | AATAAGTACAACCATGACATTGCCCTTCTGGAACTGGACGAACCCTTAGTGCTAAACAGCTACG | |
| | TTACACCTATTTGCATTGCTGACAAGGAATACACGAACATCTTCCTCAAATTTGGATCTGGCTAT | |
| | GTAAGTGGCTGGGGAAGAGTCTTCCACAAAGGGAGATCAGCTTTAGTTCTTCAGTACCTTAGAG | |
| | TTCCACTTGTTGACCGAGCCACATGTCTTCGATCTACAAAGTTCACCATCTATAACAACATGTTC | |
| | TGTGCTGGCTTCCATGAAGGAGGTAGAGATTCATGTCAAGGAGATAGTGGGGGACCCCATGTTA | |
| | CTGAAGTGGAAGGGACCAGTTTCTTAACTGGAATTATTAGCTGGGGTGAAGAGTGTGCAATGAA | |
| | AGGCAAATATGGAATATATACCAAGGTATCCCGGTATGTCAACTGGATTAAGGAAAAAACAAA | |
| | GCTCACTTAATGACCCTCGAGAATCAACCTCTGGATTACAAAATTTGTGAAAGATTGACTGGTAT | |
| | TCTTAACTATGTTGCTCCTTTTACGCTATGTGGATACGCTGCTTTAATGCCTTTGTATCATGCTAT | |
| | TGCTTCCCGTATGGCTTTCATTTTCTCCTCCTTGTATAAATCCTGGTTGCTGTCTCTTTATGAGGA | |
| | GTTGTGGCCCGTTGTCAGGCAACGTGGCGTGGTGTGCACTGTGTTTGCTGACGCAACCCCCACTG | |
| | GTTGGGGCATTGCCACCACCTGTCAGCTCCTTTCCGGGACTTTCGCTTTCCCCCTCCCTATTGCCA | |
| | CGGCGGAACTCATCGCCGCCTGCCTTGCCCGCTGCTGGACAGGGGCTCGGCTGTTGGGCACTGA | |
| | CAATTCCGTGGGGTGTGCCTTCTAGTTGCCAGCCATCTGTTGTTTGCCCCTCCCCCGTGCCTTCCT | |
| | TGACCCTGGAAGGTGCCACTCCCACTGTCCTTTCCTAATAAAATGAGGAAATTGCATCGCATTGT | |
| | CTGAGTAGGTGTCATTCTATTCTGGGGGGTGGGGTGGGGCAGGACAGCAAGGGGGAGGATTGG | |
| | GAAGACAATAGCAGGCATGCTGGGGATGCGGTGGGCTCTATGGGAAGATGTCTACTGAGCTGTG | |
| | CGATCCCTGCTGGGGACTTTCCGCTGGGGACTTTCCGCTGGGGACTTTCCGCCTTCAGCTAAGGA | |
| | AGCTACCAATATTTAGAGGTACATTTTGTTCTAGAACAAAATGTACCGGTACATTTTGTTCTGGT | |
| | ACATTTTGTTCT | |
| APOE- | CGCGTCTGCAGGCTCAGAGGCACACAGGAGTTTCTGGGCTCACCCTGCCCCCTTCCAACCCCTCA | 19 |
| AAT-hbGi- | GTTCCCATCCTCCAGCAGCTGTTTGTGTGCTGCCTCTGAAGTCCACACTGAACAAACTTCAGCCT | |
| FVIIIv3- | ACTCATGTCCCTAAAATGGGCAAACATTGCAAGCAGCAAACAGCAAACACACAGCCCTCCCTGC | |
| | CTGCTGACCTTGGAGCTGGGGCAGAGGTCAGAGACCTCTCTGGGCCCATGCCACCTCCAACATC | |
| | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGATTGAGCTGAGCACCTGCTTC | |
| | TTCCTGTGCCTGCTGAGGTTCTGCTTCTCTGCCACCAGGAGATACTACCTGGGGGCTGTGGAGCT | |
| | GAGCTGGGACTACATGCAGTCTGACCTGGGGGAGCTGCCTGTGGATGCCAGGTTCCCCCCCAGA | |
| | GTGCCCAAGAGCTTCCCCTTCAACACCTCTGTGGTGTACAAGAAGACCCTGTTTGTGGAGTTCAC | |
| | TGACCACCTGTTCAACATTGCCAAGCCCAGGCCCCCCTGGATGGGCCTGCTGGGCCCCACCATCC | |
| | AGGCTGAGGTGTATGACACTGTGGTGATCACCCTGAAGAACATGGCCAGCCACCCTGTGAGCCT | |
| | GCATGCTGTGGGGGTGAGCTACTGGAAGGCCTCTGAGGGGGCTGAGTATGATGACCAGACCAGC | |
| | CAGAGGGAGAAGGAGGATGACAAGGTGTTCCCTGGGGGCAGCCACACCTATGTGTGGCAGGTG | |
| | CTGAAGGAGAATGGCCCCATGGCCTCTGACCCCCTGTGCCTGACCTACAGCTACCTGAGCCATG | |
| | TGGACCTGGTGAAGGACCTGAACTCTGGCCTGATTGGGGCCCTGCTGGTGTGCAGGGAGGGCAG | |
| | CCTGGCCAAGGAGAAGACCCAGACCCTGCACAAGTTCATCCTGCTGTTTGCTGTGTTTGATGAG | |
| | GGCAAGAGCTGGCACTCTGAAACCAAGAACAGCCTGATGCAGGACAGGGATGCTGCCTCTGCC | |
| | AGGGCCTGGCCCAAGATGCACACTGTGAATGGCTATGTGAACAGGAGCCTGCCTGGCCTGATTG | |
| | GCTGCCACAGGAAGTCTGTGTACTGGCATGTGATTGGCATGGGCACCACCCCTGAGGTGCACAG | |
| | CATCTTCCTGGAGGGCCACACCTTCCTGGTCAGGAACCACAGGCAGGCCAGCCTGGAGATCAGC | |
| | CCCATCACCTTCCTGACTGCCCAGACCCTGCTGATGGACCTGGGCCAGTTCCTGCTGTTCTGCCA | |
| | CATCAGCAGCCACCAGCATGATGGCATGGAGGCCTATGTGAAGGTGGACAGCTGCCCTGAGGA | |
| | GCCCCAGCTGAGGATGAAGAACAATGAGGAGGCTGAGGACTATGATGATGACCTGACTGACTCT | |
| | GAGATGGATGTGGTGAGGTTTGATGATGACAACAGCCCCAGCTTCATCCAGATCAGGTCTGTGG | |
| | CCAAGAAGCACCCCAAGACCTGGGTGCACTACATTGCTGCTGAGGAGGAGGACTGGGACTATGC | |
| | CCCCCTGGTGCTGGCCCCTGATGACAGGAGCTACAAGAGCCAGTACCTGAACAATGGCCCCCAG | |
| | AGGATTGGCAGGAAGTACAAGAAGGTCAGGTTCATGGCCTACACTGATGAAACCTTCAAGACCA | |
| | GGGAGGCCATCCAGCATGAGTCTGGCATCCTGGGCCCCCTGCTGTATGGGGAGGTGGGGGACAC | |
| | CCTGCTGATCATCTTCAAGAACCAGGCCAGCAGGCCCTACAACATCTACCCCCATGGCATCACT | |
| | GATGTGAGGCCCCTGTACAGCAGGAGGCTGCCCAAGGGGGTGAAGCACCTGAAGGACTTCCCC | |
| | ATCCTGCCTGGGGAGATCTTCAAGTACAAGTGGACTGTGACTGTGGAGGATGGCCCCACCAAGT | |
| | CTGACCCCAGGTGCCTGACCAGATACTACAGCAGCTTTGTGAACATGGAGAGGGACCTGGCCTC | |
| | TGGCCTGATTGGCCCCCTGCTGATCTGCTACAAGGAGTCTGTGGACCAGAGGGGCAACCAGATC | |
| | ATGTCTGACAAGAGGAATGTGATCCTGTTCTCTGTGTTTGATGAGAACAGGAGCTGGTACCTGA | |
| | CTGAGAACATCCAGAGGTTCCTGCCCAACCCTGCTGGGGTGCAGCTGGAGGACCCTGAGTTCCA | |
| | GGCCAGCAACATCATGCACAGCATCAATGGCTATGTGTTTGACAGCCTGCAGCTGTCTGTGTGCC | |
| | TGCATGAGGTGGCCTACTGGTACATCCTGAGCATTGGGGCCCAGACTGACTTCCTGTCTGTGTTC | |
| | TTCTCTGGCTACACCTTCAAGCACAAGATGGTGTATGAGGACACCCTGACCCTGTTCCCCTTCTC | |
| | TGGGGAGACTGTGTTCATGAGCATGGAGAACCCTGGCCTGTGGATTCTGGGCTGCCACAACTCT | |
| | GACTTCAGGAACAGGGGCATGACTGCCCTGCTGAAAGTCTCCAGCTGTGACAAGAACACTGGGG | |
| | ACTACTATGAGGACAGCTATGAGGACATCTCTGCCTACCTGCTGAGCAAGAACAATGCCATTGA | |
| | GCCCAGGAGCTTCAGCCAGAATGCCACTAATGTGTCTAACAACAGCAACACCAGCAATGACAGC | |
| | AATGTGTCTCCCCCAGTGCTGAAGAGGCACCAGAGGGAGATCACCAGGACCACCCTGCAGTCTG | |
| | ACCAGGAGGAGATTGACTATGATGACACCATCTCTGTGGAGATGAAGAAGGAGGACTTTGACAT | |
| | CTACGACGAGGACGAGAACCAGAGCCCCAGGAGCTTCCAGAAGAAGACCAGGCACTACTTCAT | |
| | TGCTGCTGTGGAGAGGCTGTGGGACTATGGCATGAGCAGCAGCCCCCATGTGCTGAGGAACAGG | |
| | GCCCAGTCTGGCTCTGTGCCCCAGTTCAAGAAGGTGGTGTTCCAGGAGTTCACTGATGGCAGCTT | |
| | CACCCAGCCCCTGTACAGAGGGGAGCTGAATGAGCACCTGGGCCTGCTGGGCCCCTACATCAGG | |
| | GCTGAGGTGGAGGACAACATCATGGTGACCTTCAGGAACCAGGCCAGCAGGCCCTACAGCTTCT | |
| | ACAGCAGCCTGATCAGCTATGAGGAGGACCAGAGGCAGGGGGCTGAGCCCAGGAAGAACTTTG | |
| | TGAAGCCCAATGAAACCAAGACCTACTTCTGGAAGGTGCAGCACCACATGGCCCCCACCAAGGA | |
| | TGAGTTTGACTGCAAGGCCTGGGCCTACTTCTCTGATGTGGACCTGGAGAAGGATGTGCACTCTG | |
| | GCCTGATTGGCCCCCTGCTGGTGTGCCACACCAACACCCTGAACCCTGCCCATGGCAGGCAGGT | |
| | GACTGTGCAGGAGTTTGCCCTGTTCTTCACCATCTTTGATGAAACCAAGAGCTGGTACTTCACTG | |
| | AGAACATGGAGAGGAACTGCAGGGCCCCCTGCAACATCCAGATGGAGGACCCCACCTTCAAGG | |
| | AGAACTACAGGTTCCATGCCATCAATGGCTACATCATGGACACCCTGCCTGGCCTGGTGATGGC | |
| | CCAGGACCAGAGGATCAGGTGGTACCTGCTGAGCATGGGCAGCAATGAGAACATCCACAGCAT | |
| | CCACTTCTCTGGCCATGTGTTCACTGTGAGGAAGAAGGAGGAGTACAAGATGGCCCTGTACAAC | |
| | CTGTACCCTGGGGTGTTTGAGACTGTGGAGATGCTGCCCAGCAAGGCTGGCATCTGGAGGGTGG | |
| | AGTGCCTGATTGGGGAGCACCTGCATGCTGGCATGAGCACCCTGTTCCTGGTGTACAGCAACAA | |
| | GTGCCAGACCCCCCTGGGCATGGCCTCTGGCCACATCAGGGACTTCCAGATCACTGCCTCTGGCC | |
| | AGTATGGCCAGTGGGCCCCCAAGCTGGCCAGGCTGCACTACTCTGGCAGCATCAATGCCTGGAG | |
| | CACCAAGGAGCCCTTCAGCTGGATCAAGGTGGACCTGCTGGCCCCCATGATCATCCATGGCATC | |
| | AAGACCCAGGGGGCCAGGCAGAAGTTCAGCAGCCTGTACATCAGCCAGTTCATCATCATGTACA | |
| | GCCTGGATGGCAAGAAGTGGCAGACCTACAGGGGCAACAGCACTGGCACCCTGATGGTGTTCTT | |
| | TGGCAATGTGGACAGCTCTGGCATCAAGCACAACATCTTCAACCCCCCCATCATTGCCAGATAC | |
| | ATCAGGCTGCACCCCACCCACTACAGCATCAGGAGCACCCTGAGGATGGAGCTGATGGGCTGTG | |
| | ACCTGAACAGCTGCAGCATGCCCCTGGGCATGGAGAGCAAGGCCATCTCTGATGCCCAGATCAC | |
| | TGCCAGCAGCTACTTCACCAACATGTTTGCCACCTGGAGCCCCAGCAAGGCCAGGCTGCACCTG | |
| | CAGGGCAGGAGCAATGCCTGGAGGCCCCAGGTCAACAACCCCAAGGAGTGGCTGCAGGTGGAC | |
| | TTCCAGAAGACCATGAAGGTGACTGGGGTGACCACCCAGGGGGTGAAGAGCCTGCTGACCAGC | |
| | ATGTATGTGAAGGAGTTCCTGATCAGCAGCAGCCAGGATGGCCACCAGTGGACCCTGTTCTTCC | |
| | AGAATGGCAAGGTGAAGGTGTTCCAGGGCAACCAGGACAGCTTCACCCCTGTGGTGAACAGCCT | |
| | GGACCCCCCCCTGCTGACCAGATACCTGAGGATTCACCCCCAGAGCTGGGTGCACCAGATTGCC | |
| | CTGAGGATGGAGGTGCTGGGCTGTGAGGCCCAGGACCTGTACTGA | |
| APOE- | CGCGTCTGCAGGCTCAGAGGCACACAGGAGTTTCTGGGCTCACCCTGCCCCCTTCCAACCCCTCA | 20 |
| AAT-hBGi- | GTTCCCATCCTCCAGCAGCTGTTTGTGTGCTGCCTCTGAAGTCCACACTGAACAAACTTCAGCCT | |
| FVIIIv3- | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| bGH-poly A | CACTCGACCCCTTGGAATTTCGGTGGAGAGGAGCAGAGGTTGTCCTGGCGTGGTTTAGGTAGTG | |
| DTS | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGATTGAGCTGAGCACCTGCTTC | |
| | TTCCTGTGCCTGCTGAGGTTCTGCTTCTCTGCCACCAGGAGATACTACCTGGGGGCTGTGGAGCT | |
| | GAGCTGGGACTACATGCAGTCTGACCTGGGGGAGCTGCCTGTGGATGCCAGGTTCCCCCCCAGA | |
| | GTGCCCAAGAGCTTCCCCTTCAACACCTCTGTGGTGTACAAGAAGACCCTGTTTGTGGAGTTCAC | |
| | TGACCACCTGTTCAACATTGCCAAGCCCAGGCCCCCCTGGATGGGCCTGCTGGGCCCCACCATCC | |
| | AGGCTGAGGTGTATGACACTGTGGTGATCACCCTGAAGAACATGGCCAGCCACCCTGTGAGCCT | |
| | GCATGCTGTGGGGGTGAGCTACTGGAAGGCCTCTGAGGGGGCTGAGTATGATGACCAGACCAGC | |
| | CAGAGGGAGAAGGAGGATGACAAGGTGTTCCCTGGGGGCAGCCACACCTATGTGTGGCAGGTG | |
| | CTGAAGGAGAATGGCCCCATGGCCTCTGACCCCCTGTGCCTGACCTACAGCTACCTGAGCCATG | |
| | TGGACCTGGTGAAGGACCTGAACTCTGGCCTGATTGGGGCCCTGCTGGTGTGCAGGGAGGGCAG | |
| | CCTGGCCAAGGAGAAGACCCAGACCCTGCACAAGTTCATCCTGCTGTTTGCTGTGTTTGATGAG | |
| | GGCAAGAGCTGGCACTCTGAAACCAAGAACAGCCTGATGCAGGACAGGGATGCTGCCTCTGCC | |
| | AGGGCCTGGCCCAAGATGCACACTGTGAATGGCTATGTGAACAGGAGCCTGCCTGGCCTGATTG | |
| | GCTGCCACAGGAAGTCTGTGTACTGGCATGTGATTGGCATGGGCACCACCCCTGAGGTGCACAG | |
| | CATCTTCCTGGAGGGCCACACCTTCCTGGTCAGGAACCACAGGCAGGCCAGCCTGGAGATCAGC | |
| | CCCATCACCTTCCTGACTGCCCAGACCCTGCTGATGGACCTGGGCCAGTTCCTGCTGTTCTGCCA | |
| | CATCAGCAGCCACCAGCATGATGGCATGGAGGCCTATGTGAAGGTGGACAGCTGCCCTGAGGA | |
| | GCCCCAGCTGAGGATGAAGAACAATGAGGAGGCTGAGGACTATGATGATGACCTGACTGACTCT | |
| | GAGATGGATGTGGTGAGGTTTGATGATGACAACAGCCCCAGCTTCATCCAGATCAGGTCTGTGG | |
| | CCAAGAAGCACCCCAAGACCTGGGTGCACTACATTGCTGCTGAGGAGGAGGACTGGGACTATGC | |
| | CCCCCTGGTGCTGGCCCCTGATGACAGGAGCTACAAGAGCCAGTACCTGAACAATGGCCCCCAG | |
| | AGGATTGGCAGGAAGTACAAGAAGGTCAGGTTCATGGCCTACACTGATGAAACCTTCAAGACCA | |
| | GGGAGGCCATCCAGCATGAGTCTGGCATCCTGGGCCCCCTGCTGTATGGGGAGGTGGGGGACAC | |
| | CCTGCTGATCATCTTCAAGAACCAGGCCAGCAGGCCCTACAACATCTACCCCCATGGCATCACT | |
| | GATGTGAGGCCCCTGTACAGCAGGAGGCTGCCCAAGGGGGTGAAGCACCTGAAGGACTTCCCC | |
| | ATCCTGCCTGGGGAGATCTTCAAGTACAAGTGGACTGTGACTGTGGAGGATGGCCCCACCAAGT | |
| | CTGACCCCAGGTGCCTGACCAGATACTACAGCAGCTTTGTGAACATGGAGAGGGACCTGGCCTC | |
| | TGGCCTGATTGGCCCCCTGCTGATCTGCTACAAGGAGTCTGTGGACCAGAGGGGCAACCAGATC | |
| | ATGTCTGACAAGAGGAATGTGATCCTGTTCTCTGTGTTTGATGAGAACAGGAGCTGGTACCTGA | |
| | CTGAGAACATCCAGAGGTTCCTGCCCAACCCTGCTGGGGTGCAGCTGGAGGACCCTGAGTTCCA | |
| | GGCCAGCAACATCATGCACAGCATCAATGGCTATGTGTTTGACAGCCTGCAGCTGTCTGTGTGCC | |
| | TGCATGAGGTGGCCTACTGGTACATCCTGAGCATTGGGGCCCAGACTGACTTCCTGTCTGTGTTC | |
| | TTCTCTGGCTACACCTTCAAGCACAAGATGGTGTATGAGGACACCCTGACCCTGTTCCCCTTCTC | |
| | TGGGGAGACTGTGTTCATGAGCATGGAGAACCCTGGCCTGTGGATTCTGGGCTGCCACAACTCT | |
| | GACTTCAGGAACAGGGGCATGACTGCCCTGCTGAAAGTCTCCAGCTGTGACAAGAACACTGGGG | |
| | ACTACTATGAGGACAGCTATGAGGACATCTCTGCCTACCTGCTGAGCAAGAACAATGCCATTGA | |
| | GCCCAGGAGCTTCAGCCAGAATGCCACTAATGTGTCTAACAACAGCAACACCAGCAATGACAGC | |
| | AATGTGTCTCCCCCAGTGCTGAAGAGGCACCAGAGGGAGATCACCAGGACCACCCTGCAGTCTG | |
| | ACCAGGAGGAGATTGACTATGATGACACCATCTCTGTGGAGATGAAGAAGGAGGACTTTGACAT | |
| | CTACGACGAGGACGAGAACCAGAGCCCCAGGAGCTTCCAGAAGAAGACCAGGCACTACTTCAT | |
| | TGCTGCTGTGGAGAGGCTGTGGGACTATGGCATGAGCAGCAGCCCCCATGTGCTGAGGAACAGG | |
| | GCCCAGTCTGGCTCTGTGCCCCAGTTCAAGAAGGTGGTGTTCCAGGAGTTCACTGATGGCAGCTT | |
| | CACCCAGCCCCTGTACAGAGGGGAGCTGAATGAGCACCTGGGCCTGCTGGGCCCCTACATCAGG | |
| | GCTGAGGTGGAGGACAACATCATGGTGACCTTCAGGAACCAGGCCAGCAGGCCCTACAGCTTCT | |
| | ACAGCAGCCTGATCAGCTATGAGGAGGACCAGAGGCAGGGGGCTGAGCCCAGGAAGAACTTTG | |
| | TGAAGCCCAATGAAACCAAGACCTACTTCTGGAAGGTGCAGCACCACATGGCCCCCACCAAGGA | |
| | TGAGTTTGACTGCAAGGCCTGGGCCTACTTCTCTGATGTGGACCTGGAGAAGGATGTGCACTCTG | |
| | GCCTGATTGGCCCCCTGCTGGTGTGCCACACCAACACCCTGAACCCTGCCCATGGCAGGCAGGT | |
| | GACTGTGCAGGAGTTTGCCCTGTTCTTCACCATCTTTGATGAAACCAAGAGCTGGTACTTCACTG | |
| | AGAACATGGAGAGGAACTGCAGGGCCCCCTGCAACATCCAGATGGAGGACCCCACCTTCAAGG | |
| | AGAACTACAGGTTCCATGCCATCAATGGCTACATCATGGACACCCTGCCTGGCCTGGTGATGGC | |
| | CCAGGACCAGAGGATCAGGTGGTACCTGCTGAGCATGGGCAGCAATGAGAACATCCACAGCAT | |
| | CCACTTCTCTGGCCATGTGTTCACTGTGAGGAAGAAGGAGGAGTACAAGATGGCCCTGTACAAC | |
| | CTGTACCCTGGGGTGTTTGAGACTGTGGAGATGCTGCCCAGCAAGGCTGGCATCTGGAGGGTGG | |
| | AGTGCCTGATTGGGGAGCACCTGCATGCTGGCATGAGCACCCTGTTCCTGGTGTACAGCAACAA | |
| | GTGCCAGACCCCCCTGGGCATGGCCTCTGGCCACATCAGGGACTTCCAGATCACTGCCTCTGGCC | |
| | AGTATGGCCAGTGGGCCCCCAAGCTGGCCAGGCTGCACTACTCTGGCAGCATCAATGCCTGGAG | |
| | CACCAAGGAGCCCTTCAGCTGGATCAAGGTGGACCTGCTGGCCCCCATGATCATCCATGGCATC | |
| | AAGACCCAGGGGGCCAGGCAGAAGTTCAGCAGCCTGTACATCAGCCAGTTCATCATCATGTACA | |
| | GCCTGGATGGCAAGAAGTGGCAGACCTACAGGGGCAACAGCACTGGCACCCTGATGGTGTTCTT | |
| | TGGCAATGTGGACAGCTCTGGCATCAAGCACAACATCTTCAACCCCCCCATCATTGCCAGATAC | |
| | ATCAGGCTGCACCCCACCCACTACAGCATCAGGAGCACCCTGAGGATGGAGCTGATGGGCTGTG | |
| | ACCTGAACAGCTGCAGCATGCCCCTGGGCATGGAGAGCAAGGCCATCTCTGATGCCCAGATCAC | |
| | TGCCAGCAGCTACTTCACCAACATGTTTGCCACCTGGAGCCCCAGCAAGGCCAGGCTGCACCTG | |
| | CAGGGCAGGAGCAATGCCTGGAGGCCCCAGGTCAACAACCCCAAGGAGTGGCTGCAGGTGGAC | |
| | TTCCAGAAGACCATGAAGGTGACTGGGGTGACCACCCAGGGGGTGAAGAGCCTGCTGACCAGC | |
| | ATGTATGTGAAGGAGTTCCTGATCAGCAGCAGCCAGGATGGCCACCAGTGGACCCTGTTCTTCC | |
| | AGAATGGCAAGGTGAAGGTGTTCCAGGGCAACCAGGACAGCTTCACCCCTGTGGTGAACAGCCT | |
| | GGACCCCCCCCTGCTGACCAGATACCTGAGGATTCACCCCCAGAGCTGGGTGCACCAGATTGCC | |
| | CTGAGGATGGAGGTGCTGGGCTGTGAGGCCCAGGACCTGTACTGATAACTCGAGAATCAACCTC | |
| | TGGATTACAAAATTTGTGAAAGATTGACTGGTATTCTTAACTATGTTGCTCCTTTTACGCTATGTG | |
| | GATACGCTGCTTTAATGCCTTTGTATCATGCTATTGCTTCCCGTATGGCTTTCATTTTCTCCTCCTT | |
| | GTATAAATCCTGGTTGCTGTCTCTTTATGAGGAGTTGTGGCCCGTTGTCAGGCAACGTGGCGTGG | |
| | TGTGCACTGTGTTTGCTGACGCAACCCCCACTGGTTGGGGCATTGCCACCACCTGTCAGCTCCTT | |
| | TCCGGGACTTTCGCTTTCCCCCTCCCTATTGCCACGGCGGAACTCATCGCCGCCTGCCTTGCCCG | |
| | CTGCTGGACAGGGGCTCGGCTGTTGGGCACTGACAATTCCGTGGGGTGTGCCTTCTAGTTGCCAG | |
| | CCATCTGTTGTTTGCCCCTCCCCCGTGCCTTCCTTGACCCTGGAAGGTGCCACTCCCACTGTCCTT | |
| | TCCTAATAAAATGAGGAAATTGCATCGCATTGTCTGAGTAGGTGTCATTCTATTCTGGGGGGTGG | |
| | GGTGGGGCAGGACAGCAAGGGGGAGGATTGGGAAGACAATAGCAGGCATGCTGGGGATGCGGT | |
| | GGGCTCTATGGGAAGATGTCTACTGAGCTGTGCGATCCCTGCTGGGGACTTTCCGCTGGGGACTT | |
| | TCCGCTGGGGACTTTCCGCCTTCAGCTAAGGAAGCTACCAATATTTAGAGGTACATTTTGTTCTA | |
| | GAACAAAATGTACCGGTACATTTTGTTCTGGTACATTTTGTTCT | |
| FIX AA | MQRVNMIMAESPGLITICLLGYLLSAECTVFLDHENANKILNRPKRYNSGKLEEFVQGNLERECMEE | 21 |
| Sequence | KCSFEEAREVFENTERTTEFWKQYVDGDQCESNPCLNGGSCKDDINSYECWCPFGFEGKNCELDVT | |
| | CNIKNGRCEQFCKNSADNKVVCSCTEGYRLAENQKSCEPAVPFPCGRVSVSQTSKLTRAETVFPDVD | |
| | YVNSTEAETILDNITQSTQSFNDFTRVVGGEDAKPGQFPWQVVLNGKVDAFCGGSIVNEKWIVTAAH | |
| | CVETGVKITVVAGEHNIEETEHTEQKRNVIRIIPHHNYNAAINKYNHDIALLELDEPLVLNSYVTPICIA | |
| | DKEYTNIFLKFGSGYVSGWGRVFHKGRSALVLQYLRVPLVDRATCLRSTKFTIYNNMFCAGFHEGG | |
| | RDSCQGDSGGPHVTEVEGTSFLTGIISWGEECAMKGKYGIYTKVSRYVNWIKEKTKLT** | |
| 3′ITR | AGGAACCCCTAGTGATGGAGTTGGCCACTCCCTCTCTGCGCGCTCGCTCGCTCACTGAGGCCGG | 22 |
| | GCGACCAAAGGTCGCCCGACGCCCGGGCTTTGCCCGGGCGGCCTCAGTGAGCGAGCGAGCGCG | |
| | CAGCTGCCTGCAGG | |
| NP | TGGCTTGTTGTCCACAACCATTAAACCTTAAAAGCTTTAAAAGCCTTATATATTCTTTTTTTTCTT | 23 |
| Backbone | ATAAAACTTAAAACCTTAGAGGCTATTTAAGTTGCTGATTTATATTAATTTTATTGTTCAAACAT | |
| | GAGAGCTTAGTACGTGAAACATGAGAGCTTAGTACATTAGCCATGAGAGCTTAGTACATTAGCC | |
| | ATGAGGGTTTAGTTCATTAAACATGAGAGCTTAGTACATTAAACATGAGAGCTTAGTACATTAA | |
| | ACATGAGAGCTTAGTACATACTATCAACAGGTTGAACTGCTGATCTGTACAGTAGAATTGGTAA | |
| | AGAGAGTTGTGTAAAATATTGAGTTCGCACATCTTGTTGTCTGATTATTGATTTTTGGCGAAACC | |
| | ATTTGATCATATGACAAGATGTGTATCTACCTTAACTTAATGATTTTGATAAAAATCATTA | |
| FVIII-SQ- | MQIELSTCFFLCLLRFCFSATRRYYLGAVELSWDYMQSDLGELPVDARFPPRVPKSFPFNTSVVYKKT | 24 |
| N6-F309S- | LFVEFTDHLFNIAKPRPPWMGLLGPTIQAEVYDTVVITLKNMASHPVSLHAVGVSYWKASEGAEYD | |
| DM | DQTSQREKEDDKVFPGGSHTYVWQVLKENGPMASDPLCLTYSYLSHVDLVKDLNSGLIGALLVCRE | |
| | GSLAKEKTQTLHKFILLFAVFDEGKSWHSETKNSLMQDRDAASARAWPKMHTVNGYVNRSLPGLIG | |
| | CHRKSVYWHVIGMGTTPEVHSIFLEGHTFLVRNHRQASLEISPITFLTAQTLLMDLGQFLLSCHISSHQ | |
| | HDGMEAYVKVDSCPEEPQLRMKNNEEAEDYDDDLTDSEMDVVRFDDDNSPSFIQIRSVAKKHPKT | |
| | WVHYIAAEEEDWDYAPLVLAPDDRSYKSQYLNNGPQRIGRKYKKVRFMAYTDETFKTREAIQHESG | |
| | ILGPLLYGEVGDTLLIIFKNQASRPYNIYPHGITDVRPLYSRRLPKGVKHLKDFPILPGEIFKYKWTVTV | |
| | EDGPTKSDPRCLTRYYSSFVNMERDLASGLIGPLLICYKESVDQRGNQIMSDKRNVILFSVFDENRSW | |
| | YLTENIQRFLPNPAGVQLEDPEFQASNIMHSINGYVFDSLQLSVCLHEVAYWYILSIGAQTDFLSVFFS | |
| | GYTFKHKMVYEDTLTLFPFSGETVFMSMENPGLWILGCHNSDFRNRGMTALLKVSSCDKNTGDYYE | |
| | DSYEDISAYLLSKNNAIEPRSFSQNPPVLTRSFSQNSRHPSTRQKQFNATTIPENDIEKTDPWFAHRTP | |
| | MPKIQNVSSSDLLMLLRQSPTPHGLSLSDLQEAKYETFSDDPSPGAIDSNNSLSEMTHFRPQLHHSGD | |
| | MVFTPESGLQLRLNEKLGTTAATELKKLDFKVSSTSNNLISTIPSDNLAAGTDNTSSLGPPSMPVHYD | |
| | SQLDTTLFGKKSSPLTESGGPLSLSEENNDSKLLESGLMNSQESSWGKNVSSTREITRTTLQSDQEEID | |
| | YDDTISVEMKKEDFDIYDEDENQSPRSFQKKTRHYFIAAVERLWDYGMSSSPHVLRNRAQSGSVPQF | |
| | KKVVFQEFTDGSFTQPLYRGELNEHLGLLGPYIRAEVEDNIMVTFRNQASRPYSFYSSLISYEEDQRQ | |
| | GAEPRKNFVKPNETKTYFWKVQHHMAPTKDEFDCKAWAYFSDVDLEKDVHSGLIGPLLVCHTNTL | |
| | NPAHGRQVTVQEFALFFTIFDETKSWYFTENMERNGRAPGNIQMEDPTFKENYRFHAINGYIMDTLP | |
| | GLVMAQDQRIRWYLLSMGSNENIHSIHFSGHVFTVRKKEEYKMALYNLYPGVFETVEMLPSKAGIW | |
| | RVECLIGEHLHAGMSTLFLVYSNKCQTPLGMASGHIRDFQITASGQYGQWAPKLARLHYSGSINAWS | |
| | TKEPFS WIKVDLLAPMIIHGIKTQGARQKFSSLYISQFIIMYSLDGKKWQTYRGNSTGTLMVFFGNVD | |
| | SSGIKHNIFNPPIIARYIRLHPTHYSIRSTLRMELMGCDLNSCSMPLGMESKAISDAQITASSYFTNMFA | |
| | TWSPSKARLHLQGRSNAWRPQVNNPKEWLQVDFQKTMKVTGVTTQGVKSLLTSMYVKEFLISSSQ | |
| | DGHQWTLFFQNGKVKVFQGNQDSFTPVVNSLDPPLLTRYLRIHPQSWVHQIALRMEVLGCEAQDLY | |
| FVIII-SQ- | MQIELSTCFFLCLLRFCFSATRRYYLGAVELSWDYMQSDLGELPVDARFPPRVPKSFPFNTSVVYKKT | 25 |
| N6-F309S- | LFVEFTDHLFNIAKPRPPWMGLLGPTIQAEVYDTVVITLKNMASHPVSLHAVGVSYWKASEGAEYD | |
| DM: A1 | DQTSQREKEDDKVFPGGSHTYVWQVLKENGPMASDPLCLTYSYLSHVDLVKDLNSGLIGALLVCRE | |
| domain | GSLAKEKTQTLHKFILLFAVFDEGKSWHSETKNSLMQDRDAASARAWPKMHTVNGYVNRSLPGLIG | |
| | CHRKSVYWHVIGMGTTPEVHSIFLEGHTFLVRNHRQASLEISPITFLTAQTLLMDLGQFLLSCHISSHQ | |
| | HDGMEAYVKVDSCPEEPQLRMKNNEEAEDYDDDLTDSEMDVVRFDDDNSPSFIQIR | |
| FVIII-SQ- | SVAKKHPKTWVHYIAAEEEDWDYAPLVLAPDDRSYKSQYLNNGPQRIGRKYKKVRFMAYTDETFK | 26 |
| N6-F309S- | TREAIQHESGILGPLLYGEVGDTLLIIFKNQASRPYNIYPHGITDVRPLYSRRLPKGVKHLKDFPILPGE | |
| DM: A2 | IFKYKWTVTVEDGPTKSDPRCLTRYYSSFVNMERDLASGLIGPLLICYKESVDQRGNQIMSDKRNVIL | |
| domain | FSVFDENRSWYLTENIQRFLPNPAGVQLEDPEFQASNIMHSINGYVFDSLQLSVCLHEVAYWYILSIG | |
| | AQTDFLSVFFSGYTFKHKMVYEDTLTLFPFSGETVFMSMENPGLWILGCHNSDFRNRGMTALLKVSS | |
| | CDKNTGDYYEDSYEDISAYLLSKNNAIEPR | |
| FVIII-SQ- | SFSQNPPVLTR | 27 |
| N6-F309S- | | |
| DM: SQ | | |
| domain | | |
| FVIII-SQ- | SFSQNSRHPSTRQKQFNATTIPENDIEKTDPWFAHRTPMPKIQNVSSSDLLMLLRQSPTPHGLSLSDLQ | 28 |
| N6-F309S- | EAKYETFSDDPSPGAIDSNNSLSEMTHFRPQLHHSGDMVFTPESGLQLRLNEKLGTTAATELKKLDF | |
| DM: N6 | KVSSTSNNLISTIPSDNLAAGTDNTSSLGPPSMPVHYDSQLDTTLFGKKSSPLTESGGPLSLSEENNDS | |
| domain | KLLESGLMNSQESSWGKNVSST | |
| FVIII-SQ- | REITRTTLQSDQEEIDYDDTISVEMKKEDFDIYDEDENQSPRSFQKKTRHYFIAAVERLWDYGMSSSP | 29 |
| N6-F309S- | HVLRNRAQSGSVPQFKKVVFQEFTDGSFTQPLYRGELNEHLGLLGPYIRAEVEDNIMVTFRNQASRP | |
| DM: A3 | YSFYSSLISYEEDQRQGAEPRKNFVKPNETKTYFWKVQHHMAPTKDEFDCKAWAYFSDVDLEKDV | |
| domain | HSGLIGPLLVCHTNTLNPAHGRQVTVQEFALFFTIFDETKSWYFTENMERNGRAPGNIQMEDPTFKE | |
| | NYRFHAINGYIMDTLPGLVMAQDQRIRWYLLSMGSNENIHSIHFSGHVFTVRKKEEYKMALYNLYP | |
| | GVFETVEMLPSKAGIWRVECLIGEHLHAGMSTLFLVYSNK | |
| FVIII-SQ- | CQTPLGMASGHIRDFQITASGQYGQWAPKLARLHYSGSINAWSTKEPFSWIKVDLLAPMIIHGIKTQG | 30 |
| N6-F309S- | ARQKFSSLYISQFIIMYSLDGKKWQTYRGNSTGTLMVFFGNVDSSGIKHNIFNPPIIARYIRLHPTHYSI | |
| DM: C1 | RSTLRMELMGCDLNS | |
| domain | | |
| FVIII-SQ- | CSMPLGMESKAISDAQITASSYFTNMFATWSPSKARLHLQGRSNAWRPQVNNPKEWLQVDFQKTM | 31 |
| N6-F309S- | KVTGVTTQGVKSLLTSMYVKEFLISSSQDGHQWTLFFQNGKVKVFQGNQDSFTPVVNSLDPPLLTRY | |
| DM: C1 | LRIHPQSWVHQIALRMEVLGCEAQDLY | |
| domain | | |
| FVIII-SQ- | ATGCAGATCGAGCTGTCTACCTGCTTCTTCCTGTGCCTGCTGCGGTTCTGCTTCAGCGCCACCAG | 32 |
| N6-F309S- | AAGATATTACCTGGGCGCCGTGGAACTGAGCTGGGACTACATGCAGTCTGACCTGGGAGAGCTG | |
| DM: NA | CCCGTGGACGCTAGATTTCCTCCAAGAGTGCCCAAGAGCTTCCCCTTCAACACCTCCGTGGTGTA | |
| Coding | CAAGAAAACCCTGTTCGTGGAATTCACCGACCACCTGTTCAATATCGCCAAGCCTCGGCCTCCTT | |
| Sequence 1 | GGATGGGACTGCTGGGACCTACAATTCAGGCCGAGGTGTACGACACCGTGGTCATCACCCTGAA | |
| | GAACATGGCCAGCCATCCTGTGTCTCTGCACGCCGTGGGAGTGTCTTACTGGAAGGCTTCTGAG | |
| | GGCGCCGAGTACGACGATCAGACAAGCCAGAGAGAGAAAGAGGACGACAAGGTTTTCCCTGGC | |
| | GGCAGCCACACCTATGTCTGGCAGGTCCTGAAAGAAAACGGCCCTATGGCCTCCGATCCTCTGT | |
| | GCCTGACATACAGCTACCTGAGCCACGTGGACCTGGTCAAGGACCTGAATTCTGGCCTGATCGG | |
| | AGCCCTGCTCGTGTGTAGAGAAGGCAGCCTGGCCAAAGAGAAAACCCAGACACTGCACAAGTT | |
| | CATCCTGCTGTTCGCCGTGTTCGACGAGGGCAAGAGCTGGCACAGCGAGACAAAGAACAGCCTG | |
| | ATGCAGGACAGGGATGCCGCCTCTGCTAGAGCTTGGCCTAAGATGCACACCGTGAACGGCTACG | |
| | TGAACAGAAGCCTGCCTGGACTGATCGGCTGCCACAGAAAGTCCGTGTACTGGCACGTGATCGG | |
| | CATGGGCACAACACCTGAGGTGCACAGCATCTTTCTGGAAGGACACACCTTCCTCGTGCGGAAC | |
| | CATAGACAGGCCAGCCTGGAAATCAGCCCTATCACCTTCCTGACCGCTCAGACCCTGCTGATGG | |
| | ATCTGGGCCAGTTTCTGCTGAGCTGCCACATCAGCTCCCACCAGCACGATGGCATGGAAGCCTA | |
| | CGTGAAGGTGGACAGCTGCCCCGAAGAACCCCAGCTGCGGATGAAGAACAACGAGGAAGCCGA | |
| | GGACTACGACGACGACCTGACCGACTCTGAGATGGACGTCGTCAGATTCGACGACGATAACAGC | |
| | CCCAGCTTCATCCAGATCAGAAGCGTGGCCAAGAAGCACCCCAAGACCTGGGTGCACTATATCG | |
| | CCGCCGAGGAAGAGGACTGGGATTACGCTCCTCTGGTGCTGGCCCCTGACGACAGAAGCTACAA | |
| | GAGCCAGTACCTGAACAACGGCCCTCAGCGGATCGGCCGGAAGTATAAGAAAGTGCGGTTCAT | |
| | GGCCTACACCGACGAGACATTCAAGACCAGAGAGGCCATCCAGCACGAGAGCGGAATTCTGGG | |
| | CCCTCTGCTGTATGGCGAAGTGGGCGATACACTGCTGATCATCTTCAAGAACCAGGCCAGCAGA | |
| | CCCTACAACATCTACCCTCACGGCATCACCGATGTGCGGCCCCTGTATTCTAGAAGGCTGCCCAA | |
| | GGGCGTGAAGCACCTGAAGGACTTCCCTATCCTGCCTGGCGAGATCTTCAAGTACAAGTGGACC | |
| | GTGACCGTGGAAGATGGCCCCACCAAGAGCGACCCTAGATGTCTGACACGGTACTACAGCAGCT | |
| | TCGTGAACATGGAACGCGACCTGGCCAGCGGCCTGATTGGACCTCTGCTGATCTGCTACAAAGA | |
| | AAGCGTGGACCAGCGGGGCAACCAGATCATGAGCGACAAGCGGAACGTGATCCTGTTTAGCGT | |
| | GTTCGATGAGAACCGGTCCTGGTATCTGACCGAGAACATCCAGCGGTTTCTGCCCAATCCTGCTG | |
| | GCGTGCAGCTGGAAGATCCTGAGTTCCAGGCCTCCAACATCATGCACTCCATCAATGGCTATGT | |
| | GTTCGACAGCCTGCAGCTGAGCGTGTGCCTGCACGAAGTGGCCTACTGGTACATCCTGAGCATT | |
| | GGCGCCCAGACCGACTTCCTGTCCGTGTTCTTTAGCGGCTACACCTTCAAGCACAAGATGGTGTA | |
| | CGAGGATACCCTGACACTGTTCCCATTCAGCGGCGAGACAGTGTTCATGAGCATGGAAAACCCC | |
| | GGCCTGTGGATCCTGGGCTGTCACAACAGCGACTTCCGGAACAGAGGCATGACAGCCCTGCTGA | |
| | AGGTGTCCAGCTGCGACAAGAACACCGGCGACTACTACGAGGACAGCTATGAGGACATCAGCG | |
| | CCTACCTGCTGAGCAAGAACAATGCCATCGAGCCCAGAAGCTTCTCCCAAAATCCTCCGGTCCT | |
| | CACACGTTCCTTCAGCCAGAATAGCAGACACCCCTCCACCAGACAGAAGCAGTTCAACGCCACA | |
| | ACAATCCCCGAGAACGACATCGAGAAAACCGATCCTTGGTTTGCCCACAGAACCCCTATGCCTA | |
| | AGATCCAGAACGTGTCCTCCAGCGATCTGCTGATGCTCCTGAGACAGAGCCCTACACCTCACGG | |
| | ACTGAGCCTGTCCGATCTGCAAGAGGCCAAATACGAAACCTTCAGCGACGACCCTTCTCCTGGC | |
| | GCCATCGACAGCAACAATAGCCTGAGCGAGATGACCCACTTCAGACCACAGCTGCACCACAGCG | |
| | GCGACATGGTGTTTACACCTGAGAGCGGCCTCCAGCTGAGACTGAATGAGAAGCTGGGAACCAC | |
| | CGCCGCCACCGAGCTGAAGAAACTGGACTTCAAGGTGTCCTCTACCAGCAACAACCTGATCAGC | |
| | ACAATCCCCTCCGACAACCTGGCTGCCGGCACCGACAACACATCTTCTCTGGGCCCACCTAGCAT | |
| | GCCCGTGCACTACGATAGCCAGCTGGATACCACACTGTTCGGCAAGAAGTCTAGCCCTCTGACA | |
| | GAGTCTGGCGGCCCTCTGTCTCTGAGCGAGGAAAACAACGACAGCAAGCTGCTGGAATCCGGCC | |
| | TGATGAACAGCCAAGAGTCCTCCTGGGGCAAGAATGTGTCCAGCACCAGAGAAATCACCCGGA | |
| | CCACACTGCAGAGCGACCAAGAAGAGATCGATTACGACGATACCATCAGCGTCGAGATGAAGA | |
| | AAGAAGATTTCGACATCTACGACGAGGACGAGAATCAGAGCCCTCGGAGCTTCCAGAAGAAAA | |
| | CCAGGCACTACTTTATTGCCGCCGTCGAGCGGCTGTGGGACTACGGAATGTCTAGCTCTCCTCAC | |
| | GTGCTGCGGAATAGAGCCCAGTCTGGTAGCGTGCCCCAGTTCAAAAAGGTCGTGTTCCAAGAGT | |
| | TCACCGACGGCAGCTTCACCCAGCCACTGTATAGAGGCGAGCTGAACGAGCATCTGGGCCTGCT | |
| | GGGCCCTTATATCAGAGCCGAAGTGGAAGATAACATCATGGTCACCTTCCGGAATCAGGCCTCT | |
| | CGGCCCTACAGCTTCTACAGCTCCCTGATCTCCTACGAAGAGGACCAGAGACAGGGCGCAGAGC | |
| | CCCGGAAGAATTTCGTGAAGCCCAACGAGACTAAGACCTACTTTTGGAAGGTGCAGCACCATAT | |
| | GGCCCCTACAAAGGACGAGTTCGACTGCAAAGCCTGGGCCTACTTCTCCGATGTGGACCTTGAG | |
| | AAGGATGTGCACAGCGGACTCATCGGCCCACTGCTTGTGTGCCACACCAACACACTGAACCCCG | |
| | CTCACGGCAGACAAGTGACAGTGCAAGAATTCGCCCTGTTTTTCACCATCTTCGACGAAACGAA | |
| | GTCCTGGTACTTCACCGAAAACATGGAAAGAAACGGACGCGCGCCTGGCAACATTCAGATGGA | |
| | AGATCCCACCTTCAAAGAGAACTACCGGTTCCACGCCATCAACGGCTACATCATGGACACACTG | |
| | CCCGGCCTGGTTATGGCTCAGGATCAGAGAATCCGGTGGTATCTGCTGTCCATGGGCTCCAACG | |
| | AGAATATCCACTCCATCCACTTCAGCGGCCACGTGTTCACCGTCCGCAAGAAAGAAGAGTACAA | |
| | GATGGCCCTGTACAATCTGTACCCCGGCGTTTTCGAAACCGTTGAGATGCTGCCTAGCAAGGCC | |
| | GGAATTTGGAGAGTGGAATGTCTGATTGGAGAGCACCTCCACGCCGGGATGAGCACCCTGTTTC | |
| | TGGTGTACTCCAACAAGTGTCAGACCCCTCTCGGCATGGCCTCTGGCCACATTAGAGACTTCCAG | |
| | ATCACCGCCAGCGGACAGTATGGACAGTGGGCCCCTAAACTGGCCAGACTGCACTACTCCGGCA | |
| | GCATCAATGCCTGGTCCACCAAAGAGCCTTTCAGCTGGATCAAAGTGGACCTGCTGGCTCCCAT | |
| | GATCATCCACGGAATCAAGACCCAGGGCGCCAGACAAAAGTTCAGCAGCCTGTACATCAGCCA | |
| | GTTCATCATCATGTACAGCCTGGACGGAAAGAAGTGGCAGACCTACCGGGGCAATAGCACCGGC | |
| | ACACTGATGGTGTTCTTCGGCAACGTGGACTCCAGCGGCATTAAGCACAACATCTTCAACCCTCC | |
| | AATCATTGCCCGGTACATCCGGCTGCACCCCACACACTACAGCATCAGGTCTACCCTGAGAATG | |
| | GAACTGATGGGCTGCGACCTGAACAGCTGCAGCATGCCCCTCGGAATGGAAAGCAAGGCCATC | |
| | AGCGACGCCCAGATCACAGCCTCTAGCTACTTCACCAACATGTTCGCCACTTGGAGCCCCTCTAA | |
| | GGCCCGGCTTCATCTGCAAGGCAGAAGCAACGCTTGGAGGCCCCAAGTGAACAACCCCAAAGA | |
| | ATGGCTGCAGGTCGACTTTCAGAAAACCATGAAAGTGACAGGCGTGACCACACAGGGCGTCAA | |
| | GTCCCTGCTGACCTCTATGTACGTGAAAGAGTTTCTGATCAGCTCCAGCCAGGACGGCCACCAGT | |
| | GGACCCTGTTCTTCCAGAACGGCAAAGTGAAAGTGTTCCAGGGAAATCAGGACAGCTTCACACC | |
| | CGTGGTCAACTCCCTGGATCCTCCACTGCTGACAAGATACCTGCGGATTCACCCTCAGTCTTGGG | |
| | TGCACCAGATTGCCCTGCGGATGGAAGTGCTGGGCTGTGAAGCTCAGGACCTCTACTGATAG | |
| FVIII-SQ- | ATGCAGATTGAGCTGAGCACCTGCTTCTTCCTGTGCCTGCTGAGGTTCTGCTTCTCTGCCACCAG | 33 |
| N6-F309S- | GAGATACTACCTGGGGGCTGTGGAGCTGAGCTGGGACTACATGCAGTCTGACCTGGGGGAGCTG | |
| DM: NA | CCTGTGGATGCCAGGTTCCCCCCCAGAGTGCCCAAGAGCTTCCCCTTCAACACCTCTGTGGTGTA | |
| Coding | CAAGAAGACCCTGTTTGTGGAGTTCACTGACCACCTGTTCAACATTGCCAAGCCCAGGCCCCCCT | |
| Sequence 2 | GGATGGGCCTGCTGGGCCCCACCATCCAGGCTGAGGTGTATGACACTGTGGTGATCACCCTGAA | |
| | GAACATGGCCAGCCACCCTGTGAGCCTGCATGCTGTGGGGGTGAGCTACTGGAAGGCCTCTGAG | |
| | GGGGCTGAGTATGATGACCAGACCAGCCAGAGGGAGAAGGAGGATGACAAGGTGTTCCCTGGG | |
| | GGCAGCCACACCTATGTGTGGCAGGTGCTGAAGGAGAATGGCCCCATGGCCTCTGACCCCCTGT | |
| | GCCTGACCTACAGCTACCTGAGCCATGTGGACCTGGTGAAGGACCTGAACTCTGGCCTGATTGG | |
| | GGCCCTGCTGGTGTGCAGGGAGGGCAGCCTGGCCAAGGAGAAGACCCAGACCCTGCACAAGTT | |
| | CATCCTGCTGTTTGCTGTGTTTGATGAGGGCAAGAGCTGGCACTCTGAAACCAAGAACAGCCTG | |
| | ATGCAGGACAGGGATGCTGCCTCTGCCAGGGCCTGGCCCAAGATGCACACTGTGAATGGCTATG | |
| | TGAACAGGAGCCTGCCTGGCCTGATTGGCTGCCACAGGAAGTCTGTGTACTGGCATGTGATTGG | |
| | CATGGGCACCACCCCTGAGGTGCACAGCATCTTCCTGGAGGGCCACACCTTCCTGGTCAGGAAC | |
| | CACAGGCAGGCCAGCCTGGAGATCAGCCCCATCACCTTCCTGACTGCCCAGACCCTGCTGATGG | |
| | ACCTGGGCCAGTTCCTGCTGAGCTGCCACATCAGCAGCCACCAGCATGATGGCATGGAGGCCTA | |
| | TGTGAAGGTGGACAGCTGCCCTGAGGAGCCCCAGCTGAGGATGAAGAACAATGAGGAGGCTGA | |
| | GGACTATGATGATGACCTGACTGACTCTGAGATGGATGTGGTGAGGTTTGATGATGACAACAGC | |
| | CCCAGCTTCATCCAGATCAGGTCTGTGGCCAAGAAGCACCCCAAGACCTGGGTGCACTACATTG | |
| | CTGCTGAGGAGGAGGACTGGGACTATGCCCCCCTGGTGCTGGCCCCTGATGACAGGAGCTACAA | |
| | GAGCCAGTACCTGAACAATGGCCCCCAGAGGATTGGCAGGAAGTACAAGAAGGTCAGGTTCAT | |
| | GGCCTACACTGATGAAACCTTCAAGACCAGGGAGGCCATCCAGCATGAGTCTGGCATCCTGGGC | |
| | CCCCTGCTGTATGGGGAGGTGGGGGACACCCTGCTGATCATCTTCAAGAACCAGGCCAGCAGGC | |
| | CCTACAACATCTACCCCCATGGCATCACTGATGTGAGGCCCCTGTACAGCAGGAGGCTGCCCAA | |
| | GGGGGTGAAGCACCTGAAGGACTTCCCCATCCTGCCTGGGGAGATCTTCAAGTACAAGTGGACT | |
| | GTGACTGTGGAGGATGGCCCCACCAAGTCTGACCCCAGGTGCCTGACCAGATACTACAGCAGCT | |
| | TTGTGAACATGGAGAGGGACCTGGCCTCTGGCCTGATTGGCCCCCTGCTGATCTGCTACAAGGA | |
| | GTCTGTGGACCAGAGGGGCAACCAGATCATGTCTGACAAGAGGAATGTGATCCTGTTCTCTGTG | |
| | TTTGATGAGAACAGGAGCTGGTACCTGACTGAGAACATCCAGAGGTTCCTGCCCAACCCTGCTG | |
| | GGGTGCAGCTGGAGGACCCTGAGTTCCAGGCCAGCAACATCATGCACAGCATCAATGGCTATGT | |
| | GTTTGACAGCCTGCAGCTGTCTGTGTGCCTGCATGAGGTGGCCTACTGGTACATCCTGAGCATTG | |
| | GGGCCCAGACTGACTTCCTGTCTGTGTTCTTCTCTGGCTACACCTTCAAGCACAAGATGGTGTAT | |
| | GAGGACACCCTGACCCTGTTCCCCTTCTCTGGGGAGACTGTGTTCATGAGCATGGAGAACCCTG | |
| | GCCTGTGGATTCTGGGCTGCCACAACTCTGACTTCAGGAACAGGGGCATGACTGCCCTGCTGAA | |
| | AGTCTCCAGCTGTGACAAGAACACTGGGGACTACTATGAGGACAGCTATGAGGACATCTCTGCC | |
| | TACCTGCTGAGCAAGAACAATGCCATTGAGCCCAGGAGCTTCAGCCAGAATCCCCCAGTGCTCA | |
| | CCCGGTCCTTTAGCCAGAATTCTCGCCATCCCTCCACCCGGCAAAAGCAGTTCAACGCCACTACC | |
| | ATCCCAGAGAACGACATCGAAAAAACCGACCCCTGGTTCGCCCACAGAACTCCCATGCCAAAGA | |
| | TTCAGAATGTTTCCAGTTCTGACCTCCTTATGTTGCTGCGCCAGTCTCCGACCCCTCATGGACTGT | |
| | CCCTGAGTGACTTGCAGGAGGCCAAGTACGAGACGTTCTCTGATGACCCCAGCCCAGGCGCGAT | |
| | TGACTCAAATAACTCCCTGTCTGAGATGACACATTTTCGCCCTCAGCTCCATCACAGCGGGGACA | |
| | TGGTGTTCACTCCAGAGTCCGGACTTCAGCTGCGCCTCAACGAGAAACTCGGTACTACAGCCGC | |
| | GACAGAACTCAAAAAGCTGGATTTCAAGGTTTCCAGCACCAGCAATAACCTGATCTCTACAATT | |
| | CCCAGCGATAACCTGGCGGCTGGAACCGACAACACTTCCAGCCTGGGACCTCCGTCCATGCCTG | |
| | TGCACTACGACTCCCAGCTCGACACCACTCTGTTCGGCAAGAAAAGTAGCCCCCTGACCGAATC | |
| | CGGCGGTCCGCTGTCCCTCTCCGAAGAGAATAACGATAGCAAGCTCCTGGAATCTGGGCTTATG | |
| | AACTCTCAAGAGTCATCCTGGGGCAAGAACGTTTCATCAACTAGGGAGATCACCAGGACCACCC | |
| | TGCAGTCTGACCAGGAGGAGATTGACTATGATGACACCATCTCTGTGGAGATGAAGAAGGAGG | |
| | ACTTTGACATCTACGACGAGGACGAGAACCAGAGCCCCAGGAGCTTCCAGAAGAAGACCAGGC | |
| | ACTACTTCATTGCTGCTGTGGAGAGGCTGTGGGACTATGGCATGAGCAGCAGCCCCCATGTGCT | |
| | GAGGAACAGGGCCCAGTCTGGCTCTGTGCCCCAGTTCAAGAAGGTGGTGTTCCAGGAGTTCACT | |
| | GATGGCAGCTTCACCCAGCCCCTGTACAGAGGGGAGCTGAATGAGCACCTGGGCCTGCTGGGCC | |
| | CCTACATCAGGGCTGAGGTGGAGGACAACATCATGGTGACCTTCAGGAACCAGGCCAGCAGGC | |
| | CCTACAGCTTCTACAGCAGCCTGATCAGCTATGAGGAGGACCAGAGGCAGGGGGCTGAGCCCA | |
| | GGAAGAACTTTGTGAAGCCCAATGAAACCAAGACCTACTTCTGGAAGGTGCAGCACCACATGGC | |
| | CCCCACCAAGGATGAGTTTGACTGCAAGGCCTGGGCCTACTTCTCTGATGTGGACCTGGAGAAG | |
| | GATGTGCACTCTGGCCTGATTGGCCCCCTGCTGGTGTGCCACACCAACACCCTGAACCCTGCCCA | |
| | TGGCAGGCAGGTGACTGTGCAGGAGTTTGCCCTGTTCTTCACCATCTTTGATGAAACCAAGAGCT | |
| | GGTACTTCACTGAGAACATGGAGAGGAACGGCAGGGCCCCCGGCAACATCCAGATGGAGGACC | |
| | CCACCTTCAAGGAGAACTACAGGTTCCATGCCATCAATGGCTACATCATGGACACCCTGCCTGG | |
| | CCTGGTGATGGCCCAGGACCAGAGGATCAGGTGGTACCTGCTGAGCATGGGCAGCAATGAGAA | |
| | CATCCACAGCATCCACTTCTCTGGCCATGTGTTCACTGTGAGGAAGAAGGAGGAGTACAAGATG | |
| | GCCCTGTACAACCTGTACCCTGGGGTGTTTGAGACTGTGGAGATGCTGCCCAGCAAGGCTGGCA | |
| | TCTGGAGGGTGGAGTGCCTGATTGGGGAGCACCTGCATGCTGGCATGAGCACCCTGTTCCTGGT | |
| | GTACAGCAACAAGTGCCAGACCCCCCTGGGCATGGCCTCTGGCCACATCAGGGACTTCCAGATC | |
| | ACTGCCTCTGGCCAGTATGGCCAGTGGGCCCCCAAGCTGGCCAGGCTGCACTACTCTGGCAGCA | |
| | TCAATGCCTGGAGCACCAAGGAGCCCTTCAGCTGGATCAAGGTGGACCTGCTGGCCCCCATGAT | |
| | CATCCATGGCATCAAGACCCAGGGGGCCAGGCAGAAGTTCAGCAGCCTGTACATCAGCCAGTTC | |
| | ATCATCATGTACAGCCTGGATGGCAAGAAGTGGCAGACCTACAGGGGCAACAGCACTGGCACC | |
| | CTGATGGTGTTCTTTGGCAATGTGGACAGCTCTGGCATCAAGCACAACATCTTCAACCCCCCCAT | |
| | CATTGCCAGATACATCAGGCTGCACCCCACCCACTACAGCATCAGGAGCACCCTGAGGATGGAG | |
| | CTGATGGGCTGTGACCTGAACAGCTGCAGCATGCCCCTGGGCATGGAGAGCAAGGCCATCTCTG | |
| | ATGCCCAGATCACTGCCAGCAGCTACTTCACCAACATGTTTGCCACCTGGAGCCCCAGCAAGGC | |
| | CAGGCTGCACCTGCAGGGCAGGAGCAATGCCTGGAGGCCCCAGGTCAACAACCCCAAGGAGTG | |
| | GCTGCAGGTGGACTTCCAGAAGACCATGAAGGTGACTGGGGTGACCACCCAGGGGGTGAAGAG | |
| | CCTGCTGACCAGCATGTATGTGAAGGAGTTCCTGATCAGCAGCAGCCAGGATGGCCACCAGTGG | |
| | ACCCTGTTCTTCCAGAATGGCAAGGTGAAGGTGTTCCAGGGCAACCAGGACAGCTTCACCCCTG | |
| | TGGTGAACAGCCTGGACCCCCCCCTGCTGACCAGATACCTGAGGATTCACCCCCAGAGCTGGGT | |
| | GCACCAGATTGCCCTGAGGATGGAGGTGCTGGGCTGTGAGGCCCAGGACCTGTACTGA | |
| N6-SQ | SFSQNPPVLTRSFSQNSRHPSTRQKQFNATTIPENDIEKTDPWFAHRTPMPKIQNVSSSDLLMLLRQSP | 34 |
| domain | TPHGLSLSDLQEAKYETFSDDPSPGAIDSNNSLSEMTHFRPQLHHSGDMVFTPESGLQLRLNEKLGTT | |
| | AATELKKLDFKVSSTSNNLISTIPSDNLAAGTDNTSSLGPPSMPVHYDSQLDTTLFGKKSSPLTESGGP | |
| | LSLSEENNDSKLLESGLMNSQESSWGKNVSST | |
| FVIII | MQIELSTCFFLCLLRFCFSATRRYYLGAVELSWDYMQSDLGELPVDARFPPRVPKSFPFNTSVVYKKT | 35 |
| (SQ + N6) | LFVEFTDHLFNIAKPRPPWMGLLGPTIQAEVYDTVVITLKNMASHPVSLHAVGVSYWKASEGAEYD | |
| | DQTSQREKEDDKVFPGGSHTYVWQVLKENGPMASDPLCLTYSYLSHVDLVKDLNSGLIGALLVCRE | |
| | GSLAKEKTQTLHKFILLFAVFDEGKSWHSETKNSLMQDRDAASARAWPKMHTVNGYVNRSLPGLIG | |
| | CHRKSVYWHVIGMGTTPEVHSIFLEGHTFLVRNHRQASLEISPITFLTAQTLLMDLGQFLLFCHISSHQ | |
| | HDGMEAYVKVDSCPEEPQLRMKNNEEAEDYDDDLTDSEMDVVRFDDDNSPSFIQIRSVAKKHPKT | |
| | WVHYIAAEEEDWDYAPLVLAPDDRSYKSQYLNNGPQRIGRKYKKVRFMAYTDETFKTREAIQHESG | |
| | ILGPLLYGEVGDTLLIIFKNQASRPYNIYPHGITDVRPLYSRRLPKGVKHLKDFPILPGEIFKYKWTVTV | |
| | EDGPTKSDPRCLTRYYSSFVNMERDLASGLIGPLLICYKESVDQRGNQIMSDKRNVILFSVFDENRSW | |
| | YLTENIQRFLPNPAGVQLEDPEFQASNIMHSINGYVFDSLQLSVCLHEVAYWYILSIGAQTDFLSVFFS | |
| | GYTFKHKMVYEDTLTLFPFSGETVFMSMENPGLWILGCHNSDFRNRGMTALLKVSSCDKNTGDYYE | |
| | DSYEDISAYLLSKNNAIEPRSFSQNPPVLTRSFSQNSRHPSTRQKQFNATTIPENDIEKTDPWFAHRTP | |
| | MPKIQNVSSSDLLMLLRQSPTPHGLSLSDLQEAKYETFSDDPSPGAIDSNNSLSEMTHFRPQLHHSGD | |
| | MVFTPESGLQLRLNEKLGTTAATELKKLDFKVSSTSNNLISTIPSDNLAAGTDNTSSLGPPSMPVHYD | |
| | SQLDTTLFGKKSSPLTESGGPLSLSEENNDSKLLESGLMNSQESSWGKNVSSTREITRTTLQSDQEEID | |
| | YDDTISVEMKKEDFDIYDEDENQSPRSFQKKTRHYFIAAVERLWDYGMSSSPHVLRNRAQSGSVPQF | |
| | KKVVFQEFTDGSFTQPLYRGELNEHLGLLGPYIRAEVEDNIMVTFRNQASRPYSFYSSLISYEEDQRQ | |
| | GAEPRKNFVKPNETKTYFWKVQHHMAPTKDEFDCKAWAYFSDVDLEKDVHSGLIGPLLVCHTNTL | |
| | NPAHGRQVTVQEFALFFTIFDETKSWYFTENMERNCRAPCNIQMEDPTFKENYRFHAINGYIMDTLP | |
| | GLVMAQDQRIRWYLLSMGSNENIHSIHFSGHVFTVRKKEEYKMALYNLYPGVFETVEMLPSKAGIW | |
| | RVECLIGEHLHAGMSTLFLVYSNKCQTPLGMASGHIRDFQITASGQYGQWAPKLARLHYSGSINAWS | |
| | TKEPFS WIKVDLLAPMIIHGIKTQGARQKFSSLYISQFIIMYSLDGKKWQTYRGNSTGTLMVFFGNVD | |
| | SSGIKHNIFNPPIIARYIRLHPTHYSIRSTLRMELMGCDLNSCSMPLGMESKAISDAQITASSYFTNMFA | |
| | TWSPSKARLHLQGRSNAWRPQVNNPKEWLQVDFQKTMKVTGVTTQGVKSLLTSMYVKEFLISSSQ | |
| | DGHQWTLFFQNGKVKVFQGNQDSFTPVVNSLDPPLLTRYLRIHPQSWVHQIALRMEVLGCEAQDLY | |
| FVIII A1 | MQIELSTCFFLCLLRFCFSATRRYYLGAVELSWDYMQSDLGELPVDARFPPRVPKSFPFNTSVVYKKT | 36 |
| | LFVEFTDHLFNIAKPRPPWMGLLGPTIQAEVYDTVVITLKNMASHPVSLHAVGVSYWKASEGAEYD | |
| | DQTSQREKEDDKVFPGGSHTYVWQVLKENGPMASDPLCLTYSYLSHVDLVKDLNSGLIGALLVCRE | |
| | GSLAKEKTQTLHKFILLFAVFDEGKSWHSETKNSLMQDRDAASARAWPKMHTVNGYVNRSLPGLIG | |
| | CHRKSVYWHVIGMGTTPEVHSIFLEGHTFLVRNHRQASLEISPITFLTAQTLLMDLGQFLLFCHISSHQ | |
| | HDGMEAYVKVDSCPEEPQLRMKNNEEAEDYDDDLTDSEMDVVRFDDDNSPSFIQIR | |
| FVIII A3 | REITRTTLQSDQEEIDYDDTISVEMKKEDFDIYDEDENQSPRSFQKKTRHYFIAAVERLWDYGMSSSP | 37 |
| | HVLRNRAQSGSVPQFKKVVFQEFTDGSFTQPLYRGELNEHLGLLGPYIRAEVEDNIMVTFRNQASRP | |
| | YSFYSSLISYEEDQRQGAEPRKNFVKPNETKTYFWKVQHHMAPTKDEFDCKAWAYFSDVDLEKDV | |
| | HSGLIGPLLVCHTNTLNPAHGRQVTVQEFALFFTIFDETKSWYFTENMERNCRAPCNIQMEDPTFKEN | |
| | YRFHAINGYIMDTLPGLVMAQDQRIRWYLLSMGSNENIHSIHFSGHVFTVRKKEEYKMALYNLYPG | |
| | VFETVEMLPSKAGIWRVECLIGEHLHAGMSTLFLVYSNK | |
| NP-ITR- | GAATCAACCTCTGGATTACAAAATTTGTGAAAGATTGACTGGTATTCTTAACTATGTTGCTCCTT | 38 |
| ApoE-AAT- | TTACGCTATGTGGATACGCTGCTTTAATGCCTTTGTATCATGCTATTGCTTCCCGTATGGCTTTCA | |
| hBGi- | TTTTCTCCTCCTTGTATAAATCCTGGTTGCTGTCTCTTTATGAGGAGTTGTGGCCCGTTGTCAGGC | |
| FVIII-SQ- | AACGTGGCGTGGTGTGCACTGTGTTTGCTGACGCAACCCCCACTGGTTGGGGCATTGCCACCACC | |
| N6-F309S- | TGTCAGCTCCTTTCCGGGACTTTCGCTTTCCCCCTCCCTATTGCCACGGCGGAACTCATCGCCGCC | |
| DM- | TGCCTTGCCCGCTGCTGGACAGGGGCTCGGCTGTTGGGCACTGACAATTCCGTGGGGTGTGCCTT | |
| WPRE3- | CTAGTTGCCAGCCATCTGTTGTTTGCCCCTCCCCCGTGCCTTCCTTGACCCTGGAAGGTGCCACTC | |
| DTS-ITR | CCACTGTCCTTTCCTAATAAAATGAGGAAATTGCATCGCATTGTCTGAGTAGGTGTCATTCTATT | |
| | CTGGGGGGTGGGGTGGGGCAGGACAGCAAGGGGGAGGATTGGGAAGACAATAGCAGGCATGCT | |
| | GGGGATGCGGTGGGCTCTATGGGCACGTGCGGAGCTGTGCGATCCCTGCTGGGGACTTTCCGCT | |
| | GGGGACTTTCCGCTGGGGACTTTCCGCCTTCAGCTAAGGAAGCTACCAATATTTAGAGGTACATT | |
| | TTGTTCTAGAACAAAATGTACCGGTACATTTTGTTCTGGTACATTTTGTTCTATCGATCGAGCGG | |
| | CCGCAGGAACCCCTAGTGATGGAGTTGGCCACTCCCTCTCTGCGCGCTCGCTCGCTCACTGAGGC | |
| | CGGGCGACCAAAGGTCGCCCGACGCCCGGGCTTTGCCCGGGCGGCCTCAGTGAGCGAGCGAGC | |
| | GCGCAGCTGCCTGCAGGGGCTTGTTGTCCACAACCATTAAACCTTAAAAGCTTTAAAAGCCTTAT | |
| | ATATTCTTTTTTTTCTTATAAAACTTAAAACCTTAGAGGCTATTTAAGTTGCTGATTTATATTAAT | |
| | TTTATTGTTCAAACATGAGAGCTTAGTACGTGAAACATGAGAGCTTAGTACATTAGCCATGAGA | |
| | GCTTAGTACATTAGCCATGAGGGTTTAGTTCATTAAACATGAGAGCTTAGTACATTAAACATGA | |
| | GAGCTTAGTACATTAAACATGAGAGCTTAGTACATACTATCAACAGGTTGAACTGCTGATCTGT | |
| | ACAGTAGAATTGGTAAAGAGAGTTGTGTAAAATATTGAGTTCGCACATCTTGTTGTCTGATTATT | |
| | GATTTTTGGCGAAACCATTTGATCATATGACAAGATGTGTATCTACCTTAACTTAATGATTTTGA | |
| | TAAAAATCATTACCTGCAGGCAGCTGCGCGCTCGCTCGCTCACTGAGGCCGCCCGGGCGTCGGG | |
| | CGACCTTTGGTCGCCCGGCCTCAGTGAGCGAGCGCGCAGAGAGGGAGTGGCCAACTCCATCACT | |
| | AGGGGTTCCTGCGGCCGCACGCGTCTAGTTATTAATAGTAATCGAATTCGCGTCTGCAGGCTCAG | |
| | AGGCACACAGGAGTTTCTGGGCTCACCCTGCCCCCTTCCAACCCCTCAGTTCCCATCCTCCAGCA | |
| | GCTGTTTGTGTGCTGCCTCTGAAGTCCACACTGAACAAACTTCAGCCTACTCATGTCCCTAAAAT | |
| | GGGCAAACATTGCAAGCAGCAAACAGCAAACACACAGCCCTCCCTGCCTGCTGACCTTGGAGCT | |
| | GGGGCAGAGGTCAGAGACCTCTCTGGGCCCATGCCACCTCCAACATCCACTCGACCCCTTGGAA | |
| | TTTCGGTGGAGAGGAGCAGAGGTTGTCCTGGCGTGGTTTAGGTAGTGTGAGAGGGGTCGACTGG | |
| | ACACAGGACGCTGTGGTTTCTGAGCCAGGGGGCGACTCAGATCCCAGCCAGTGGACTTAGCCCC | |
| | TGTTTGCTCCTCCGATAACTGGGGTGACCTTGGTTAATATTCACCAGCAGCCTCCCCCGTTGCCC | |
| | CTCTGGATCCACTGCTTAAATACGGACGAGGACAGGGCCCTGTCTCCTCAGCTTCAGGCACCAC | |
| | CACTGACCTGGGACAGTGAATCGTAAGTACTAGCAGCTACAATCCAGCTACCATTCTGCTTTTAT | |
| | TTTATGGTTGGGATAAGGCTGGATTATTCTGAGTCCAAGCTAGGCCCTTTTGCTAATCATGTTCA | |
| | TACCTCTTATCTTCCTCCCACAGCTCCTGGGCAACGTGCTGGTCTGTGTGCTGGCCCATCACTTTG | |
| | GCAAAGAATTGCGATCGCCACCATGCAGATTGAGCTGAGCACCTGCTTCTTCCTGTGCCTGCTGA | |
| | GGTTCTGCTTCTCTGCCACCAGGAGATACTACCTGGGGGCTGTGGAGCTGAGCTGGGACTACAT | |
| | GCAGTCTGACCTGGGGGAGCTGCCTGTGGATGCCAGGTTCCCCCCCAGAGTGCCCAAGAGCTTC | |
| | CCCTTCAACACCTCTGTGGTGTACAAGAAGACCCTGTTTGTGGAGTTCACTGACCACCTGTTCAA | |
| | CATTGCCAAGCCCAGGCCCCCCTGGATGGGCCTGCTGGGCCCCACCATCCAGGCTGAGGTGTAT | |
| | GACACTGTGGTGATCACCCTGAAGAACATGGCCAGCCACCCTGTGAGCCTGCATGCTGTGGGGG | |
| | TGAGCTACTGGAAGGCCTCTGAGGGGGCTGAGTATGATGACCAGACCAGCCAGAGGGAGAAGG | |
| | AGGATGACAAGGTGTTCCCTGGGGGCAGCCACACCTATGTGTGGCAGGTGCTGAAGGAGAATG | |
| | GCCCCATGGCCTCTGACCCCCTGTGCCTGACCTACAGCTACCTGAGCCATGTGGACCTGGTGAAG | |
| | GACCTGAACTCTGGCCTGATTGGGGCCCTGCTGGTGTGCAGGGAGGGCAGCCTGGCCAAGGAGA | |
| | AGACCCAGACCCTGCACAAGTTCATCCTGCTGTTTGCTGTGTTTGATGAGGGCAAGAGCTGGCA | |
| | CTCTGAAACCAAGAACAGCCTGATGCAGGACAGGGATGCTGCCTCTGCCAGGGCCTGGCCCAAG | |
| | ATGCACACTGTGAATGGCTATGTGAACAGGAGCCTGCCTGGCCTGATTGGCTGCCACAGGAAGT | |
| | CTGTGTACTGGCATGTGATTGGCATGGGCACCACCCCTGAGGTGCACAGCATCTTCCTGGAGGG | |
| | CCACACCTTCCTGGTCAGGAACCACAGGCAGGCCAGCCTGGAGATCAGCCCCATCACCTTCCTG | |
| | ACTGCCCAGACCCTGCTGATGGACCTGGGCCAGTTCCTGCTGAGCTGCCACATCAGCAGCCACC | |
| | AGCATGATGGCATGGAGGCCTATGTGAAGGTGGACAGCTGCCCTGAGGAGCCCCAGCTGAGGA | |
| | TGAAGAACAATGAGGAGGCTGAGGACTATGATGATGACCTGACTGACTCTGAGATGGATGTGGT | |
| | GAGGTTTGATGATGACAACAGCCCCAGCTTCATCCAGATCAGGTCTGTGGCCAAGAAGCACCCC | |
| | AAGACCTGGGTGCACTACATTGCTGCTGAGGAGGAGGACTGGGACTATGCCCCCCTGGTGCTGG | |
| | CCCCTGATGACAGGAGCTACAAGAGCCAGTACCTGAACAATGGCCCCCAGAGGATTGGCAGGA | |
| | AGTACAAGAAGGTCAGGTTCATGGCCTACACTGATGAAACCTTCAAGACCAGGGAGGCCATCCA | |
| | GCATGAGTCTGGCATCCTGGGCCCCCTGCTGTATGGGGAGGTGGGGGACACCCTGCTGATCATC | |
| | TTCAAGAACCAGGCCAGCAGGCCCTACAACATCTACCCCCATGGCATCACTGATGTGAGGCCCC | |
| | TGTACAGCAGGAGGCTGCCCAAGGGGGTGAAGCACCTGAAGGACTTCCCCATCCTGCCTGGGGA | |
| | GATCTTCAAGTACAAGTGGACTGTGACTGTGGAGGATGGCCCCACCAAGTCTGACCCCAGGTGC | |
| | CTGACCAGATACTACAGCAGCTTTGTGAACATGGAGAGGGACCTGGCCTCTGGCCTGATTGGCC | |
| | CCCTGCTGATCTGCTACAAGGAGTCTGTGGACCAGAGGGGCAACCAGATCATGTCTGACAAGAG | |
| | GAATGTGATCCTGTTCTCTGTGTTTGATGAGAACAGGAGCTGGTACCTGACTGAGAACATCCAG | |
| | AGGTTCCTGCCCAACCCTGCTGGGGTGCAGCTGGAGGACCCTGAGTTCCAGGCCAGCAACATCA | |
| | TGCACAGCATCAATGGCTATGTGTTTGACAGCCTGCAGCTGTCTGTGTGCCTGCATGAGGTGGCC | |
| | TACTGGTACATCCTGAGCATTGGGGCCCAGACTGACTTCCTGTCTGTGTTCTTCTCTGGCTACAC | |
| | CTTCAAGCACAAGATGGTGTATGAGGACACCCTGACCCTGTTCCCCTTCTCTGGGGAGACTGTGT | |
| | TCATGAGCATGGAGAACCCTGGCCTGTGGATTCTGGGCTGCCACAACTCTGACTTCAGGAACAG | |
| | GGGCATGACTGCCCTGCTGAAAGTCTCCAGCTGTGACAAGAACACTGGGGACTACTATGAGGAC | |
| | AGCTATGAGGACATCTCTGCCTACCTGCTGAGCAAGAACAATGCCATTGAGCCCAGGAGCTTCA | |
| | GCCAGAATCCCCCAGTGCTCACCCGGTCCTTTAGCCAGAATTCTCGCCATCCCTCCACCCGGCAA | |
| | AAGCAGTTCAACGCCACTACCATCCCAGAGAACGACATCGAAAAAACCGACCCCTGGTTCGCCC | |
| | ACAGAACTCCCATGCCAAAGATTCAGAATGTTTCCAGTTCTGACCTCCTTATGTTGCTGCGCCAG | |
| | TCTCCGACCCCTCATGGACTGTCCCTGAGTGACTTGCAGGAGGCCAAGTACGAGACGTTCTCTGA | |
| | TGACCCCAGCCCAGGCGCGATTGACTCAAATAACTCCCTGTCTGAGATGACACATTTTCGCCCTC | |
| | AGCTCCATCACAGCGGGGACATGGTGTTCACTCCAGAGTCCGGACTTCAGCTGCGCCTCAACGA | |
| | GAAACTCGGTACTACAGCCGCGACAGAACTCAAAAAGCTGGATTTCAAGGTTTCCAGCACCAGC | |
| | AATAACCTGATCTCTACAATTCCCAGCGATAACCTGGCGGCTGGAACCGACAACACTTCCAGCC | |
| | TGGGACCTCCGTCCATGCCTGTGCACTACGACTCCCAGCTCGACACCACTCTGTTCGGCAAGAAA | |
| | AGTAGCCCCCTGACCGAATCCGGCGGTCCGCTGTCCCTCTCCGAAGAGAATAACGATAGCAAGC | |
| | TCCTGGAATCTGGGCTTATGAACTCTCAAGAGTCATCCTGGGGCAAGAACGTTTCATCAACTAG | |
| | GGAGATCACCAGGACCACCCTGCAGTCTGACCAGGAGGAGATTGACTATGATGACACCATCTCT | |
| | GTGGAGATGAAGAAGGAGGACTTTGACATCTACGACGAGGACGAGAACCAGAGCCCCAGGAGC | |
| | TTCCAGAAGAAGACCAGGCACTACTTCATTGCTGCTGTGGAGAGGCTGTGGGACTATGGCATGA | |
| | GCAGCAGCCCCCATGTGCTGAGGAACAGGGCCCAGTCTGGCTCTGTGCCCCAGTTCAAGAAGGT | |
| | GGTGTTCCAGGAGTTCACTGATGGCAGCTTCACCCAGCCCCTGTACAGAGGGGAGCTGAATGAG | |
| | CACCTGGGCCTGCTGGGCCCCTACATCAGGGCTGAGGTGGAGGACAACATCATGGTGACCTTCA | |
| | GGAACCAGGCCAGCAGGCCCTACAGCTTCTACAGCAGCCTGATCAGCTATGAGGAGGACCAGA | |
| | GGCAGGGGGCTGAGCCCAGGAAGAACTTTGTGAAGCCCAATGAAACCAAGACCTACTTCTGGA | |
| | AGGTGCAGCACCACATGGCCCCCACCAAGGATGAGTTTGACTGCAAGGCCTGGGCCTACTTCTC | |
| | TGATGTGGACCTGGAGAAGGATGTGCACTCTGGCCTGATTGGCCCCCTGCTGGTGTGCCACACC | |
| | AACACCCTGAACCCTGCCCATGGCAGGCAGGTGACTGTGCAGGAGTTTGCCCTGTTCTTCACCAT | |
| | CTTTGATGAAACCAAGAGCTGGTACTTCACTGAGAACATGGAGAGGAACGGCAGGGCCCCCGG | |
| | CAACATCCAGATGGAGGACCCCACCTTCAAGGAGAACTACAGGTTCCATGCCATCAATGGCTAC | |
| | ATCATGGACACCCTGCCTGGCCTGGTGATGGCCCAGGACCAGAGGATCAGGTGGTACCTGCTGA | |
| | GCATGGGCAGCAATGAGAACATCCACAGCATCCACTTCTCTGGCCATGTGTTCACTGTGAGGAA | |
| | GAAGGAGGAGTACAAGATGGCCCTGTACAACCTGTACCCTGGGGTGTTTGAGACTGTGGAGATG | |
| | CTGCCCAGCAAGGCTGGCATCTGGAGGGTGGAGTGCCTGATTGGGGAGCACCTGCATGCTGGCA | |
| | TGAGCACCCTGTTCCTGGTGTACAGCAACAAGTGCCAGACCCCCCTGGGCATGGCCTCTGGCCA | |
| | CATCAGGGACTTCCAGATCACTGCCTCTGGCCAGTATGGCCAGTGGGCCCCCAAGCTGGCCAGG | |
| | CTGCACTACTCTGGCAGCATCAATGCCTGGAGCACCAAGGAGCCCTTCAGCTGGATCAAGGTGG | |
| | ACCTGCTGGCCCCCATGATCATCCATGGCATCAAGACCCAGGGGGCCAGGCAGAAGTTCAGCAG | |
| | CCTGTACATCAGCCAGTTCATCATCATGTACAGCCTGGATGGCAAGAAGTGGCAGACCTACAGG | |
| | GGCAACAGCACTGGCACCCTGATGGTGTTCTTTGGCAATGTGGACAGCTCTGGCATCAAGCACA | |
| | ACATCTTCAACCCCCCCATCATTGCCAGATACATCAGGCTGCACCCCACCCACTACAGCATCAGG | |
| | AGCACCCTGAGGATGGAGCTGATGGGCTGTGACCTGAACAGCTGCAGCATGCCCCTGGGCATGG | |
| | AGAGCAAGGCCATCTCTGATGCCCAGATCACTGCCAGCAGCTACTTCACCAACATGTTTGCCAC | |
| | CTGGAGCCCCAGCAAGGCCAGGCTGCACCTGCAGGGCAGGAGCAATGCCTGGAGGCCCCAGGT | |
| | CAACAACCCCAAGGAGTGGCTGCAGGTGGACTTCCAGAAGACCATGAAGGTGACTGGGGTGAC | |
| | CACCCAGGGGGTGAAGAGCCTGCTGACCAGCATGTATGTGAAGGAGTTCCTGATCAGCAGCAGC | |
| | CAGGATGGCCACCAGTGGACCCTGTTCTTCCAGAATGGCAAGGTGAAGGTGTTCCAGGGCAACC | |
| | AGGACAGCTTCACCCCTGTGGTGAACAGCCTGGACCCCCCCCTGCTGACCAGATACCTGAGGAT | |
| | TCACCCCCAGAGCTGGGTGCACCAGATTGCCCTGAGGATGGAGGTGCTGGGCTGTGAGGCCCAG | |
| | GACCTGTACTGACTCGA | |
| ApoE-AAT- | CGCGTCTGCAGGCTCAGAGGCACACAGGAGTTTCTGGGCTCACCCTGCCCCCTTCCAACCCCTCA | 39 |
| hBGi- | GTTCCCATCCTCCAGCAGCTGTTTGTGTGCTGCCTCTGAAGTCCACACTGAACAAACTTCAGCCT | |
| FVIII-N6- | ACTCATGTCCCTAAAATGGGCAAACATTGCAAGCAGCAAACAGCAAACACACAGCCCTCCCTGC | |
| F309S-DM- | CTGCTGACCTTGGAGCTGGGGCAGAGGTCAGAGACCTCTCTGGGCCCATGCCACCTCCAACATC | |
| WPRE3- | CACTCGACCCCTTGGAATTTCGGTGGAGAGGAGCAGAGGTTGTCCTGGCGTGGTTTAGGTAGTG | |
| DTS | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCGTAAGTACTAGCAGCTACAATCCAGCTACCATTCTGCTTTTATTTTAT | |
| | GGTTGGGATAAGGCTGGATTATTCTGAGTCCAAGCTAGGCCCTTTTGCTAATCATGTTCATACCT | |
| | CTTATCTTCCTCCCACAGATGCAGATTGAGCTGAGCACCTGCTTCTTCCTGTGCCTGCTGAGGTTC | |
| | TGCTTCTCTGCCACCAGGAGATACTACCTGGGGGCTGTGGAGCTGAGCTGGGACTACATGCAGT | |
| | CTGACCTGGGGGAGCTGCCTGTGGATGCCAGGTTCCCCCCCAGAGTGCCCAAGAGCTTCCCCTTC | |
| | AACACCTCTGTGGTGTACAAGAAGACCCTGTTTGTGGAGTTCACTGACCACCTGTTCAACATTGC | |
| | CAAGCCCAGGCCCCCCTGGATGGGCCTGCTGGGCCCCACCATCCAGGCTGAGGTGTATGACACT | |
| | GTGGTGATCACCCTGAAGAACATGGCCAGCCACCCTGTGAGCCTGCATGCTGTGGGGGTGAGCT | |
| | ACTGGAAGGCCTCTGAGGGGGCTGAGTATGATGACCAGACCAGCCAGAGGGAGAAGGAGGATG | |
| | ACAAGGTGTTCCCTGGGGGCAGCCACACCTATGTGTGGCAGGTGCTGAAGGAGAATGGCCCCAT | |
| | GGCCTCTGACCCCCTGTGCCTGACCTACAGCTACCTGAGCCATGTGGACCTGGTGAAGGACCTG | |
| | AACTCTGGCCTGATTGGGGCCCTGCTGGTGTGCAGGGAGGGCAGCCTGGCCAAGGAGAAGACCC | |
| | AGACCCTGCACAAGTTCATCCTGCTGTTTGCTGTGTTTGATGAGGGCAAGAGCTGGCACTCTGAA | |
| | ACCAAGAACAGCCTGATGCAGGACAGGGATGCTGCCTCTGCCAGGGCCTGGCCCAAGATGCAC | |
| | ACTGTGAATGGCTATGTGAACAGGAGCCTGCCTGGCCTGATTGGCTGCCACAGGAAGTCTGTGT | |
| | ACTGGCATGTGATTGGCATGGGCACCACCCCTGAGGTGCACAGCATCTTCCTGGAGGGCCACAC | |
| | CTTCCTGGTCAGGAACCACAGGCAGGCCAGCCTGGAGATCAGCCCCATCACCTTCCTGACTGCC | |
| | CAGACCCTGCTGATGGACCTGGGCCAGTTCCTGCTGAGCTGCCACATCAGCAGCCACCAGCATG | |
| | ATGGCATGGAGGCCTATGTGAAGGTGGACAGCTGCCCTGAGGAGCCCCAGCTGAGGATGAAGA | |
| | ACAATGAGGAGGCTGAGGACTATGATGATGACCTGACTGACTCTGAGATGGATGTGGTGAGGTT | |
| | TGATGATGACAACAGCCCCAGCTTCATCCAGATCAGGTCTGTGGCCAAGAAGCACCCCAAGACC | |
| | TGGGTGCACTACATTGCTGCTGAGGAGGAGGACTGGGACTATGCCCCCCTGGTGCTGGCCCCTG | |
| | ATGACAGGAGCTACAAGAGCCAGTACCTGAACAATGGCCCCCAGAGGATTGGCAGGAAGTACA | |
| | AGAAGGTCAGGTTCATGGCCTACACTGATGAAACCTTCAAGACCAGGGAGGCCATCCAGCATGA | |
| | GTCTGGCATCCTGGGCCCCCTGCTGTATGGGGAGGTGGGGGACACCCTGCTGATCATCTTCAAG | |
| | AACCAGGCCAGCAGGCCCTACAACATCTACCCCCATGGCATCACTGATGTGAGGCCCCTGTACA | |
| | GCAGGAGGCTGCCCAAGGGGGTGAAGCACCTGAAGGACTTCCCCATCCTGCCTGGGGAGATCTT | |
| | CAAGTACAAGTGGACTGTGACTGTGGAGGATGGCCCCACCAAGTCTGACCCCAGGTGCCTGACC | |
| | AGATACTACAGCAGCTTTGTGAACATGGAGAGGGACCTGGCCTCTGGCCTGATTGGCCCCCTGC | |
| | TGATCTGCTACAAGGAGTCTGTGGACCAGAGGGGCAACCAGATCATGTCTGACAAGAGGAATGT | |
| | GATCCTGTTCTCTGTGTTTGATGAGAACAGGAGCTGGTACCTGACTGAGAACATCCAGAGGTTCC | |
| | TGCCCAACCCTGCTGGGGTGCAGCTGGAGGACCCTGAGTTCCAGGCCAGCAACATCATGCACAG | |
| | CATCAATGGCTATGTGTTTGACAGCCTGCAGCTGTCTGTGTGCCTGCATGAGGTGGCCTACTGGT | |
| | ACATCCTGAGCATTGGGGCCCAGACTGACTTCCTGTCTGTGTTCTTCTCTGGCTACACCTTCAAG | |
| | CACAAGATGGTGTATGAGGACACCCTGACCCTGTTCCCCTTCTCTGGGGAGACTGTGTTCATGAG | |
| | CATGGAGAACCCTGGCCTGTGGATTCTGGGCTGCCACAACTCTGACTTCAGGAACAGGGGCATG | |
| | ACTGCCCTGCTGAAAGTCTCCAGCTGTGACAAGAACACTGGGGACTACTATGAGGACAGCTATG | |
| | AGGACATCTCTGCCTACCTGCTGAGCAAGAACAATGCCATTGAGCCCAGGAGCTTCAGCCAGAA | |
| | TCCCCCAGTGCTCACCCGGTCCTTTAGCCAGAATTCTCGCCATCCCTCCACCCGGCAAAAGCAGT | |
| | TCAACGCCACTACCATCCCAGAGAACGACATCGAAAAAACCGACCCCTGGTTCGCCCACAGAAC | |
| | TCCCATGCCAAAGATTCAGAATGTTTCCAGTTCTGACCTCCTTATGTTGCTGCGCCAGTCTCCGA | |
| | CCCCTCATGGACTGTCCCTGAGTGACTTGCAGGAGGCCAAGTACGAGACGTTCTCTGATGACCC | |
| | CAGCCCAGGCGCGATTGACTCAAATAACTCCCTGTCTGAGATGACACATTTTCGCCCTCAGCTCC | |
| | ATCACAGCGGGGACATGGTGTTCACTCCAGAGTCCGGACTTCAGCTGCGCCTCAACGAGAAACT | |
| | CGGTACTACAGCCGCGACAGAACTCAAAAAGCTGGATTTCAAGGTTTCCAGCACCAGCAATAAC | |
| | CTGATCTCTACAATTCCCAGCGATAACCTGGCGGCTGGAACCGACAACACTTCCAGCCTGGGAC | |
| | CTCCGTCCATGCCTGTGCACTACGACTCCCAGCTCGACACCACTCTGTTCGGCAAGAAAAGTAGC | |
| | CCCCTGACCGAATCCGGCGGTCCGCTGTCCCTCTCCGAAGAGAATAACGATAGCAAGCTCCTGG | |
| | AATCTGGGCTTATGAACTCTCAAGAGTCATCCTGGGGCAAGAACGTTTCATCAACTAGGGAGAT | |
| | CACCAGGACCACCCTGCAGTCTGACCAGGAGGAGATTGACTATGATGACACCATCTCTGTGGAG | |
| | ATGAAGAAGGAGGACTTTGACATCTACGACGAGGACGAGAACCAGAGCCCCAGGAGCTTCCAG | |
| | AAGAAGACCAGGCACTACTTCATTGCTGCTGTGGAGAGGCTGTGGGACTATGGCATGAGCAGCA | |
| | GCCCCCATGTGCTGAGGAACAGGGCCCAGTCTGGCTCTGTGCCCCAGTTCAAGAAGGTGGTGTT | |
| | CCAGGAGTTCACTGATGGCAGCTTCACCCAGCCCCTGTACAGAGGGGAGCTGAATGAGCACCTG | |
| | GGCCTGCTGGGCCCCTACATCAGGGCTGAGGTGGAGGACAACATCATGGTGACCTTCAGGAACC | |
| | AGGCCAGCAGGCCCTACAGCTTCTACAGCAGCCTGATCAGCTATGAGGAGGACCAGAGGCAGG | |
| | GGGCTGAGCCCAGGAAGAACTTTGTGAAGCCCAATGAAACCAAGACCTACTTCTGGAAGGTGCA | |
| | GCACCACATGGCCCCCACCAAGGATGAGTTTGACTGCAAGGCCTGGGCCTACTTCTCTGATGTG | |
| | GACCTGGAGAAGGATGTGCACTCTGGCCTGATTGGCCCCCTGCTGGTGTGCCACACCAACACCC | |
| | TGAACCCTGCCCATGGCAGGCAGGTGACTGTGCAGGAGTTTGCCCTGTTCTTCACCATCTTTGAT | |
| | GAAACCAAGAGCTGGTACTTCACTGAGAACATGGAGAGGAACGGCAGGGCCCCCGGCAACATC | |
| | CAGATGGAGGACCCCACCTTCAAGGAGAACTACAGGTTCCATGCCATCAATGGCTACATCATGG | |
| | ACACCCTGCCTGGCCTGGTGATGGCCCAGGACCAGAGGATCAGGTGGTACCTGCTGAGCATGGG | |
| | CAGCAATGAGAACATCCACAGCATCCACTTCTCTGGCCATGTGTTCACTGTGAGGAAGAAGGAG | |
| | GAGTACAAGATGGCCCTGTACAACCTGTACCCTGGGGTGTTTGAGACTGTGGAGATGCTGCCCA | |
| | GCAAGGCTGGCATCTGGAGGGTGGAGTGCCTGATTGGGGAGCACCTGCATGCTGGCATGAGCAC | |
| | CCTGTTCCTGGTGTACAGCAACAAGTGCCAGACCCCCCTGGGCATGGCCTCTGGCCACATCAGG | |
| | GACTTCCAGATCACTGCCTCTGGCCAGTATGGCCAGTGGGCCCCCAAGCTGGCCAGGCTGCACT | |
| | ACTCTGGCAGCATCAATGCCTGGAGCACCAAGGAGCCCTTCAGCTGGATCAAGGTGGACCTGCT | |
| | GGCCCCCATGATCATCCATGGCATCAAGACCCAGGGGGCCAGGCAGAAGTTCAGCAGCCTGTAC | |
| | ATCAGCCAGTTCATCATCATGTACAGCCTGGATGGCAAGAAGTGGCAGACCTACAGGGGCAACA | |
| | GCACTGGCACCCTGATGGTGTTCTTTGGCAATGTGGACAGCTCTGGCATCAAGCACAACATCTTC | |
| | AACCCCCCCATCATTGCCAGATACATCAGGCTGCACCCCACCCACTACAGCATCAGGAGCACCC | |
| | TGAGGATGGAGCTGATGGGCTGTGACCTGAACAGCTGCAGCATGCCCCTGGGCATGGAGAGCA | |
| | AGGCCATCTCTGATGCCCAGATCACTGCCAGCAGCTACTTCACCAACATGTTTGCCACCTGGAGC | |
| | CCCAGCAAGGCCAGGCTGCACCTGCAGGGCAGGAGCAATGCCTGGAGGCCCCAGGTCAACAAC | |
| | CCCAAGGAGTGGCTGCAGGTGGACTTCCAGAAGACCATGAAGGTGACTGGGGTGACCACCCAG | |
| | GGGGTGAAGAGCCTGCTGACCAGCATGTATGTGAAGGAGTTCCTGATCAGCAGCAGCCAGGATG | |
| | GCCACCAGTGGACCCTGTTCTTCCAGAATGGCAAGGTGAAGGTGTTCCAGGGCAACCAGGACAG | |
| | CTTCACCCCTGTGGTGAACAGCCTGGACCCCCCCCTGCTGACCAGATACCTGAGGATTCACCCCC | |
| | AGAGCTGGGTGCACCAGATTGCCCTGAGGATGGAGGTGCTGGGCTGTGAGGCCCAGGACCTGTA | |
| | CTGAAATCAACCTCTGGATTACAAAATTTGTGAAAGATTGACTGGTATTCTTAACTATGTTGCTC | |
| | CTTTTACGCTATGTGGATACGCTGCTTTAATGCCTTTGTATCATGCTATTGCTTCCCGTATGGCTT | |
| | TCATTTTCTCCTCCTTGTATAAATCCTGGTTGCTGTCTCTTTATGAGGAGTTGTGGCCCGTTGTCA | |
| | GGCAACGTGGCGTGGTGTGCACTGTGTTTGCTGACGCAACCCCCACTGGTTGGGGCATTGCCAC | |
| | CACCTGTCAGCTCCTTTCCGGGACTTTCGCTTTCCCCCTCCCTATTGCCACGGCGGAACTCATCGC | |
| | CGCCTGCCTTGCCCGCTGCTGGACAGGGGCTCGGCTGTTGGGCACTGACAATTCCGTGGGAGCT | |
| | GTGCGATCCCTGCTGGGGACTTTCCGCTGGGGACTTTCCGCTGGGGACTTTCCGCCTTCAGCTAA | |
| | GGAAGCTACCAATATTTAGAGGTACATTTTGTTCTAGAACAAAATGTACCGGTACATTTTGTTCT | |
| | GGTACATTTTGTTCT | |
| APOE- | GAGCTGTGCGATCCCTGCTGGGGACTTTCCGCTGGGGACTTTCCGCTGGGGACTTTCCGCCTTCA | 40 |
| AAT-hBGi- | GCTAAGGAAGCTACCAATATTTAGAGGTACATTTTGTTCTAGAACAAAATGTACCGGTACATTTT | |
| FIX-bGH- | GTTCTGGTACATTTTGTTCTTGTGCCTTCTAGTTGCCAGCCATCTGTTGTTTGCCCCTCCCCCGTG | |
| poly A-DTS | CCTTCCTTGACCCTGGAAGGTGCCACTCCCACTGTCCTTTCCTAATAAAATGAGGAAATTGCATC | |
| | GCATTGTCTGAGTAGGTGTCATTCTATTCTGGGGGGTGGGGTGGGGCAGGACAGCAAGGGGGAG | |
| | GATTGGGAAGACAATAGCAGGCATGCTGGGGATGCGGTGGGCTCTATGGATGCAGCGCGTGAA | |
| | CATGATCATGGCAGAATCACCAGGCCTCATCACCATCTGCCTTTTAGGATATCTACTCAGTGCTG | |
| | AATGTACAGTTTTTCTTGATCATGAAAACGCCAACAAAATTCTGAATCGGCCAAAGAGGTATAA | |
| | TTCAGGTAAATTGGAAGAGTTTGTTCAAGGGAACCTTGAGAGAGAATGTATGGAAGAAAAGTGT | |
| | AGTTTTGAAGAAGCACGAGAAGTTTTTGAAAACACTGAAAGAACAACTGAATTTTGGAAGCAGT | |
| | ATGTTGATGGAGATCAGTGTGAGTCCAATCCATGTTTAAATGGCGGCAGTTGCAAGGATGACAT | |
| | TAATTCCTATGAATGTTGGTGTCCCTTTGGATTTGAAGGAAAGAACTGTGAATTAGATGTAACAT | |
| | GTAACATTAAGAATGGCAGATGCGAGCAGTTTTGTAAAAATAGTGCTGATAACAAGGTGGTTTG | |
| | CTCCTGTACTGAGGGATATCGACTTGCAGAAAACCAGAAGTCCTGTGAACCAGCAGTGCCATTT | |
| | CCATGTGGAAGAGTTTCTGTTTCACAAACTTCTAAGCTCACCCGTGCTGAGACTGTTTTTCCTGA | |
| | TGTGGACTATGTAAATTCTACTGAAGCTGAAACCATTTTGGATAACATCACTCAAAGCACCCAAT | |
| | CATTTAATGACTTCACTCGGGTTGTTGGTGGAGAAGATGCCAAACCAGGTCAATTCCCTTGGCAG | |
| | GTTGTTTTGAATGGTAAAGTTGATGCATTCTGTGGAGGCTCTATCGTTAATGAAAAATGGATTGT | |
| | AACTGCTGCCCACTGTGTTGAAACTGGTGTTAAAATTACAGTTGTCGCAGGTGAACATAATATTG | |
| | AGGAGACAGAACATACAGAGCAAAAGCGAAATGTGATTCGAATTATTCCTCACCACAACTACA | |
| | ATGCAGCTATTAATAAGTACAACCATGACATTGCCCTTCTGGAACTGGACGAACCCTTAGTGCTA | |
| | AACAGCTACGTTACACCTATTTGCATTGCTGACAAGGAATACACGAACATCTTCCTCAAATTTGG | |
| | ATCTGGCTATGTAAGTGGCTGGGGAAGAGTCTTCCACAAAGGGAGATCAGCTTTAGTTCTTCAG | |
| | TACCTTAGAGTTCCACTTGTTGACCGAGCCACATGTCTTCGATCTACAAAGTTCACCATCTATAA | |
| | CAACATGTTCTGTGCTGGCTTCCATGAAGGAGGTAGAGATTCATGTCAAGGAGATAGTGGGGGA | |
| | CCCCATGTTACTGAAGTGGAAGGGACCAGTTTCTTAACTGGAATTATTAGCTGGGGTGAAGAGT | |
| | GTGCAATGAAAGGCAAATATGGAATATATACCAAGGTATCCCGGTATGTCAACTGGATTAAGGA | |
| | AAAAACAAAGCTCACTTAATGAGTAAGTACTAGCAGCTACAATCCAGCTACCATTCTGCTTTTAT | |
| | TTTATGGTTGGGATAAGGCTGGATTATTCTGAGTCCAAGCTAGGCCCTTTTGCTAATCATGTTCA | |
| | TACCTCTTATCTTCCTCCCACAGCGCGTCTGCAGGCTCAGAGGCACACAGGAGTTTCTGGGCTCA | |
| | CCCTGCCCCCTTCCAACCCCTCAGTTCCCATCCTCCAGCAGCTGTTTGTGTGCTGCCTCTGAAGTC | |
| | CACACTGAACAAACTTCAGCCTACTCATGTCCCTAAAATGGGCAAACATTGCAAGCAGCAAACA | |
| | GCAAACACACAGCCCTCCCTGCCTGCTGACCTTGGAGCTGGGGCAGAGGTCAGAGACCTCTCTG | |
| | GGCCCATGCCACCTCCAACATCCACTCGACCCCTTGGAATTTCGGTGGAGAGGAGCAGAGGTTG | |
| | TCCTGGCGTGGTTTAGGTAGTGTGAGAGGGGTCGACTGGACACAGGACGCTGTGGTTTCTGAGC | |
| | CAGGGGGCGACTCAGATCCCAGCCAGTGGACTTAGCCCCTGTTTGCTCCTCCGATAACTGGGGT | |
| | GACCTTGGTTAATATTCACCAGCAGCCTCCCCCGTTGCCCC | |
| APOE- | CGCGTCTGCAGGCTCAGAGGCACACAGGAGTTTCTGGGCTCACCCTGCCCCCTTCCAACCCCTCA | 41 |
| AAT-hBGi- | GTTCCCATCCTCCAGCAGCTGTTTGTGTGCTGCCTCTGAAGTCCACACTGAACAAACTTCAGCCT | |
| FIX | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| bGH-poly A | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCGTAAGTACTAGCAGCTACAATCCAGCTACCATTCTGCTTTTATTTTAT | |
| | GGTTGGGATAAGGCTGGATTATTCTGAGTCCAAGCTAGGCCCTTTTGCTAATCATGTTCATACCT | |
| | CTTATCTTCCTCCCACAGATGCAGCGCGTGAACATGATCATGGCAGAATCACCAGGCCTCATCAC | |
| | CATCTGCCTTTTAGGATATCTACTCAGTGCTGAATGTACAGTTTTTCTTGATCATGAAAACGCCA | |
| | ACAAAATTCTGAATCGGCCAAAGAGGTATAATTCAGGTAAATTGGAAGAGTTTGTTCAAGGGAA | |
| | CCTTGAGAGAGAATGTATGGAAGAAAAGTGTAGTTTTGAAGAAGCACGAGAAGTTTTTGAAAAC | |
| | ACTGAAAGAACAACTGAATTTTGGAAGCAGTATGTTGATGGAGATCAGTGTGAGTCCAATCCAT | |
| | GTTTAAATGGCGGCAGTTGCAAGGATGACATTAATTCCTATGAATGTTGGTGTCCCTTTGGATTT | |
| | GAAGGAAAGAACTGTGAATTAGATGTAACATGTAACATTAAGAATGGCAGATGCGAGCAGTTTT | |
| | GTAAAAATAGTGCTGATAACAAGGTGGTTTGCTCCTGTACTGAGGGATATCGACTTGCAGAAAA | |
| | CCAGAAGTCCTGTGAACCAGCAGTGCCATTTCCATGTGGAAGAGTTTCTGTTTCACAAACTTCTA | |
| | AGCTCACCCGTGCTGAGACTGTTTTTCCTGATGTGGACTATGTAAATTCTACTGAAGCTGAAACC | |
| | ATTTTGGATAACATCACTCAAAGCACCCAATCATTTAATGACTTCACTCGGGTTGTTGGTGGAGA | |
| | AGATGCCAAACCAGGTCAATTCCCTTGGCAGGTTGTTTTGAATGGTAAAGTTGATGCATTCTGTG | |
| | GAGGCTCTATCGTTAATGAAAAATGGATTGTAACTGCTGCCCACTGTGTTGAAACTGGTGTTAAA | |
| | ATTACAGTTGTCGCAGGTGAACATAATATTGAGGAGACAGAACATACAGAGCAAAAGCGAAAT | |
| | GTGATTCGAATTATTCCTCACCACAACTACAATGCAGCTATTAATAAGTACAACCATGACATTGC | |
| | CCTTCTGGAACTGGACGAACCCTTAGTGCTAAACAGCTACGTTACACCTATTTGCATTGCTGACA | |
| | AGGAATACACGAACATCTTCCTCAAATTTGGATCTGGCTATGTAAGTGGCTGGGGAAGAGTCTT | |
| | CCACAAAGGGAGATCAGCTTTAGTTCTTCAGTACCTTAGAGTTCCACTTGTTGACCGAGCCACAT | |
| | GTCTTCGATCTACAAAGTTCACCATCTATAACAACATGTTCTGTGCTGGCTTCCATGAAGGAGGT | |
| | AGAGATTCATGTCAAGGAGATAGTGGGGGACCCCATGTTACTGAAGTGGAAGGGACCAGTTTCT | |
| | TAACTGGAATTATTAGCTGGGGTGAAGAGTGTGCAATGAAAGGCAAATATGGAATATATACCAA | |
| | GGTATCCCGGTATGTCAACTGGATTAAGGAAAAAACAAAGCTCACTTAATGAAATCAACCTCTG | |
| | GATTACAAAATTTGTGAAAGATTGACTGGTATTCTTAACTATGTTGCTCCTTTTACGCTATGTGG | |
| | ATACGCTGCTTTAATGCCTTTGTATCATGCTATTGCTTCCCGTATGGCTTTCATTTTCTCCTCCTTG | |
| | TATAAATCCTGGTTGCTGTCTCTTTATGAGGAGTTGTGGCCCGTTGTCAGGCAACGTGGCGTGGT | |
| | GTGCACTGTGTTTGCTGACGCAACCCCCACTGGTTGGGGCATTGCCACCACCTGTCAGCTCCTTT | |
| | CCGGGACTTTCGCTTTCCCCCTCCCTATTGCCACGGCGGAACTCATCGCCGCCTGCCTTGCCCGC | |
| | TGCTGGACAGGGGCTCGGCTGTTGGGCACTGACAATTCCGTGGTGTGCCTTCTAGTTGCCAGCCA | |
| | TCTGTTGTTTGCCCCTCCCCCGTGCCTTCCTTGACCCTGGAAGGTGCCACTCCCACTGTCCTTTCC | |
| | TAATAAAATGAGGAAATTGCATCGCATTGTCTGAGTAGGTGTCATTCTATTCTGGGGGGTGGGG | |
| | TGGGGCAGGACAGCAAGGGGGAGGATTGGGAAGACAATAGCAGGCATGCTGGGGATGCGGTGG | |
| | GCTCTATGG | |
| ITR-APOE- | GCTCACTCACTCACTCACTGAGGCCTGCAGAGCAAAGCTCTGCAGTCTGGGGACCTTTGGTCCCC | 42 |
| AAT-hBGi- | AGGCCTCAGTGAGTGAGTGAGTGAGCAGAGAGGGAGTGGCCAACTCCATCACTAGGGGTTCCTC | |
| FIX-bGH- | GCGTCTGCAGGCTCAGAGGCACACAGGAGTTTCTGGGCTCACCCTGCCCCCTTCCAACCCCTCAG | |
| poly A-ITR | TTCCCATCCTCCAGCAGCTGTTTGTGTGCTGCCTCTGAAGTCCACACTGAACAAACTTCAGCCTA | |
| | CTCATGTCCCTAAAATGGGCAAACATTGCAAGCAGCAAACAGCAAACACACAGCCCTCCCTGCC | |
| | TGCTGACCTTGGAGCTGGGGCAGAGGTCAGAGACCTCTCTGGGCCCATGCCACCTCCAACATCC | |
| | ACTCGACCCCTTGGAATTTCGGTGGAGAGGAGCAGAGGTTGTCCTGGCGTGGTTTAGGTAGTGT | |
| | GAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCGTAAGTACTAGCAGCTACAATCCAGCTACCATTCTGCTTTTATTTTAT | |
| | GGTTGGGATAAGGCTGGATTATTCTGAGTCCAAGCTAGGCCCTTTTGCTAATCATGTTCATACCT | |
| | CTTATCTTCCTCCCACAGATGCAGCGCGTGAACATGATCATGGCAGAATCACCAGGCCTCATCAC | |
| | CATCTGCCTTTTAGGATATCTACTCAGTGCTGAATGTACAGTTTTTCTTGATCATGAAAACGCCA | |
| | ACAAAATTCTGAATCGGCCAAAGAGGTATAATTCAGGTAAATTGGAAGAGTTTGTTCAAGGGAA | |
| | CCTTGAGAGAGAATGTATGGAAGAAAAGTGTAGTTTTGAAGAAGCACGAGAAGTTTTTGAAAAC | |
| | ACTGAAAGAACAACTGAATTTTGGAAGCAGTATGTTGATGGAGATCAGTGTGAGTCCAATCCAT | |
| | GTTTAAATGGCGGCAGTTGCAAGGATGACATTAATTCCTATGAATGTTGGTGTCCCTTTGGATTT | |
| | GAAGGAAAGAACTGTGAATTAGATGTAACATGTAACATTAAGAATGGCAGATGCGAGCAGTTTT | |
| | GTAAAAATAGTGCTGATAACAAGGTGGTTTGCTCCTGTACTGAGGGATATCGACTTGCAGAAAA | |
| | CCAGAAGTCCTGTGAACCAGCAGTGCCATTTCCATGTGGAAGAGTTTCTGTTTCACAAACTTCTA | |
| | AGCTCACCCGTGCTGAGACTGTTTTTCCTGATGTGGACTATGTAAATTCTACTGAAGCTGAAACC | |
| | ATTTTGGATAACATCACTCAAAGCACCCAATCATTTAATGACTTCACTCGGGTTGTTGGTGGAGA | |
| | AGATGCCAAACCAGGTCAATTCCCTTGGCAGGTTGTTTTGAATGGTAAAGTTGATGCATTCTGTG | |
| | GAGGCTCTATCGTTAATGAAAAATGGATTGTAACTGCTGCCCACTGTGTTGAAACTGGTGTTAAA | |
| | ATTACAGTTGTCGCAGGTGAACATAATATTGAGGAGACAGAACATACAGAGCAAAAGCGAAAT | |
| | GTGATTCGAATTATTCCTCACCACAACTACAATGCAGCTATTAATAAGTACAACCATGACATTGC | |
| | CCTTCTGGAACTGGACGAACCCTTAGTGCTAAACAGCTACGTTACACCTATTTGCATTGCTGACA | |
| | AGGAATACACGAACATCTTCCTCAAATTTGGATCTGGCTATGTAAGTGGCTGGGGAAGAGTCTT | |
| | CCACAAAGGGAGATCAGCTTTAGTTCTTCAGTACCTTAGAGTTCCACTTGTTGACCGAGCCACAT | |
| | GTCTTCGATCTACAAAGTTCACCATCTATAACAACATGTTCTGTGCTGGCTTCCATGAAGGAGGT | |
| | AGAGATTCATGTCAAGGAGATAGTGGGGGACCCCATGTTACTGAAGTGGAAGGGACCAGTTTCT | |
| | TAACTGGAATTATTAGCTGGGGTGAAGAGTGTGCAATGAAAGGCAAATATGGAATATATACCAA | |
| | GGTATCCCGGTATGTCAACTGGATTAAGGAAAAAACAAAGCTCACTTAATGATGTGCCTTCTAG | |
| | TTGCCAGCCATCTGTTGTTTGCCCCTCCCCCGTGCCTTCCTTGACCCTGGAAGGTGCCACTCCCAC | |
| | TGTCCTTTCCTAATAAAATGAGGAAATTGCATCGCATTGTCTGAGTAGGTGTCATTCTATTCTGG | |
| | GGGGTGGGGTGGGGCAGGACAGCAAGGGGGAGGATTGGGAAGACAATAGCAGGCATGCTGGG | |
| | GATGCGGTGGGCTCTATGGAGGAACCCCTAGTGATGGAGTTGGCCACTCCCTCTCTGCTCACTCA | |
| | CTCACTCACTGAGGCCTGGGGACCAAAGGTCCCCAGACTGCAGAGCTTTGCTCTGCAGGCCTCA | |
| | GTGAGTGAGTGAGTGAGCAGA | |
| ApoE-AAT- | CGCGTCTGCAGGCTCAGAGGCACACAGGAGTTTCTGGGCTCACCCTGCCCCCTTCCAACCCCTCA | 43 |
| hBGi- | GTTCCCATCCTCCAGCAGCTGTTTGTGTGCTGCCTCTGAAGTCCACACTGAACAAACTTCAGCCT | |
| FVIII-v3- | ACTCATGTCCCTAAAATGGGCAAACATTGCAAGCAGCAAACAGCAAACACACAGCCCTCCCTGC | |
| WPRE3- | CTGCTGACCTTGGAGCTGGGGCAGAGGTCAGAGACCTCTCTGGGCCCATGCCACCTCCAACATC | |
| DTS | CACTCGACCCCTTGGAATTTCGGTGGAGAGGAGCAGAGGTTGTCCTGGCGTGGTTTAGGTAGTG | |
| | TGAGAGGGGTCGACTGGACACAGGACGCTGTGGTTTCTGAGCCAGGGGGCGACTCAGATCCCAG | |
| | CCAGTGGACTTAGCCCCTGTTTGCTCCTCCGATAACTGGGGTGACCTTGGTTAATATTCACCAGC | |
| | AGCCTCCCCCGTTGCCCCTCTGGATCCACTGCTTAAATACGGACGAGGACAGGGCCCTGTCTCCT | |
| | CAGCTTCAGGCACCACCACTGACCTGGGACAGTGAATCGTAAGTACTAGCAGCTACAATCCAGC | |
| | TACCATTCTGCTTTTATTTTATGGTTGGGATAAGGCTGGATTATTCTGAGTCCAAGCTAGGCCCTT | |
| | TTGCTAATCATGTTCATACCTCTTATCTTCCTCCCACAGCTCCTGGGCAACGTGCTGGTCTGTGTG | |
| | CTGGCCCATCACTTTGGCAAAGAATTGCGATCGCCACCATGCAGATTGAGCTGAGCACCTGCTTC | |
| | TTCCTGTGCCTGCTGAGGTTCTGCTTCTCTGCCACCAGGAGATACTACCTGGGGGCTGTGGAGCT | |
| | GAGCTGGGACTACATGCAGTCTGACCTGGGGGAGCTGCCTGTGGATGCCAGGTTCCCCCCCAGA | |
| | GTGCCCAAGAGCTTCCCCTTCAACACCTCTGTGGTGTACAAGAAGACCCTGTTTGTGGAGTTCAC | |
| | TGACCACCTGTTCAACATTGCCAAGCCCAGGCCCCCCTGGATGGGCCTGCTGGGCCCCACCATCC | |
| | AGGCTGAGGTGTATGACACTGTGGTGATCACCCTGAAGAACATGGCCAGCCACCCTGTGAGCCT | |
| | GCATGCTGTGGGGGTGAGCTACTGGAAGGCCTCTGAGGGGGCTGAGTATGATGACCAGACCAGC | |
| | CAGAGGGAGAAGGAGGATGACAAGGTGTTCCCTGGGGGCAGCCACACCTATGTGTGGCAGGTG | |
| | CTGAAGGAGAATGGCCCCATGGCCTCTGACCCCCTGTGCCTGACCTACAGCTACCTGAGCCATG | |
| | TGGACCTGGTGAAGGACCTGAACTCTGGCCTGATTGGGGCCCTGCTGGTGTGCAGGGAGGGCAG | |
| | CCTGGCCAAGGAGAAGACCCAGACCCTGCACAAGTTCATCCTGCTGTTTGCTGTGTTTGATGAG | |
| | GGCAAGAGCTGGCACTCTGAAACCAAGAACAGCCTGATGCAGGACAGGGATGCTGCCTCTGCC | |
| | AGGGCCTGGCCCAAGATGCACACTGTGAATGGCTATGTGAACAGGAGCCTGCCTGGCCTGATTG | |
| | GCTGCCACAGGAAGTCTGTGTACTGGCATGTGATTGGCATGGGCACCACCCCTGAGGTGCACAG | |
| | CATCTTCCTGGAGGGCCACACCTTCCTGGTCAGGAACCACAGGCAGGCCAGCCTGGAGATCAGC | |
| | CCCATCACCTTCCTGACTGCCCAGACCCTGCTGATGGACCTGGGCCAGTTCCTGCTGTTCTGCCA | |
| | CATCAGCAGCCACCAGCATGATGGCATGGAGGCCTATGTGAAGGTGGACAGCTGCCCTGAGGA | |
| | GCCCCAGCTGAGGATGAAGAACAATGAGGAGGCTGAGGACTATGATGATGACCTGACTGACTCT | |
| | GAGATGGATGTGGTGAGGTTTGATGATGACAACAGCCCCAGCTTCATCCAGATCAGGTCTGTGG | |
| | CCAAGAAGCACCCCAAGACCTGGGTGCACTACATTGCTGCTGAGGAGGAGGACTGGGACTATGC | |
| | CCCCCTGGTGCTGGCCCCTGATGACAGGAGCTACAAGAGCCAGTACCTGAACAATGGCCCCCAG | |
| | AGGATTGGCAGGAAGTACAAGAAGGTCAGGTTCATGGCCTACACTGATGAAACCTTCAAGACCA | |
| | GGGAGGCCATCCAGCATGAGTCTGGCATCCTGGGCCCCCTGCTGTATGGGGAGGTGGGGGACAC | |
| | CCTGCTGATCATCTTCAAGAACCAGGCCAGCAGGCCCTACAACATCTACCCCCATGGCATCACT | |
| | GATGTGAGGCCCCTGTACAGCAGGAGGCTGCCCAAGGGGGTGAAGCACCTGAAGGACTTCCCC | |
| | ATCCTGCCTGGGGAGATCTTCAAGTACAAGTGGACTGTGACTGTGGAGGATGGCCCCACCAAGT | |
| | CTGACCCCAGGTGCCTGACCAGATACTACAGCAGCTTTGTGAACATGGAGAGGGACCTGGCCTC | |
| | TGGCCTGATTGGCCCCCTGCTGATCTGCTACAAGGAGTCTGTGGACCAGAGGGGCAACCAGATC | |
| | ATGTCTGACAAGAGGAATGTGATCCTGTTCTCTGTGTTTGATGAGAACAGGAGCTGGTACCTGA | |
| | CTGAGAACATCCAGAGGTTCCTGCCCAACCCTGCTGGGGTGCAGCTGGAGGACCCTGAGTTCCA | |
| | GGCCAGCAACATCATGCACAGCATCAATGGCTATGTGTTTGACAGCCTGCAGCTGTCTGTGTGCC | |
| | TGCATGAGGTGGCCTACTGGTACATCCTGAGCATTGGGGCCCAGACTGACTTCCTGTCTGTGTTC | |
| | TTCTCTGGCTACACCTTCAAGCACAAGATGGTGTATGAGGACACCCTGACCCTGTTCCCCTTCTC | |
| | TGGGGAGACTGTGTTCATGAGCATGGAGAACCCTGGCCTGTGGATTCTGGGCTGCCACAACTCT | |
| | GACTTCAGGAACAGGGGCATGACTGCCCTGCTGAAAGTCTCCAGCTGTGACAAGAACACTGGGG | |
| | ACTACTATGAGGACAGCTATGAGGACATCTCTGCCTACCTGCTGAGCAAGAACAATGCCATTGA | |
| | GCCCAGGAGCTTCAGCCAGAATGCCACTAATGTGTCTAACAACAGCAACACCAGCAATGACAGC | |
| | AATGTGTCTCCCCCAGTGCTGAAGAGGCACCAGAGGGAGATCACCAGGACCACCCTGCAGTCTG | |
| | ACCAGGAGGAGATTGACTATGATGACACCATCTCTGTGGAGATGAAGAAGGAGGACTTTGACAT | |
| | CTACGACGAGGACGAGAACCAGAGCCCCAGGAGCTTCCAGAAGAAGACCAGGCACTACTTCAT | |
| | TGCTGCTGTGGAGAGGCTGTGGGACTATGGCATGAGCAGCAGCCCCCATGTGCTGAGGAACAGG | |
| | GCCCAGTCTGGCTCTGTGCCCCAGTTCAAGAAGGTGGTGTTCCAGGAGTTCACTGATGGCAGCTT | |
| | CACCCAGCCCCTGTACAGAGGGGAGCTGAATGAGCACCTGGGCCTGCTGGGCCCCTACATCAGG | |
| | GCTGAGGTGGAGGACAACATCATGGTGACCTTCAGGAACCAGGCCAGCAGGCCCTACAGCTTCT | |
| | ACAGCAGCCTGATCAGCTATGAGGAGGACCAGAGGCAGGGGGCTGAGCCCAGGAAGAACTTTG | |
| | TGAAGCCCAATGAAACCAAGACCTACTTCTGGAAGGTGCAGCACCACATGGCCCCCACCAAGGA | |
| | TGAGTTTGACTGCAAGGCCTGGGCCTACTTCTCTGATGTGGACCTGGAGAAGGATGTGCACTCTG | |
| | GCCTGATTGGCCCCCTGCTGGTGTGCCACACCAACACCCTGAACCCTGCCCATGGCAGGCAGGT | |
| | GACTGTGCAGGAGTTTGCCCTGTTCTTCACCATCTTTGATGAAACCAAGAGCTGGTACTTCACTG | |
| | AGAACATGGAGAGGAACTGCAGGGCCCCCTGCAACATCCAGATGGAGGACCCCACCTTCAAGG | |
| | AGAACTACAGGTTCCATGCCATCAATGGCTACATCATGGACACCCTGCCTGGCCTGGTGATGGC | |
| | CCAGGACCAGAGGATCAGGTGGTACCTGCTGAGCATGGGCAGCAATGAGAACATCCACAGCAT | |
| | CCACTTCTCTGGCCATGTGTTCACTGTGAGGAAGAAGGAGGAGTACAAGATGGCCCTGTACAAC | |
| | CTGTACCCTGGGGTGTTTGAGACTGTGGAGATGCTGCCCAGCAAGGCTGGCATCTGGAGGGTGG | |
| | AGTGCCTGATTGGGGAGCACCTGCATGCTGGCATGAGCACCCTGTTCCTGGTGTACAGCAACAA | |
| | GTGCCAGACCCCCCTGGGCATGGCCTCTGGCCACATCAGGGACTTCCAGATCACTGCCTCTGGCC | |
| | AGTATGGCCAGTGGGCCCCCAAGCTGGCCAGGCTGCACTACTCTGGCAGCATCAATGCCTGGAG | |
| | CACCAAGGAGCCCTTCAGCTGGATCAAGGTGGACCTGCTGGCCCCCATGATCATCCATGGCATC | |
| | AAGACCCAGGGGGCCAGGCAGAAGTTCAGCAGCCTGTACATCAGCCAGTTCATCATCATGTACA | |
| | GCCTGGATGGCAAGAAGTGGCAGACCTACAGGGGCAACAGCACTGGCACCCTGATGGTGTTCTT | |
| | TGGCAATGTGGACAGCTCTGGCATCAAGCACAACATCTTCAACCCCCCCATCATTGCCAGATAC | |
| | ATCAGGCTGCACCCCACCCACTACAGCATCAGGAGCACCCTGAGGATGGAGCTGATGGGCTGTG | |
| | ACCTGAACAGCTGCAGCATGCCCCTGGGCATGGAGAGCAAGGCCATCTCTGATGCCCAGATCAC | |
| | TGCCAGCAGCTACTTCACCAACATGTTTGCCACCTGGAGCCCCAGCAAGGCCAGGCTGCACCTG | |
| | CAGGGCAGGAGCAATGCCTGGAGGCCCCAGGTCAACAACCCCAAGGAGTGGCTGCAGGTGGAC | |
| | TTCCAGAAGACCATGAAGGTGACTGGGGTGACCACCCAGGGGGTGAAGAGCCTGCTGACCAGC | |
| | ATGTATGTGAAGGAGTTCCTGATCAGCAGCAGCCAGGATGGCCACCAGTGGACCCTGTTCTTCC | |
| | AGAATGGCAAGGTGAAGGTGTTCCAGGGCAACCAGGACAGCTTCACCCCTGTGGTGAACAGCCT | |
| | GGACCCCCCCCTGCTGACCAGATACCTGAGGATTCACCCCCAGAGCTGGGTGCACCAGATTGCC | |
| | CTGAGGATGGAGGTGCTGGGCTGTGAGGCCCAGGACCTGTACTGAAATCAACCTCTGGATTACA | |
| | AAATTTGTGAAAGATTGACTGGTATTCTTAACTATGTTGCTCCTTTTACGCTATGTGGATACGCT | |
| | GCTTTAATGCCTTTGTATCATGCTATTGCTTCCCGTATGGCTTTCATTTTCTCCTCCTTGTATAAAT | |
| | CCTGGTTGCTGTCTCTTTATGAGGAGTTGTGGCCCGTTGTCAGGCAACGTGGCGTGGTGTGCACT | |
| | GTGTTTGCTGACGCAACCCCCACTGGTTGGGGCATTGCCACCACCTGTCAGCTCCTTTCCGGGAC | |
| | TTTCGCTTTCCCCCTCCCTATTGCCACGGCGGAACTCATCGCCGCCTGCCTTGCCCGCTGCTGGAC | |
| | AGGGGCTCGGCTGTTGGGCACTGACAATTCCGTGGGAGCTGTGCGATCCCTGCTGGGGACTTTC | |
| | CGCTGGGGACTTTCCGCTGGGGACTTTCCGCCTTCAGCTAAGGAAGCTACCAATATTTAGAGGTA | |
| | CATTTTGTTCTAGAACAAAATGTACCGGTACATTTTGTTCTGGTACATTTTGTTCT | |
| ITR-ApoE- | CCTGCAGGCAGCTGCGCGCTCGCTCGCTCACTGAGGCCGCCCGGGCGTCGGGCGACCTTTGGTC | 44 |
| AAT-hBGi- | GCCCGGCCTCAGTGAGCGAGCGCGCAGAGAGGGAGTGGCCAACTCCATCACTAGGGGTTCCTGC | |
| FVIII-SQ- | GGCCGCACGCGTCTAGTTATTAATAGTAATCGAATTCGCGTCTGCAGGCTCAGAGGCACACAGG | |
| N6-F309S- | AGTTTCTGGGCTCACCCTGCCCCCTTCCAACCCCTCAGTTCCCATCCTCCAGCAGCTGTTTGTGTG | |
| DM- | CTGCCTCTGAAGTCCACACTGAACAAACTTCAGCCTACTCATGTCCCTAAAATGGGCAAACATTG | |
| WPRE3- | CAAGCAGCAAACAGCAAACACACAGCCCTCCCTGCCTGCTGACCTTGGAGCTGGGGCAGAGGTC | |
| DTS-ITR | AGAGACCTCTCTGGGCCCATGCCACCTCCAACATCCACTCGACCCCTTGGAATTTCGGTGGAGA | |
| | GGAGCAGAGGTTGTCCTGGCGTGGTTTAGGTAGTGTGAGAGGGGTCGACTGGACACAGGACGCT | |
| | GTGGTTTCTGAGCCAGGGGGCGACTCAGATCCCAGCCAGTGGACTTAGCCCCTGTTTGCTCCTCC | |
| | GATAACTGGGGTGACCTTGGTTAATATTCACCAGCAGCCTCCCCCGTTGCCCCTCTGGATCCACT | |
| | GCTTAAATACGGACGAGGACAGGGCCCTGTCTCCTCAGCTTCAGGCACCACCACTGACCTGGGA | |
| | CAGTGAATCGTAAGTACTAGCAGCTACAATCCAGCTACCATTCTGCTTTTATTTTATGGTTGGGA | |
| | TAAGGCTGGATTATTCTGAGTCCAAGCTAGGCCCTTTTGCTAATCATGTTCATACCTCTTATCTTC | |
| | CTCCCACAGCTCCTGGGCAACGTGCTGGTCTGTGTGCTGGCCCATCACTTTGGCAAAGAATTGCG | |
| | ATCGCCACCATGCAGATTGAGCTGAGCACCTGCTTCTTCCTGTGCCTGCTGAGGTTCTGCTTCTCT | |
| | GCCACCAGGAGATACTACCTGGGGGCTGTGGAGCTGAGCTGGGACTACATGCAGTCTGACCTGG | |
| | GGGAGCTGCCTGTGGATGCCAGGTTCCCCCCCAGAGTGCCCAAGAGCTTCCCCTTCAACACCTCT | |
| | GTGGTGTACAAGAAGACCCTGTTTGTGGAGTTCACTGACCACCTGTTCAACATTGCCAAGCCCA | |
| | GGCCCCCCTGGATGGGCCTGCTGGGCCCCACCATCCAGGCTGAGGTGTATGACACTGTGGTGAT | |
| | CACCCTGAAGAACATGGCCAGCCACCCTGTGAGCCTGCATGCTGTGGGGGTGAGCTACTGGAAG | |
| | GCCTCTGAGGGGGCTGAGTATGATGACCAGACCAGCCAGAGGGAGAAGGAGGATGACAAGGTG | |
| | TTCCCTGGGGGCAGCCACACCTATGTGTGGCAGGTGCTGAAGGAGAATGGCCCCATGGCCTCTG | |
| | ACCCCCTGTGCCTGACCTACAGCTACCTGAGCCATGTGGACCTGGTGAAGGACCTGAACTCTGG | |
| | CCTGATTGGGGCCCTGCTGGTGTGCAGGGAGGGCAGCCTGGCCAAGGAGAAGACCCAGACCCT | |
| | GCACAAGTTCATCCTGCTGTTTGCTGTGTTTGATGAGGGCAAGAGCTGGCACTCTGAAACCAAG | |
| | AACAGCCTGATGCAGGACAGGGATGCTGCCTCTGCCAGGGCCTGGCCCAAGATGCACACTGTGA | |
| | ATGGCTATGTGAACAGGAGCCTGCCTGGCCTGATTGGCTGCCACAGGAAGTCTGTGTACTGGCA | |
| | TGTGATTGGCATGGGCACCACCCCTGAGGTGCACAGCATCTTCCTGGAGGGCCACACCTTCCTG | |
| | GTCAGGAACCACAGGCAGGCCAGCCTGGAGATCAGCCCCATCACCTTCCTGACTGCCCAGACCC | |
| | TGCTGATGGACCTGGGCCAGTTCCTGCTGAGCTGCCACATCAGCAGCCACCAGCATGATGGCAT | |
| | GGAGGCCTATGTGAAGGTGGACAGCTGCCCTGAGGAGCCCCAGCTGAGGATGAAGAACAATGA | |
| | GGAGGCTGAGGACTATGATGATGACCTGACTGACTCTGAGATGGATGTGGTGAGGTTTGATGAT | |
| | GACAACAGCCCCAGCTTCATCCAGATCAGGTCTGTGGCCAAGAAGCACCCCAAGACCTGGGTGC | |
| | ACTACATTGCTGCTGAGGAGGAGGACTGGGACTATGCCCCCCTGGTGCTGGCCCCTGATGACAG | |
| | GAGCTACAAGAGCCAGTACCTGAACAATGGCCCCCAGAGGATTGGCAGGAAGTACAAGAAGGT | |
| | CAGGTTCATGGCCTACACTGATGAAACCTTCAAGACCAGGGAGGCCATCCAGCATGAGTCTGGC | |
| | ATCCTGGGCCCCCTGCTGTATGGGGAGGTGGGGGACACCCTGCTGATCATCTTCAAGAACCAGG | |
| | CCAGCAGGCCCTACAACATCTACCCCCATGGCATCACTGATGTGAGGCCCCTGTACAGCAGGAG | |
| | GCTGCCCAAGGGGGTGAAGCACCTGAAGGACTTCCCCATCCTGCCTGGGGAGATCTTCAAGTAC | |
| | AAGTGGACTGTGACTGTGGAGGATGGCCCCACCAAGTCTGACCCCAGGTGCCTGACCAGATACT | |
| | ACAGCAGCTTTGTGAACATGGAGAGGGACCTGGCCTCTGGCCTGATTGGCCCCCTGCTGATCTG | |
| | CTACAAGGAGTCTGTGGACCAGAGGGGCAACCAGATCATGTCTGACAAGAGGAATGTGATCCTG | |
| | TTCTCTGTGTTTGATGAGAACAGGAGCTGGTACCTGACTGAGAACATCCAGAGGTTCCTGCCCA | |
| | ACCCTGCTGGGGTGCAGCTGGAGGACCCTGAGTTCCAGGCCAGCAACATCATGCACAGCATCAA | |
| | TGGCTATGTGTTTGACAGCCTGCAGCTGTCTGTGTGCCTGCATGAGGTGGCCTACTGGTACATCC | |
| | TGAGCATTGGGGCCCAGACTGACTTCCTGTCTGTGTTCTTCTCTGGCTACACCTTCAAGCACAAG | |
| | ATGGTGTATGAGGACACCCTGACCCTGTTCCCCTTCTCTGGGGAGACTGTGTTCATGAGCATGGA | |
| | GAACCCTGGCCTGTGGATTCTGGGCTGCCACAACTCTGACTTCAGGAACAGGGGCATGACTGCC | |
| | CTGCTGAAAGTCTCCAGCTGTGACAAGAACACTGGGGACTACTATGAGGACAGCTATGAGGACA | |
| | TCTCTGCCTACCTGCTGAGCAAGAACAATGCCATTGAGCCCAGGAGCTTCAGCCAGAATCCCCC | |
| | AGTGCTCACCCGGTCCTTTAGCCAGAATTCTCGCCATCCCTCCACCCGGCAAAAGCAGTTCAACG | |
| | CCACTACCATCCCAGAGAACGACATCGAAAAAACCGACCCCTGGTTCGCCCACAGAACTCCCAT | |
| | GCCAAAGATTCAGAATGTTTCCAGTTCTGACCTCCTTATGTTGCTGCGCCAGTCTCCGACCCCTC | |
| | ATGGACTGTCCCTGAGTGACTTGCAGGAGGCCAAGTACGAGACGTTCTCTGATGACCCCAGCCC | |
| | AGGCGCGATTGACTCAAATAACTCCCTGTCTGAGATGACACATTTTCGCCCTCAGCTCCATCACA | |
| | GCGGGGACATGGTGTTCACTCCAGAGTCCGGACTTCAGCTGCGCCTCAACGAGAAACTCGGTAC | |
| | TACAGCCGCGACAGAACTCAAAAAGCTGGATTTCAAGGTTTCCAGCACCAGCAATAACCTGATC | |
| | TCTACAATTCCCAGCGATAACCTGGCGGCTGGAACCGACAACACTTCCAGCCTGGGACCTCCGT | |
| | CCATGCCTGTGCACTACGACTCCCAGCTCGACACCACTCTGTTCGGCAAGAAAAGTAGCCCCCT | |
| | GACCGAATCCGGCGGTCCGCTGTCCCTCTCCGAAGAGAATAACGATAGCAAGCTCCTGGAATCT | |
| | GGGCTTATGAACTCTCAAGAGTCATCCTGGGGCAAGAACGTTTCATCAACTAGGGAGATCACCA | |
| | GGACCACCCTGCAGTCTGACCAGGAGGAGATTGACTATGATGACACCATCTCTGTGGAGATGAA | |
| | GAAGGAGGACTTTGACATCTACGACGAGGACGAGAACCAGAGCCCCAGGAGCTTCCAGAAGAA | |
| | GACCAGGCACTACTTCATTGCTGCTGTGGAGAGGCTGTGGGACTATGGCATGAGCAGCAGCCCC | |
| | CATGTGCTGAGGAACAGGGCCCAGTCTGGCTCTGTGCCCCAGTTCAAGAAGGTGGTGTTCCAGG | |
| | AGTTCACTGATGGCAGCTTCACCCAGCCCCTGTACAGAGGGGAGCTGAATGAGCACCTGGGCCT | |
| | GCTGGGCCCCTACATCAGGGCTGAGGTGGAGGACAACATCATGGTGACCTTCAGGAACCAGGCC | |
| | AGCAGGCCCTACAGCTTCTACAGCAGCCTGATCAGCTATGAGGAGGACCAGAGGCAGGGGGCT | |
| | GAGCCCAGGAAGAACTTTGTGAAGCCCAATGAAACCAAGACCTACTTCTGGAAGGTGCAGCACC | |
| | ACATGGCCCCCACCAAGGATGAGTTTGACTGCAAGGCCTGGGCCTACTTCTCTGATGTGGACCT | |
| | GGAGAAGGATGTGCACTCTGGCCTGATTGGCCCCCTGCTGGTGTGCCACACCAACACCCTGAAC | |
| | CCTGCCCATGGCAGGCAGGTGACTGTGCAGGAGTTTGCCCTGTTCTTCACCATCTTTGATGAAAC | |
| | CAAGAGCTGGTACTTCACTGAGAACATGGAGAGGAACGGCAGGGCCCCCGGCAACATCCAGAT | |
| | GGAGGACCCCACCTTCAAGGAGAACTACAGGTTCCATGCCATCAATGGCTACATCATGGACACC | |
| | CTGCCTGGCCTGGTGATGGCCCAGGACCAGAGGATCAGGTGGTACCTGCTGAGCATGGGCAGCA | |
| | ATGAGAACATCCACAGCATCCACTTCTCTGGCCATGTGTTCACTGTGAGGAAGAAGGAGGAGTA | |
| | CAAGATGGCCCTGTACAACCTGTACCCTGGGGTGTTTGAGACTGTGGAGATGCTGCCCAGCAAG | |
| | GCTGGCATCTGGAGGGTGGAGTGCCTGATTGGGGAGCACCTGCATGCTGGCATGAGCACCCTGT | |
| | TCCTGGTGTACAGCAACAAGTGCCAGACCCCCCTGGGCATGGCCTCTGGCCACATCAGGGACTT | |
| | CCAGATCACTGCCTCTGGCCAGTATGGCCAGTGGGCCCCCAAGCTGGCCAGGCTGCACTACTCT | |
| | GGCAGCATCAATGCCTGGAGCACCAAGGAGCCCTTCAGCTGGATCAAGGTGGACCTGCTGGCCC | |
| | CCATGATCATCCATGGCATCAAGACCCAGGGGGCCAGGCAGAAGTTCAGCAGCCTGTACATCAG | |
| | CCAGTTCATCATCATGTACAGCCTGGATGGCAAGAAGTGGCAGACCTACAGGGGCAACAGCACT | |
| | GGCACCCTGATGGTGTTCTTTGGCAATGTGGACAGCTCTGGCATCAAGCACAACATCTTCAACCC | |
| | CCCCATCATTGCCAGATACATCAGGCTGCACCCCACCCACTACAGCATCAGGAGCACCCTGAGG | |
| | ATGGAGCTGATGGGCTGTGACCTGAACAGCTGCAGCATGCCCCTGGGCATGGAGAGCAAGGCC | |
| | ATCTCTGATGCCCAGATCACTGCCAGCAGCTACTTCACCAACATGTTTGCCACCTGGAGCCCCAG | |
| | CAAGGCCAGGCTGCACCTGCAGGGCAGGAGCAATGCCTGGAGGCCCCAGGTCAACAACCCCAA | |
| | GGAGTGGCTGCAGGTGGACTTCCAGAAGACCATGAAGGTGACTGGGGTGACCACCCAGGGGGT | |
| | GAAGAGCCTGCTGACCAGCATGTATGTGAAGGAGTTCCTGATCAGCAGCAGCCAGGATGGCCAC | |
| | CAGTGGACCCTGTTCTTCCAGAATGGCAAGGTGAAGGTGTTCCAGGGCAACCAGGACAGCTTCA | |
| | CCCCTGTGGTGAACAGCCTGGACCCCCCCCTGCTGACCAGATACCTGAGGATTCACCCCCAGAG | |
| | CTGGGTGCACCAGATTGCCCTGAGGATGGAGGTGCTGGGCTGTGAGGCCCAGGACCTGTACTGA | |
| | CTCGA | |
| WPRE3 + bG | AATCAACCTCTGGATTACAAAATTTGTGAAAGATTGACTGGTATTCTTAACTATGTTGCTCCTTTT | 45 |
| hPolyA | ACGCTATGTGGATACGCTGCTTTAATGCCTTTGTATCATGCTATTGCTTCCCGTATGGCTTTCATT | |
| | TTCTCCTCCTTGTATAAATCCTGGTTGCTGTCTCTTTATGAGGAGTTGTGGCCCGTTGTCAGGCAA | |
| | CGTGGCGTGGTGTGCACTGTGTTTGCTGACGCAACCCCCACTGGTTGGGGCATTGCCACCACCTG | |
| | TCAGCTCCTTTCCGGGACTTTCGCTTTCCCCCTCCCTATTGCCACGGCGGAACTCATCGCCGCCTG | |
| | CCTTGCCCGCTGCTGGACAGGGGCTCGGCTGTTGGGCACTGACAATTCCGTGGTGTGCCTTCTAG | |
| | TTGCCAGCCATCTGTTGTTTGCCCCTCCCCCGTGCCTTCCTTGACCCTGGAAGGTGCCACTCCCAC | |
| | TGTCCTTTCCTAATAAAATGAGGAAATTGCATCGCATTGTCTGAGTAGGTGTCATTCTATTCTGG | |
| | GGGGTGGGGTGGGGCAGGACAGCAAGGGGGAGGATTGGGAAGACAATAGCAGGCATGCTGGG | |
| | GATGCGGTGGGCTCTATGG | |
| FVIII v3 | FVIIIVTRANSLATIQNMQIELSTCFFLCLLRFCFSATRRYYLGAVELSWDYMQSDLGELPVDARFPPR | 46 |
| AA | VPKSFPFNTSVVYKKTLFVEFTDHLFNIAKPRPPWMGLLGPTIQAEVYDTVVITLKNMASHPVSLHA | |
| | VGVSYWKASEGAEYDDQTSQREKEDDKVFPGGSHTYVWQVLKENGPMASDPLCLTYSYLSHVDLV | |
| | KDLNSGLIGALLVCREGSLAKEKTQTLHKFILLFAVFDEGKSWHSETKNSLMQDRDAASARAWPKM | |
| | HTVNGYVNRSLPGLIGCHRKSVYWHVIGMGTTPEVHSIFLEGHTFLVRNHRQASLEISPITFLTAQTLL | |
| | MDLGQFLLFCHISSHQHDGMEAYVKVDSCPEEPQLRMKNNEEAEDYDDDLTDSEMDVVRFDDDNS | |
| | PSFIQIRSVAKKHPKTWVHYIAAEEEDWDYAPLVLAPDDRSYKSQYLNNGPQRIGRKYKKVRFMAY | |
| | TDETFKTREAIQHESGILGPLLYGEVGDTLLIIFKNQASRPYNIYPHGITDVRPLYSRRLPKGVKHLKD | |
| | FPILPGEIFKYKWTVTVEDGPTKSDPRCLTRYYSSFVNMERDLASGLIGPLLICYKESVDQRGNQIMS | |
| | DKRNVILFSVFDENRSWYLTENIQRFLPNPAGVQLEDPEFQASNIMHSINGYVFDSLQLSVCLHEVAY | |
| | WYILSIGAQTDFLSVFFSGYTFKHKMVYEDTLTLFPFSGETVFMSMENPGLWILGCHNSDFRNRGMT | |
| | ALLKVSSCDKNTGDYYEDSYEDISAYLLSKNNAIEPRSFSQNATNVSNNSNTSNDSNVSPPVLKRHQ | |
| | REITRTTLQSDQEEIDYDDTISVEMKKEDFDIYDEDENQSPRSFQKKTRHYFIAAVERLWDYGMSSSP | |
| | HVLRNRAQSGSVPQFKKVVFQEFTDGSFTQPLYRGELNEHLGLLGPYIRAEVEDNIMVTFRNQASRP | |
| | YSFYSSLISYEEDQRQGAEPRKNFVKPNETKTYFWKVQHHMAPTKDEFDCKAWAYFSDVDLEKDV | |
| | HSGLIGPLLVCHTNTLNPAHGRQVTVQEFALFFTIFDETKSWYFTENMERNCRAPCNIQMEDPTFKEN | |
| | YRFHAINGYIMDTLPGLVMAQDQRIRWYLLSMGSNENIHSIHFSGHVFTVRKKEEYKMALYNLYPG | |
| | VFETVEMLPSKAGIWRVECLIGEHLHAGMSTLFLVYSNKCQTPLGMASGHIRDFQITASGQYGQWAP | |
| | KLARLHYSGSINAWSTKEPFSWIKVDLLAPMIIHGIKTQGARQKFSSLYISQFIIMYSLDGKKWQTYR | |
| | GNSTGTLMVFFGNVDSSGIKHNIFNPPIIARYIRLHPTHYSIRSTLRMELMGCDLNSCSMPLGMESKAI | |
| | SDAQITASSYFTNMFATWSPSKARLHLQGRSNAWRPQVNNPKEWLQVDFQKTMKVTGVTTQGVKS | |
| | LLTSMYVKEFLISSSQDGHQWTLFFQNGKVKVFQGNQDSFTPVVNSLDPPLLTRYLRIHPQSWVHQI | |
| | ALRMEVLGCEAQDL | |

What is claimed is:

1. A nucleic acid composition comprising a nucleic acid sequence having at least 90% sequence identity to SEQ ID NO: 6.

2. The nucleic acid composition of claim 1, wherein the nucleic acid sequence is SEQ ID NO: 6.

3. The nucleic acid composition of claim 1, further comprising a therapeutic nucleic acid sequence, wherein the nucleic acid sequence having at least 90% sequence identity to SEQ ID NO: 6 increases expression of the therapeutic nucleic acid sequence in a cell by at least 1.25-fold as compared to an otherwise identical control composition lacking the nucleic acid sequence having at least 90% sequence identity to SEQ ID NO: 6.

4. The nucleic acid composition of claim 1, wherein the nucleic acid sequence having at least 90% sequence identity to SEQ ID NO: 6 is a nuclear targeting sequence.

5. The nucleic acid composition of claim 1, wherein the nucleic acid composition is an isolated nucleic acid molecule.

6. The nucleic acid composition of claim 1, wherein the nucleic acid composition further comprises one or more Inverted Terminal Repeat (ITR) sequences.

7. The nucleic acid composition of claim 1, wherein the nucleic acid composition is a non-viral vector.

8. A kit comprising a sonoactive agent and a nucleic acid composition, wherein the nucleic acid composition comprises a nucleic acid sequence having at least 90% sequence identity to SEQ ID NO: 6.

9. The kit of claim 8, wherein the nucleic acid sequence is SEQ ID NO: 6.

10. The kit of claim 8, nucleic acid composition further comprises a therapeutic nucleic acid sequence, wherein the nucleic acid sequence having at least 90% sequence identity to SEQ ID NO: 6 increases expression of the therapeutic nucleic acid sequence in a cell by at least 1.25-fold as compared to an otherwise identical control composition lacking the nucleic acid sequence having at least 90% sequence identity to SEQ ID NO: 6.

11. The kit of claim 8, wherein the nucleic acid sequence having at least 90% sequence identity to SEQ ID NO: 6 is a nuclear targeting sequence.

12. The kit of claim 8, wherein the nucleic acid composition is an isolated nucleic acid molecule.

13. The kit of claim 8, wherein the nucleic acid composition further comprises one or more Inverted Terminal Repeat (ITR) sequences.

14. The kit of claim 8, wherein the nucleic acid composition is a non-viral vector.

15. The kit of claim 8, wherein the sonoactive agent comprises a shell filled with a perfluorinated gas.

16. The kit of claim 8, wherein the sonoactive agent comprises protein-stabilized microstructures.

17. The kit of claim 8, wherein the sonoactive agent comprises lipid-stabilized microstructures.

18. The kit of claim 17, wherein the lipid-stabilized microstructures comprise a lipid stabilized shell surrounding a perfluorinated gas core.

19. The kit of claim 18, wherein the lipid stabilized shell comprises a monomolecular membrane of hydrogenated egg yolk phosphatidyl serine, wherein the perfluorinated gas core comprises perfluorobutane gas.

20. The kit of claim 8, further comprising instructions for administering ultrasound acoustic energy to a subject to facilitate delivery of the nucleic acid composition to a subject.
